(19) United States
(12) Patent Application Publication
(10) Pub. No.: US 2025/0041402 A1
(43) Pub. Date: Feb. 6, 2025

(54) RECOMBINANT NEWCASTLE DISEASE VIRUS EXPRESSING SPIKE PROTEIN OF SARS-COV-2 DELTA VARIANT AND USES THEREOF

(71) Applicant: ICAHN SCHOOL OF MEDICINE AT MOUNT SINAI, New York, NY (US)

(72) Inventors: Peter PALESE, New York, NY (US); Florian KRAMMER, New York, NY (US); Adolfo GARCIA-SASTRE, New York, NY (US); Weina SUN, New York, NY (US)

(21) Appl. No.: 18/697,359

(22) PCT Filed: Sep. 29, 2022
(86) PCT No.: PCT/US2022/077254

§ 371 Date: Mar. 29, 2024

Related U.S. Application Data
(60) Provisional application No. 63/318,643, filed on Mar. 10, 2022.
(60) Provisional application No. 63/251,020, filed on Sep. 30, 2021.

(51) Int. Cl. A61K 39/215; A61K 39/00; A61P 37/04; C07K 14/005; C12N 7/00

(52) U.S. Cl. CPC A61K 39/215; A61P 37/04; C07K 14/005; C12N 7/00; A61K 2039/5256; A61K 2039/575; C12N 2760/18021; C12N 2770/20022; C12N 2770/20034; C12N 2770/20071

(57) ABSTRACT

Described herein are recombinant Newcastle disease viruses (“NDVs”) comprising a packaged genome, wherein the packaged genome comprises a transgene comprising a nucleotide sequence encoding a protein comprising a SARS-CoV-2 delta variant spike protein or portion thereof. Also described herein are recombinant NDVs comprising a packaged genome, wherein the packaged genome comprises a transgene encoding a chimeric F protein, wherein the chimeric F protein comprises a SARS-CoV-2 delta variant spike protein ectodomain and NDV F protein transmembrane and cytoplasmic domains. The recombinant NDVs and compositions thereof are useful for the immunizing against SARS-CoV-2 delta variant as well as the prevention of COVID-19.

### CROSS-REFERENCE TO RELATED APPLICATIONS

This application claims the benefit of U.S. Provisional Patent Application No. 63/318,643, filed Mar. 10, 2022 and U.S. Provisional Patent Application No. 63/251,020, filed Sep. 30, 2021, the disclosure of each of which is incorporated by reference herein in its entirety.

### STATEMENT REGARDING FEDERALLY SPONSORED RESEARCH

This invention was made with government support under grant HHSN272201400008C awarded by the National Institutes of Health. The government has certain rights in the invention.

### SEQUENCE LISTING

This application contains a computer readable Sequence Listing which has been submitted in XML file format with this application, the entire content of which is incorporated by reference herein in its entirety. The Sequence Listing XML file submitted with this application is entitled “06923-386-228_SEQ_LISTING.xml”, was created on Sep. 23, 2022, and is 97,417 bytes in size.

### 1. INTRODUCTION

In one aspect, described herein are recombinant Newcastle disease virus (“NDV”) comprising a packaged genome, wherein the packaged genome comprises a transgene encoding a protein comprising a spike protein of a severe acute respiratory syndrome coronavirus 2 (“SARS-CoV-2”) delta variant or a portion thereof (e.g., ectodomain or receptor binding domain of SARS-CoV-2 delta variant spike protein). In a specific embodiment, described herein are recombinant NDV comprising a packaged genome, wherein the packaged genome comprises a transgene comprising a codon-optimized nucleic acid sequence encoding a protein comprising a spike protein of a SARS-CoV-2 delta variant or portion thereof (e.g., ectodomain or receptor binding domain of SARS-CoV-2 spike protein). In specific embodiments, the SARS-CoV-2 delta variant spike protein ectodomain does not comprise the amino acid substitution of P681R. In a specific embodiment, described herein are recombinant NDV comprising a packaged genome, wherein the packaged genome comprises a transgene encoding a chimeric F protein, wherein the chimeric F protein comprises a spike protein ectodomain of a SARS-CoV-2 delta variant and NDV F protein transmembrane and cytoplasmic domains. In certain embodiments, the ectodomain of the spike protein of the SARS-CoV-2 delta variant lacks a polybasic cleavage site and/or comprises a certain number of amino acid substitutions (e.g., 6 amino acid substitutions) to proline. In specific embodiments, the SARS-CoV-2 delta variant spike protein ectodomain does not comprise the amino acid substitution of P681R. In some embodiments, the ectodomain of the SARS-CoV-2 delta variant spike protein is encoded by a codon-optimized nucleic acid sequence. Also described herein are compositions comprising such recombinant NDV and the use of such recombinant NDV to induce an immune response to SARS-CoV-2 delta variant spike protein, and in immunoassays to detect the presence of antibody that binds to SARS-CoV-2 delta variant spike protein.

### 2. BACKGROUND

There is an urgent need to develop vaccines to prevent COVID-19 and reduce severe acute respiratory syndrome coronavirus-2 (SARS-CoV-2) infection. As of May 7, 2020, more than 3,815,561 people have tested positive for SARS-CoV-2. As of Sep. 28, 2021, SARS-CoV-2 has resulted in approximately 233,548,694 infections globally with more than 4,777,758 deaths, and continues to pose a threat to public health.

While COVID-19 vaccines have been widely rolled out in high income countries, all currently used vaccines are injected, which does not induce optimal mucosal immunity. This lack of mucosal immunity allows for breakthrough infections, especially when immunity wanes over time. In addition, a large proportion of the global population has still no access to vaccines. Further, booster doses are needed in certain segments of the population. However, mRNA vaccines currently authorized for booster doses induce strong side effects (reactogenicity) like injection site pain and transient influenza-like symptoms. While these side effects are manageable, they are unpleasant and can result in sick days for vaccines (carrying economic costs). Thus, there is a need for COVID-19 vaccines.

### 3. SUMMARY

In one aspect, described herein are recombinant Newcastle disease virus (“NDV”) comprising a transgene encoding a protein comprising a spike protein of a severe acute respiratory syndrome coronavirus 2 (“SARS-CoV-2”) delta variant or a portion thereof (e.g., ectodomain or receptor binding domain of SARS-CoV-2 delta variant spike protein). In a specific aspect, presented herein are recombinant Newcastle disease virus (“NDV”) comprising a packaged genome, wherein the packaged genome comprises a transgene comprising a nucleotide sequence encoding a protein, wherein the protein comprises a spike protein of a SARS-CoV-2 delta variant or a portion thereof (e.g., the ectodomain, S1 domain, S2 domain, or receptor binding domain of the SARS-CoV-2 delta variant spike protein). In a specific embodiment, the SARS-CoV-2 delta variant is of the B1.617.2 sublineage. In certain embodiments, the SARS-CoV-2 delta variant is of the AY sublineage. In another specific embodiment, the protein comprising the spike protein of the SARS-CoV-2 delta variant or a portion thereof (e.g., the ectodomain, S1 domain, S2 domain, or receptor binding domain of the SARS-CoV-2 delta variant spike protein) is expressed by cells infected with the recombinant NDV and the protein comprising the spike protein of the SARS-CoV-2 delta variant or a portion thereof (e.g., the ectodomain, S1 domain, S2 domain, or receptor binding domain of the SARS-CoV-2 delta variant spike protein) is incorporated into the NDV virion.

In a specific embodiment, provided herein is a recombinant Newcastle disease virus (“NDV”) comprising a packaged genome, wherein the packaged genome comprises a transgene comprising a nucleotide sequence encoding a protein, wherein the protein comprises a SARS-CoV-2 delta variant spike protein ectodomain. In certain embodiments, provided herein is a recombinant Newcastle disease virus (“NDV”) comprising a packaged genome, wherein the packaged genome comprises a transgene comprising a nucleotide sequence encoding a SARS-CoV-2 delta variant spike protein ectodomain. In a specific embodiment, the SARS-CoV-2 delta variant is of the B1.617.2 sublineage. In certain embodiments, the SARS-CoV-2 delta variant is of the AY sublineage. In a specific embodiment, the protein comprising the ectodomain of the SARS-CoV-2 delta variant spike protein is expressed by cells infected with the recombinant NDV. In another specific embodiment, the protein comprising the ectodomain of the SARS-CoV-2 delta variant spike protein is expressed by cells infected with the recombinant NDV and the protein comprising the ectodomain of the SARS-CoV-2 delta variant spike protein is incorporated into the NDV virion.

In a specific embodiment, provided herein is a recombinant Newcastle disease virus (“NDV”) comprising a packaged genome, wherein the packaged genome comprises a transgene comprising a nucleotide sequence encoding a protein, wherein the protein comprises a derivative of a SARS-CoV-2 delta variant spike protein ectodomain, wherein the derivative comprises a SARS-CoV-2 delta variant spike protein ectodomain lacking a polybasic cleavage site (e.g., as a result of one, two, or more amino acid substitutions in polybasic cleavage site). The derivative of the SARS-CoV-2 delta variant spike protein ectodomain may lack the polybasic cleavage site as a result of a substitution of amino acid residues RRAR to A at amino acid residues corresponding to amino acid residues 682 to 685 of the SARS-CoV-2 spike protein found at GenBank Accession No. MN908947.3. In some embodiments, the derivative of the SARS-CoV-2 delta variant spike protein ectodomain comprises the amino acid sequence of SEQ ID NO:12 with one, two, three, four, five, six, seven, eight, or all of the following amino acid modifications: T19R, G142D, delE156, delF157, R158G, L452R, T478K, P681R, and D950N. In specific embodiments, the derivative of the SARS-CoV-2 delta variant spike protein ectodomain does not comprise the amino acid substitution of P681R. In a specific embodiment, the derivative of the SARS-CoV-2 delta variant spike protein ectodomain comprises the amino acid sequence of SEQ ID NO:12 with one, two, three, four, five, six, seven, or all of the following amino acid modifications: T19R, G142D, delE156, delF157, R158G, L452R, T478K, and D950N. In certain embodiments, the derivative of the SARS-CoV-2 delta variant spike protein ectodomain comprises the amino acid sequence of SEQ ID NO:13. In another specific embodiment, the derivative of the SARS-CoV-2 delta variant spike protein ectodomain comprises the amino acid sequence of SEQ ID NO:12 with one, two, three, four, five, six, seven, eight, nine, ten, eleven, twelve, thirteen, fourteen, or all of the following amino acid modifications: T19R, T95I, G142D, delE156, delF157, R158G, A222V, W258L, K417N, L452R, T478K, D614G, P681R, and D950N. In a specific embodiment, the derivative does not comprise the amino acid substitution of P681R. In some embodiments, the derivative of the SARS-CoV-2 delta variant spike protein ectodomain comprises the amino acid sequence of SEQ ID NO:12 with one, two, three, four, five, six, seven, eight, nine, ten, eleven, twelve, thirteen, or all of the following amino acid modifications: T19R, T95I, G142D, delE156, delF157, R158G, A222V, W258L, K417N, L452R, T478K, D614G, and D950N. In a specific embodiment, the protein comprising the derivative of the ectodomain of the SARS-CoV-2 delta variant spike protein is expressed by cells infected with the recombinant NDV. In another specific embodiment, the protein comprising the derivative of the ectodomain of the SARS-CoV-2 delta variant spike protein is expressed by cells infected with the recombinant NDV and the protein comprising the ectodomain of the SARS-CoV-2 delta variant spike protein is incorporated into the NDV virion.

In another aspect, described herein are recombinant NDV comprising a transgene encoding a chimeric F protein, wherein the chimeric F protein comprises a spike protein ectodomain of a SARS-CoV-2 delta variant or a derivative thereof and NDV F protein transmembrane and cytoplasmic domains. In a specific embodiment, described herein are recombinant NDV comprising a packaged genome, wherein the packaged genome comprises a transgene encoding a chimeric F protein, wherein the chimeric F protein comprises a spike protein ectodomain of a SARS-CoV-2 delta variant and NDV F protein transmembrane and cytoplasmic domains. In a specific embodiment, the chimeric F protein does not include the SARS-CoV-2 spike protein transmembrane and cytoplasmic domains. In certain embodiments, the NDV F protein transmembrane and cytoplasmic domains are fused to the SARS-CoV-2 spike protein ectodomain through a linker sequence (e.g., GGGGS (SEQ ID NO:7)). In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 2, 3, 4, 5, 6, 7, 8 or more. In a specific embodiment, the linker comprises the sequence GGGGS (SEQ ID NO:7). In some embodiments, the NDV F protein transmembrane and cytoplasmic domains are fused directly to the SARS-CoV-2 spike protein ectodomain. In a specific embodiment, the NDV F protein and chimeric F protein are expressed by cells infected with the recombinant NDV. In another specific embodiment, the chimeric F protein is expressed by cells infected with the recombinant NDV and the chimeric F protein is incorporated into the NDV virion.

In another embodiment, provided herein is a recombinant NDV comprising a packaged genome, wherein the packaged genome comprises a transgene encoding a chimeric F protein, wherein the chimeric F protein comprises a derivative of a spike protein ectodomain of a SARS-CoV-2 delta variant and NDV F protein transmembrane and cytoplasmic domains, and wherein the derivative of the SARS-CoV-2 delta variant spike protein ectodomain lacks a polybasic cleavage site (e.g., as a result of one, two, or more amino acid substitutions in polybasic cleavage site). In a specific embodiment, amino acid residues RRAR at amino acid positions 682 to 685 of the SARS-CoV-2 spike protein found at GenBank Accession No. MN908947.3 are substituted for alanine. In a specific embodiment, the chimeric F protein does not include the SARS-CoV-2 spike protein transmembrane and cytoplasmic domains. The derivative of the SARS-CoV-2 delta variant spike protein ectodomain may lack the polybasic cleavage site as a result of amino acid residues 682 to 685 of the polybasic cleavage site being substituted with a single alanine. In a specific embodiment, the SARS-CoV-2 delta variant is of the B1.617.2 sublineage. In certain embodiments, the SARS-CoV-2 delta variant is of the AY sublineage. In some embodiments, the derivative of the SARS-CoV-2 delta variant spike protein ectodomain comprises the amino acid sequence of SEQ ID NO:12 with one, two, three, four, five, six, seven, eight, or all of the following amino acid modifications: T19R, G142D, delE156, delF157, R158G, L452R, T478K, D614G, P681R, and D950N. In specific embodiments, the derivative of the SARS-CoV-2 delta variant spike protein ectodomain does not comprise the amino acid substitution of P681R. In a specific embodiment, the derivative of the SARS-CoV-2 delta variant spike protein ectodomain comprises the amino acid sequence of SEQ ID NO:12 with one, two, three, four, five, six, seven, or all of the following amino acid modifications: T19R, G142D, delE156, delF157, R158G, L452R, T478K, D614G, and D950N. In certain embodiments, the derivative of the SARS-CoV-2 delta variant spike protein ectodomain comprises the amino acid sequence of SEQ ID NO:13. In another specific embodiment, the derivative of the SARS-CoV-2 delta variant spike protein ectodomain comprises the amino acid sequence of SEQ ID NO:12 with one, two, three, four, five, six, seven, eight, nine, ten, eleven, twelve, thirteen, fourteen, or all of the following amino acid modifications: T19R, T95I, G142D, delE156, delF157, R158G, A222V, W258L, K417N, L452R, T478K, D614G, P681R, and D950N. In a specific embodiment, the derivative does not comprise the amino acid substitution of P681R. In some embodiments, the derivative of the SARS-CoV-2 delta variant spike protein ectodomain comprises the amino acid sequence of SEQ ID NO:12 with one, two, three, four, five, six, seven, eight, nine, ten, eleven, twelve, thirteen, or all of the following amino acid modifications: T19R, T95I, G142D, delE156, delF157, R158G, A222V, W258L, K417N, L452R, T478K, D614G, and D950N. In some embodiments, the derivative of the SARS-CoV-2 delta variant ectodomain comprises the amino acid sequence of SEQ ID NO:16 with one, two, three, four, five, six, seven, eight, or all of the following amino acid modifications: T6R, G129D, delE143, delF144, R145G, L439R, T465K, D601G, and D937N. In certain embodiments, the NDV F protein transmembrane and cytoplasmic domains are fused to the derivative SARS-CoV-2 delta variant spike protein ectodomain through a linker sequence (e.g., GGGGS (SEQ ID NO:7)). In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 2, 3, 4, 5, 6, 7, 8 or more. In a specific embodiment, the linker comprises the sequence GGGGS (SEQ ID NO:7). In some embodiments, the NDV F protein transmembrane and cytoplasmic domains are fused to directly to the derivative SARS-CoV-2 delta variant spike protein ectodomain. In a specific embodiment, the NDV F protein and chimeric F protein are expressed by cells infected with the recombinant NDV. In another specific embodiment, the chimeric F protein is expressed by cells infected with the recombinant NDV and the chimeric F protein is incorporated into the NDV virion.

In a specific embodiment, provided herein is a recombinant NDV comprising a transgene encoding a chimeric F protein, wherein the chimeric F protein comprises an amino acid sequence at least 99% identical to the amino acid sequence set forth in SEQ ID NO:6 or 18. In another specific embodiment, provided herein is a recombinant NDV comprising a transgene encoding a chimeric F protein, wherein the chimeric F protein comprises an amino acid sequence at least 99.5% identical to the amino acid sequence set forth in SEQ ID NO:6 or 18. In another specific embodiment, provided herein is a recombinant NDV comprising a transgene encoding a chimeric F protein, wherein the chimeric F protein comprises the amino acid sequence set forth in SEQ ID NO:6 or 18. In a preferred embodiment, a transgene comprises a codon-optimized version of a nucleic acid sequence encoding a derivative of the SARS-CoV-2 delta variant spike protein ectodomain comprising the amino acid sequence of SEQ ID NO:13 or 17. In another preferred embodiment, a transgene comprises a codon-optimized version of a nucleic acid sequence encoding the chimeric F protein comprising the amino acid sequence set forth in SEQ ID NO:6 or 18. In another specific embodiment, a transgene comprises an RNA sequence corresponding to the negative sense of the cDNA sequence of SEQ ID NO:5. In a specific embodiment, the NDV F protein and chimeric F protein are expressed by cells infected with the recombinant NDV. In another specific embodiment, the chimeric F protein is expressed by cells infected with the recombinant NDV and the chimeric F protein is incorporated into the NDV virion.

In another embodiment, provided herein is a recombinant NDV comprising a packaged genome, wherein the packaged genome comprises a transgene, wherein the transgene comprises a nucleotide sequence encoding a chimeric F protein, wherein the chimeric F protein comprises a derivative SARS-CoV-2 delta virus spike protein ectodomain and NDV F protein transmembrane and cytoplasmic domains, wherein the derivative comprises a SARS-CoV-2 delta spike protein comprising a SARS-CoV-2 delta variant ectodomain in which: (1) amino acid residues corresponding to amino acid residues 817, 892, 899, 942, 986, and 987 of the SARS-CoV-2 spike protein found at GenBank Accession No. MN908947.3 are substituted with prolines, (2) amino acid residues corresponding to amino acid residues 682 to 685 of the SARS-CoV-2 spike protein found at GenBank Accession No. MN908947.3 are substituted such that the polybasic cleavage site is inactivated, and (3) two, three, four, five, six, seven, or more amino acid residues corresponding to two, three, four, five, six, seven, eight or more of amino acid residues 19, 142, 156, 157, 158, 452, 478, 614, 681, and 950 of the spike protein found at GenBank Accession No. MN908947.3 are: 19R, 142D, del156, del157, 158G, 452R, 478K, 614G, 681R and 950N. In a specific embodiment, the derivative does not comprise the amino acid substitution of P681R. In another embodiment, provided herein is a recombinant NDV comprising a packaged genome, wherein the packaged genome comprises a transgene, wherein the transgene comprises a nucleotide sequence encoding a chimeric F protein, wherein the chimeric F protein comprises a derivative SARS-CoV-2 delta virus spike protein ectodomain and NDV F protein transmembrane and cytoplasmic domains, wherein the derivative comprises a SARS-CoV-2 delta spike protein comprising a SARS-CoV-2 delta variant ectodomain in which: (1) amino acid residues corresponding to amino acid residues 817, 892, 899, 942, 986, and 987 of the SARS-CoV-2 spike protein found at GenBank Accession No. MN908947.3 are substituted with prolines, (2) amino acid residues corresponding to amino acid residues 682 to 685 of the polybasic cleavage site of the SARS-CoV-2 spike protein found at GenBank Accession No. MN908947.3 are substituted such that the polybasic cleavage site is inactivated, and (3) amino acid residues corresponding to amino acid residues 19, 142, 156, 157, 158, 452, 478, 614, 681, and 950 of the spike protein found at GenBank Accession No. MN908947.3 as follows: R, D, deleted, deleted, G, R, K, G, R, and N, respectively. In a specific embodiment, the derivative does not comprise the amino acid substitution of P681R. In another embodiment, provided herein is a recombinant NDV comprising a packaged genome, wherein the packaged genome comprises a transgene, wherein the transgene comprises a nucleotide sequence encoding a chimeric F protein, wherein the chimeric F protein comprises a derivative SARS-CoV-2 delta virus spike protein ectodomain and NDV F protein transmembrane and cytoplasmic domains, wherein the derivative comprises a SARS-CoV-2 delta spike protein comprising a SARS-CoV-2 delta variant ectodomain in which: (1) amino acid residues corresponding to amino acid residues 817, 892, 899, 942, 986, and 987 of the SARS-CoV-2 spike protein found at GenBank Accession No. MN908947.3 are substituted with prolines, (2) amino acid residues corresponding to amino acid residues 682 to 685 of the polybasic cleavage site of the SARS-CoV-2 spike protein found at GenBank Accession No. MN908947.3 are substituted such that the polybasic cleavage site is inactivated, and (3) amino acid residues corresponding to amino acid residues 19, 142, 156, 157, 158, 452, 478, 614, and 950 of the spike protein found at GenBank Accession No. MN908947.3 are as follows: R, D, deleted, deleted, G, R, K, G, and N, respectively. In specific embodiments, a polybasic cleavage site is inactivated if it cannot be cleaved by, e.g., furin. In a specific embodiment, amino acid residues corresponding to amino acid residues 682 to 685 of the polybasic cleavage site of the SARS-CoV-2 spike protein found at GenBank Accession No. MN908947.3 are substituted with a single alanine. In certain embodiments, the NDV F protein transmembrane and cytoplasmic domains are fused to the derivative SARS-CoV-2 delta variant spike protein ectodomain through a linker sequence (e.g., GGGGS (SEQ ID NO:7)). In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 2, 3, 4, 5, 6, 7, 8 or more. In a specific embodiment, the linker comprises the sequence GGGGS (SEQ ID NO:7). In some embodiments, the NDV F protein transmembrane and cytoplasmic domains are fused to directly to the derivative SARS-CoV-2 delta variant spike protein ectodomain. In a specific embodiment, the NDV F protein and chimeric F protein are expressed by cells infected with the recombinant NDV. In another specific embodiment, the chimeric F protein is expressed by cells infected with the recombinant NDV and the chimeric F protein is incorporated into the NDV virion.

In another embodiment, provided herein is a recombinant Newcastle disease virus (NDV) comprising a packaged genome, wherein the packaged genome comprises a transgene, wherein the transgene comprises a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises an amino acid sequence at least 99% identical to the amino acid sequence of SEQ ID NO:13 or 17. In another embodiment, provided herein is a recombinant Newcastle disease virus (NDV) comprising a packaged genome, wherein the packaged genome comprises a transgene, wherein the transgene comprises a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises an amino acid sequence at least 99.5% identical to the amino acid sequence of SEQ ID NO:13 or 17. In another embodiment, provided herein is a recombinant Newcastle disease virus (NDV) comprising a packaged genome, wherein the packaged genome comprises a transgene, wherein the transgene comprises a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:13 or 17. In certain embodiments, the NDV F protein transmembrane and cytoplasmic domains are fused to the derivative SARS-CoV-2 delta variant spike protein ectodomain through a linker sequence (e.g., GGGGS (SEQ ID NO:7)). In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 2, 3, 4, 5, 6, 7, 8 or more. In a specific embodiment, the linker comprises the sequence GGGGS (SEQ ID NO:7). In some embodiments, the NDV F protein transmembrane and cytoplasmic domains are fused to directly to the derivative SARS-CoV-2 delta variant spike protein ectodomain. In a specific embodiment, the NDV F protein and chimeric F protein are expressed by cells infected with the recombinant NDV. In another specific embodiment, the chimeric F protein is expressed by cells infected with the recombinant NDV and the chimeric F protein is incorporated into the NDV virion.

In another aspect, described herein are recombinant NDV comprising a chimeric F protein, wherein the chimeric F protein comprises a spike protein ectodomain of a SARS-CoV-2 delta variant or a derivative thereof and NDV F protein transmembrane and cytoplasmic domains. In a specific embodiment, described herein are recombinant NDV comprising a chimeric F protein, wherein the chimeric F protein comprises a spike protein ectodomain of a SARS-CoV-2 delta variant and NDV F protein transmembrane and cytoplasmic domains. In a specific embodiment, the chimeric F protein does not include the SARS-CoV-2 spike protein transmembrane and cytoplasmic domains. In certain embodiments, the NDV F protein transmembrane and cytoplasmic domains are fused to the SARS-CoV-2 spike protein ectodomain through a linker sequence (e.g., GGGGS (SEQ ID NO:7)). In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 2, 3, 4, 5, 6, 7, 8 or more. In a specific embodiment, the linker comprises the sequence GGGGS (SEQ ID NO:7). In some embodiments, the NDV F protein transmembrane and cytoplasmic domains are fused directly to the SARS-CoV-2 spike protein ectodomain. In a specific embodiment, the NDV F protein and chimeric F protein is incorporated into the NDV virion.

In another embodiment, provided herein is a recombinant NDV comprising a chimeric F protein, wherein the chimeric F protein comprises a derivative of a spike protein ectodomain of a SARS-CoV-2 delta variant and an NDV F protein transmembrane and cytoplasmic domains, and wherein the derivative of the SARS-CoV-2 delta variant spike protein ectodomain lacks a polybasic cleavage site (e.g., as a result of one, two, or more amino acid substitutions in polybasic cleavage site). In a specific embodiment, amino acid residues RRAR at amino acid positions corresponding to amino acid positions 682 to 685 of GenBank Accession No. MN908947.3 are substituted with an alanine. In a specific embodiment, the chimeric F protein does not include the SARS-CoV-2 spike protein transmembrane and cytoplasmic domains. The derivative of the SARS-CoV-2 delta variant spike protein ectodomain may lack the polybasic cleavage site as a result of amino acid residues 682 to 685 of the polybasic cleavage site being substituted with a single alanine. In a specific embodiment, the SARS-CoV-2 delta variant is of the B1.617.2 sublineage. In certain embodiments, the SARS-CoV-2 delta variant is of the AY sublineage. In some embodiments, the derivative of the SARS-CoV-2 delta variant spike protein ectodomain comprises the amino acid sequence of SEQ ID NO:12 with one, two, three, four, five, six, seven, eight, or all of the following amino acid modifications: T19R, G142D, delE156, delF157, R158G, L452R, T478K, D614G, P681R, and D950N. In a specific embodiment, the derivative does not comprise the amino acid substitution of P681R. In specific embodiments, the derivative of the SARS-CoV-2 delta variant spike protein ectodomain does not comprise the amino acid substitution of P681R. In a specific embodiment, the derivative of the SARS-CoV-2 delta variant spike protein ectodomain comprises the amino acid sequence of SEQ ID NO:12 with one, two, three, four, five, six, seven, or all of the following amino acid modifications: T19R, G142D, delE156, delF157, R158G, L452R, T478K, D614G, and D950N. In certain embodiments, the derivative of the SARS-CoV-2 delta variant spike protein ectodomain comprises the amino acid sequence of SEQ ID NO:13. In another specific embodiment, the derivative of the SARS-CoV-2 delta variant spike protein ectodomain comprises the amino acid sequence of SEQ ID NO:12 with one, two, three, four, five, six, seven, eight, nine, ten, eleven, twelve, thirteen, fourteen, or all of the following amino acid modifications: T19R, T95I, G142D, delE156, delF157, R158G, A222V, W258L, K417N, L452R, T478K, D614G, P681R, and D950N. In a specific embodiment, the derivative does not comprise the amino acid substitution of P681R. In some embodiments, the derivative of the SARS-CoV-2 delta variant spike protein ectodomain comprises the amino acid sequence of SEQ ID NO:12 with one, two, three, four, five, six, seven, eight, nine, ten, eleven, twelve, thirteen, or all of the following amino acid modifications: T19R, T95I, G142D, delE156, delF157, R158G, A222V, W258L, K417N, L452R, T478K, D614G, and D950N. In certain embodiments, the NDV F protein transmembrane and cytoplasmic domains are fused to the SARS-CoV-2 spike protein ectodomain through a linker sequence (e.g., GGGGS (SEQ ID NO:7)). In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 2, 3, 4, 5, 6, 7, 8 or more. In a specific embodiment, the linker comprises the sequence GGGGS (SEQ ID NO:7). In some embodiments, the NDV F protein transmembrane and cytoplasmic domains are fused to directly to the derivative of the SARS-CoV-2 delta variant spike protein ectodomain. In a specific embodiment, the NDV F protein and chimeric F protein is incorporated into the NDV virion.

In a specific embodiment, provided herein is a recombinant NDV comprising a chimeric F protein, wherein the chimeric F protein comprises an amino acid sequence at least 99% identical to the amino acid sequence set forth in SEQ ID NO:6 or 18. In another specific embodiment, provided herein is a recombinant NDV comprising a chimeric F protein, wherein the chimeric F protein comprises an amino acid sequence at least 99.5% identical to the amino acid sequence set forth in SEQ ID NO:6 or 18. In another specific embodiment, provided herein is a recombinant NDV comprising a chimeric F protein, wherein the chimeric F protein comprises the amino acid sequence set forth in SEQ ID NO:6 or 18. In a specific embodiment, the NDV F protein and chimeric F protein are expressed by cells infected with the recombinant NDV. In a specific embodiment, the NDV F protein and chimeric F protein is incorporated into the NDV virion.

In another embodiment, provided herein is a recombinant NDV comprising a chimeric F protein, wherein the chimeric F protein comprises a derivative SARS-CoV-2 delta virus spike protein ectodomain and NDV F protein transmembrane and cytoplasmic domains, wherein the derivative comprises a SARS-CoV-2 delta spike protein comprising a SARS-CoV-2 delta variant ectodomain in which: (1) amino acid residues corresponding to amino acid residues 817, 892, 899, 942, 986, and 987 of the SARS-CoV-2 spike protein found at GenBank Accession No. MN908947.3 are substituted with prolines, (2) amino acid residues corresponding to amino acid residues 682 to 685 of the polybasic cleavage site of the SARS-CoV-2 spike protein found at GenBank Accession No. MN908947.3 are substituted such that the polybasic cleavage site is inactivated, and (3) two, three, four, five, six, seven, or more amino acid residues corresponding to two, three, four, five, six, seven, or more of amino acid residues 19, 142, 156, 157, 158, 452, 478, 614, 681, and 950 of the spike protein found at GenBank Accession No. MN908947.3 are as follows: 19R, 142D, deleted, deleted, 158G, 452R, 478K, 614G, 681R, and 950N. In a specific embodiment, the derivative does not comprise an amino acid substitution of P681R. In another embodiment, provided herein is a recombinant NDV comprising a chimeric F protein, wherein the chimeric F protein comprises a derivative SARS-CoV-2 delta virus spike protein ectodomain and NDV F protein transmembrane and cytoplasmic domains, wherein the derivative comprises a SARS-CoV-2 delta spike protein comprising a SARS-CoV-2 delta variant ectodomain in which: (1) amino acid residues corresponding to amino acid residues 817, 892, 899, 942, 986, and 987 of the SARS-CoV-2 spike protein found at GenBank Accession No. MN908947.3 are substituted with prolines, (2) amino acid residues corresponding to amino acid residues 682 to 685 of the polybasic cleavage site of the SARS-CoV-2 spike protein found at GenBank Accession No. MN908947.3 are substituted such that the polybasic cleavage site is inactivated, and (3) amino acid residues corresponding to amino acid residues 19, 142, 156, 157, 158, 452, 478, 614, 681, and 950 of the spike protein found at GenBank Accession No. MN908947.3 are: R, D, deleted, deleted, G, R, K, G, R, and N, respectively. In another embodiment, provided herein is a recombinant NDV comprising a chimeric F protein, wherein the chimeric F protein comprises a derivative SARS-CoV-2 delta virus spike protein ectodomain and NDV F protein transmembrane and cytoplasmic domains, wherein the derivative comprises a SARS-CoV-2 delta spike protein comprising a SARS-CoV-2 delta variant ectodomain in which: (1) amino acid residues corresponding to amino acid residues 817, 892, 899, 942, 986, and 987 of the SARS-CoV-2 spike protein found at GenBank Accession No. MN908947.3 are substituted with prolines, (2) amino acid residues corresponding to amino acid residues 682 to 685 of the polybasic cleavage site of the SARS-CoV-2 spike protein found at GenBank Accession No. MN908947.3 are substituted such that the polybasic cleavage site is inactivated, and (3) amino acid residues corresponding to amino acid residues 19, 142, 156, 157, 158, 452, 478, 614, and 950 of the spike protein found at GenBank Accession No. MN908947.3 are: R, D, deleted, deleted, G, R, K, R, and N, respectively. In specific embodiments, a polybasic cleavage site is inactivated if the site cannot be cleaved by, e.g., furin. In a specific embodiment, amino acid residues corresponding to amino acid residues 682 to 685 of the polybasic cleavage site of the SARS-CoV-2 spike protein found at GenBank Accession No. MN908947.3 are substituted with a single alanine. In certain embodiments, the NDV F protein transmembrane and cytoplasmic domains are fused to the derivative of the SARS-CoV-2 spike protein ectodomain through a linker sequence (e.g., GGGGS (SEQ ID NO:7)). In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 2, 3, 4, 5, 6, 7, 8 or more. In a specific embodiment, the linker comprises the sequence GGGGS (SEQ ID NO:7). In some embodiments, the NDV F protein transmembrane and cytoplasmic domains are fused directly to the derivative of the SARS-CoV-2 spike protein ectodomain. In a specific embodiment, the NDV F protein and chimeric F protein is incorporated into the NDV virion.

In another embodiment, provided herein is a recombinant Newcastle disease virus (NDV) comprising a chimeric F protein, wherein the chimeric F protein comprises a derivative of a SARS-CoV-2 delta virus spike protein ectodomain and NDV F protein transmembrane and cytoplasmic domains, and wherein the derivative comprises an amino acid sequence at least 99% identical to the amino acid sequence of SEQ ID NO:13 or 17. In another embodiment, provided herein is a recombinant Newcastle disease virus (NDV) comprising a chimeric F protein, wherein the chimeric F protein comprises a derivative of a SARS-CoV-2 delta virus spike protein ectodomain and NDV F protein transmembrane and cytoplasmic domains, and wherein the derivative comprises an amino acid sequence at least 99.5% identical to the amino acid sequence of SEQ ID NO:13 or 17. In another embodiment, provided herein is a recombinant Newcastle disease virus (NDV) comprising a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:13 or 17. In certain embodiments, the NDV F protein transmembrane and cytoplasmic domains are fused to the derivative SARS-CoV-2 delta variant spike protein ectodomain through a linker sequence (e.g., GGGGS (SEQ ID NO:7)). In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 2, 3, 4, 5, 6, 7, 8 or more. In a specific embodiment, the linker comprises the sequence GGGGS (SEQ ID NO:7). In some embodiments, the NDV F protein transmembrane and cytoplasmic domains are fused to directly to the derivative SARS-CoV-2 delta variant spike protein ectodomain. In a specific embodiment, the NDV F protein and chimeric F protein is incorporated into the NDV virion.

In another embodiment, provided herein is a recombinant NDV comprising a chimeric F protein, wherein the chimeric F protein is encoded by a nucleotide sequence comprising the nucleotide sequence of SEQ ID NO:5. In another embodiment, provided herein is a recombinant NDV comprising a chimeric F protein, wherein the chimeric F protein is encoded by a nucleotide sequence comprising the nucleotide sequence of SEQ ID NO:21. encoded by a nucleotide sequence comprising the nucleotide sequence of SEQ ID NO:5. In another embodiment, provided herein is a recombinant NDV comprising a chimeric F protein, wherein the chimeric F protein comprises the amino acid sequence of SEQ ID NO: 6 or 18. In a specific embodiment, the NDV F protein and chimeric F protein is incorporated into the NDV virion.

The recombinant NDV may have the backbone of any NDV type or strain, including, but not limited to, naturally-occurring strains, variants or mutants, mutagenized viruses, reassortants or genetically engineered viruses, or any combination thereof. In a specific embodiment, the recombinant NDV comprises an NDV backbone which is lentogenic. In another specific embodiment, the recombinant NDV comprises an NDV backbone of the NDV LaSota strain. See, e.g., SEQ ID NO: 1 for a cDNA sequence of the genomic sequence of NDV LaSota strain. See also SEQ ID NO:3 for another cDNA sequence of the genomic sequence of NDV. In another specific embodiment, the recombinant NDV comprises an NDV backbone of the NDV Hitchner B1 strain. See, e.g., SEQ ID NO:2 for a cDNA sequence of the genomic sequence of NDV Hitchner strain. In another specific embodiment, the recombinant NDV comprises an NDV backbone of a lentogenic strain other than the NDV Hitchner B1 strain.

The transgene encoding a protein comprising a SARS-CoV-2 delta variant spike protein or a chimeric F protein may be incorporated into the genome of any NDV type or strain. In a specific embodiment, the transgene is incorporated into the genome of a lentogenic NDV. In another specific embodiment, the transgene is incorporated in the genome of NDV strain LaSota. See, e.g., SEQ ID NO: 1 for a cDNA sequence of the genomic sequence of NDV LaSota strain. See also SEQ ID NO:3 for another cDNA sequence of the genomic sequence of NDV. Another example of an NDV strain into which the transgene may be incorporated is the NDV Hitchner B1 strain. In a specific embodiment, the transgene may be incorporated into the genomic sequence of NDV Hitchner B1 strain. See, e.g., SEQ ID NO:2 for a cDNA sequence of the genomic sequence of NDV Hitchner B1 strain. In a specific embodiment, the transgene may be incorporated into the genome of a lentogenic strain other than the NDV Hitchner B1 strain. The transgene may be incorporated into the NDV genome between two transcription units (e.g., between NDV P and M genes, or between NP and P genes). In certain embodiment, the genome of the recombinant NDV does not comprise a heterologous sequence encoding a heterologous protein other than a protein comprising the SARS-CoV-2 delta variant spike protein or a portion thereof (e.g., the ectodomain or receptor binding domain of a SARS-CoV-2 spike protein), or a chimeric F protein. In some embodiments, the genome of the recombinant NDV does not comprise a transgene other than a transgene comprising a nucleotide sequence encoding a protein comprising a SARS-CoV-2 delta variant spike protein or a portion thereof (e.g., the ectodomain or receptor binding domain of a SARS-CoV-2 spike protein), or a chimeric F protein.

In another aspect, provided herein are compositions (e.g., immunogenic compositions) comprising a recombinant NDV described herein. In some embodiments, the recombinant NDV is a live virus. In other embodiments, the recombinant NDV is inactivated. The recombinant NDV may be inactivated using techniques knowns to one of skill in the art or described herein. A composition (e.g., immunogenic compositions) may further comprise pharmaceutically acceptable carrier. In certain embodiments, a composition (e.g., immunogenic compositions) may further comprise an adjuvant known to one of skill in the art or described herein. The compositions may be used in a method to induce an immune response to SARS-CoV-2 delta variant spike protein, to immunize against SARS-CoV-2 delta variant, and/or to prevent COVID-19.

In another aspect, presented herein are methods for inducing an immune response to a SARS-CoV-2 spike protein comprising administering to a subject (e.g., a human subject) a recombinant NDV described herein or a composition comprising a recombinant NDV described herein. In a specific embodiment, presented herein are methods for inducing an immune response to a SARS-CoV-2 delta variant spike protein comprising administering to a subject (e.g., a human subject) a recombinant NDV described herein or a composition comprising a recombinant NDV described herein. The composition may comprise an inactivated NDV. Alternatively, the composition may comprise live NDV. See, e.g., Section 5.4 regarding compositions. The recombinant NDV or a composition thereof may be administered by any route. In a specific embodiment, the recombinant NDV or a composition thereof is administered to a subject intranasally or intramuscularly. In certain embodiments, the recombinant NDV or a composition thereof is administered intranasally by a nasal spray.

In another aspect, presented herein are methods for immunizing against SARS-CoV-2 comprising administering to a subject (e.g., a human subject) a recombinant NDV described herein or a composition comprising a recombinant NDV described herein. In a specific embodiment, presented herein are methods for immunizing against SARS-CoV-2 delta variant comprising administering to a subject (e.g., a human subject) a recombinant NDV described herein or a composition comprising a recombinant NDV described herein. The composition may comprise an inactivated NDV. Alternatively, the composition may comprise live NDV. See, e.g., Section 5.4 regarding compositions. The recombinant NDV or a composition thereof may be administered by any route. In a specific embodiment, the recombinant NDV or a composition thereof is administered to a subject intranasally or intramuscularly. In certain embodiments, the recombinant NDV or a composition thereof is administered intranasally by a nasal spray.

In another aspect, presented herein are methods for preventing COVID-19 comprising administering to a subject (e.g., a human subject) a recombinant NDV described herein or a composition comprising a recombinant NDV described herein. The composition may comprise an inactivated NDV. Alternatively, the composition may comprise live NDV. See, e.g., Section 5.4 regarding compositions. The recombinant NDV or a composition thereof may be administered by any route. In a specific embodiment, the recombinant NDV or a composition thereof is administered to a subject intranasally or intramuscularly. In certain embodiments, the recombinant NDV or a composition thereof is administered intranasally by a nasal spray.

In a specific embodiment, provided herein is a method for boosting antibody titer to SARS-CoV-2 spike protein in a subject (e.g., a human) previously vaccinated for COVID-19 or previously infected with SARS-CoV-2, comprising administering to the subject the recombinant NDV described herein or the immunogenic composition described herein. In another specific embodiment, provided herein is a method for boosting immunity to SARS-CoV-2 in a subject (e.g., a human) previously vaccinated for COVID-19 or previously infected with SARS-CoV-2, comprising administering to the subject the recombinant NDV described herein or the immunogenic composition described herein.

The recombinant NDV described herein may be administered to a subject in combination with one or more other therapies. The recombinant NDV and one or more other therapies may be administered by the same or different routes of administration to the subject. In a specific embodiment, the recombinant NDV is administered to a subject intranasally or intramuscularly. See, e.g., Sections 5.1, and 6, infra for information regarding recombinant NDV, Section 5.5.3 for information regarding other therapies, Section 5.4, infra, for information regarding compositions and routes of administration, and Sections 5.5.1, infra, for information regarding methods of immunizing against SARS-CoV-2.

In another aspect, provided herein is a transgene comprising a nucleotide sequence encoding a chimeric F protein described herein. In a specific embodiment, provided herein is a transgene comprising a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:12 with one, two, three, four, five, six, seven, or all of the following amino acid modifications: T19R, G142D, delE156, delF157, R158G, L452R, T478K, D614G, P681R, and D950N. In specific embodiments, the derivative does not comprise the amino acid substitution of P681R. In a specific embodiment, provided herein is a transgene comprising a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:12 with one, two, three, four, five, six, seven, or all of the following amino acid modifications: T19R, G142D, delE156, delF157, R158G, L452R, T478K, D614G, and D950N. In another specific embodiment, provided herein is a transgene comprising a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:12 with the following amino acid modifications: T19R, G142D, delE156, delF157, R158G, L452R, T478K, D614, P681R, and D950N. In specific embodiments, the derivative does not comprise the amino acid substitution of P681R. In another specific embodiment, provided herein is a transgene comprising a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:12 with the following amino acid modifications: T19R, G142D, delE156, delF157, R158G, L452R, T478K, D614, and D950N. In another specific embodiment, provided herein is a transgene comprising a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:16 with one, two, three, four, five, six, seven, eight, or all of the following amino acid modifications: T6R, G129D, delE143, delF144, R145G, L439R, T465K, D601G, and D937N. In specific embodiments, the derivative of the SARS-CoV-2 delta virus spike protein ectodomain is linked via a linker to the NDV F protein transmembrane and cytoplasmic domains. In specific embodiments, the transgene further comprises a gene end sequence, a gene start sequence, and a Kozak sequence at the 5′ end.

In another specific embodiment, provided herein is a transgene comprising a nucleotide sequence encoding a chimeric F protein, wherein the chimeric F protein comprises a derivative SARS-CoV-2 delta virus spike protein ectodomain and NDV F protein transmembrane and cytoplasmic domains, wherein the derivative comprises a SARS-CoV-2 delta spike protein comprising a SARS-CoV-2 delta variant ectodomain in which: (1) amino acid residues corresponding to amino acid residues 817, 892, 899, 942, 986, and 987 of the SARS-CoV-2 spike protein found at GenBank Accession No. MN908947.3 are substituted with prolines, (2) amino acid residues corresponding to amino acid residues 682 to 685 of the polybasic cleavage site of the SARS-CoV-2 spike protein found at GenBank Accession No. MN908947.3 are substituted such that the polybasic cleavage site is inactivated, and (3) two, three, four, five, six, seven, or more amino acid residues corresponding to two, three, four, five, six, seven, or more of amino acid residues 19, 142, 156, 157, 158, 452, 478, 614, 681, and 950 of the spike protein found at GenBank Accession No. MN908947.3 are as follows: 19R, 142D, del156, del157, 158G, 452R, 478K, 614G, 681R, and 950N. In a specific embodiment, the derivative does not comprise the amino acid substitution of 681R. In another specific embodiment, provided herein is a transgene comprising a nucleotide sequence encoding a chimeric F protein, wherein the chimeric F protein comprises a derivative SARS-CoV-2 delta virus spike protein ectodomain and NDV F protein transmembrane and cytoplasmic domains, wherein the derivative comprises a SARS-CoV-2 delta spike protein comprising a SARS-CoV-2 delta variant ectodomain in which: (1) amino acid residues corresponding to amino acid residues 817, 892, 899, 942, 986, and 987 of the SARS-CoV-2 spike protein found at GenBank Accession No. MN908947.3 are substituted with prolines, (2) amino acid residues corresponding to amino acid residues 682 to 685 of the polybasic cleavage site of the SARS-CoV-2 spike protein found at GenBank Accession No. MN908947.3 are substituted such that the polybasic cleavage site is inactivated, and (3) amino acid residues corresponding to amino acid residues 19, 142, 156, 157, 158, 452, 478, 614, 681, and 950 of the spike protein found at GenBank Accession No. MN908947.3 are: R, D, deleted, deleted, G, R, K, G, R, and N, respectively. In a specific embodiment, the derivative does not comprise the amino acid substitution of 681R. In another specific embodiment, provided herein is a transgene comprising a nucleotide sequence encoding a chimeric F protein, wherein the chimeric F protein comprises a derivative SARS-CoV-2 delta virus spike protein ectodomain and NDV F protein transmembrane and cytoplasmic domains, wherein the derivative comprises a SARS-CoV-2 delta spike protein comprising a SARS-CoV-2 delta variant ectodomain in which: (1) amino acid residues corresponding to amino acid residues 817, 892, 899, 942, 986, and 987 of the SARS-CoV-2 spike protein found at GenBank Accession No. MN908947.3 are substituted with prolines, (2) amino acid residues corresponding to amino acid residues 682 to 685 of the polybasic cleavage site of the SARS-CoV-2 spike protein found at GenBank Accession No. MN908947.3 are substituted such that the polybasic cleavage site is inactivated, and (3) amino acid residues corresponding to amino acid residues 19, 142, 156, 157, 158, 452, 478, 614, and 950 of the spike protein found at GenBank Accession No. MN908947.3 are as follows: R, D, deleted, deleted, G, R, K, G, and N. In specific embodiments, a polybasic cleavage site is inactivated if it cannot be cleaved by, e.g., furin. In specific embodiments, the amino acid residues corresponding to amino acid residues 682 to 685 of the polybasic cleavage site of the SARS-CoV-2 spike protein found at GenBank Accession No. MN908947.3 are substituted with a single alanine. In specific embodiments, the derivative of the SARS-CoV-2 delta virus spike protein ectodomain is linked via a linker to the NDV F protein transmembrane and cytoplasmic domains. In specific embodiments, the transgene further comprises a gene end sequence, a gene start sequence, and a Kozak sequence at the 5′ end.

In another specific embodiment, provided herein is a transgene comprising a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises an amino acid sequence at least 99% identical to the amino acid sequence of SEQ ID NO:13 or 17. In another specific embodiment, provided herein is a transgene comprising a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises an amino acid sequence at least 99.5% identical to the amino acid sequence of SEQ ID NO:13 or 17. In another specific embodiment, provided herein is a transgene comprising a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:13 or 17. In specific embodiments, the derivative of the SARS-CoV-2 delta virus spike protein ectodomain is linked via a linker to the NDV F protein transmembrane and cytoplasmic domains. In specific embodiments, the transgene further comprises a gene end sequence, a gene start sequence, and a Kozak sequence at the 5′ end.

In another specific embodiment, provided herein is a transgene comprising a nucleotide sequence encoding a chimeric F protein, wherein the chimeric F protein comprises an amino acid sequence that is at least 99% identical to the amino acid sequence of SEQ ID NO:6 or 18. In another specific embodiment, provided herein is a transgene comprising a nucleotide sequence encoding a chimeric F protein, wherein the chimeric F protein comprises an amino acid sequence that is at least 99.5% identical to the amino acid sequence of SEQ ID NO:6 or 18. In another specific embodiment, provided herein is a transgene comprising a nucleotide sequence encoding a chimeric F protein, wherein the chimeric F protein comprises the amino acid sequence of SEQ ID NO:6 or 18. In another specific embodiment, provided herein is a transgene comprising a nucleotide sequence encoding a chimeric F protein, wherein the chimeric F protein is encoded by a nucleotide sequence comprising the nucleotide sequence of SEQ ID NO:5 or 21. In specific embodiments, the transgene further comprises a gene end sequence, a gene start sequence, and a Kozak sequence at the 5′ end.

In another aspect, provided herein is a vector comprising a transgene described herein. In some embodiments, the vector is a viral vector or a plasmid. In a specific embodiment, provided herein is a recombinant NDV comprising genome that comprises a transgene described herein.

In another aspect, provided herein is a nucleotide sequence comprising a NDV genome and a transgene described herein. The nucleotide sequence may comprise a nucleic acid sequence of an NDV genome known in the art or described (see, e.g., Section 5.1 or the Examples below; see also SEQ ID NO: 1, 2 or 3) and a nucleic acid sequence of a transgene described herein. In a specific embodiment, provided herein is a nucleotide sequence comprising a transgene described herein and (1) a NDV F transcription unit, (2) a NDV NP transcription unit, (3) a NDV M transcription unit, (4) a NDV L transcription unit, (5) a NDV P transcription unit, and (6) a NDV HN transcription unit. In another specific embodiment, provided herein is a nucleotide sequence comprising a transgene described herein and (1) a NDV F transcription unit, (2) a NDV NP transcription unit, (3) a NDV M transcription unit, (4) a NDV L transcription unit, (5) a NDV P transcription unit, and (6) a NDV HN transcription unit, wherein the NDV F transcription unit encodes a NDV F protein comprising a leucine to alanine amino acid substitution at the amino residue corresponding to amino acid residue 289 of the F protein of the LaSota NDV strain. In a specific embodiment, the nucleotide sequence is isolated.

In another embodiment, provided herein is a nucleotide sequence comprising an NDV genome and a transgene, wherein the transgene comprises a codon-optimized nucleotide sequence encoding a protein comprising a SARS-CoV-2 delta variant spike protein or a portion thereof (e.g., the receptor binding domain or ectodomain of the SARS-CoV-2 spike protein), and a gene end sequence, a gene start sequence, and a Kozak sequence at the 5′ end. In some embodiments, the gene end sequence and the gene start sequence comprise the nucleotide sequences set forth in SEQ ID NO: 9 and 10, respectively. In certain embodiments, the additional nucleotides are present at the 3′ end in order to follow the “rule of six.” In some embodiments, the transgene is between the NDV P and M genes, or between the NDV NP and P genes. In a specific embodiment, the nucleotide sequence is isolated.

In another embodiment, provided herein is a nucleotide sequence comprising an NDV genome and a transgene, wherein the transgene comprises a nucleotide sequence encoding a chimeric F protein described herein and a gene end sequence, a gene start sequence, and a Kozak sequence at the 5′ end. In certain embodiments, additional nucleotides are present at the 3′ end in order to follow the “rule of six.” In some embodiments, the transgene is between the NDV P and M genes, or between the NDV NP and P genes. In a specific embodiment, the nucleotide sequence is isolated.

In another aspect, provided herein is a vector comprising a nucleotide sequence, wherein the nucleotide sequence comprises a NDV genome and a transgene described herein. In some embodiments, the vector is a viral vector or a plasmid. In specific embodiments, provided herein is a recombinant NDV comprising a nucleotide sequence, wherein the nucleotide sequence comprises a NDV genome and a transgene described herein.

In one embodiment, provided herein is a nucleic acid sequence encoding a protein comprising (or consisting of) the SARS-CoV-2 delta variant spike protein or a portion thereof (e.g., ectodomain, S1, S2, or receptor binding domain). In another embodiment, provided herein is a nucleic acid sequence encoding a protein comprising (or consisting of) a derivative of a SARS-CoV-2 delta variant spike protein or a portion thereof (e.g., ectodomain, S1, S2, or receptor binding domain). In some embodiments, the protein further comprises a trimerization domain (e.g., a T4 foldon trimerization domain). The trimerization domain may be at the C-terminus of the SARS-CoV-2 delta variant spike protein or a portion thereof. In some embodiments, the protein further comprises a C-terminus thrombin cleavage site and a T4 foldon trimerization domain, and optionally a tag (e.g., a His tag (e.g., a hexahistidine tag) or a Flag tag). In specific embodiments, the nucleic acid sequence is isolated. In some embodiments, provided herein is a vector comprising the nucleic acid sequence. In some embodiments, the vector is a viral vector or a plasmid. In some embodiments, the plasmid is one described herein (e.g., in Section 6). In a specific embodiment, the viral vector is a recombinant NDV.

In another embodiment, provided herein is a nucleic acid sequence encoding a protein comprising a derivative of a SARS-CoV-2 spike protein ectodomain, and at the C-terminus a thrombin cleavage site and a T4 foldon trimerization domain, wherein the derivative lacks the polybasic cleavage site (RRAR) and carries two stabilizing mutations (K986P and V987P as counted based on the SARS-CoV-2 spike protein found at GenBank Accession MN908947.3). In another embodiment, provided herein is a nucleic acid sequence encoding a protein comprising a derivative of a SARS-CoV-2 spike protein ectodomain, and trimerization domain (e.g., a T4 foldon trimerization domain) at the C-terminus. In another embodiment, provided herein is a nucleic acid sequence encoding a protein comprising a derivative of a SARS-CoV-2 spike protein ectodomain, and at the C-terminus a thrombin cleavage site, a T4 foldon trimerization domain, and a hexahistidine tag, wherein the derivative lacks the polybasic cleavage site (RRAR) and carries two stabilizing mutations (K986P and V987P as counted based on the SARS-CoV-2 spike protein found at GenBank Accession MN908947.3). In some embodiments, provided herein is a nucleic acid sequence encoding a recombinant protein described in Section 6. In specific embodiments, the nucleic acid sequence is isolated. In some embodiments, provided herein is a vector comprising the nucleic acid sequence. In some embodiments, the vector is a viral vector or a plasmid. In some embodiments, the plasmid is one described herein (e.g., in Section 6). In a specific embodiment, the viral vector is a recombinant NDV.

In a specific embodiment, provided herein is a nucleic acid sequence encoding a protein comprising (or consisting of) the amino acid sequence of SEQ ID NO: 13 or 17. In another specific embodiment, provided herein is a nucleic acid sequence encoding a protein comprising (or consisting of) an amino acid sequence that is at least 99% or at least 99.5% identical to SEQ ID NO: 13 or 17. In some embodiments, the nucleic acid sequence comprises (or consists of) the nucleotide sequence of SEQ ID NO:19 or 20. In some embodiments, the nucleic acid sequence further comprises a trimerization domain (e.g., a T4 foldon domain). In some embodiments, the nucleic acid sequence comprises a nucleotide sequence encoding a protein comprising (or consisting of) the amino acid sequence of SEQ ID NO: 13 or 17, or a nucleotide sequence encoding a protein comprising (or consisting of) an amino acid sequence that is at least 99% or at least 99.5% identical to SEQ ID NO: 13 or 17, and at the C-terminus a thrombin cleavage site, a T4 foldon trimerization domain, and a hexahistidine tag. In specific embodiments, the nucleic acid sequence is isolated. In some embodiments, provided herein is a vector comprising the nucleic acid sequence. In some embodiments, the vector is a viral vector or a plasmid. In some embodiments, the vector is a plasmid described in Section 6. In a specific embodiment, the viral vector is a recombinant NDV.

In a specific embodiment, provided herein is a nucleic acid sequence comprising the nucleotide sequence encoding a protein comprising (or consisting of) the amino acid sequence of SEQ ID NO: 6 or 18. In another specific embodiment, provided herein is a nucleic acid sequence comprising a nucleotide sequence encoding a protein comprising (or consisting of) of an amino acid sequence that is at least 99% or at least 99.5% identical to SEQ ID NO: 6 or 18. In some embodiments, the nucleic acid sequence comprises (or consists of) the nucleotide sequence of SEQ ID NO:5 or 21. In specific embodiments, the nucleic acid sequence is isolated. In some embodiments, provided herein is a vector comprising the nucleic acid sequence. In some embodiments, the vector is a viral vector or a plasmid. In a specific embodiment, the viral vector is a recombinant NDV.

In certain embodiments, an “isolated” nucleic acid sequence refers to a nucleic acid molecule which is separated from other nucleic acid molecules which are present in the natural source of the nucleic acid. In other words, the isolated nucleic acid sequence can comprise heterologous nucleic acids that are not associated with it in nature. In other embodiments, an “isolated” nucleic acid sequence, such as a cDNA or RNA sequence, can be substantially free of other cellular material, or culture medium when produced by recombinant techniques, or substantially free of chemical precursors or other chemicals when chemically synthesized. The term “substantially free of cellular material” includes preparations of nucleic acid sequences in which the nucleic acid sequence is separated from cellular components of the cells from which it is isolated or recombinantly produced. Thus, nucleic acid sequence that is substantially free of cellular material includes preparations of nucleic acid sequence having less than about 30%, 20%, 10%, or 5% (by dry weight) of other nucleic acids. The term “substantially free of culture medium” includes preparations of nucleic acid sequence in which the culture medium represents less than about 50%, 20%, 10%, or 5% of the volume of the preparation. The term “substantially free of chemical precursors or other chemicals” includes preparations in which the nucleic acid sequence is separated from chemical precursors or other chemicals which are involved in the synthesis of the nucleic acid sequence. In specific embodiments, such preparations of the nucleic acid sequence have less than about 50%, 30%, 20%, 10%, 5% (by dry weight) of chemical precursors or compounds other than the nucleic acid sequence of interest.

In another aspect, provided herein is a protein (e.g., a chimeric F protein) described herein. In a specific embodiment, provided herein is a protein encoded by a transgene described herein (e.g., in Section 5.1.2 or 6). In specific embodiments, the protein is a recombinant protein.

In one embodiment, provided herein is a protein comprising (or consisting of) the SARS-CoV-2 delta variant spike protein or a portion thereof (e.g., ectodomain, S1, S2, or receptor binding domain). In another embodiment, provided herein is a protein comprising (or consisting of) a derivative of a SARS-CoV-2 delta variant spike protein or a portion thereof (e.g., ectodomain, S1, S2, or receptor binding domain). The protein may further comprise at the C-terminus a thrombin cleavage site and a T4 foldon trimerization domain, and optionally a tag (e.g., a His, such as a hexahistidine tag) or Flag tag). In specific embodiments, the protein is a recombinant protein.

In another embodiment, provided herein is a protein comprising a derivative of a SARS-CoV-2 spike protein ectodomain, and at the C-terminus a thrombin cleavage site and a T4 foldon trimerization domain, wherein the derivative lacks the polybasic cleavage site (RRAR) and carries two stabilizing mutations (K986P and V987P as counted based on the SARS-CoV-2 spike protein found at GenBank Accession MN908947.3). In another embodiment, provided herein is a protein comprising a derivative of a SARS-CoV-2 spike protein ectodomain, and at the C-terminus a thrombin cleavage site, a T4 foldon trimerization domain, and a hexahistidine tag, wherein the derivative lacks the polybasic cleavage site (RRAR) and carries two stabilizing mutations (K986P and V987P as counted based on the SARS-CoV-2 spike protein found at GenBank Accession MN908947.3). In specific embodiments, the protein is a recombinant protein.

In another embodiment, provided herein is a protein comprising a derivative of a SARS-CoV-2 spike protein ectodomain, and at the C-terminus a thrombin cleavage site and a T4 foldon trimerization domain, wherein the derivative comprises: (1) amino acid residues corresponding to amino acid residues 817, 892, 899, 942, 986, and 987 of the SARS-CoV-2 spike protein found at GenBank Accession No. MN908947.3 substituted with prolines; and (2) amino acid residues corresponding to amino acid residues 682 to 685 of the polybasic cleavage site of the SARS-CoV-2 spike protein found at GenBank Accession No. MN908947.3 substituted such that the polybasic cleavage site is inactivated. In another embodiment, provided herein is a protein comprising a derivative of a SARS-CoV-2 spike protein ectodomain, and at the C-terminus a thrombin cleavage site, a T4 foldon trimerization domain, and a hexahistidine tag, wherein the derivative comprises: (1) amino acid residues corresponding to amino acid residues 817, 892, 899, 942, 986, and 987 of the SARS-CoV-2 spike protein found at GenBank Accession No. MN908947.3 substituted with prolines; and (2) amino acid residues corresponding to amino acid residues 682 to 685 of the polybasic cleavage site of the SARS-CoV-2 spike protein found at GenBank Accession No. MN908947.3 substituted such that the polybasic cleavage site is inactivated. In specific embodiments, the protein is a recombinant protein. In some embodiments, provided herein is a recombinant protein described in Section 6.

In a specific embodiment, provided herein is a protein comprising (or consisting of) the amino acid sequence of SEQ ID NO: 13 or 17. In another specific embodiment, provided herein is a protein comprising (or consisting of) an amino acid sequence that is at least 99% or at least 99.5% identical to SEQ ID NO: 13 or 17. In some embodiments, the protein further comprises a trimerization domain (e.g., a T4 foldon domain) at the C-terminus. In some embodiments, provided herein is a protein comprising (or consisting of) the amino acid sequence of SEQ ID NO: 13 or 17, or an amino acid sequence that is at least 99% or at least 99.5% identical to SEQ ID NO: 13 or 17, and at the C-terminus a thrombin cleavage site, a T4 foldon trimerization domain, and a hexahistidine tag. In specific embodiments, the protein is a recombinant protein.

In another aspect, provided herein a protein comprising an ectodomain of a SARS-CoV-2 delta variant or a derivative thereof. In a specific embodiment, provided herein is a protein comprising (or consisting of) a derivative of an ectodomain of a SARS-CoV-2 delta variant, wherein the derivative comprises the amino acid sequence of SEQ ID NO: 13 or 17. In another specific embodiment, provided herein is a protein comprising (or consisting of) a derivative of an ectodomain of a SARS-CoV-2 delta variant, wherein the derivative comprises an amino acid sequence that is at least 95%, at least 96%, at least 97%, at least 98%, or at least 99% identical to SEQ ID NO:13 or 17. In specific embodiments, the derivative comprises: (1) amino acid substitutions to proline at two, three, four, five or all of the following amino acid positions 817, 892, 899, 942, 986, and 987 (as counted based on the SARS-CoV-2 spike protein found at GenBank Accession MN908947.3); and (2) RRAR to A amino acid substitution at amino acid positions 682 to 685 (as counted based on the SARS-CoV-2 spike protein found at GenBank Accession MN908947.3). In specific embodiments, the derivative comprises: (1) amino acid substitutions to proline at two, three, four, five or all of the following amino acid positions 817, 892, 899, 942, 986, and 987 (as counted based on the SARS-CoV-2 spike protein found at GenBank Accession MN908947.3); (2) RRAR to A amino acid substitution at amino acid positions 682 to 685 (as counted based on the SARS-CoV-2 spike protein found at GenBank Accession MN908947.3); and (3) amino acid substitutions at two, three, four, five, six, seven, eight, or all of the following amino acid positions 19, 142, 156, 157, 158, 452, 478, 614, and 950 (as counted based on the SARS-CoV-2 spike protein found at GenBank Accession MN908947.3), wherein the substitutions are selected from T19R, G142D, E156G, delF157, delR158, L452R, T478K, D614G, and D950N. In specific embodiments, the derivative comprises: (1) amino acid substitutions to proline at amino acid positions 817, 892, 899, 942, 986, and 987 (as counted based on the SARS-CoV-2 spike protein found at GenBank Accession MN908947.3); (2) RRAR to A amino acid substitution at amino acid positions 682 to 685 (as counted based on the SARS-CoV-2 spike protein found at GenBank Accession MN908947.3); and (3) amino acid substitutions at the following amino acid positions 19, 142, 156, 157, 158, 452, 478, 614, and 950 (as counted based on the SARS-CoV-2 spike protein found at GenBank Accession MN908947.3), wherein the substitutions are T19R, G142D, E156G, delF157, delR158, L452R, T478K, D614G, and D950N. In specific embodiments, the derivative comprises a proline at amino acid position 681 (as counted based on the SARS-CoV-2 spike protein found at GenBank Accession MN908947.3). In specific embodiments, the protein is a recombinant protein.

Techniques known to one of skill in the art or described herein (e.g., in Section 6) may be used to produce a recombinant protein. For example, standard cellular and molecular biology techniques, including molecular genetics techniques, may be used to generate nucleotide or nucleic acid sequences and proteins. Such techniques may include, e.g., molecular cloning, PCR, transfection or transformation, ligation, restriction enzyme digests, and gel electrophoresis. In specific embodiments, a protein is isolated from cells or other substrates using techniques known to one of skill in the art (e.g., column chromatography, size exclusion chromatography, ion exchange column chromatography, and affinity chromatography).

In another aspect, provided herein is a nucleotide sequence encoding a protein described herein. In a specific embodiment, the nucleotide sequence is isolated. In another aspect provided herein is a vector comprising a nucleotide sequence encoding a protein described herein. The vector may be a plasmid (e.g., a plasmid described herein) or a viral vector (e.g., NDV). In another aspect, provided herein are cells (e.g., cells described herein, e.g., Section 5.3, or 6) or embryonated eggs (e.g., embryonated eggs, such as described herein (e.g., Section 5.3), e.g., chicken embryonated eggs) comprising a nucleotide sequence encoding a protein described herein. In a specific embodiment, provided herein is a cell line (e.g., a cell line described herein) comprising a nucleotide sequence encoding a protein described herein. In another specific embodiment, provided herein is an ex vivo embryonated egg comprising a nucleotide sequence encoding a protein described herein. In another specific embodiment, provided herein are cells or embryonated eggs (e.g., chicken embryonated eggs) that express a recombinant protein described herein. In a specific embodiment, provided herein is a cell line (e.g., a cell line described herein) that expresses a protein described herein. In another specific embodiment, provided herein is an ex vivo embryonated egg (e.g., an embryonated egg, such as described herein (e.g., Section 5.3), e.g., a chicken embryonated egg) that expresses a protein described herein.

In a specific embodiment, provided herein is a protein comprising (or consisting of) the amino acid sequence of SEQ ID NO: 6 or 18. In another specific embodiment, provided herein is a protein comprising (or consisting of) of an amino acid sequence that is at least 99% or at least 99.5% identical to SEQ ID NO: 6 or 18. In specific embodiments, the protein is a recombinant protein.

In another aspect, provided herein is a composition comprising a protein described herein, a nucleotide sequence described herein, or a nucleic acid sequence described herein. The composition may comprises a pharmaceutically acceptable carrier. The composition may be used to induce an immune response, immunize a subject (e.g., a human subject), or prevent COVID-19. The composition may also be used in an immunoassay (e.g., an ELISA) to, e.g., measure anti-SARS-CoV-2 spike protein antibody.

In another aspect, provided herein is a method of inducing an immune response in a subject (e.g., a human), comprising administering to the subject a protein described herein, or a composition comprising a protein described herein. In another aspect, provided herein is a method of inducing an immune response in a subject (e.g., a human), comprising administering to the subject a nucleotide sequence described herein, or nucleic acid sequence described herein, or a composition comprising a nucleotide sequence described herein or nucleic acid sequence described herein.

In another aspect, provided herein is a method of immunizing a subject (e.g., a human) against SARS-CoV-2, comprising administering to the subject a protein described herein, or a composition comprising a protein described herein. In another aspect, provided herein is a method of immunizing a subject (e.g., a human) against SARS-CoV-2, comprising administering to the subject a nucleotide sequence described herein or nucleic acid sequence described herein, or a composition comprising a nucleotide sequence described herein or nucleic acid sequence described herein.

In another aspect, provided herein is a method of preventing COVID-19 in a subject (e.g., a human), comprising administering to the subject a protein described herein, or a composition comprising a protein described herein. In another aspect, provided herein is a method of preventing COVID-19 in a subject (e.g., a human), comprising administering to the subject a nucleotide sequence described herein or nucleic acid sequence described herein, or a composition comprising a nucleotide sequence described herein or nucleic acid sequence described herein.

In another aspect, provided herein are kits comprising a transgene described herein, a nucleotide sequence described herein, a recombinant NDV described herein, or the vector described herein. In one embodiment, provided herein is a kit comprising a transgene described herein. In another embodiment, provided herein is a kit comprising a nucleotide sequence, wherein the nucleotide sequence comprises a transgene described herein and a NDV genome. In another embodiment, provided herein is a vector described herein. In another embodiment, provided herein is a kit comprising a recombinant NDV described herein. In another embodiment, provided herein is a kit comprising a protein described herein.

### 3.1 Terminology

As used herein, the term “about” or “approximately” when used in conjunction with a number refers to any number within 1, 5 or 10% of the referenced number.

As used herein, the terms “antibody” and “antibodies” refer to molecules that contain an antigen binding site, e.g., immunoglobulins. Antibodies include, but are not limited to, monoclonal antibodies, bispecific antibodies, multispecific antibodies, human antibodies, humanized antibodies, synthetic antibodies, chimeric antibodies, polyclonal antibodies, single domain antibodies, camelized antibodies, single-chain Fvs (scFv), single chain antibodies, Fab fragments, F(ab′) fragments, disulfide-linked bispecific Fvs (sdFv), intrabodies, and anti-idiotypic (anti-Id) antibodies (including, e.g., anti-Id and anti-anti-Id antibodies to antibodies), and epitope-binding fragments of any of the above. In particular, antibodies include immunoglobulin molecules and immunologically active fragments of immunoglobulin molecules. Immunoglobulin molecules can be of any type (e.g., IgG, IgE, IgM, IgD, IgA and IgY), class (e.g., IgG1, IgG2, IgG3, IgG4, IgA1 and IgA2) or subclass.

As used herein, the term “heterologous” in the context of NDV refers an entity not found in nature to be associated with (e.g., encoded by, expressed by the genome of, or both) a naturally-occurring NDV. In a specific embodiment, a heterologous sequence encodes a protein that is not found associated with naturally-occurring NDV.

As used herein, the term “elderly human” refers to a human 65 years or older.

As used herein, the term “human adult” refers to a human that is 18 years or older.

As used herein, the term “human child” refers to a human that is 1 year to 18 years old.

As used herein, the term “human toddler” refers to a human that is 1 year to 3 years old.

As used herein, the term “human infant” refers to a newborn to 1 year old year human.

As used herein, the phrases “IFN deficient systems” or “IFN-deficient substrates” refer to systems, e.g., cells, cell lines and animals, such as mice, chickens, turkeys, rabbits, rats, horses etc., which do not produce one, two or more types of IFN, or do not produce any type of IFN, or produce low levels of one, two or more types of IFN, or produce low levels of any IFN (i.e., a reduction in any IFN expression of 5-10%, 10-20%, 20-30%, 30-40%, 40-50%, 50-60%, 60-70%, 70-80%, 80-90% or more when compared to IFN-competent systems under the same conditions), do not respond or respond less efficiently to one, two or more types of IFN, or do not respond to any type of IFN, have a delayed response to one, two or more types of IFN, are deficient in the activity of antiviral genes induced by one, two or more types of IFN, or induced by any type of IFN, or any combination thereof. The IFN (interferon) may be or include IFN-α, IFN-β, and IFN-γ.

As used herein, the terms “subject” or “patient” are used interchangeably. As used herein, the terms “subject” and “subjects” refers to an animal. In some embodiments, the subject is a mammal including a non-primate (e.g., a camel, donkey, zebra, bovine, horse, horse, cat, dog, rat, and mouse) and a primate (e.g., a monkey, chimpanzee, and a human). In some embodiments, the subject is a non-human mammal. In certain embodiments, the subject is a pet (e.g., dog or cat) or farm animal (e.g., a horse, pig or cow). In specific embodiments, the subject is a human. In certain embodiments, the mammal (e.g., human) is 4 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100 years old. In specific embodiments, the subject is an animal that is not avian.

As used herein, the term “in combination” in the context of the administration of (a) therapy(ies) to a subject, refers to the use of more than one therapy. The use of the term “in combination” does not restrict the order in which therapies are administered to a subject. A first therapy can be administered prior to, concomitantly with, or subsequent to the administration of a second therapy to a subject.

As used herein, the terms “SARS-CoV-2 spike protein” and “spike protein of SARS-CoV-2” includes a SARS-CoV-2 spike protein known to those of skill in the art. See, e.g., GenBank Accession Nos. MN908947.3, MT447160, MT44636, MT446360, MT444593, MT444529, MT370887, and MT334558 for examples of amino acid sequences of SARS-CoV-2 spike protein and nucleotide sequences encoding SARS-CoV-2 spike protein. A typical spike protein comprises domains known to those of skill in the art including an S1 domain, a receptor binding domain, an S2 domain, a transmembrane domain and a cytoplasmic domain. See, e.g., Wrapp et al., 2020, Science 367: 1260-1263 and Duan et al., 2020, Front. Immunol., Vol. 11, Article 576622 for a description of SARS-CoV-2 spike protein (in particular, the structure of such protein). The spike protein may be characterized has having a signal peptide, a receptor binding domain, an ectodomain, an S1 domain, an S2 domain, a transmembrane domain, and endodomain (or cytoplasmic).

As used herein, the terms “SARS-CoV-2 delta variant spike protein” and “spike protein of SARS-CoV-2 delta variant” includes a SARS-CoV-2 delta variant spike protein known to those of skill in the art. See, e.g., GISAID Accession Numbers EPI_ISL_1740580 (hCoV-19/England/CAMC-151FDF0/2021), EPI_ISL_1733902 (hCoV-19/USA/CA-CDC-FG-021941/2021), EPI_ISL_1731755 (hCoV-19/India/CG-AIIMS-Raipur-L15928/2021), and EPI_ISL_4634223 hCoV-19/Spain/CT-LabRefCat-1699309/2021 for SARS-CoV-2 delta variants. In certain embodiments, a SARS-CoV-2 delta variant may be found at GISAID Accession No. EPI_ISL_4634223 (hCoV-19/Spain/CT-LabRefCat-1699309/2021). In a specific embodiment, the SARS-CoV-2 delta variant is of the B1.617.2 sublineage. In certain embodiments, the SARS-CoV-2 delta variant is of the AY sublineage. In certain embodiments, the SARS-CoV-2 delta variant is of the AY.4, AY.25, AY.12, AY.3, AY.9, AY.3, AY.9, AY.5, AY.6, AY.20, AY.7.1, AY.23, AY.14, AY.10, AY.7, AY.13, AY.15, AY.16, AY.19, AY.2, AY.8, AY.11, AY.1, AY.21, AY.22, AY.7.2, AY.5.1, or AY.5.2 lineage. The term “SARS-CoV-2 delta virus spike protein” is also sometimes used herein to refer to the spike protein of a SARS-CoV-2 delta variant.

As used herein, the terms “therapies” and “therapy” can refer to any protocol(s), method(s), agent(s) or a combination thereof that can be used in the treatment or prevention of COVID-19, or vaccination. In certain embodiments, the term “therapy” refers to a recombinant NDV described herein. In other embodiments, the term “therapy” refers to an agent that is not a recombinant NDV described herein.

As used herein, the term “wild-type” in the context of nucleotide and amino acid sequences refers to the nucleotide and amino acid sequences of viral strains found in nature. In particular, the sequences described as wild-type herein are sequences that have been reported in public databases as sequences from natural viral isolates.

### 4. BRIEF DESCRIPTION OF THE FIGURES

FIGS. 1A-1B. Design of the NDV-HXP-S (Delta) constructs. (FIG. 1A) Mutations introduced in to NDV-HXP-S (Delta). *The P681 was not mutated to R in the vaccine constructs. (FIG. 1B) Schematic illustration of the design of two types of NDV-HXP-S (Delta) constructs.

FIGS. 2A-2B. Characterization of the NDV-HXP-S (Delta) variant by SDS-PAGE (FIG. 2A) NP/P construct and (FIG. 2B) P/M construct. NDV proteins L, HN, NP, F1, NP, P, M as well as the spike protein (S) are identified.

FIGS. 3A-3D. Design, production, and characterization of NDV-HXP-S variant vaccines (FIG. 3A) Structure and design of the NDV-HXP-S construct. The different SARS-CoV-2 spike sequences were introduced between the P and M genes of LaSota L289A NDV strain. The ectodomain of the spike was connected to the transmembrane domain and cytoplasmic tail (TM/CT) of the F protein of the NDV. The original polybasic cleavage site was removed by mutating RRAR to A. The HexaPro (F817P, A892P, A899P, A942P, K986P and V987P) stabilizing mutations were introduced. The sequence was codon-optimized for mammalian host expression. Original Wuhan HXP-S sequence is aligned with the new Delta variant construct. Changes in the N-terminal domain (NTD), the Receptor Binding Domain (RBD) and in the Spike 2 (S2) with respect to the original Wuhan HXP-S sequence are indicated. (FIG. 3B) NDV-HXP-S Delta variant was rescued by reverse genetics as previously described (Ayllon et al., J Vis Esp, 2013). Cells were co-transfected with the expression plasmid required for replication and transcription of the NDV viral genome (NP, P, and L), together with the full-length NDV cDNA. After 2 or 3 days, the tissue culture supernatants were inoculated into eight- or nine-day-old specific pathogen free (SPF) embryonated chicken eggs. Antigen identity was confirmed by biochemical methods and sequencing. The genetic stability of the recombinant virus was evaluated across multiples passages on ten days old-SPF embryonated chicken eggs. NDV-HXP-S vaccine was inactivated with BPL and purified by sucrose cushion ultracentrifugation (Sun et al., 2021, Nat. Commun. 12:6197) (FIG. 3C) Comparison of NDV-HXP-S Delta virus versus NDV-HXP-S Delta with P681R mutation. The spike protein and NDV HN proteins were detected by western blot using an anti-spike 2B3E5 mouse monoclonal antibody and an anti-HN 8H2 mouse monoclonal antibody, respectively. (FIG. 3D) Protein staining of NDV-HXP-S Delta variant vaccine resolved on 4-20% SDS-PAGE. The viral proteins were visualized by Coomassie Blue staining (L, S0, HN, N, P and M). The uncleaved S0 spike protein is noted with an approximate size of 200 kDa.

FIGS. 4A-4D. NDV-HXP-S vaccination regimens induce protection against phylogenetically distant SARS-CoV-2 variants. (FIGS. 4A and 4B) Design of the study and groups. Eight to ten-week old female BALB/c mice were used either vaccinated with 1 μg of total dose of inactivated NDV-HXP-S Wuhan or Delta variant vaccines or WT NDV (negative control). Two immunizations were performed via the intramuscular route (IM) at D0 and D21. At D44, mice were treated with Ad5-hACE2. At D49, mice were challenged with USA-WA1/2020, Delta (B.1.617.2) or Mu (B.1.621) strains and at day two after challenge, lungs were harvested and homogenized in 1 mL PBS and titers were measured by plaque assay. (FIG. 4C) Viral titers after challenge (n=5). Viral titers were measured by plaque assay on Vero E6 cells for Wuhan challenge and Vero TMPRSS2 cells for Delta and Mu challenges and plotted as GMT of PFU/mL (limit of detection equals to 50 PFU/mL; a titer of 25 PFU/mL was assigned to negative samples). The error bars represent geometric standard deviation. Statistical differences were analyzed by ordinary one-way ANOVA corrected for Dunnett's multiple comparisons test. The p values and geometric mean fold titers over the control are indicated. (*p<0.05; **p<0.01; ***p<0.001; ****p<0.0001) (FIG. 4D) Panel of neutralizing activity. Post-boost pooled sera were tested in microneutralization (MNT) assays against USA-WA/2020 strain (WT), Delta (B.1.617.2) variant, Beta (B.1.351) variant and Omicron (B.1.1.529) variant in technical duplicates. GMT serum dilutions inhibiting 50% of the infection (ID50) were plotted (limit of detection equals to 10 and was assigned to negative samples). Geometric mean fold change is noted.

FIGS. 5A-5C. NDV-HXP-S Delta vaccination induces high serum antibody titers against phylogenetically distant SARS-CoV-2 variants. Heatmap of spike-specific (FIG. 5A) or RBD-specific (FIG. 5B) serum IgG against spike variants (n=10). Wuhan, Delta, Alpha, Beta, Gamma and Omicron spikes or RBDs were used to measure the antibody binding of different immunization assays (please consult FIGS. 6A and 6B to see individual ELISA chart). (FIG. 5C) Wuhan S2-specific serum IgG (pooled of ten samples in triplicate). Antibodies in post-boost (D43) sera were measured by ELISAs. GMT AUC is depicted.

FIGS. 6A and 6B. Spike- and RBD-specific antibody titers after vaccination. Panel of spike-specific (FIG. 6A) or RBD-specific (FIG. 6B) serum IgG against spike variants (n=10) linked to FIGS. 5A and 5B, respectively. Wuhan, Delta, Alpha, Beta, Gamma and Omicron spikes or RBDs were used to measure the antibody binding of different immunization assays. Antibodies in post-boost (D43) sera were measured by ELISAs. GMT AUC is depicted. The error bars represent geometric SD. Geometric mean fold change is noted.

FIG. 7. NDV-HXP-S Delta vaccination induces serum antibody titers against phylogenetically distant SARS-CoV-2 variants. Heatmaps of Omicron BA.1 RBD-specific serum IgG against spike variants (n=10) and Omicron BA.2 RBD-specific serum IgG against spike variants (n=10). Omicron BA.1 and BA.2 RBDs were used to measure antibody binding following different immunization assays (please consult FIG. 8 to see individual ELISA charts).

FIG. 8. RBD-specific antibody titers after vaccination. Omicron BA.1 and BA.2 RBDs and N-terminal domain Omicron BA.1 were used to measure the antibody binding following different immunization assays. Antibodies in post-boost (D43) sera were measured by ELISAs. GMT AUC is depicted. The error bars represent geometric SD. Geometric mean fold change is noted.

### 5. DETAILED DESCRIPTION

### 5.1 Recombinant Newcastle Disease Virus

In one aspect, provided herein are recombinant NDV described herein that may be used to immunize a subject (e.g., a human subject) against SARS-CoV-2 delta variant. The recombinant NDV may be administered as a live virus or an inactivated virus.

### 5.1.1 NDV

Newcastle disease virus (NDV) is a member of the Avulavirus genus in the Paramyxoviridae family, which has been shown to infect a number of avian species (Alexander, D J (1988). Newcastle disease, Newcastle disease virus—an avian paramyxovirus. Kluwer Academic Publishers: Dordrecht, The Netherlands. pp 1-22). NDV possesses a single-stranded RNA genome in negative sense and does not undergo recombination with the host genome or with other viruses (Alexander, D J (1988). Newcastle disease, Newcastle disease virus—an avian paramyxovirus. Kluwer Academic Publishers: Dordrecht, The Netherlands. pp 1-22). The genomic RNA contains genes in the order of 3′-NP-P-M-F-HN-L-5′. Two additional proteins, V and W, are produced by NDV from the P gene by alternative mRNAs that are generated by RNA editing. The genomic RNA also contains a leader sequence at the 3′ end.

The structural elements of the virion include the virus envelope which is a lipid bilayer derived from the cell plasma membrane. The glycoprotein, hemagglutinin-neuraminidase (HN) protrudes from the envelope allowing the virus to contain both hemagglutinin (e.g., receptor binding/fusogenic) and neuraminidase activities. The fusion glycoprotein (F), which also interacts with the viral membrane, is first produced as an inactive precursor, then cleaved post-translationally to produce two disulfide linked polypeptides. The active F protein is involved in penetration of NDV into host cells by facilitating fusion of the viral envelope with the host cell plasma membrane. The matrix protein (M), is involved with viral assembly, and interacts with both the viral membrane as well as the nucleocapsid proteins.

The main protein subunit of the nucleocapsid is the nucleocapsid protein (NP) which confers helical symmetry on the capsid. In association with the nucleocapsid are the P and L proteins. The phosphoprotein (P), which is subject to phosphorylation, is thought to play a regulatory role in transcription, and may also be involved in methylation, phosphorylation and polyadenylation. The L gene, which encodes an RNA-dependent RNA polymerase, is required for viral RNA synthesis together with the P protein. The L protein, which takes up nearly half of the coding capacity of the viral genome is the largest of the viral proteins, and plays an important role in both transcription and replication.

Any NDV type or strain may be serve as the “backbone” that is engineered to encode a transgene described herein, including, but not limited to, naturally-occurring strains, variants or mutants, mutagenized viruses, reassortants and/or genetically engineered viruses. See, e.g., Section 5.1.2 and Section 6 for examples of transgenes. In a specific embodiment, the nucleotide sequence is incorporated into the genome of a lentogenic NDV. In another specific embodiment, the nucleotide sequence is incorporated in the genome of NDV strain LaSota. In another example of an NDV strain into which the nucleotide sequence may be incorporated is the NDV Hitchner B1 strain. In some embodiments, a lentogenic strain other than NDV Hitchner B1 strain is used as the backbone into which a nucleotide sequence may be incorporated. The nucleotide sequence may be incorporated into the NDV genome between two transcription units (e.g., between the NDV M and P transcription units, between the NDV NP and P transcription units, or between the NDV HN and L transcription units). In a specific embodiment, a transgene described herein is incorporated into the genome of a lentogenic NDV. In another specific embodiment, a transgene described herein is incorporated into the genome of NDV strain LaSota. In another embodiment, a transgene described herein is incorporated into the genome of NDV Hitchner B1 strain. In some embodiments, a lentogenic strain other than NDV Hitchner B1 strain is used as the backbone into which a nucleotide sequence may be incorporated. The transgene may be incorporated into the NDV genome between two transcription units (e.g., between the NDV M and P transcription units, between the NDV NP and P transcription units, or between the NDV HN and L transcription units).

In a specific embodiment, the NDV that is engineered to encode a transgene described herein is a naturally-occurring strain. Specific examples of NDV strains include, but are not limited to, Hitchner B1 strain (see, e.g., GenBank No. AF309418 or NC_002617) and LaSota strain (see, e.g., GenBank Nos. AY845400, AF07761.1 and JF950510.1 and GI No. 56799463). In a specific embodiment, the NDV that is engineered to encode a transgene described herein is the Hitchner B1 strain. In another embodiment, the NDV that is engineered to encode a transgene described herein is a B1 strain as identified by GenBank No. AF309418 or NC_002617. In a specific embodiment, the nucleotide sequence of the Hitchner B1 genome comprises an RNA sequence corresponding to the negative sense of the cDNA sequence set forth in SEQ ID NO:2. In another specific embodiment, the NDV that is engineered to encode a transgene described herein is the LaSota strain. In another embodiment, the NDV that is engineered to encode a transgene described herein is a LaSota strain as identified by AY845400, AF07761.1 or JF950510.1. In a specific embodiment, a NDV that is engineered to comprise a transgene described herein is a naturally-occurring strain. Specific examples of NDV strains include, but are not limited to, Hitchner B1 strain (see, e.g., GenBank No. AF309418 or NC_002617) and LaSota strain (see, e.g., GenBank Nos. AY845400, AF07761.1 and JF950510.1 and GI No. 56799463). In a specific embodiment, the NDV that is engineered to comprise a transgene described herein is the Hitchner B1 strain. In another embodiment, the NDV that is engineered to comprise a transgene described herein is a B1 strain as identified by GenBank No. AF309418 or NC_002617. In a specific embodiment, the nucleotide sequence of the Hitchner B1 genome comprises an RNA sequence corresponding to the negative sense of the cDNA sequence set forth in SEQ ID NO:2. In another specific embodiment, the NDV that is engineered to comprise a transgene described herein is the LaSota strain. In another embodiment, the NDV that is engineered to comprise a transgene described herein is a LaSota strain as identified by AY845400, AF07761.1 or JF950510.1. In a specific embodiment, the nucleotide sequence of the LaSota genome comprises an RNA sequence corresponding to the negative sense of the cDNA sequence set forth in SEQ ID NO:1. In another specific embodiment, the nucleotide sequence of the LaSota genome comprises an RNA sequence corresponding to the negative sense of the cDNA sequence set forth in SEQ ID NO:3. One skilled in the art will understand that the NDV genomic RNA sequence is an RNA sequence corresponding to the negative sense of a cDNA sequence encoding the NDV genome. Thus, any program that generates converts a nucleotide sequence to its reverse complement sequence may be utilized to convert a cDNA sequence encoding an NDV genome into the genomic RNA sequence (see, e.g., www.bioinformatics.org/sms/rev_comp.html, www.fr33.net/seqedit.php, and DNAStar). Accordingly, the nucleotide sequences provided in Tables 1-3, infra, may be readily converted to the negative-sense RNA sequence of the NDV genome by one of skill in the art.

In a specific embodiment, the NDV that is engineered to encode a transgene described herein comprises a genome encoding an NDV F protein in which a leucine amino acid residue at amino acid position 289 of NDV F protein is substituted for alanine (as described by, e.g., Sergel et al., 2000, Journal of Virology 74: 5101-5107). In another specific embodiment, the NDV that is engineered to encode a transgene described herein comprises a genome encoding an NDV F protein in which a leucine amino acid residue at amino acid position 289 of NDV F protein (as counted by the LaSota strain F protein) is substituted for alanine. In another specific embodiment, the NDV that is engineered to encode a transgene described herein comprises a genome comprises a nucleotide sequence encoding an NDV F protein in which leucine at the amino acid position corresponding to amino acid residue 289 of LaSota NDV F protein is substituted for alanine. In another specific embodiment, the NDV that is engineered to encode a transgene described herein comprises a genome comprising a nucleotide sequence encoding an NDV F protein in which leucine at the amino acid residue 289 of LaSota NDV F protein is substituted for alanine. In another specific embodiment, the NDV that is engineered to encode a transgene described herein is the LaSota strain (e.g., GenBank Accession Nos. AY845400, AF07761.1 or JF950510.1) and the genome of the LaSota strain encodes an NDV F protein in which a leucine amino acid residue at amino acid position 289 of NDV F protein is substituted for alanine. In another specific embodiment, the NDV that is engineered to encode a transgene described herein is the LaSota strain (e.g., GenBank Accession Nos. AY845400, AF07761.1 or JF950510.1) and the genome of the LaSota strain comprises a nucleotide sequence encoding LaSota NDV F protein in which leucine at amino acid residue 289 of the NDV F protein is substituted for alanine. In another specific embodiment, the NDV that is engineered to encode a transgene described herein is the Hitchner B1 strain (e.g., GenBank No. AF309418 or NC_002617) and the genome of the Hitchner B1 strain encodes an NDV F protein in which a leucine amino acid residue at amino acid position 289 of NDV F protein is substituted for alanine.

In a specific embodiment, the NDV that is engineered to comprise a transgene described herein comprises a genome encoding an NDV F protein in which a leucine amino acid residue at amino acid position 289 of NDV F protein is substituted for alanine (as described by, e.g., Sergel et al., 2000, Journal of Virology 74: 5101-5107). In another specific embodiment, the NDV that is engineered to comprise a transgene described herein comprises a genome encoding an NDV F protein in which a leucine amino acid residue at amino acid position 289 of NDV F protein (as counted by the LaSota strain F protein) is substituted for alanine. In another specific embodiment, the NDV that is engineered to comprise a transgene described herein comprises a genome comprises a nucleotide sequence encoding an NDV F protein in which leucine at the amino acid position corresponding to amino acid residue 289 of LaSota NDV F protein is substituted for alanine. In another specific embodiment, the NDV that is engineered to comprise a transgene described herein comprises a genome comprises a nucleotide sequence encoding an NDV F protein in which leucine at the amino acid residue 289 of LaSota NDV F protein is substituted for alanine. In another specific embodiment, the NDV that is engineered to comprise a transgene described herein is the LaSota strain (e.g., GenBank Accession Nos. AY845400, AF07761.1 or JF950510.1) and the genome of the LaSota strain encodes an NDV F protein in which a leucine amino acid residue at the amino acid position 289 of NDV F protein is substituted for alanine. In another specific embodiment, the NDV that is engineered to comprise a transgene described herein is the LaSota strain (e.g., GenBank Accession Nos. AY845400, AF07761.1 or JF950510.1) and the genome of the LaSota strain comprises a nucleotide sequence encoding LaSota NDV F protein in which leucine at amino acid residue 289 of the NDV F protein is substituted for alanine. In another specific embodiment, the NDV that is engineered to comprise a transgene described herein is the Hitchner B1 strain (e.g., GenBank No. AF309418 or NC_002617) and the genome of the Hitchner B1 strain encodes an NDV F protein in which a leucine amino acid residue at amino acid position corresponding to amino acid residue 289 of LaSota NDV F protein is substituted for alanine.

In some embodiments, the NDV that is engineered to encode a transgene described herein is the Fuller strain. In certain embodiments, the NDV that is engineered to encode a transgene described herein is the Ulster strain. In some embodiments, the NDV that is engineered to encode a transgene described herein is the Roakin strain. In certain embodiments, the NDV that is engineered to encode a transgene described herein is the Komarov strain. In certain embodiments, the NDV that is engineered to encode a transgene described herein is an NDV is the r73T-R1 16 virus.

In some embodiments, the NDV that is engineered to comprise a transgene described herein is the Fuller strain. In certain embodiments, the NDV that is engineered to encode a transgene described herein is the Ulster strain. In some embodiments, the NDV that is engineered to comprise a transgene described herein is the Roakin strain. In certain embodiments, the NDV that is engineered to comprise a transgene described herein is the Komarov strain. In certain embodiments, the NDV that is engineered to comprise a transgene described herein is an NDV is the r73T-R1 16 virus.

In specific embodiments, the NDV that is engineered to encode a transgene described herein is not pathogenic in birds as assessed by a technique known to one of skill. In certain specific embodiments, the NDV that is engineered to encode a transgene described herein is not pathogenic as assessed by intracranial injection of 1-day-old chicks with the virus, and disease development and death as scored for 8 days. In some embodiments, the NDV that is engineered to encode a transgene described herein has an intracranial pathogenicity index of less than 0.7, less than 0.6, less than 0.5, less than 0.4, less than 0.3, less than 0.2 or less than 0.1. In certain embodiments, the NDV that is engineered to encode a transgene described herein has an intracranial pathogenicity index of zero. See, e.g., OIE Terrestrial Manual 2012, Chapter 2.3.14, entitled “Newcastle Disease (Infection With Newcastle Disease Virus) for a description of this assay, which is found at the following website www.oie.int/fileadmin/Home/eng/Health_standards/tahm/2.03.14_NEWCASTLE_DIS.pdf, which is incorporated herein by reference in its entirety.

In certain embodiments, the NDV that is engineered to encode a transgene described herein is a mesogenic strain that has been genetically engineered so as not be a considered pathogenic in birds as assessed by techniques known to one skilled in the art.

In preferred embodiments, the NDV that is engineered to encode a transgene described herein is non-pathogenic in humans. In preferred embodiments, the NDV that is engineered to encode a transgene described herein is non-pathogenic in humans and avians. In certain embodiments, the NDV that is engineered to encode a transgene described herein is attenuated such that the NDV remains, at least partially, infectious and can replicate in vivo, but only generate low titers resulting in subclinical levels of infection that are non-pathogenic (see, e.g., Khattar et al., 2009, J. Virol. 83:7779-7782). Such attenuated NDVs may be especially suited for embodiments wherein the virus is administered to a subject in order to act as an immunogen, e.g., a live vaccine. The viruses may be attenuated by any method known in the art. In a specific embodiment, the NDV genome comprises sequences necessary for infection and replication of the virus such that progeny is produced and the infection level is subclinical. In certain embodiments, NDV is attenuated by introducing one, two, or more mutations (e.g., amino acid substitutions) in the NDV V protein.

In specific embodiments, the NDV that is engineered to comprise a transgene described herein is not pathogenic in birds as assessed by a technique known to one of skill. In certain specific embodiments, the NDV that is engineered to comprise a transgene described herein is not pathogenic as assessed by intracranial injection of 1-day-old chicks with the virus, and disease development and death as scored for 8 days. In some embodiments, the NDV that is engineered to comprise a transgene described herein has an intracranial pathogenicity index of less than 0.7, less than 0.6, less than 0.5, less than 0.4, less than 0.3, less than 0.2 or less than 0.1. In certain embodiments, the NDV that is engineered to comprise a transgene described herein has an intracranial pathogenicity index of zero. See, e.g., OIE Terrestrial Manual 2012, Chapter 2.3.14, entitled “Newcastle Disease (Infection With Newcastle Disease Virus) for a description of this assay, which is found at the following website www.oie.int/fileadmin/Home/eng/Health_standards/tahm/2.03.14_NEWCASTLE_DIS.pdf, which is incorporated herein by reference in its entirety.

In certain embodiments, the NDV that is engineered to comprise a transgene described herein is a mesogenic strain that has been genetically engineered so as not be a considered pathogenic in birds as assessed by techniques known to one skilled in the art.

In preferred embodiments, the NDV that is engineered to comprise a transgene described herein is non-pathogenic in humans. In preferred embodiments, the NDV that is engineered to comprise a transgene described herein is non-pathogenic in humans and avians.

In a specific embodiment, provided herein is a nucleic acid sequence comprising (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a transgene described herein. In certain embodiments, the NDV transcription units are LaSota NDV transcription units. In a specific embodiment, provided herein is a nucleic acid sequence comprising (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a transgene described herein, wherein the NDV F transcription unit encodes an NDV F protein with an amino acid substitution of leucine to alanine at the amino acid residue corresponding to amino acid position 289 of LaSota NDV F protein. In another specific embodiment, provided herein is a nucleic acid sequence comprising (1) an NDV F transcription unit, (2) an NDV NP transcription unit, (3) an NDV P transcription unit, (4) an NDV M transcription unit, (5) an NDV HN transcription unit, (6) an NDV L transcription unit, and (7) a transgene described herein, wherein the NDV F transcription unit encodes an NDV F protein with an amino acid substitution of leucine to alanine at amino acid position 289 of LaSota NDV F protein. In certain embodiments, the NDV transcription units are LaSota NDV transcription units. In certain embodiments, the nucleic acid sequence is part of a vector (e.g., a plasmid, such as described in the Example below). In specific embodiments, the nucleic acid sequence is isolated.

In a specific embodiment, provided herein is a nucleic acid sequence comprising (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a transgene described herein. In another specific embodiment, provided herein is a nucleic acid sequence comprising (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a transgene described herein, wherein the NDV F comprises an amino acid substitution of leucine to alanine at the amino acid position corresponding to amino acid residue 289 of LaSota NDV F. In another specific embodiment, provided herein is a nucleic acid sequence comprising (1) a nucleotide sequence encoding NDV F, (2) a nucleotide sequence encoding NDV NP, (3) a nucleotide sequence encoding NDV P, (4) a nucleotide sequence encoding NDV M, (5) a nucleotide sequence encoding NDV HN, (6) a nucleotide sequence encoding NDV L, and (7) a transgene described herein, wherein the NDV F comprises an amino acid substitution of leucine to alanine at the amino acid position 289 of LaSota NDV F. In certain embodiments, the NDV proteins are LaSota NDV proteins. In another specific embodiment, provided herein is a nucleic acid sequence comprising a nucleotide sequence of an NDV genome known in the art or described (see, e.g., Section 5.1 or the Example below; see also SEQ ID NO: 1, 2 or 3) and a transgene described herein. In certain embodiments, the nucleic acid sequence is part of a vector (e.g., a plasmid, such as described in the Examples below). In a specific embodiment, the nucleotide sequence is isolated.

In specific embodiments, a nucleic acid sequence or nucleotide sequence described herein is a recombinant nucleic acid sequence or recombinant nucleotide sequence. In certain embodiments, a nucleotide sequence or nucleic acid sequence described herein may be a DNA molecule (e.g., cDNA), an RNA molecule, or a combination of a DNA and RNA molecule. In some embodiments, a nucleotide sequence or nucleic acid sequence described herein may comprise analogs of DNA or RNA molecules. Such analogs can be generated using, for example, nucleotide analogs, which include, but are not limited to, inosine, methylcytosine, pseudouridine, or tritylated bases. Such analogs can also comprise DNA or RNA molecules comprising modified backbones that lend beneficial attributes to the molecules such as, for example, nuclease resistance or an increased ability to cross cellular membranes. The nucleic acid or nucleotide sequences can be single-stranded, double-stranded, may contain both single-stranded and double-stranded portions, and may contain triple-stranded portions. In a specific embodiment, a nucleotide sequence or nucleic acid sequence described herein is a negative sense single-stranded RNA. In another specific embodiment, a nucleotide sequence or nucleic acid sequence described herein is a positive sense single-stranded RNA. In another specific embodiment, a nucleotide sequence or nucleic acid sequence described herein is a cDNA.

### 5.1.2 SARS-Cov-2 Delta Variant Spike Protein/Chimeric F Protein with the SARS-Cov-2 Delta Variant Spike Protein Ectodomain

In a specific embodiment, provided herein is a transgene comprising a nucleotide sequence encoding a protein comprising a SARS-CoV-2 delta spike protein or portion thereof (e.g., ectodomain, S1 domain, S2 domain, or receptor binding domain of the SARS-CoV-2 spike protein). In another specific embodiment, provided herein is a transgene comprising a nucleotide sequence encoding a protein comprising a SARS-CoV-2 delta variant spike protein or portion thereof (e.g., ectodomain, S1 domain, S2 domain, or receptor binding domain of the SARS-CoV-2 delta variant spike protein).

In a specific embodiment, a transgene comprising a nucleotide sequence encoding a protein comprising a SARS-CoV-2 delta variant spike protein or portion thereof (e.g., ectodomain, S1 domain, S2 domain, or receptor binding domain of the SARS-CoV-2 delta variant spike protein) is incorporated into the genome of any NDV type or strain. (e.g., NDV LaSota strain) See, e.g., Section 5.1.1, supra, for types and strains of NDV that may be used. The transgene encoding a SARS-CoV-2 delta variant spike protein or portion thereof (e.g., ectodomain, S1 domain, S2 domain, or receptor binding domain of the SARS-CoV-2 delta variant spike protein) may inserted into any NDV type or strain (e.g., NDV LaSota strain). In a specific embodiment, a transgene encoding a SARS-CoV-2 delta variant spike protein or portion thereof (e.g., ectodomain, S1 domain, S2 domain, or receptor binding domain of the SARS-CoV-2 delta variant spike protein) is incorporated into the genome of any NDV type or strain (e.g., NDV LaSota strain). In a specific embodiment, the SARS-CoV-2 delta variant is of the B1.617.2 sublineage. In certain embodiments, the SARS-CoV-2 delta variant is of the AY sublineage. See, e.g., Section 3.1 for exemplary sequences for SARS-CoV-2 delta variant spike proteins or portion thereof (e.g., ectodomain, S1 domain, S2 domain, or receptor binding domain of the SARS-CoV-2 delta variant spike protein) and exemplary nucleic acid sequences encoding SARS-CoV-2 delta variant spike protein or portion thereof (e.g., ectodomain, S1 domain, S2 domain, or receptor binding domain of the SARS-CoV-2 delta variant spike protein). One of skill in the art would be able to use such sequence information to produce a transgene for incorporation into the genome of any NDV type or strain. Given the degeneracy of the nucleic acid code, there are a number of different nucleic acid sequences that may encode the same SARS-CoV-2 delta variant spike protein or portion thereof (e.g., ectodomain, S1 domain, S2 domain or receptor binding domain of the SARS-CoV-2 spike protein). In a specific embodiment, a transgene encoding a SARS-CoV-2 delta variant spike protein or portion thereof (e.g., ectodomain, S1 domain, S2 domain, or receptor binding domain of the SARS-CoV-2 delta variant spike protein) is codon optimized. See, e.g., Section 5.1.4, infra, for a discussion regarding codon optimization. In certain embodiments, the transgene encoding a SARS-CoV-2 delta variant spike protein or a portion thereof (e.g., ectodomain, S1 domain, S2 domain, or receptor binding domain of SARS-CoV-2 delta variant spike protein) is without the SARS-CoV-2 delta variant spike protein signal peptide. The transgene encoding a SARS-CoV-2 delta variant spike protein or portion thereof (e.g., ectodomain, S1 domain, S2 domain, or receptor binding domain of the SARS-CoV-2 spike protein) may be incorporated between any two NDV transcription units (e.g., between the NDV P and M transcription units, between the NDV NP and P transcription units, or between the HN and L transcription units).

In a specific embodiment, provided herein is a transgene comprising a nucleotide sequence encoding a protein, wherein the protein comprises a SARS-CoV-2 delta variant spike protein or portion thereof (e.g., ectodomain, S1 domain, S2 domain, or receptor binding domain of the SARS-CoV-2 delta variant spike protein) is incorporated into the genome of any NDV type or strain. (e.g., NDV LaSota strain) See, e.g., Section 5.1.1, supra, for types and strains of NDV that may be used. The transgene encoding a protein comprising a SARS-CoV-2 delta variant spike protein or portion thereof (e.g., ectodomain, S1 domain, S2 domain, or receptor binding domain of the SARS-CoV-2 delta variant spike protein) may inserted into any NDV type or strain (e.g., NDV LaSota strain). In a specific embodiment, a transgene encoding a protein comprising a SARS-CoV-2 delta variant spike protein or portion thereof (e.g., ectodomain, S1 domain, S2 domain, or receptor binding domain of the SARS-CoV-2 delta variant spike protein) is incorporated into the genome of any NDV type or strain (e.g., NDV LaSota strain). In a specific embodiment, the SARS-CoV-2 delta variant is of the B1.617.2 sublineage. In certain embodiments, the SARS-CoV-2 delta variant is of the AY sublineage. See, e.g., Section 3.1 for exemplary sequences for SARS-CoV-2 delta variant spike proteins or portion thereof (e.g., ectodomain, S1 domain, S2 domain, or receptor binding domain of the SARS-CoV-2 delta variant spike protein) and exemplary nucleic acid sequences encoding SARS-CoV-2 delta variant spike protein or portion thereof (e.g., ectodomain, S1 domain, S2 domain, or receptor binding domain of the SARS-CoV-2 delta variant spike protein). One of skill in the art would be able to use such sequence information to produce a transgene for incorporation into the genome of any NDV type or strain. Given the degeneracy of the nucleic acid code, there are a number of different nucleic acid sequences that may encode the same SARS-CoV-2 delta variant spike protein or portion thereof (e.g., ectodomain, S1 domain, S2 domain or receptor binding domain of the SARS-CoV-2 spike protein). In a specific embodiment, a transgene encoding a SARS-CoV-2 delta variant spike protein or portion thereof (e.g., ectodomain, S1 domain, S2 domain, or receptor binding domain of the SARS-CoV-2 delta variant spike protein), or a protein comprising a SARS-CoV-2 delta variant spike protein or a portion thereof is codon optimized. See, e.g., Section 5.1.4, infra, for a discussion regarding codon optimization. In certain embodiments, the transgene encoding a protein comprising a SARS-CoV-2 delta variant spike protein or a portion thereof (e.g., ectodomain, S1 domain, S2 domain, or receptor binding domain of SARS-CoV-2 delta variant spike protein) is without the SARS-CoV-2 delta variant spike protein signal peptide. The transgene encoding a SARS-CoV-2 delta variant spike protein or portion thereof (e.g., ectodomain, S1 domain, S2 domain, or receptor binding domain of the SARS-CoV-2 spike protein), or a protein comprising a SARS-CoV-2 delta variant spike protein or a portion thereof may be incorporated between any two NDV transcription units (e.g., between the NDV P and M transcription units, between the NDV NP and P transcription units, or between the HN and L transcription units).

In certain embodiments, a portion of a SARS-CoV-2 delta variant spike protein comprises the receptor binding domain of the SARS-CoV-2 delta variant spike protein. In some embodiments, a portion of a SARS-CoV-2 delta variant spike protein comprises the receptor binding domain of the SARS-CoV-2 delta variant spike protein and 5, 10, 15, 20, 30, 40, 50, 75 or more amino acid residues to N-terminus of the receptor binding domain of the SARS-CoV-2 delta variant spike protein, or 5, 10, 15, 20, 30, 40, 50, 75 or more amino acid residues C-terminus to the receptor binding domain of the SARS-CoV-2 delta variant spike protein, or 5, 10, 15, 20, 30, 40, 50, 75 or more amino acid residues N-terminus to the receptor binding domain of the SARS-CoV-2 delta variant spike protein and 5, 10, 15, 20, 30, 40, 50, 75 or more amino acid residues C-terminus to the receptor binding domain of the SARS-CoV-2 delta variant spike protein. In some embodiments, a portion of a SARS-CoV-2 spike protein comprises the receptor binding domain of the SARS-CoV-2 delta variant spike protein and 5 to 25, 5 to 50, 25 to 50, 25 to 75, or 50 to 75 amino acid residues N-terminus to the receptor binding domain of the SARS-CoV-2 delta variant spike protein, 5 to 25, 5 to 50, 25 to 50, 25 to 75, or 50 to 75 amino acid residues C-terminus to the receptor binding domain of the SARS-CoV-2 delta variant spike protein, or 5 to 25, 5 to 50, 25 to 50, 25 to 75, or 50 to 75 amino acid residues N-terminus to the receptor binding domain of SARS-CoV-2 delta variant spike protein and 5 to 25, 5 to 50, 25 to 50, 25 to 75, or 50 to 75 amino acid residues C-terminus to the receptor binding domain of the SARS-CoV-2 delta variant spike protein.

In certain embodiments, a portion of a SARS-CoV-2 delta variant spike protein comprises the S1 domain of the SARS-CoV-2 delta variant spike protein. In some embodiments, a portion of a SARS-CoV-2 delta variant spike protein comprises the S1 domain of the SARS-CoV-2 delta variant spike protein and 5, 10, 15, 20, 30, 40, 50, 75 or more amino acid residues to N-terminus of the S1 domain of the SARS-CoV-2 delta variant spike protein, or 5, 10, 15, 20, 30, 40, 50, 75 or more amino acid residues C-terminus to the S1 domain of the SARS-CoV-2 delta variant spike protein, or 5, 10, 15, 20, 30, 40, 50, 75 or more amino acid residues N-terminus to the S1 domain of the SARS-CoV-2 delta variant spike protein and 5, 10, 15, 20, 30, 40, 50, 75 or more amino acid residues C-terminus to the S1 domain of the SARS-CoV-2 delta variant spike protein. In some embodiments, a portion of a SARS-CoV-2 spike protein comprises the S1 domain of the SARS-CoV-2 delta variant spike protein and 5 to 25, 5 to 50, 25 to 50, 25 to 75, or 50 to 75 amino acid residues N-terminus to the S1 domain of the SARS-CoV-2 delta variant spike protein, 5 to 25, 5 to 50, 25 to 50, 25 to 75, or 50 to 75 amino acid residues C-terminus to the S1 domain of the SARS-CoV-2 delta variant spike protein, or 5 to 25, 5 to 50, 25 to 50, 25 to 75, or 50 to 75 amino acid residues N-terminus to the S1 domain of SARS-CoV-2 delta variant spike protein and 5 to 25, 5 to 50, 25 to 50, 25 to 75, or 50 to 75 amino acid residues C-terminus to the S1 domain of the SARS-CoV-2 delta variant spike protein.

In certain embodiments, a portion of a SARS-CoV-2 delta variant spike protein comprises the S2 domain of the SARS-CoV-2 delta variant spike protein. In some embodiments, a portion of a SARS-CoV-2 delta variant spike protein comprises the S2 domain of the SARS-CoV-2 delta variant spike protein and 5, 10, 15, 20, 30, 40, 50, 75 or more amino acid residues to N-terminus of the S2 domain of the SARS-CoV-2 delta variant spike protein, or 5, 10, 15, 20, 30, 40, 50, 75 or more amino acid residues C-terminus to the S2 domain of the SARS-CoV-2 delta variant spike protein, or 5, 10, 15, 20, 30, 40, 50, 75 or more amino acid residues N-terminus to the S2 domain of the SARS-CoV-2 delta variant spike protein and 5, 10, 15, 20, 30, 40, 50, 75 or more amino acid residues C-terminus to the S2 domain of the SARS-CoV-2 delta variant spike protein. In some embodiments, a portion of a SARS-CoV-2 spike protein comprises the S2 domain of the SARS-CoV-2 delta variant spike protein and 5 to 25, 5 to 50, 25 to 50, 25 to 75, or 50 to 75 amino acid residues N-terminus to the S2 domain of the SARS-CoV-2 delta variant spike protein, 5 to 25, 5 to 50, 25 to 50, 25 to 75, or 50 to 75 amino acid residues C-terminus to the S2 domain of the SARS-CoV-2 delta variant spike protein, or 5 to 25, 5 to 50, 25 to 50, 25 to 75, or 50 to 75 amino acid residues N-terminus to the S2 domain of SARS-CoV-2 delta variant spike protein and 5 to 25, 5 to 50, 25 to 50, 25 to 75, or 50 to 75 amino acid residues C-terminus to the S2 domain of the SARS-CoV-2 delta variant spike protein.

In certain embodiments, a portion of a SARS-CoV-2 delta variant spike protein comprises the S1 domain and S2 domain of the SARS-CoV-2 delta variant spike protein. In some embodiments, a portion of a SARS-CoV-2 delta variant spike protein comprises the S1 domain and S2 domain of the SARS-CoV-2 delta variant spike protein and 5, 10, 15, 20, 30, 40, 50, 75 or more amino acid residues to N-terminus of the S1 domain of the SARS-CoV-2 delta variant spike protein, or 5, 10, 15, 20, 30, 40, 50, 75 or more amino acid residues C-terminus to the S2 domain of the SARS-CoV-2 delta variant spike protein, or 5, 10, 15, 20, 30, 40, 50, 75 or more amino acid residues N-terminus to the S1 domain of the SARS-CoV-2 delta variant spike protein and 5, 10, 15, 20, 30, 40, 50, 75 or more amino acid residues C-terminus to the S2 domain of the SARS-CoV-2 delta variant spike protein. In some embodiments, a portion of a SARS-CoV-2 spike protein comprises the S1 domain and S2 domain of the SARS-CoV-2 delta variant spike protein and 5 to 25, 5 to 50, 25 to 50, 25 to 75, or 50 to 75 amino acid residues N-terminus to the S1 domain of the SARS-CoV-2 delta variant spike protein, 5 to 25, 5 to 50, 25 to 50, 25 to 75, or 50 to 75 amino acid residues C-terminus to the S2 domain of the SARS-CoV-2 delta variant spike protein, or 5 to 25, 5 to 50, 25 to 50, 25 to 75, or 50 to 75 amino acid residues N-terminus to the S1 domain of SARS-CoV-2 delta variant spike protein and 5 to 25, 5 to 50, 25 to 50, 25 to 75, or 50 to 75 amino acid residues C-terminus to the S2 domain of the SARS-CoV-2 delta variant spike protein.

In certain embodiments, a portion of a SARS-CoV-2 delta variant spike protein comprises the ectodomain of the SARS-CoV-2 delta variant spike protein. In some embodiments, a portion of a SARS-CoV-2 delta variant spike protein comprises the ectodomain of the SARS-CoV-2 delta variant spike protein and 5, 10, 15, 20, 30, 40, 50, 75 or more amino acid residues to N-terminus of the ectodomain of the SARS-CoV-2 delta variant spike protein, or 5, 10, 15, 20, 30, 40, 50, 75 or more amino acid residues C-terminus to the ectodomain of the SARS-CoV-2 delta variant spike protein, or 5, 10, 15, 20, 30, 40, 50, 75 or more amino acid residues N-terminus to the ectodomain of the SARS-CoV-2 delta variant spike protein and 5, 10, 15, 20, 30, 40, 50, 75 or more amino acid residues C-terminus to the ectodomain of the SARS-CoV-2 delta variant spike protein. In some embodiments, a portion of a SARS-CoV-2 spike protein comprises the ectodomain of the SARS-CoV-2 delta variant spike protein and 5 to 25, 5 to 50, 25 to 50, 25 to 75, or 50 to 75 amino acid residues N-terminus to the ectodomain of the SARS-CoV-2 delta variant spike protein, 5 to 25, 5 to 50, 25 to 50, 25 to 75, or 50 to 75 amino acid residues ectodomain to the ectodomain of the SARS-CoV-2 delta variant spike protein, or 5 to 25, 5 to 50, 25 to 50, 25 to 75, or 50 to 75 amino acid residues N-terminus to the ectodomain of SARS-CoV-2 delta variant spike protein and 5 to 25, 5 to 50, 25 to 50, 25 to 75, or 50 to 75 amino acid residues C-terminus to the ectodomain of the SARS-CoV-2 delta variant spike protein.

In certain embodiments, a portion of a SARS-CoV-2 delta variant spike protein comprises 200, 220, 222, 250, 300, 350, 400, or more amino acid residues. In some embodiments, a portion of a SARS-CoV-2 delta variant spike protein comprises 450, 500, 550, 600, 650, 700, 750, 800, 850, 900, 950, 1000, 1100, 1200 or more. In some embodiments, a portion of a SARS-CoV-2 delta variant spike protein comprises 200 to 400, 200 to 600, 300 to 400, 500 to 800, 500 to 1000, 800 to 1000, 800 to 1100, 800 to 1200, or 1000 to 1200 amino acid residues. In some embodiments, a portion of a SARS-CoV-2 delta variant spike protein comprises at least 200, at least 300, at least 400, at least 500, at least 550, at least 600, at least 650, at least 700, at least 750, at least 800, at least 850, at least 900, at least 950, at least 1000, at least 1100, or at least 1200 amino acid residues, but less than the full length SARS-CoV-2 delta variant spike protein.

In another embodiment, described herein is a transgene comprising a nucleotide sequence encoding a full-length SARS-CoV-2 delta variant spike protein or a fragment thereof. In a specific embodiment, the SARS-CoV-2 delta variant is of the B1.617.2 sublineage. In certain embodiments, the SARS-CoV-2 delta variant is of the AY sublineage. In certain embodiments, the protein further comprises a domain(s) that facilitate purification, folding and cleavage of portions of a polypeptide. For example, a His tag (His-His-His-His-His-His) (SEQ ID NO:25), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In certain embodiments, a fragment of the SARS-CoV-2 delta variant spike protein is at least 1000, 1025, 1075, 1100, 1125, 1150, 1200 or 1215 amino acid residues in length. In some embodiments, a fragment of the SARS-CoV-2 delta variant spike protein is 200 to 400, 200 to 600, 300 to 400, 500 to 800, 500 to 1000, 800 to 1000, 800 to 1100, 800 to 1200, or 1000 to 1200 contiguous amino acid residues in length. In some embodiments, a fragment of the SARS-CoV-2 delta variant spike protein is at least 200, at least 300, at least 400, at least 500, at least 550, at least 600, at least 650, at least 700, at least 750, at least 800, at least 850, at least 900, at least 950, at least 1000, at least 1100, or at least 1200 contiguous amino acid residues in length, but less than the full length SARS-CoV-2 delta variant spike protein.

In another embodiment, provided herein is a transgene comprising a nucleotide sequence that is at least 85%, at least 90%, at least 95%, at least 96%, at least 97%, at least 98% or at least 99% identical to the nucleotide sequence of a SAR-CoV-2 delta variant spike protein, or a portion thereof (e.g., ectodomain, S1 domain, S2 domain, or a receptor binding domain), or a fragment thereof. In another embodiment, provided herein is a transgene comprising a nucleotide sequence that is at least 95%, at least 96%, at least 97%, at least 98% or at least 99% identical to the nucleotide sequence of a SAR-CoV-2 delta variant spike protein, or a portion thereof (e.g., ectodomain, S1 domain, S2 domain, or a receptor binding domain), or a fragment thereof. In another embodiment, provided herein is a transgene comprising a nucleotide sequence that is at least 96%, at least 97%, at least 98% or at least 99% identical to the nucleotide sequence of a SAR-CoV-2 delta variant spike protein, or a portion thereof (e.g., ectodomain, S1 domain, S2 domain, or a receptor binding domain), or a fragment thereof. Methods/techniques known in the art may be used to determine sequence identity (see, e.g., “Best Fit” or “Gap” program of the Sequence Analysis Software Package, version 10; Genetics Computer Group, Inc.). In certain embodiments, the protein further comprises one or more polypeptide domains. The one or more polypeptide domains may be at the C-terminus or N-terminus. In a specific embodiment, the one or more polypeptide domains are at the C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His) (SEQ ID NO:25), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n (SEQ ID NO:25), wherein n is 6. In certain embodiments, a fragment of the SARS-CoV-2 spike protein is at least 250, at least 500, at least 750, at least 1000, at least 1025, at least 1075, at least 1100, at least 1125, at least 1150, at least 1175, at least 1200, or at least 1215 amino acid residues in length. In some embodiments, a fragment of the SARS-CoV-2 delta variant spike protein is 200 to 400, 200 to 600, 300 to 400, 500 to 800, 500 to 1000, 800 to 1000, 800 to 1100, 800 to 1200, or 1000 to 1200 contiguous amino acid residues in length. In some embodiments, a fragment of the SARS-CoV-2 delta variant spike protein is at least 200, at least 300, at least 400, at least 500, at least 550, at least 600, at least 650, at least 700, at least 750, at least 800, at least 850, at least 900, at least 950, at least 1000, at least 1100, or at least 1200 contiguous amino acid residues in length, but less than the full length SARS-CoV-2 delta variant spike protein.

Techniques known to one of skill in the art can be used to determine the percent identity between two amino acid sequences or between two nucleotide sequences. Generally, to determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length. In a certain embodiment, the percent identity is determined over the entire length of an amino acid sequence or nucleotide sequence. In some embodiments, the length of sequence identity comparison may be over the full-length of the two sequences being compared (e.g., the full-length of a gene coding sequence, or a fragment thereof). In some embodiments, a fragment of a nucleotide sequence is at least 25, at least 50, at least 75, or at least 100 nucleotides. Similarly, “percent sequence identity” may be readily determined for amino acid sequences, over the full-length of a protein, or a fragment thereof. In some embodiments, a fragment of a protein comprises at least 20, at least 30, at least 40, at least 50 or more contiguous amino acids of the protein. In certain embodiments, a fragment of a protein comprises at least 75, at least 100, at least 125, at least 150 or more contiguous amino acids of the protein. In some embodiments, a fragment of the SARS-CoV-2 delta variant spike protein is 75 to 200, 100 to 200, 125 to 200, 200 to 300, or 100 to 300 contiguous amino acid residues of the protein.

The determination of percent identity between two sequences (e.g., amino acid sequences or nucleic acid sequences) can be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264 2268, modified as in Karlin and Altschul, 1993, Proc. Natl. Acad. Sci. U.S.A. 90:5873 5877. Such an algorithm is incorporated into the NBLAST and XBLAST programs of Altschul et al., 1990, J. Mol. Biol. 215:403. BLAST nucleotide searches can be performed with the NBLAST nucleotide program parameters set, e.g., for score=100, wordlength=12 to obtain nucleotide sequences homologous to nucleic acid molecules described herein. BLAST protein searches can be performed with the XBLAST program parameters set, e.g., to score 50, wordlength=3 to obtain amino acid sequences homologous to a protein molecule described herein. To obtain gapped alignments for comparison purposes, Gapped BLAST can be utilized as described in Altschul et al., 1997, Nucleic Acids Res. 25:3389 3402. Alternatively, PSI BLAST can be used to perform an iterated search which detects distant relationships between molecules (Id.). When utilizing BLAST, Gapped BLAST, and PSI Blast programs, the default parameters of the respective programs (e.g., of XBLAST and NBLAST) can be used (see, e.g., National Center for Biotechnology Information (NCBI) on the worldwide web, ncbi.nlm.nih.gov). Another preferred, non-limiting example of a mathematical algorithm utilized for the comparison of sequences is the algorithm of Myers and Miller, 1988, CABIOS 4:11 17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

In another embodiment, provided herein is a transgene comprising a nucleotide sequence encoding a protein comprising (or consisting of) a SARS-CoV-2 delta variant spike protein with 1, 2, 3, 4, 5, 6, 7, 8, 9, 10, 11, or 12 amino acids substituted with another amino acid (e.g., a conservative amino acid substitution). In another embodiment, provided herein is a transgene comprising a nucleotide sequence encoding a protein comprising (or consisting of) a SARS-CoV-2 delta variant spike protein with 1, 2, 3, 4, 5, 6, 7, 8, 9, 10, 11, or 12 amino acids at the C-terminus substituted with another amino acid (e.g., a conservative amino acid substitution). In another embodiment, provided herein is a transgene comprising a nucleotide sequence encoding a protein comprising (or consisting of) a SARS-CoV-2 delta variant spike protein with 1, 2, 3, 4, 5, 6, 7, 8, 9, 10, 11, or 12 amino acids at the N-terminus substituted with another amino acid (e.g., a conservative amino acid substitution). In another embodiment, provided herein is a transgene comprising a nucleotide sequence encoding a protein comprising (or consisting of) a SARS-CoV-2 delta variant spike protein with 1, 2, 3, 4, 5, 6, 7, 8, 9, 10, 11, or 12 amino acids at the N-terminus substituted with another amino acid (e.g., a conservative amino acid substitution) and 1, 2, 3, 4, 5, 6, 7, 8, 9, 10, 11, or 12 amino acids at the C-terminus substituted with another amino acid (e.g., a conservative amino acid substitution). In a specific embodiment, the N-terminus is the first 100 amino acid residues of the SARS-CoV-2 delta variant spike protein. In a specific embodiment, the C-terminus is the last 100 amino acid residues of the SARS-CoV-2 delta variant spike protein. In specific embodiments, the SARS-CoV-2 delta variant spike protein is the mature form of the protein. In other embodiments, the SARS-CoV-2 delta variant spike protein is the immature form of the protein. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophilic (Cys, Ser, Thr), acidic (Asp, Glu), basic (Asn, Gln, His, Lys, Arg), conformation disruptors (Gly, Pro) and aromatic (Trp, Tyr, Phe). In certain embodiments, the protein further comprise one or more polypeptide domains. The one or more polypeptide domains may be at the C-terminus or N-terminus. In a specific embodiment, the one or more polypeptide domains are at the C-terminus Useful polypeptide domains include domains that facilitate purification, folding and cleavage of portions of a polypeptide. For example, a His tag (His-His-His-His-His-His) (SEQ ID NO:25), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n (SEQ ID NO:25), wherein n is 6.

In another embodiment, provided herein is a transgene comprising a nucleotide sequence encoding a protein comprising (or consisting of) a SARS-CoV-2 delta variant spike protein with 1, 2, 3, 4, 5, 6, 7, 8, 9, 10, 11, or 12 amino acids deleted. In another embodiment, provided herein is a transgene comprising a nucleotide sequence encoding a protein comprising (or consisting of) a SARS-CoV-2 spike protein with 1, 2, 3, 4, 5, 6, 7, 8, 9, 10, 11, or 12 amino acids deleted from the C-terminus. In another embodiment, provided herein is a transgene comprising a nucleotide sequence encoding a protein comprising (or consisting of) a SARS-CoV-2 spike protein with 1, 2, 3, 4, 5, 6, 7, 8, 9, 10, 11, or 12 amino acids deleted from the N-terminus. In a specific embodiment, the N-terminus is the first 100 amino acid residues of the SARS-CoV-2 delta variant spike protein. In a specific embodiment, the C-terminus is the last 100 amino acid residues of the SARS-CoV-2 delta variant spike protein. In specific embodiments, the SARS-CoV-2 delta variant spike protein is the mature form of the protein. In other embodiments, the SARS-CoV-2 delta variant spike protein is the immature form of the protein. In certain embodiments, the protein further comprises one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His) (SEQ ID NO:25), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n (SEQ ID NO:25), wherein n is 6.

In another embodiment, provided herein is a transgene comprising a nucleotide sequence encoding a protein comprising (or consisting of) a SARS-CoV-2 delta variant spike protein with 1, 2, 3, 4, 5, 6, 7, 8, 9, 10, 11, 12, 13, 14, or 15 mutations (e.g., amino acid substitutions, amino acid deletions, amino acid additions, or a combination thereof). In some embodiments, one or more of the mutations are at the N-terminus, the C-terminus, or both the N- and C-termini. In another embodiment, provided herein is a transgene comprising a nucleotide sequence encoding a protein comprising (or consisting of) a SARS-CoV-2 delta variant spike protein with 1, 2, 3, 4, 5, 6, 7, 8, 9, 10, 11, or 12 amino acids deleted and 1, 2, 3, 4, 5, 6, 7, 8, 9, 10, 11 or 12 amino acid substitutions. In a specific embodiment, the N-terminus is the first 100 amino acid residues of the SARS-CoV-2 delta variant spike protein. In a specific embodiment, the C-terminus is the last 100 amino acid residues of the SARS-CoV-2 delta variant spike protein. In specific embodiments, the SARS-CoV-2 delta variant spike protein is the mature form of the protein. In other embodiments, the SARS-CoV-2 delta variant spike protein is the immature form of the protein. In certain embodiments, the protein further comprises one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His) (SEQ ID NO:25), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n (SEQ ID NO:25), wherein n is 6.

In another embodiment, described herein is a transgene comprising a nucleotide sequence encoding a protein comprising (or consisting of) the receptor binding domain of a SARS-CoV-2 delta variant spike protein. In certain embodiments, protein further comprise one or more polypeptide domains. Useful polypeptide domains include domains that facilitate purification, folding and cleavage of portions of a polypeptide. For example, a His tag (His-His-His-His-His-His) (SEQ ID NO:25), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In a specific embodiment, a protein comprises or consists of the receptor binding domain of a SARS-CoV-2 delta variant spike protein and a His tag (e.g., a (His)n (SEQ ID NO:25), where n is 6). In certain embodiments, a protein comprising (or consisting) of the receptor binding domain of a SARS-CoV-2 delta variant spike polypeptide is a secreted polypeptide. In a specific embodiment, when designing a protein comprising SARS-CoV-2 delta variant spike polypeptide receptor binding domain, care is taken to maintain the stability of the resulting protein.

In another embodiment, provided herein is a transgene comprising a nucleotide sequence encoding a protein comprising (or consisting of) a SARS-CoV-2 delta variant spike protein receptor binding domain with 1, 2, 3, 4, 5, 6, 7, 8, 9, 10, 11, or 12 amino acids substituted with another amino acid (e.g., a conservative amino acid substitution). In another embodiment, provided herein is a transgene comprising a nucleotide sequence encoding a protein comprising (or consisting of) a SARS-CoV-2 delta variant spike protein receptor binding domain with 1, 2, 3, 4, 5, 6, 7, 8, 9, 10, 11, or 12 amino acids at the C-terminus of the receptor binding domain substituted with another amino acid (e.g., a conservative amino acid substitution). In another embodiment, provided herein is a transgene comprising a nucleotide sequence encoding a protein comprising (or consisting of) a SARS-CoV-2 delta variant spike protein receptor binding domain with 1, 2, 3, 4, 5, 6, 7, 8, 9, 10, 11, or 12 amino acids at the N-terminus of the receptor binding domain substituted with another amino acid (e.g., a conservative amino acid substitution). In another embodiment, provided herein is a transgene comprising a nucleotide sequence encoding a protein comprising (or consisting of) a SARS-CoV-2 delta variant spike protein receptor binding domain with 1, 2, 3, 4, 5, 6, 7, 8, 9, 10, 11, or 12 amino acids at the N-terminus of the receptor binding domain substituted with another amino acid (e.g., a conservative amino acid substitution) and 1, 2, 3, 4, 5, 6, 7, 8, 9, 10, 11, or 12 amino acids at the C-terminus of the receptor binding domain substituted with another amino acid (e.g., a conservative amino acid substitution). In a specific embodiment, the N-terminus is the first 25 amino acid residues of the receptor binding domain of the SARS-CoV-2 delta variant spike protein. In a specific embodiment, the C-terminus is the last 25 amino acid residues of the receptor binding domain of the SARS-CoV-2 delta variant spike protein. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophilic (Cys, Ser, Thr), acidic (Asp, Glu), basic (Asn, Gln, His, Lys, Arg), conformation disruptors (Gly, Pro) and aromatic (Trp, Tyr, Phe). In certain embodiments, the protein further comprises one or more polypeptide domains. The one or more polypeptide domains may be at the C-terminus or N-terminus. In a specific embodiment, the one or more polypeptide domains are at the C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His) (SEQ ID NO:25), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n (SEQ ID NO:25), wherein n is 6.

In another embodiment, provided herein is a transgene comprising a nucleotide sequence encoding a protein comprising (or consisting of) a SARS-CoV-2 delta variant spike protein receptor binding domain with 1, 2, 3, 4, 5, 6, 7, 8, 9, 10, 11, or 12 amino acids deleted from the C-terminus of the receptor binding domain. In another embodiment, provided herein is a transgene comprising a nucleotide sequence encoding a protein comprising (or consisting of) a SARS-CoV-2 delta variant spike protein receptor binding domain with 1, 2, 3, 4, 5, 6, 7, 8, 9, 10, 11, or 12 amino acids deleted from the N-terminus of the receptor binding domain. In another embodiment, provided herein is a transgene comprising a nucleotide sequence encoding a protein comprising (or consisting of) a SARS-CoV-2 delta variant spike protein receptor binding domain with 1, 2, 3, 4, 5, 6, 7, 8, 9, 10, 11, or 12 amino acids deleted from the N-terminus of the receptor binding domain and 1, 2, 3, 4, 5, 6, 7, 8, 9, 10, 11, or 12 amino acids deleted from the C-terminus of the receptor binding domain. In a specific embodiment, the N-terminus is the first 25 amino acid residues of the receptor binding domain of the SARS-CoV-2 delta variant spike protein. In a specific embodiment, the C-terminus is the last 25 amino acid residues of the receptor binding domain of the SARS-CoV-2 delta variant spike protein. In certain embodiments, the protein further comprises one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His) (SEQ ID NO:25), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n (SEQ ID NO:25), wherein n is 6.

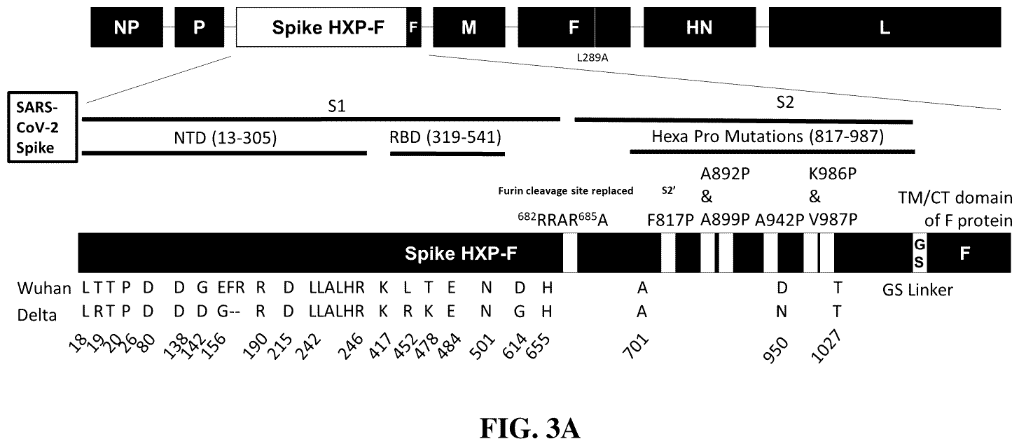

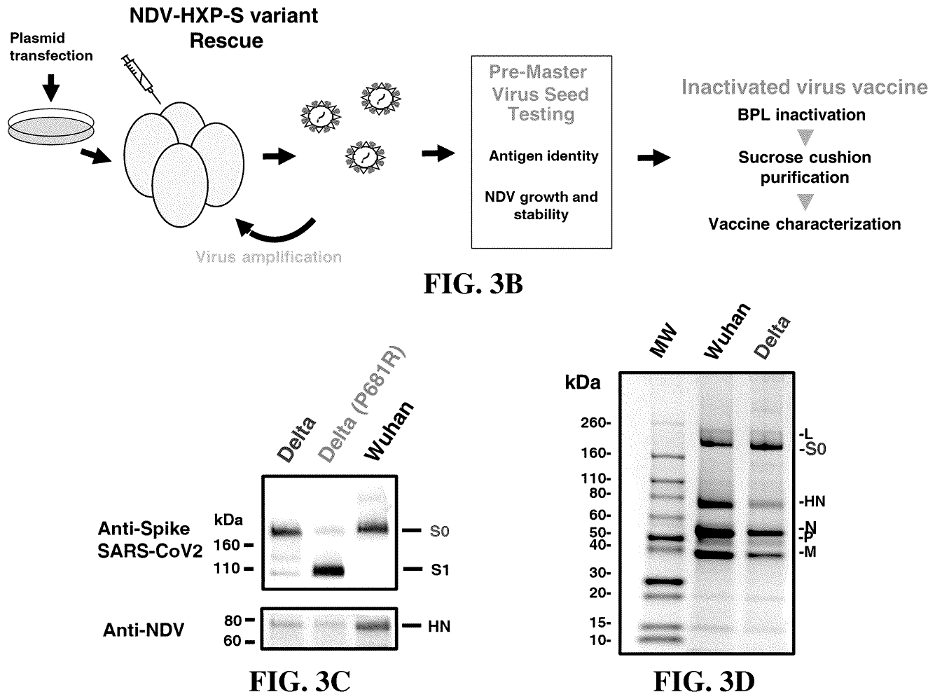

In another embodiment, described herein is a transgene comprising a nucleotide sequence encoding a protein comprising (or consisting of) the ectodomain of a delta variant SARS-CoV-2 spike protein. In another embodiment, described herein is a transgene comprising a nucleotide sequence encoding a protein comprising (or consisting of) a derivative of the ectodomain of a delta variant SARS-CoV-2 spike protein. In some embodiments, described herein is a transgene comprising a nucleotide sequence encoding a protein comprising (or consisting of) a derivative of the ectodomain of a delta variant SARS-CoV-2 spike protein, wherein the derivative lacks the polybasic cleavage site of the ectodomain (e.g., one, two or more residues corresponding to amino acid residues 682 to 685 (RRAR) of the SARS-CoV-2 spike protein found at GenBank Accession No. MN908947.3 are substituted for other amino acid residues). In a specific embodiment, amino acid residues corresponding to amino acid residues 682 to 685 (RRAR) of the SARS-CoV-2 spike protein found at GenBank Accession No. MN908947.3 are substituted with a single alanine. In some embodiments, the derivative of the SARS-CoV-2 delta variant spike protein ectodomain comprises the amino acid sequence of SEQ ID NO:12 with one, two, three, four, five, six, seven, eight, or all of the following amino acid modifications: T19R, G142D, delE156, delF157, R158G, L452R, T478K, D614G, P681R, and D950N. In specific embodiments, the derivative of the SARS-CoV-2 delta variant spike protein ectodomain does not comprise the amino acid substitution of P681R. In a specific embodiment, the derivative of the SARS-CoV-2 delta variant spike protein ectodomain comprises the amino acid sequence of SEQ ID NO:12 with one, two, three, four, five, six, seven, or all of the following amino acid modifications: T19R, G142D, delE156, delF157, R158G, L452R, T478K, D614G, and D950N. In a specific embodiment, the derivative of the SARS-CoV-2 delta variant spike protein ectodomain comprises the amino acid sequence of SEQ ID NO:12 with the following amino acid modifications: T19R, G142D, delE156, delF157, R158G, L452R, T478K, D614G, and D950N. In a specific embodiment, the derivative of the SARS-CoV-2 delta variant spike protein ectodomain comprises the amino acid sequence of SEQ ID NO:12 with the amino acid modifications shown in FIG. 3A for Delta. In another specific embodiment, the derivative of the SARS-CoV-2 delta variant spike protein ectodomain comprises the amino acid sequence of SEQ ID NO:12 with one, two, three, four, five, six, seven, eight, nine, ten, eleven, twelve, thirteen, fourteen, or all of the following amino acid modifications: T19R, T95I, G142D, delE156, delF157, R158G, A222V, W258L, K417N, L452R, T478K, D614G, P681R, and D950N. In specific embodiments, the derivative of the SARS-CoV-2 delta variant spike protein ectodomain does not comprise the amino acid substitution of P681R. In some embodiments, the derivative of the SARS-CoV-2 delta variant spike protein ectodomain comprises the amino acid sequence of SEQ ID NO:12 with one, two, three, four, five, six, seven, eight, nine, ten, eleven, twelve, thirteen, or all of the following amino acid modifications: T19R, T95I, G142D, delE156, delF157, R158G, A222V, W258L, K417N, L452R, T478K, D614G, and D950N. In certain embodiments, the derivative of the SARS-CoV-2 delta variant spike protein ectodomain comprises the amino acid sequence of SEQ ID NO:12 with the following amino acid modifications: T19R, T95I, G142D, delE156, delF157, R158G, A222V, W258L, K417N, L452R, T478K, D614G, and D950N. In some embodiments, the derivative of the SARS-CoV-2 delta variant spike ectodomain comprises the amino acid sequence of SEQ ID NO:16 with one, two, three, four, five, six, seven, eight, or all of the following amino acid modifications: T6R, G129D, delE143, delF144, R145G, L439R, T465K, D601G, and D937N. In certain embodiments, protein further comprises one or more polypeptide domains. Useful polypeptide domains include domains that facilitate purification, folding and cleavage of portions of a polypeptide. For example, a His tag (His-His-His-His-His-His) (SEQ ID NO:25), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In a specific embodiment, a protein comprises or consists of the ectodomain of a SARS-CoV-2 spike protein and a His tag (e.g., a (His)n (SEQ ID NO:25), where n is 6). In certain embodiments, a protein comprising (or consisting) of the ectodomain of a SARS-CoV-2 delta variant spike polypeptide or a derivative thereof is a secreted polypeptide. In specific embodiments, a protein comprising the ectodomain of a SARS-CoV-2 delta variant spike protein or a derivative thereof comprises NDV F protein transmembrane and cytoplasmic domains. In certain embodiments, a protein comprising the ectodomain of a SARS-CoV-2 delta variant spike polypeptide or a derivative thereof comprises one or more trimerization domains known to one of skill in the art (e.g., a T4 foldon trimerization domain), and optionally a tag (e.g., a His tag or Flag tag). In some embodiments, a protein comprising the ectodomain of a SARS-CoV-2 delta variant spike protein or derivative thereof comprises C-terminus a thrombin cleavage site and a T4 foldon trimerization domain. In some embodiments, a protein comprising the ectodomain of a SARS-CoV-2 delta variant spike protein or derivative thereof comprises C-terminus a thrombin cleavage site, a T4 foldon trimerization domain, and a hexahistidine tag. In a specific embodiment, when designing a protein comprising SARS-CoV-2 delta variant spike polypeptide ectodomain or a derivative thereof, care is taken to maintain the stability of the resulting protein.

In some embodiments, described herein is a transgene comprising a polynucleotide sequence encoding a derivative of the ectodomain of a SARS-CoV-2 spike protein, wherein the polynucleotide sequence comprises a nucleotide sequence at least 80%, at least 85%, or at least 90% identical to the nucleotide sequence of SEQ ID NO:19 or 20. In some embodiments, described herein is a transgene comprising a polynucleotide sequence encoding a derivative of the ectodomain of a SARS-CoV-2 spike protein, wherein the polynucleotide sequence comprises a nucleotide sequence at least 95%, at least 98%, or at least 99% identical to the nucleotide sequence of SEQ ID NO:19 or 20. In some embodiments, described herein is a transgene comprising a polynucleotide sequence encoding a derivative of the ectodomain of a SARS-CoV-2 spike protein, wherein the polynucleotide sequence comprises the nucleotide sequence of SEQ ID NO:19 or 20.

In another embodiment, provided herein is a transgene comprising a nucleotide sequence encoding a protein comprising (or consisting of) a SARS-CoV-2 delta variant spike protein ectodomain with 1, 2, 3, 4, 5, 6, 7, 8, 9, 10, 11, or 12 amino acids substituted with another amino acid (e.g., a conservative amino acid substitution). In another embodiment, provided herein is a transgene comprising a nucleotide sequence encoding a protein comprising (or consisting of) a SARS-CoV-2 delta variant spike protein ectodomain with 1, 2, 3, 4, 5, 6, 7, 8, 9, 10, 11, or 12 amino acids at the C-terminus of the ectodomain substituted with another amino acid (e.g., a conservative amino acid substitution). In another embodiment, provided herein is a transgene comprising a nucleotide sequence encoding a protein comprising (or consisting of) a SARS-CoV-2 delta variant spike protein ectodomain with 1, 2, 3, 4, 5, 6, 7, 8, 9, 10, 11, or 12 amino acids at the N-terminus of the ectodomain substituted with another amino acid (e.g., a conservative amino acid substitution). In another embodiment, provided herein is a transgene comprising a nucleotide sequence encoding a protein comprising (or consisting of) a SARS-CoV-2 delta variant spike protein ectodomain with 1, 2, 3, 4, 5, 6, 7, 8, 9, 10, 11, or 12 amino acids at the N-terminus of the ectodomain substituted with another amino acid (e.g., a conservative amino acid substitution) and 1, 2, 3, 4, 5, 6, 7, 8, 9, 10, 11, or 12 amino acids at the C-terminus of the ectodomain substituted with another amino acid (e.g., a conservative amino acid substitution). In a specific embodiment, the C-terminus of the ectodomain is the last 100 amino acid residues. In a specific embodiment, the N-terminus of the ectodomain is the first 100 amino acid residues. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophilic (Cys, Ser, Thr), acidic (Asp, Glu), basic (Asn, Gln, His, Lys, Arg), conformation disruptors (Gly, Pro) and aromatic (Trp, Tyr, Phe). In certain embodiments, the SARS-CoV-2 spike protein ectodomain lacks the polybasic cleavage site (e.g., one, two or more residues corresponding to amino acid residues 682 to 685 (RRAR) of the SARS-CoV-2 spike protein found at GenBank Accession No. MN908947.3 are substituted for other amino acid residues). In a specific embodiment, amino acid residues corresponding to amino acid residues 682 to 685 (RRAR) of the SARS-CoV-2 spike protein found at GenBank Accession No. MN908947.3 are substituted with a single alanine. In certain embodiments, the protein further comprises one or more polypeptide domains. The one or more polypeptide domains may be at the C-terminus or N-terminus. In a specific embodiment, the one or more polypeptide domains are at the C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His) (SEQ ID NO:25), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n (SEQ ID NO:25), wherein n is 6. In certain embodiments, a protein comprising (or consisting) of the ectodomain of a SARS-CoV-2 delta variant spike polypeptide is a secreted polypeptide. In certain embodiments, a protein comprising the ectodomain of a SARS-CoV-2 delta variant spike polypeptide comprises one or more trimerization domains known to one of skill in the art (e.g., a T4 foldon trimerization domain), and optionally a tag (e.g., a His tag or Flag tag). In some embodiments, a protein comprising the ectodomain of a SARS-CoV-2 delta variant spike protein or derivative thereof comprises C-terminus a thrombin cleavage site and a T4 foldon trimerization domain. In some embodiments, a protein comprising the ectodomain of a SARS-CoV-2 delta variant spike protein or derivative thereof comprises C-terminus a thrombin cleavage site, a T4 foldon trimerization domain, and a hexahistidine tag. In specific embodiments, a protein comprising the ectodomain of a SARS-CoV-2 delta variant spike polypeptide comprises NDV F protein transmembrane and cytoplasmic domains.

In another embodiment, provided herein is a transgene comprising a nucleotide sequence encoding a protein comprising (or consisting of) an amino acid sequence at least 85%, at least 90%, at least 95%, at least 96%, at least 97%, at least 98%, at least 99%, or at least 99.5% identical to the amino acid sequence of SEQ ID NO:13. In another embodiment, provided herein is a transgene comprising a nucleotide sequence encoding a protein comprising (or consisting of) an amino acid sequence at least 96%, at least 97%, at least 98%, at least 99%, or at least 99.5% identical to the amino acid sequence of SEQ ID NO:13. In another embodiment, provided herein is a transgene comprising a nucleotide sequence encoding a protein comprising (or consisting of) the amino acid sequence of SEQ ID NO:13. In another embodiment, provided herein is a transgene comprising a nucleotide sequence encoding a protein comprising (or consisting of) the amino acid sequence of SEQ ID NO:13 minus the signal peptide. See SEQ ID NO:15 for the signal peptide. In another embodiment, provided herein is a transgene comprising a nucleotide sequence encoding a protein comprising (or consisting of) an amino acid sequence at least 85%, at least 90%, at least 95%, at least 96%, at least 97%, at least 98%, at least 99%, or at least 99.5% identical to the amino acid sequence of SEQ ID NO:17. In another embodiment, provided herein is a transgene comprising a nucleotide sequence encoding a protein comprising (or consisting of) an amino acid sequence at least 96%, at least 97%, at least 98%, at least 99%, or at least 99.5% identical to the amino acid sequence of SEQ ID NO:17. In another embodiment, provided herein is a transgene comprising a nucleotide sequence encoding a protein comprising (or consisting of) the amino acid sequence of SEQ ID NO:17. In certain embodiments, the protein further comprises one or more polypeptide domains. The one or more polypeptide domains may be at the C-terminus, N-terminus, or C-terminus and N-terminus In a specific embodiment, the one or more polypeptide domains are at the C-terminus. Useful polypeptide domains include domains that facilitate purification, folding and cleavage of portions of a polypeptide. For example, a His tag (His-His-His-His-His-His) (SEQ ID NO:25), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n (SEQ ID NO:25), wherein n is 6. In certain embodiments, the protein is a secreted polypeptide. In some embodiments, a protein further comprises NDV F protein transmembrane and cytoplasmic domains. In some embodiments, a protein further comprises one or more trimerization domains known to one of skill in the art (e.g., a T4 foldon trimerization domain), and optionally a tag (e.g., a His tag or Flag tag). In some embodiments, a protein comprising further comprises C-terminus a thrombin cleavage site and a T4 foldon trimerization domain. In some embodiments, a protein further comprises C-terminus a thrombin cleavage site, a T4 foldon trimerization domain, and a hexahistidine tag.

In another embodiment, provided herein is a transgene comprising a nucleotide sequence encoding a protein comprising (or consisting of) an amino acid sequence at least 85%, at least 90%, at least 95%, at least 96%, at least 97%, at least 98%, at least 99%, or at least 99.5% identical to the amino acid sequence of SEQ ID NO:13. In another embodiment, provided herein is a transgene comprising a nucleotide sequence encoding a protein comprising (or consisting of) an amino acid sequence at least 96%, at least 97%, at least 98%, at least 99%, or at least 99.5% identical to the amino acid sequence of SEQ ID NO:13. In another embodiment, provided herein is a transgene comprising a nucleotide sequence encoding a protein comprising (or consisting of) the amino acid sequence of SEQ ID NO:13. In another embodiment, provided herein is a transgene comprising a nucleotide sequence encoding a protein comprising (or consisting of) the amino acid sequence of SEQ ID NO:13 minus the signal peptide. See SEQ ID NO:15 for the signal peptide. In another embodiment, provided herein is a transgene comprising a nucleotide sequence encoding a protein comprising (or consisting of) an amino acid sequence at least 85%, at least 90%, at least 95%, at least 96%, at least 97%, at least 98%, at least 99%, or at least 99.5% identical to the amino acid sequence of SEQ ID NO:17. In another embodiment, provided herein is a transgene comprising a nucleotide sequence encoding a protein comprising (or consisting of) an amino acid sequence at least 96%, at least 97%, at least 98%, at least 99%, or at least 99.5% identical to the amino acid sequence of SEQ ID NO:17. In another embodiment, provided herein is a transgene comprising a nucleotide sequence encoding a protein comprising (or consisting of) the amino acid sequence of SEQ ID NO:17. In certain embodiments, the protein further comprises one or more polypeptide domains. The one or more polypeptide domains may be at the C-terminus or N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His) (SEQ ID NO:25), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n (SEQ ID NO:25), wherein n is 6. In certain embodiments, the protein is a secreted polypeptide. In certain embodiments, a protein further comprises one or more trimerization domains known to one of skill in the art (e.g., a T4 foldon trimerization domain), and optionally a tag (e.g., a His tag or Flag tag). In some embodiments, a protein further comprises C-terminus a thrombin cleavage site and a T4 foldon trimerization domain. In some embodiments, a protein further comprises C-terminus a thrombin cleavage site, a T4 foldon trimerization domain, and a hexahistidine tag.

In another embodiment, provided herein is a transgene comprising a nucleotide sequence encoding a protein comprising (or consisting of) an amino acid sequence at least 85%, at least 90%, at least 95%, at least 96%, at least 97%, at least 98%, at least 99%, or at least 99.5% identical to the amino acid sequence of SEQ ID NO:13 or 17, wherein the protein comprises: (1) amino acid substitutions to proline at two, three, four, five or all of the following amino acid positions 817, 892, 899, 942, 986, and 987 (as counted based on the SARS-CoV-2 spike protein found at GenBank Accession MN908947.3); and (2) RRAR to A amino acid substitution at amino acid positions 682 to 685 (as counted based on the SARS-CoV-2 spike protein found at GenBank Accession MN908947.3). In another embodiment, provided herein is a transgene comprising a nucleotide sequence encoding a protein comprising (or consisting of) an amino acid sequence at least 85%, at least 90%, at least 95%, at least 96%, at least 97%, at least 98%, at least 99%, or at least 99.5% identical to the amino acid sequence of SEQ ID NO:13 or 17, wherein the protein comprises: (1) amino acid substitutions to proline at two, three, four, five or all of the following amino acid positions 817, 892, 899, 942, 986, and 987 (as counted based on the SARS-CoV-2 spike protein found at GenBank Accession MN908947.3); (2) RRAR to A amino acid substitution at amino acid positions 682 to 685 (as counted based on the SARS-CoV-2 spike protein found at GenBank Accession MN908947.3); and (3) amino acid substitutions at two, three, four, five, six, seven, eight, or all of the following amino acid positions 19, 142, 156, 157, 158, 452, 478, 614, and 950 (as counted based on the SARS-CoV-2 spike protein found at GenBank Accession MN908947.3), wherein the substitutions are selected from T19R, G142D, E156G, delF157, delR158, L452R, T478K, D614G, and D950N. In another embodiment, provided herein is a transgene comprising a nucleotide sequence encoding a protein comprising (or consisting of) an amino acid sequence at least 85%, at least 90%, at least 95%, at least 96%, at least 97%, at least 98%, at least 99%, or at least 99.5% identical to the amino acid sequence of SEQ ID NO:13 or 17, wherein the protein comprises: (1) amino acid substitutions to proline at amino acid positions 817, 892, 899, 942, 986, and 987 (as counted based on the SARS-CoV-2 spike protein found at GenBank Accession MN908947.3); (2) RRAR to A amino acid substitution at amino acid positions 682 to 685 (as counted based on the SARS-CoV-2 spike protein found at GenBank Accession MN908947.3); and (3) amino acid substitutions at the following amino acid positions 19, 142, 156, 157, 158, 452, 478, 614, and 950 (as counted based on the SARS-CoV-2 spike protein found at GenBank Accession MN908947.3), wherein the substitutions are T19R, G142D, E156G, delF157, delR158, L452R, T478K, D614G, and D950N. In specific embodiments, the protein comprises a proline at amino acid position 681 (as counted based on the SARS-CoV-2 spike protein found at GenBank Accession MN908947.3).

In another embodiment, provided herein is a transgene comprising a nucleotide sequence encoding a protein comprising (or consisting of) a SARS-CoV-2 delta variant spike protein ectodomain with 1, 2, 3, 4, 5, 6, 7, 8, 9, 10, 11, or 12 amino acid substitutions and 1, 2, 3, 4, 5, 6, 7, 8, 9, 10, 11, or 12 amino acids deleted. In another embodiment, provided herein is a transgene comprising a nucleotide sequence encoding a protein comprising (or consisting of) a SARS-CoV-2 delta variant spike protein ectodomain with 1, 2, 3, 4, 5, 6, 7, 8, 9, 10, 11, or 12 amino acids deleted from the C-terminus. In another embodiment, provided herein is a transgene comprising a nucleotide sequence encoding a protein comprising (or consisting of) a SARS-CoV-2 delta variant spike protein ectodomain with 1, 2, 3, 4, 5, 6, 7, 8, 9, 10, 11, or 12 amino acids deleted from the N-terminus. In certain embodiments, the SARS-CoV-2 delta variant spike protein ectodomain lacks the polybasic cleavage site (e.g., amino acid residues 682 to 685 (RRAR) are substituted with a single alanine). In a specific embodiment, the C-terminus of the ectodomain is the last 100 amino acid residues. In a specific embodiment, the N-terminus of the ectodomain is the first 100 amino acid residues. In certain embodiments, the protein further comprise one or more polypeptide domains. The one or more polypeptide domains may be at the C-terminus or N-terminus. In a specific embodiment, the one or more polypeptide domains are at the C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His) (SEQ ID NO:25), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n (SEQ ID NO:25), wherein n is 6. In some embodiments, a protein that comprises the ectodomain of a SARS-CoV-2 delta variant spike polypeptide comprises NDV F protein transmembrane and cytoplasmic domains. In certain embodiments, a protein that comprises the ectodomain of a SARS-CoV-2 delta variant spike polypeptide comprises one or more trimerization domains known to one of skill in the art (e.g., a T4 foldon trimerization domain), and optionally a tag (e.g., a His tag or Flag tag). In some embodiments, a protein comprising the ectodomain of a SARS-CoV-2 delta variant spike protein comprises C-terminus a thrombin cleavage site and a T4 foldon trimerization domain. In some embodiments, a protein comprising the ectodomain of a SARS-CoV-2 delta variant spike protein comprises C-terminus a thrombin cleavage site, a T4 foldon trimerization domain, and a hexahistidine tag.

In another embodiment, provided herein is a transgene comprising a nucleotide sequence encoding a protein comprising (or consisting of) a SARS-CoV-2 delta variant spike protein ectodomain with 1, 2, 3, 4, 5, 6, 7, 8, 9, 10, 11, or 12 amino acids deleted. In another embodiment, provided herein is a transgene comprising a nucleotide sequence encoding a protein comprising (or consisting of) a SARS-CoV-2 delta variant spike protein ectodomain with 1, 2, 3, 4, 5, 6, 7, 8, 9, 10, 11, or 12 amino acids deleted from the C-terminus. In another embodiment, provided herein is a transgene comprising a nucleotide sequence encoding a protein comprising (or consisting of) a SARS-CoV-2 delta variant spike protein ectodomain with 1, 2, 3, 4, 5, 6, 7, 8, 9, 10, 11, or 12 amino acids deleted from the N-terminus. In certain embodiments, the SARS-CoV-2 delta variant spike protein ectodomain lacks the polybasic cleavage site (e.g., one, two or more residues corresponding to amino acid residues 682 to 685 (RRAR) of the SARS-CoV-2 spike protein found at GenBank Accession No. MN908947.3 are substituted for other amino acid residues). In a specific embodiment, amino acid residues corresponding to amino acid residues 682 to 685 (RRAR) of the SARS-CoV-2 spike protein found at GenBank Accession No. MN908947.3 are substituted with a single alanine. In certain embodiments, the protein further comprise one or more polypeptide domains. The one or more polypeptide domains may be at the C-terminus or N-terminus. In a specific embodiment, the one or more polypeptide domains are at the C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His) (SEQ ID NO:25), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In one embodiment, the His tag has the sequence (His)n (SEQ ID NO:25), wherein n is 6. In some embodiments, a protein that comprises the ectodomain of a SARS-CoV-2 delta variant spike polypeptide comprises NDV F protein transmembrane and cytoplasmic domains. In certain embodiments, a protein that comprises the ectodomain of a SARS-CoV-2 delta variant spike polypeptide comprises one or more trimerization domains known to one of skill in the art (e.g., a T4 foldon trimerization domain), and optionally a tag (e.g., a His tag or Flag tag). In some embodiments, a protein comprising the ectodomain of a SARS-CoV-2 delta variant spike protein comprises C-terminus a thrombin cleavage site and a T4 foldon trimerization domain. In some embodiments, a protein comprising the ectodomain of a SARS-CoV-2 delta variant spike protein comprises C-terminus a thrombin cleavage site, a T4 foldon trimerization domain, and a hexahistidine tag.

In another embodiment, provided herein is a transgene comprising a nucleotide sequence encoding a protein comprising (or consisting of) a SARS-CoV-2 delta variant spike protein ectodomain with 1, 2, 3, 4, 5, 6, 7, 8, 9, 10, 11, 12, 13, 14, or 15 mutations (e.g., amino acid substitutions, amino acid deletions, amino acid additions, or a combination thereof). In another embodiment, provided herein is a transgene comprising a nucleotide sequence encoding a protein comprising (or consisting of) a SARS-CoV-2 delta variant spike protein ectodomain with 1, 2, 3, 4, 5, 6, 7, 8, 9, 10, 11, or 12 amino acid substitutions and 1, 2, 3, 4, 5, 6, 7, 8, 9, 10, 11, or 12 amino acids deleted. In certain embodiments, the SARS-CoV-2 spike protein ectodomain lacks the polybasic cleavage site (e.g., one, two or more residues corresponding to amino acid residues 682 to 685 (RRAR) of the SARS-CoV-2 spike protein found at GenBank Accession No. MN908947.3 are substituted for other amino acid residues). In a specific embodiment, amino acid residues corresponding to amino acid residues 682 to 685 (RRAR) of the SARS-CoV-2 spike protein found at GenBank Accession No. MN908947.3 are substituted with a single alanine. In certain embodiments, the protein further comprise one or more polypeptide domains. The one or more polypeptide domains may be at the C-terminus or N-terminus. In a specific embodiment, the one or more polypeptide domains are at the C-terminus and N-terminus. In a specific embodiment, the one or more polypeptide domains are at the C-terminus. Useful polypeptide domains include domains that facilitate purification, folding and cleavage of portions of a polypeptide. For example, a His tag (His-His-His-His-His-His) (SEQ ID NO:25), FLAG epitope or other purification tag can facilitate purification of the protein provided herein. In some embodiments, the His tag has the sequence, (His)n, wherein n is 2, 3, 4, 5, 6, 7, 8, 9, 10, 11, 12, 13, 14, 15, 16, 17, 18, 19, 20 or greater. In some embodiments, a protein that comprises the ectodomain of a SARS-CoV-2 delta spike protein further comprises NDV F protein transmembrane and cytoplasmic domains. In certain embodiments, a protein that comprises the ectodomain of a SARS-CoV-2 delta variant spike polypeptide comprises one or more trimerization domains known to one of skill in the art (e.g., a T4 foldon trimerization domain), and optionally a tag (e.g., a His tag or Flag tag). In some embodiments, a protein comprising the ectodomain of a SARS-CoV-2 delta variant spike protein comprises C-terminus a thrombin cleavage site and a T4 foldon trimerization domain. In some embodiments, a protein comprising the ectodomain of a SARS-CoV-2 delta variant spike protein comprises C-terminus a thrombin cleavage site, a T4 foldon trimerization domain, and a hexahistidine tag.

In another embodiment, provided herein is a transgene comprising a nucleotide sequence encoding a protein, wherein the protein comprises a spike protein ectodomain that is at least 85%, at least 90%, at least 95%, at least 96%, at least 97%, at least 98%, at least 99%, or at least 99.5% identical to the amino acid sequence of SEQ ID NO:13. In another embodiment, provided herein is a transgene comprising a nucleotide sequence encoding a protein, wherein the protein comprises a spike protein ectodomain that is at least 95%, at least 96%, at least 97%, at least 98%, at least 99%, or at least 99.5% identical to the amino acid sequence of SEQ ID NO:13. In another embodiment, provided herein is a transgene comprising a nucleotide sequence encoding a protein, wherein the protein comprises a spike protein ectodomain that is at least 97%, at least 98%, at least 99%, or at least 99.5% identical to the amino acid sequence of SEQ ID NO:13. In another embodiment, provided herein is a transgene comprising a nucleotide sequence encoding a protein, wherein the protein comprises a spike protein ectodomain that is at least 85%, at least 90%, at least 95%, at least 96%, at least 97%, at least 98%, at least 99%, or at least 99.5% identical to the amino acid sequence of SEQ ID NO:13 without the signal sequence. In another embodiment, provided herein is a transgene comprising a nucleotide sequence encoding a protein, wherein the protein comprises a spike protein ectodomain that is at least 95%, at least 96%, at least 97%, at least 98%, at least 99%, or at least 99.5% identical to the amino acid sequence of SEQ ID NO:13 without the signal sequence. In another embodiment, provided herein is a transgene comprising a nucleotide sequence encoding a protein, wherein the protein comprises a spike protein ectodomain that is at least 97%, at least 98%, at least 99%, or at least 99.5% identical to the amino acid sequence of SEQ ID NO:13 without the signal sequence. In another embodiment, provided herein is a transgene comprising a nucleotide sequence encoding a protein, wherein the protein comprises a spike protein ectodomain that is at least 95%, at least 96%, at least 97%, at least 98%, at least 99%, or at least 99.5% identical to the amino acid sequence of SEQ ID NO:17. In another embodiment, provided herein is a transgene comprising a nucleotide sequence encoding a protein, wherein the protein comprises a spike protein ectodomain that is at least 97%, at least 98%, at least 99%, or at least 99.5% identical to the amino acid sequence of SEQ ID NO:17. Methods/techniques known in the art may be used to determine sequence identity (see, e.g., “Best Fit” or “Gap” program of the Sequence Analysis Software Package, version 10; Genetics Computer Group, Inc.). In another embodiment, provided herein is a transgene comprising a nucleotide sequence encoding a protein, wherein the protein comprises the amino acid sequence of SEQ ID NO:13 or 17.

In another embodiment, a SARS-CoV-2 delta variant spike protein ectodomain or a derivative thereof comprises an amino acid sequence that is at least 85%, at least 90%, at least 95%, at least 96%, at least 97%, at least 98%, at least 99%, or at least 99.5% identical to the amino acid sequence of SEQ ID NO:13 or 17. In another embodiment, a SARS-CoV-2 delta variant spike protein ectodomain or a derivative thereof comprises an amino acid sequence that is at least 95%, at least 96%, at least 97%, at least 98%, at least 99%, or at least 99.5% identical to the amino acid sequence of SEQ ID NO:13 or 17. In another embodiment, a SARS-CoV-2 delta variant spike protein ectodomain or a derivative thereof comprises an amino acid sequence that is at least 97%, at least 98%, at least 99%, or at least 99.5% identical to the amino acid sequence of SEQ ID NO:13 or 17. In another embodiment, provided herein is a SARS-CoV-2 delta variant spike protein ectodomain or a derivative thereof comprises an amino acid sequence that is at least 85%, at least 90%, at least 95%, at least 96%, at least 97%, at least 98%, at least 99%, or at least 99.5% identical to the amino acid sequence of SEQ ID NO:13 without the signal sequence. In another embodiment, a SARS-CoV-2 delta variant spike protein ectodomain or a derivative thereof comprises an amino acid sequence that is at least 95%, at least 96%, at least 97%, at least 98%, at least 99%, or at least 99.5% identical to the amino acid sequence of SEQ ID NO:13 without the signal sequence. In another embodiment, a SARS-CoV-2 delta variant spike protein ectodomain or a derivative thereof comprises an amino acid sequence that is at least 97%, at least 98%, at least 99%, or at least 99.5% identical to the amino acid sequence of SEQ ID NO:13 without the signal sequence. Methods/techniques known in the art may be used to determine sequence identity (see, e.g., “Best Fit” or “Gap” program of the Sequence Analysis Software Package, version 10; Genetics Computer Group, Inc.). In another embodiment, a SARS-CoV-2 delta variant spike protein ectodomain or a derivative thereof comprises the amino acid sequence of SEQ ID NO:13 or 17.

In another embodiment, described herein are transgenes comprising a nucleotide sequence encoding a chimeric F protein, wherein the chimeric F protein comprises a SARS-CoV-2 delta variant spike protein ectodomain described herein and NDV F protein transmembrane and cytoplasmic domains. In some embodiments, the entire NDV F protein transmembrane and cytoplasmic domains is included in a chimeric F protein. In some embodiments, the entire NDV F protein transmembrane and cytoplasmic domains is not included in a chimeric F protein. For example, a few amino acid residues (e.g., 1-5, 1-10, or 5-15 amino acid residues) upstream to the NDV F protein transmembrane may be included in a chimeric F protein and/or a few amino acid residues (e.g., 1-5, 1-10, or 5-15 amino acid residues) downstream of the NDV F protein cytoplasmic domain may be included in a chimeric F protein. For example, a few amino acid residues (e.g., 1-5 amino acid residues) less than the entire NDV F protein transmembrane may be included in a chimeric F protein and/or a few amino acid residues (e.g., 1-5 amino acid residues) less than the entire NDV F protein cytoplasmic domain may be included. In specific embodiments, described herein are transgenes comprising a nucleotide sequence encoding a chimeric F protein, wherein the chimeric F protein comprises a SARS-CoV-2 delta variant spike protein ectodomain described herein, a NDV F protein transmembrane domain plus or minus 1, 2, 3, 4, or 5 amino acid residues, and a NDV F protein cytoplasmic domain plus or minus 1, 2, 3, 4, or 5 amino acid residues. In specific embodiments, the entire transmembrane and cytoplasmic domains of the SARS-CoV-2 delta variant spike protein are not present in the chimeric F protein. The ectodomain, transmembrane and cytoplasmic domains of the SARS-CoV-2 delta variant spike protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the SARS-CoV-2 delta variant spike protein and NDV F protein. See, e.g., Table 2, infra, with the transmembrane and cytoplasmic domains of an NDV F protein indicated. In specific embodiments, the SARS-CoV-2 spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:7). In some embodiments, the NDV F protein transmembrane and cytoplasmic domains are fused directly to the SARS-CoV-2 spike protein ectodomain. In certain embodiments, the transgene encoding the chimeric F protein is codon optimized. See, e.g., Section 5.1.4, infra, for a discussion regarding codon optimization.

In another embodiment, described herein are transgenes comprising a nucleotide sequence encoding a chimeric F protein, wherein the chimeric F protein comprises a derivative of a SARS-CoV-2 delta variant spike protein ectodomain and NDV F protein transmembrane and cytoplasmic domains. In specific embodiments, the entire NDV F protein transmembrane and cytoplasmic domains is included in a chimeric F protein. In a specific embodiment, the NDV F protein transmembrane and cytoplasmic domains comprise the amino acid sequence of SEQ ID NO:22. In some embodiments, the entire NDV F protein transmembrane and cytoplasmic domains is not included in a chimeric F protein. For example, a few amino acid residues (e.g., 1, 2, 3, 4, 5, 6, 7, 8, 9, 10, 11, 12, 13, 14, 15, 1-5, 1-10, or 5-15 amino acid residues) upstream to the NDV F protein transmembrane may be included in a chimeric F protein and/or a few amino acid residues (e.g., 1-5, 1-10, or 5-15 amino acid residues) downstream of the NDV F protein cytoplasmic domain may be included in a chimeric F protein. For example, a few amino acid residues (e.g., 1, 2, 3, 4, 5, or 1-5 amino acid residues) less than the entire NDV F protein transmembrane may be included in a chimeric F protein and/or a few amino acid residues (e.g., 1, 2, 3, 4, 5, or 1-5 amino acid residues) less than the entire NDV F protein cytoplasmic domain may be included. In another embodiment, described herein are transgenes comprising a nucleotide sequence encoding a chimeric F protein, wherein the chimeric F protein comprises a derivative of a SARS-CoV-2 delta variant spike protein ectodomain, a NDV F protein transmembrane domain plus or minus 1, 2, 3, 4, or 5 amino acid residues, and a NDV F protein cytoplasmic domain plus or minus 1, 2, 3, 4, or 5 amino acid residues. In specific embodiments, the chimeric F protein does not include the SARS-CoV-2 delta variant spike protein transmembrane and cytoplasmic domains. In some embodiments, 1, 2, or 3 amino acid residues of the transmembrane domain and/or cytoplasmic domain of the SARS-CoV-2 (e.g., Wuhan strain or delta variant) spike protein are present in the chimeric F protein. In specific embodiments, the derivative of the SARS-CoV-2 delta variant spike protein ectodomain lacks the polybasic cleavage site (e.g., one, two or more residues corresponding to amino acid residues 682 to 685 (RRAR) of the SARS-CoV-2 spike protein found at GenBank Accession No. MN908947.3 are substituted for other amino acid residues). In a specific embodiment, amino acid residues corresponding to amino acid residues 682 to 685 (RRAR) of the SARS-CoV-2 spike protein found at GenBank Accession No. MN908947.3 are substituted with a single alanine. See, e.g., Table 2, infra, with the transmembrane and cytoplasmic domains of an NDV F protein indicated. In some embodiments, the derivative comprises amino acid substitutions corresponding to the following amino acid residues of the SARS-CoV-2 spike protein found at GenBank Accession No. MN908947.3: F817P, A892P, A899P, A942P, K986P, and V987P. In a specific embodiment, the derivative comprises amino acid residues corresponding to amino acid residues 682 to 685 (RRAR) of the SARS-CoV-2 spike protein found at GenBank Accession No. MN908947.3 substituted with a single alanine, and amino acid substitutions corresponding to the following amino acid residues of the SARS-CoV-2 spike protein found at GenBank Accession No. MN908947.3: F817P, A892P, A899P, A942P, K986P, and V987P. In specific embodiments, the derivative of the SARS-CoV-2 spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:7). In some embodiments, the NDV F protein transmembrane and cytoplasmic domains are fused directly to the derivative of the SARS-CoV-2 spike protein ectodomain. In specific embodiment, described herein is a transgene comprising a nucleotide sequence encoding a chimeric F protein, wherein the transgene comprises an RNA sequence corresponding to the negative sense of the cDNA sequence of SEQ ID NO: 5 or 21. In another specific embodiment, described herein is a transgene comprising a nucleotide sequence encoding a chimeric F protein, wherein the chimeric F protein comprises the amino acid sequence of SEQ ID NO:13 or 17 and NDV F protein transmembrane and cytoplasmic domains. In certain embodiments, the transgene encoding the chimeric F protein is codon optimized. See, e.g., Section 5.1.4, infra, for a discussion regarding codon optimization. In a preferred embodiment, a transgene comprises a codon-optimized version of a nucleic acid sequence encoding the chimeric F protein. In certain embodiments, a transgene comprises a codon-optimized version of a nucleic acid sequence encoding the derivative of the ectodomain of the SARS-CoV-2 delta variant spike protein. In a specific embodiment, a transgene described herein comprises a nucleotide sequence encoding the amino acid sequence set forth in SEQ ID NO:6. In another specific embodiment, a transgene described herein comprises a nucleotide sequence encoding the amino acid sequence set forth in SEQ ID NO:18. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, described herein are transgenes comprising a nucleotide sequence encoding a chimeric F protein, wherein the chimeric F protein comprises a SARS-CoV-2 delta variant spike protein ectodomain plus or minus 1, 2, 3, 4, 5, 6, 7, 8 or more amino acid residues at C-terminus of the ectodomain and NDV F protein transmembrane and cytoplasmic domains. In other words, the portion of the SARS-CoV-2 delta variant spike protein encoded by the chimeric F protein does not include the full-length SARS-CoV-2 delta variant spike protein transmembrane and cytoplasmic domains. The ectodomain, transmembrane and cytoplasmic domains of the SARS-CoV-2 delta variant spike protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the SARS-CoV-2 delta variant spike protein and NDV F protein. See, e.g., Table 2, infra, with the transmembrane and cytoplasmic domains of an NDV F protein indicated. In specific embodiments, the SARS-CoV-2 delta variant spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:7). In some embodiments, the NDV F protein transmembrane and cytoplasmic domains are fused to directly to the SARS-CoV-2 delta variant spike protein ectodomain. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, described herein are transgenes comprising a nucleotide sequence encoding a chimeric F protein, wherein the chimeric F protein comprises a derivative of a SARS-CoV-2 delta variant spike protein ectodomain and NDV F protein transmembrane and cytoplasmic domains, wherein the derivative comprises plus or minus 1, 2, 3, 4, 5, 6, 7, 8 or more amino acid residues at C-terminus of the ectodomain. In specific embodiments, the derivative of the SARS-CoV-2 delta variant spike protein ectodomain lacks the polybasic cleavage site (e.g., one, two or more residues corresponding to amino acid residues 682 to 685 (RRAR) of the SARS-CoV-2 spike protein found at GenBank Accession No. MN908947.3 are substituted for other amino acid residues). In specific embodiments, the lack of a polybasic cleavage means that the polybasic site is altered such that it cannot be cleaved by, e.g., furin. In a specific embodiment, amino acid residues corresponding to amino acid residues 682 to 685 (RRAR) of the SARS-CoV-2 spike protein found at GenBank Accession No. MN908947.3 are substituted with a single alanine. In some embodiments, the derivative comprises the following amino acid substitutions at amino acid residues corresponding to the following amino acid residues of the SARS-CoV-2 spike protein found at GenBank Accession No. MN908947.3: F817P, A892P, A899P, A942P, K986P, and V987P. In a specific embodiment, the derivative comprises an amino acid substitution at amino acid residues corresponding to amino acid residues 682 to 685 (RRAR) of the SARS-CoV-2 spike protein found at GenBank Accession No. MN908947.3 with a single alanine, and the following amino acid substitutions at amino acid residues corresponding to the following amino acid residues of the SARS-CoV-2 spike protein found at GenBank Accession No. MN908947.3: F817P, A892P, A899P, A942P, K986P, and V987P. The ectodomain, transmembrane and cytoplasmic domains of the SARS-CoV-2 delta variant spike protein and NDV F protein may be determined using techniques known to one of skill in the art. For example, published information, GenBank or websites such as VIPR virus pathogen website (www.viprbrc.org), DTU Bioinformatics domain website (www.cbs.dtu.dk/services/TMHMM/) or programs available to determine the transmembrane domain may be used to determine the ectodomain, transmembrane and cytoplasmic domains of the SARS-CoV-2 delta variant spike protein and NDV F protein. See, e.g., Table 2, infra, with the transmembrane and cytoplasmic domains of an NDV F protein indicated. In specific embodiments, the derivative of the SARS-CoV-2 delta variant spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:7). In some embodiments, the NDV F protein transmembrane and cytoplasmic domains are fused to directly to the derivative of the SARS-CoV-2 delta variant spike protein ectodomain. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a SARS-CoV-2 delta variant spike protein ectodomain with 1, 2, 3, 4, 5, 6, 7, 8, 9, 10, 11, or 12 amino acids substituted with another amino acid (e.g., a conservative amino acid substitution) and NDV F protein transmembrane and cytoplasmic domains. In another embodiment, provided herein is a transgene comprising a nucleotide sequence encoding a chimeric F protein comprising (or consisting of) a SARS-CoV-2 delta variant spike protein ectodomain with 1, 2, 3, 4, 5, 6, 7, 8, 9, 10, 11, or 12 amino acids at the C-terminus of the ectodomain substituted with another amino acid (e.g., a conservative amino acid substitution) and NDV F protein transmembrane and cytoplasmic domains. In another embodiment, provided herein is a transgene comprising a nucleotide sequence encoding a chimeric F protein comprising (or consisting of) a SARS-CoV-2 delta variant spike protein ectodomain with 1, 2, 3, 4, 5, 6, 7, 8, 9, 10, 11, or 12 amino acids at the N-terminus of the ectodomain substituted with another amino acid (e.g., a conservative amino acid substitution) and NDV F protein transmembrane and cytoplasmic domains. In another embodiment, provided herein is a transgene comprising a nucleotide sequence encoding a chimeric F protein comprising (or consisting of) a SARS-CoV-2 delta variant spike protein ectodomain with 1, 2, 3, 4, 5, 6, 7, 8, 9, 10, 11, or 12 amino acids at the N-terminus substituted with another amino acid (e.g., a conservative amino acid substitution) and 1, 2, 3, 4, 5, 6, 7, 8, 9, 10, 11, or 12 amino acids at the C-terminus substituted with another amino acid (e.g., a conservative amino acid substitution), and NDV F protein transmembrane and cytoplasmic domains. In a specific embodiment, the C-terminus is the last 100 amino acid residues of the ectodomain. In a specific embodiment, the N-terminus is the first 100 amino acid residues of the ectodomain. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophilic (Cys, Ser, Thr), acidic (Asp, Glu), basic (Asn, Gln, His, Lys, Arg), conformation disruptors (Gly, Pro) and aromatic (Trp, Tyr, Phe). In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In specific embodiments, the NDV F protein transmembrane and cytoplasmic domains of the chimeric protein are from a different NDV strain than the transcription units of the NDV genome. In a specific embodiment, the NDV genome is of the LaSota strain.

In another embodiment, provided herein is a transgene comprising a chimeric F protein, wherein the chimeric F protein comprises (or consists of) a derivative of a SARS-CoV-2 delta variant spike protein ectodomain with 1, 2, 3, 4, 5, 6, 7, 8, 9, 10, 11, or 12 amino acids substituted with another amino acid (e.g., a conservative amino acid substitution) and NDV F protein transmembrane and cytoplasmic domains. In specific embodiments, the derivative of the SARS-CoV-2 delta variant spike protein ectodomain lacks the polybasic cleavage site (e.g., one, two or more residues corresponding to amino acid residues 682 to 685 (RRAR) of the SARS-CoV-2 spike protein found at GenBank Accession No. MN908947.3 are substituted for other amino acid residues). In a specific embodiment, amino acid residues corresponding to amino acid residues 682 to 685 (RRAR) of the SARS-CoV-2 spike protein found at GenBank Accession No. MN908947.3 are substituted with a single alanine. In some embodiments, the derivative comprises the following amino acid substitutions at the following amino acid residues corresponding to amino acid residues of the SARS-CoV-2 spike protein found at GenBank Accession No. MN908947.3: F817P, A892P, A899P, A942P, K986P, and V987P. In a specific embodiment, the derivative comprises an amino acid substitution at amino acid residues corresponding to amino acid residues 682 to 685 (RRAR) of the SARS-CoV-2 spike protein found at GenBank Accession No. MN908947.3 with a single alanine, and the following amino acid substitutions at the following amino acid residues corresponding to amino acid residues of the SARS-CoV-2 spike protein found at GenBank Accession No. MN908947.3: F817P, A892P, A899P, A942P, K986P, and V987P. In specific embodiments, the derivative of the SARS-CoV-2 delta variant spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:7). In other embodiments, the derivative of the SARS-CoV-2 delta variant spike protein ectodomain is fused directly to the NDV F protein transmembrane and cytoplasmic domains. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophilic (Cys, Ser, Thr), acidic (Asp, Glu), basic (Asn, Gln, His, Lys, Arg), conformation disruptors (Gly, Pro) and aromatic (Trp, Tyr, Phe). In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In specific embodiments, the NDV F protein transmembrane and cytoplasmic domains of the chimeric protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a SARS-CoV-2 delta variant spike protein ectodomain with 1, 2, 3, 4, 5, 6, 7, 8, 9, 10, 11, or 12 amino acids deleted, and NDV F protein transmembrane and cytoplasmic domains. In another embodiment, provided herein is a transgene comprising a nucleotide sequence encoding a chimeric F protein comprising (or consisting of) a SARS-CoV-2 delta variant spike protein ectodomain with 1, 2, 3, 4, 5, 6, 7, 8, 9, 10, 11, or 12 amino acids deleted from the C-terminus, and NDV F protein transmembrane and cytoplasmic domains. In another embodiment, provided herein is a transgene comprising a nucleotide sequence encoding a chimeric F protein comprising (or consisting of) a SARS-CoV-2 delta variant spike protein ectodomain with 1, 2, 3, 4, 5, 6, 7, 8, 9, 10, 11, or 12 amino acids deleted from the N-terminus, and NDV F protein transmembrane and cytoplasmic domains. In another embodiment, provided herein is a transgene comprising a nucleotide sequence encoding a chimeric F protein comprising (or consisting of) a SARS-CoV-2 delta variant spike protein ectodomain with 1, 2, 3, 4, 5, 6, 7, 8, 9, 10, 11, or 12 amino acids deleted from the N-terminus and 1, 2, 3, 4, 5, 6, 7, 8, 9, 10, 11, or 12 amino acids deleted from the C-terminus, and NDV F protein transmembrane and cytoplasmic domains. In a specific embodiment, the C-terminus is the last 100 amino acid residues of the ectodomain. In a specific embodiment, the N-terminus is the first 100 amino acid residues of the ectodomain. In specific embodiments, the SARS-CoV-2 delta variant spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:7). In other embodiments, the SARS-CoV-2 delta variant spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a derivative of a SARS-CoV-2 delta variant spike protein ectodomain with 1, 2, 3, 4, 5, 6, 7, 8, 9, 10, 11, or 12 amino acids deleted, and NDV F protein transmembrane and cytoplasmic domains. In another embodiment, provided herein is a transgene comprising a nucleotide sequence encoding a chimeric F protein comprising (or consisting of) a derivative of a SARS-CoV-2 delta variant spike protein ectodomain with 1, 2, 3, 4, 5, 6, 7, 8, 9, 10, 11, or 12 amino acids deleted from the C-terminus, and NDV F protein transmembrane and cytoplasmic domains. In another embodiment, provided herein is a transgene comprising a nucleotide sequence encoding a chimeric F protein comprising (or consisting of) a derivative of a SARS-CoV-2 delta variant spike protein ectodomain with 1, 2, 3, 4, 5, 6, 7, 8, 9, 10, 11, or 12 amino acids deleted from the N-terminus, and NDV F protein transmembrane and cytoplasmic domains. In another embodiment, provided herein is a transgene comprising a nucleotide sequence encoding a chimeric F protein comprising (or consisting of) a derivative of a SARS-CoV-2 delta variant spike protein ectodomain with 1, 2, 3, 4, 5, 6, 7, 8, 9, 10, 11, or 12 amino acids deleted from the N-terminus and 1, 2, 3, 4, 5, 6, 7, 8, 9, 10, 11, or 12 amino acids deleted from the C-terminus, and NDV F protein transmembrane and cytoplasmic domains. In a specific embodiment, the C-terminus is the last 100 amino acid residues of the ectodomain. In a specific embodiment, the N-terminus is the first 100 amino acid residues of the ectodomain. In certain embodiments, the derivative of the SARS-CoV-2 delta variant spike protein ectodomain lacks the polybasic cleavage site (e.g., one, two or more residues corresponding to amino acid residues 682 to 685 (RRAR) of the SARS-CoV-2 spike protein found at GenBank Accession No. MN908947.3 are substituted for other amino acid residues). In a specific embodiment, amino acid residues corresponding to amino acid residues 682 to 685 (RRAR) of the SARS-CoV-2 spike protein found at GenBank Accession No. MN908947.3 are substituted with a single alanine. In some embodiments, the derivative comprises the following amino acid substitutions at amino acid residues corresponding to the following amino acid residues of the SARS-CoV-2 spike protein found at GenBank Accession No. MN908947.3: F817P, A892P, A899P, A942P, K986P, and V987P. In a specific embodiment, the derivative comprises an amino acid substitution at amino acid residues corresponding to amino acid residues 682 to 685 (RRAR) of the SARS-CoV-2 spike protein found at GenBank Accession No. MN908947.3 with a single alanine, and the following amino acid substitutions at amino acid residues corresponding to the following amino acid residues of the SARS-CoV-2 spike protein found at GenBank Accession No. MN908947.3: F817P, A892P, A899P, A942P, K986P, and V987P. In specific embodiments, the derivative of the SARS-CoV-2 delta variant spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:7). In other embodiments, the derivative of the SARS-CoV-2 delta variant spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a SARS-CoV-2 delta variant spike protein ectodomain with 1, 2, 3, 4, 5, 6, 7, 8, 9, 10, 11, 12, 13, 14, or 15 mutations (e.g., amino acid substitutions, amino acid deletions, amino acid additions, or a combination thereof), and NDV F protein transmembrane and cytoplasmic domains. In another embodiment, provided herein is a transgene comprising a nucleotide sequence encoding a chimeric F protein comprising (or consisting of) a SARS-CoV-2 delta variant spike protein ectodomain with 1, 2, 3, 4, 5, 6, 7, 8, 9, 10, 11, or 12 amino acid substitutions and 1, 2, 3, 4, 5, 6, 7, 8, 9, 10, 11, or 12 amino acids deleted, and NDV F protein transmembrane and cytoplasmic domains. In specific embodiments, the SARS-CoV-2 delta variant spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:7). In other embodiments, the SARS-CoV-2 delta variant spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a derivative of a SARS-CoV-2 delta variant spike protein ectodomain with 1, 2, 3, 4, 5, 6, 7, 8, 9, 10, 11, 12, 13, 14, or 15 mutations (e.g., amino acid substitutions, amino acid deletions, amino acid additions, or a combination thereof), and NDV F protein transmembrane and cytoplasmic domains. In another embodiment, provided herein is a transgene comprising a nucleotide sequence encoding a chimeric F protein comprising (or consisting of) a derivative of a SARS-CoV-2 delta variant spike protein ectodomain with 1, 2, 3, 4, 5, 6, 7, 8, 9, 10, 11, or 12 amino acid substitutions and 1, 2, 3, 4, 5, 6, 7, 8, 9, 10, 11, or 12 amino acids deleted, and NDV F protein transmembrane and cytoplasmic domains. In certain embodiments, the derivative of the SARS-CoV-2 delta variant spike protein ectodomain lacks the polybasic cleavage site (e.g., one, two or more residues corresponding to amino acid residues 682 to 685 (RRAR) of the SARS-CoV-2 spike protein found at GenBank Accession No. MN908947.3 are substituted for other amino acid residues). In specific embodiments, the lack of a polybasic cleavage means that the polybasic site is altered such that it cannot be cleaved by, e.g., furin. In a specific embodiment, amino acid residues corresponding to amino acid residues 682 to 685 (RRAR) of the SARS-CoV-2 spike protein found at GenBank Accession No. MN908947.3 are substituted with a single alanine. In some embodiments, the derivative comprises the following amino acid substitutions at amino acid residues corresponding to amino acid residues of the SARS-CoV-2 spike protein found at GenBank Accession No. MN908947.3: F817P, A892P, A899P, A942P, K986P, and V987P. In a specific embodiment, the derivative comprises an amino acid substitution at amino acid residues corresponding to amino acid residues 682 to 685 (RRAR) of the SARS-CoV-2 spike protein found at GenBank Accession No. MN908947.3 with a single alanine, and the following amino acid substitutions at amino acid residues corresponding to the following amino acid residues of the SARS-CoV-2 spike protein found at GenBank Accession No. MN908947.3: F817P, A892P, A899P, A942P, K986P, and V987P. In specific embodiments, the derivative of the SARS-CoV-2 delta variant spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:7). In other embodiments, the derivative of the SARS-CoV-2 delta variant spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, described herein are transgenes comprising a nucleotide sequence encoding a chimeric F protein, wherein the chimeric F protein comprises a derivative of a SARS-CoV-2 delta variant spike protein ectodomain and NDV F protein transmembrane and cytoplasmic domains, wherein the derivative comprises amino acid residues corresponding to amino acid residues 817, 892, 899, 942, 986, and 987 of the spike protein found at GenBank Accession No. MN908947.3 substituted with prolines, and wherein the derivative lacks a polybasic cleavage site. In specific embodiments, the lack of a polybasic cleavage means that the polybasic site is altered such that it cannot be cleaved by, e.g., furin. The SARS-CoV-2 delta variant spike protein ectodomain may lack the polybasic cleavage site as a result of amino acid residues 682 to 685 of the polybasic cleavage site being substituted with a single alanine. See, e.g., Table 2, infra, with the transmembrane and cytoplasmic domains of an NDV F protein indicated. In specific embodiments, the derivative of the SARS-CoV-2 delta variant spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:7). In some embodiments, the NDV F protein transmembrane and cytoplasmic domains are fused directly to the derivative of the SARS-CoV-2 spike protein ectodomain. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units, or between the NDV HN and L transcription units).

In another embodiment, provided herein is a transgene comprising a nucleotide sequence that is at least 85%, at least 90%, at least 95%, at least 96%, at least 97%, at least 98% or at least 99% identical to the nucleotide sequence of SEQ ID NO:5. In another embodiment, provided herein is a transgene comprising a nucleotide sequence that is at least 95%, at least 96%, at least 97%, at least 98% or at least 99% identical to the nucleotide sequence of SEQ ID NO:5. In another embodiment, provided herein is a transgene comprising a nucleotide sequence that is at least 97%, at least 98% or at least 99% identical to the nucleotide sequence of SEQ ID NO:5. In another embodiment, provided herein is a transgene comprising a nucleotide sequence that is at least 85%, at least 90%, at least 95%, at least 96%, at least 97%, at least 98% or at least 99% identical to the nucleotide sequence of SEQ ID NO:5 without the signal sequence. In another embodiment, provided herein is a transgene comprising a nucleotide sequence that is at least 95%, at least 96%, at least 97%, at least 98% or at least 99% identical to the nucleotide sequence of SEQ ID NO:5 without the signal sequence. In another embodiment, provided herein is a transgene comprising a nucleotide sequence that is at least 97%, at least 98%, at least 99%, or at least 99.5% identical to the nucleotide sequence of SEQ ID NO:5 without the signal sequence. In another embodiment, provided herein is a transgene comprising a nucleotide sequence encoding a chimeric F protein comprising (or consisting of) an amino acid sequence that is at least 85%, at least 90%, at least 95%, at least 96%, at least 97%, at least 98%, least 99%, or at least 99.5% identical to the amino acid sequence of SEQ ID NO:6. In another embodiment, provided herein is a transgene comprising a nucleotide sequence encoding a chimeric F protein comprising (or consisting of) the amino acid sequence of SEQ ID NO:6. In another embodiment, provided herein is a transgene comprising a nucleotide sequence encoding a chimeric F protein comprising (or consisting of) an amino acid sequence that is at least 95%, at least 96%, at least 97%, at least 98%, at least 99%, or at least 99.5% identical to the amino acid sequence of SEQ ID NO:6. In another embodiment, provided herein is a transgene comprising a nucleotide sequence encoding a chimeric F protein comprising (or consisting of) an amino acid sequence that is at least 97%, at least 98% or at least 99% identical to the amino acid sequence of SEQ ID NO:6. In another embodiment, provided herein is a transgene comprising a nucleotide sequence encoding a chimeric F protein comprising (or consisting of) an amino acid sequence that is at least 85%, at least 90%, at least 95%, at least 96%, at least 97%, at least 98%, at least 99%, or at least 99.5% identical to the amino acid sequence of SEQ ID NO:6 without the signal sequence. In another embodiment, provided herein is a transgene comprising a nucleotide sequence encoding a protein comprising (or consisting of) an amino acid sequence that is at least 95%, at least 96%, at least 97%, at least 98% or at least 99% identical to the amino acid sequence of SEQ ID NO:6 without the signal sequence. In another embodiment, provided herein is a transgene comprising a nucleotide sequence encoding a chimeric F protein comprising (or consisting of) an amino acid sequence that is at least 97%, at least 98%, at least 99%, or at least 99.5% identical to the amino acid sequence of SEQ ID NO:6 without the signal sequence. In another embodiment, provided herein is a transgene comprising a nucleotide sequence encoding a chimeric F protein comprising (or consisting of) the amino acid sequence of SEQ ID NO:6 without the signal sequence. Methods/techniques known in the art may be used to determine sequence identity (see, e.g., “Best Fit” or “Gap” program of the Sequence Analysis Software Package, version 10; Genetics Computer Group, Inc.). In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In specific embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) an amino acid sequence that is at least 85%, at least 90%, at least 95%, at least 96%, at least 97%, at least 98%, least 99%, or at least 99.5% identical to the amino acid sequence of SEQ ID NO:6, wherein the chimeric F protein comprises at least one, at least two, at least three, at least four, or more of the following amino acid modifications: T19R, G142D, delE156, R158G, L452R, T478K, and D950N. In another embodiment, provided herein is a transgene comprising a nucleotide sequence encoding a chimeric F protein comprising (or consisting of) an amino acid sequence that is at least 95%, at least 96%, at least 97%, at least 98%, at least 99%, or at least 99.5% identical to the amino acid sequence of SEQ ID NO:6. In another embodiment, provided herein is a transgene comprising a nucleotide sequence encoding a chimeric F protein comprising (or consisting of) an amino acid sequence that is at least 95%, at least 96%, at least 97%, at least 98%, at least 99%, or at least 99.5% identical to the amino acid sequence of SEQ ID NO:18. In another embodiment, provided herein is a transgene comprising a nucleotide sequence encoding a chimeric F protein comprising (or consisting of) an amino acid sequence that is at least 97%, at least 98%, or at least 99% identical to the amino acid sequence of SEQ ID NO:6, wherein the chimeric F protein comprises at least one, at least two, at least three, at least four, or more of the following amino acid modifications: T19R, G142D, delE156, R158G, L452R, T478K, and D950N. In some embodiments, SEQ ID NO:6 lacks the signal sequence. See SEQ ID NO:15 for signal sequence. Methods/techniques known in the art may be used to determine sequence identity (see, e.g., “Best Fit” or “Gap” program of the Sequence Analysis Software Package, version 10; Genetics Computer Group, Inc.). In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In specific embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein, wherein the chimeric F protein comprises (or consists of) a SARS-CoV-2 spike protein ectodomain and NDV F protein transmembrane and cytoplasmic domains, and wherein the SARS-CoV-2 spike protein ectodomain comprises amino acid sequence that is at least 85%, 90%, 95%, 96%, 97%, 98% or 99% identical to the amino acid sequence of SEQ ID NO:13, or SEQ ID NO:13 without the signal sequence. In another embodiment, provided herein is a transgene comprising a nucleotide sequence encoding a chimeric F protein, wherein the chimeric F protein comprises (or consists of) a SARS-CoV-2 spike protein ectodomain and NDV F protein transmembrane and cytoplasmic domains, and wherein the SARS-CoV-2 spike protein ectodomain comprises amino acid sequence that is at least 85%, 90%, 95%, 96%, 97%, 98% or 99% identical to the amino acid sequence of SEQ ID NO:17. In another embodiment, provided herein is a transgene comprising a nucleotide sequence encoding a chimeric F protein, wherein the chimeric F protein comprises (or consists of) a SARS-CoV-2 spike protein ectodomain and NDV F protein transmembrane and cytoplasmic domains, and wherein the SARS-CoV-2 spike protein ectodomain comprises amino acid sequence that is at least 95%, at least 96%, at least 97%, at least 98% or at least 99% identical to the amino acid sequence of SEQ ID NO:13, or SEQ ID NO:13 without the signal peptide. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising a derivative of a SARS-CoV-2 delta variant spike protein ectodomain and NDV F protein transmembrane and cytoplasmic domains, wherein the derivative comprises a SARS-CoV-2 delta variant spike protein ectodomain with 1, 2, 3, 4, 5, 6, 7, 8, 9, 10, 11, 12, 13, 14, or 15 amino acids substituted with another amino acid (e.g., a conservative amino acid substitution) and lacks a polybasic cleavage site (e.g., as a result of one, two, or more amino acid substitutions in polybasic cleavage site), and wherein amino acid residues corresponding to amino acid residues 817, 892, 899, 942, 986, and 987 of the spike protein found at GenBank Accession No. MN908947.3 are substituted with prolines. The SARS-CoV-2 delta variant spike protein ectodomain may lack the polybasic cleavage site as a result of a substitution of amino acid residues RRAR to A at amino acid residues corresponding to amino acid residues 682 to 685 of GenBank Accession No. MN908947.3. In a specific embodiment, the SARS-CoV-2 delta variant is of the B.1.617.2 sublineage. In certain embodiments, the SARS-CoV-2 delta variant is of the AY sublineage. In some embodiments, the derivative of the SARS-CoV-2 delta variant spike protein ectodomain comprises the amino acid sequence of SEQ ID NO:12 with one, two, three, four, five, six, or all of the following amino acid modifications: T19R, G142D, R158G, L452R, T478K, D614G, P681R, and D950N. In specific embodiments, the derivative of the SARS-CoV-2 delta variant spike protein ectodomain does not comprise the amino acid substitution of P681R. In a specific embodiment, the derivative of the SARS-CoV-2 delta variant spike protein ectodomain comprises the amino acid sequence of SEQ ID NO:12 with one, two, three, four, five, or all of the following amino acid modifications: T19R, G142D, R158G, L452R, T478K, D614G, and D950N. In a specific embodiment, the derivative of the SARS-CoV-2 delta variant spike protein ectodomain comprises the amino acid sequence of SEQ ID NO:12 with the following amino acid modifications: T19R, G142D, R158G, L452R, T478K, D614G, and D950N. In certain embodiments, the derivative of the SARS-CoV-2 delta variant spike protein ectodomain comprises the amino acid sequence of SEQ ID NO:13. In certain embodiments, the derivative of the SARS-CoV-2 delta variant spike protein ectodomain comprises the amino acid sequence of SEQ ID NO:17. In another specific embodiment, the derivative of the SARS-CoV-2 delta variant spike protein ectodomain comprises the amino acid sequence of SEQ ID NO:12 with one, two, three, four, five, six, seven, eight, nine, ten, eleven, twelve, or all of the following amino acid modifications: T19R, T95I, G142D, R158G, A222V, W258L, K417N, L452R, T478K, D614G, P681R, and D950N. In some embodiments, the derivative SARS-CoV-2 delta variant spike protein ectodomain comprises the amino acid sequence of SEQ ID NO:12 with one, two, three, four, five, six, seven, eight, nine, ten, eleven, or all of the following amino acid modifications: T19R, T95I, G142D, R158G, A222V, W258L, K417N, L452R, T478K, D614G, and D950N. In some embodiments, the derivative SARS-CoV-2 delta variant spike protein ectodomain comprises the amino acid sequence of SEQ ID NO:12 with one, two, three, four, five, six, seven, eight, nine, ten, eleven, or all of the following amino acid modifications: T19R, G142D, delE156, delF157, R158G, L452R, T478K, D614G, and D950N. In specific embodiments, the derivative SARS-CoV-2 delta variant spike protein ectodomain comprises the amino acid sequence of SEQ ID NO:12 with all of the following amino acid modifications: T19R, G142D, delE156, delF157, R158G, L452R, T478K, D614G, and D950N. In a specific embodiment, the derivative SARS-CoV-2 delta variant spike protein ectodomain comprises the amino acid sequence of SEQ ID NO:12 with all of the amino acid modifications for Delta shown in FIG. 3A. In specific embodiments, the derivative of the SARS-CoV-2 spike protein ectodomain is fused to the NDV F protein transmembrane and cytoplasmic domains via a linker (e.g., GGGGS (SEQ ID NO:7)).

In certain embodiments, the derivative of the SARS-CoV-2 spike protein ectodomain comprises the amino acid sequence of SEQ ID NO:12. In some embodiments, the derivative of the SARS-CoV-2 spike protein ectodomain does not comprise the amino acid sequence of SEQ ID NO:12. In certain embodiments, the derivative of the SARS-CoV-2 spike protein ectodomain comprises the amino acid sequence of SEQ ID NO:16. In some embodiments, the derivative of the SARS-CoV-2 spike protein ectodomain does not comprise the amino acid sequence of SEQ ID NO:16. In certain embodiments, the derivative of the SARS-CoV-2 spike protein ectodomain comprises the amino acid sequence of SEQ ID NO:13. In certain embodiments, the derivative of the SARS-CoV-2 spike protein ectodomain comprises the amino acid sequence of SEQ ID NO:17.

In certain embodiments, the derivative of the SARS-CoV-2 spike protein ectodomain is encoded by nucleotide sequence that is at least 80%, at least 85%, or at least 90% identical to the nucleotide sequence of SEQ ID NO:19 or 20. In certain embodiments, the derivative of the SARS-CoV-2 spike protein ectodomain is encoded by a nucleotide sequence that is at least 95%, at least 98%, or at least 99% identical to the nucleotide sequence of SEQ ID NO:19 or 20. In certain embodiments, the derivative of the SARS-CoV-2 spike protein ectodomain is encoded by the nucleotide sequence of SEQ ID NO: 19 or 20.

In specific embodiments, the derivative of the SARS-CoV-2 spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:7). In other embodiments, the derivative of the SARS-CoV-2 delta variant spike protein ectodomain is fused directly to the NDV F protein transmembrane and cytoplasmic domains. Examples of conservative amino acid substitutions include, e.g., replacement of an amino acid of one class with another amino acid of the same class. In a particular embodiment, a conservative substitution does not alter the structure or function, or both, of a polypeptide. Classes of amino acids may include hydrophobic (Met, Ala, Val, Leu, Ile), neutral hydrophilic (Cys, Ser, Thr), acidic (Asp, Glu), basic (Asn, Gln, His, Lys, Arg), conformation disruptors (Gly, Pro) and aromatic (Trp, Tyr, Phe). In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a derivative of a SARS-CoV-2 delta variant spike protein ectodomain and NDV F protein transmembrane and cytoplasmic domains, wherein the derivative comprises a SARS-CoV-2 spike protein ectodomain with 1, 2, 3, 4, 5, 6, 7, 8, 9, 10, 11, or 12 amino acids deleted and lacks a polybasic cleavage site (e.g., as a result of one, two, or more amino acid substitutions in polybasic cleavage site), and wherein amino acid residues corresponding to amino acid residues 817, 892, 899, 942, 986, and 987 of the spike protein found at GenBank Accession No. MN908947.3 are substituted with prolines. In specific embodiments, the lack of a polybasic cleavage means that the polybasic site is altered such that it cannot be cleaved by, e.g., furin. The derivative of the SARS-CoV-2 delta variant spike protein ectodomain may lack the polybasic cleavage site as a result of a substitution of amino acid residues RRAR to A at amino acid residues corresponding to amino acid residues 682 to 685 of GenBank Accession No. MN908947.3. In a specific embodiment, the SARS-CoV-2 delta variant is of the B.1.617.2 sublineage. In certain embodiments, the SARS-CoV-2 delta variant is of the AY sublineage. In some embodiments, the derivative of the SARS-CoV-2 delta variant spike protein ectodomain comprises the amino acid sequence of SEQ ID NO:12 with one or both of the following amino acid deletions: delE156 and delF157. In specific embodiments, the derivative of the SARS-CoV-2 delta variant spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:7). In other embodiments, the derivative of the SARS-CoV-2 delta variant spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units). In specific embodiments, the NDV F protein transmembrane and cytoplasmic domains of the chimeric F protein are from the same NDV strain as the transcription units of the NDV genome. In specific embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene comprising a nucleotide sequence encoding a chimeric F protein comprising (or consisting of) a derivative of a SARS-CoV-2 delta variant spike protein ectodomain and NDV F protein transmembrane and cytoplasmic domains, wherein the derivative comprises a SARS-CoV-2 delta variant spike protein with 1, 2, 3, 4, 5, 6, 7, 8, 9, 10, 11, 12, 13, 14 or 15 mutations (e.g. amino acid substitutions, amino acid additions, amino acid deletions or a combination thereof) and lacks a polybasic cleavage site (e.g., as a result of one, two, or more amino acid substitutions in polybasic cleavage site), and wherein amino acid residues corresponding to amino acid residues 817, 892, 899, 942, 986, and 987 of the spike protein found at GenBank Accession No. MN908947.3 are substituted with prolines. In specific embodiments, the lack of a polybasic cleavage means that the polybasic site is altered such that it cannot be cleaved by, e.g., furin. The derivative of the SARS-CoV-2 delta variant spike protein ectodomain may lack the polybasic cleavage site as a result of a substitution of amino acid residues RRAR to A at amino acid residues corresponding to amino acid residues 682 to 685 of GenBank Accession No. MN908947.3. In a specific embodiment, the SARS-CoV-2 delta variant is of the B.1.617.2 sublineage. In certain embodiments, the SARS-CoV-2 delta variant is of the AY sublineage. In some embodiments, the derivative of the SARS-CoV-2 delta variant spike protein ectodomain comprises the amino acid sequence of SEQ ID NO:12 with one, two, three, four, five, six, seven, eight, or all of the following amino acid modifications: T19R, G142D, delE156, delF157, R158G, L452R, T478K, D614G, P681R, and D950N. In specific embodiments, the derivative of the SARS-CoV-2 delta variant spike protein ectodomain does not comprise the amino acid substitution of P681R. In a specific embodiment, the derivative of the SARS-CoV-2 delta variant spike protein ectodomain comprises the amino acid sequence of SEQ ID NO:12 with one, two, three, four, five, six, seven, or all of the following amino acid modifications: T19R, G142D, delE156, delF157, R158G, L452R, T478K, D614G, and D950N. In a specific embodiment, the derivative of the SARS-CoV-2 delta variant spike protein ectodomain comprises the amino acid sequence of SEQ ID NO:12 with the following amino acid modifications: T19R, G142D, delE156, delF157, R158G, L452R, T478K, D614G, and D950N. In certain embodiments, the derivative of the SARS-CoV-2 delta variant spike protein ectodomain comprises the amino acid sequence of SEQ ID NO:13. In another specific embodiment, the derivative of the SARS-CoV-2 delta variant spike protein ectodomain comprises the amino acid sequence of SEQ ID NO:12 with one, two, three, four, five, six, seven, eight, nine, ten, eleven, twelve, thirteen, fourteen, or all of the following amino acid modifications: T19R, T95I, G142D, delE156, delF157, R158G, A222V, W258L, K417N, L452R, T478K, D614G, P681R, and D950N. In specific embodiments, the derivative of the SARS-CoV-2 delta variant spike protein ectodomain does not comprise the amino acid substitution of P681R. In some embodiments, the derivative of the SARS-CoV-2 delta variant spike protein ectodomain comprises the amino acid sequence of SEQ ID NO:12 with one, two, three, four, five, six, seven, eight, nine, ten, eleven, twelve, thirteen, or all of the following amino acid modifications: T19R, T95I, G142D, delE156, delF157, R158G, A222V, W258L, K417N, L452R, T478K, D614G, and D950N. In specific embodiments, the derivative of the SARS-CoV-2 delta variant spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:7). In other embodiments, the derivative of the SARS-CoV-2 delta variant spike protein is fused directly to the NDV F protein transmembrane and cytoplasmic domains. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another specific embodiment, provided herein is a transgene comprising a nucleotide sequence that can hybridize under high, moderate or typical stringency hybridization conditions to the nucleic acid sequence set forth in SEQ ID NO: 5. In another specific embodiment, provided herein is a transgene comprising a nucleotide sequence that can hybridize under high, moderate to typical stringency hybridization conditions to a nucleic acid sequence encoding the protein set forth in SEQ ID NO: 6. In another specific embodiment, provided herein is a transgene comprising a nucleotide sequence that can hybridize under high, moderate to typical stringency hybridization conditions to a nucleic acid sequence encoding the protein set forth in SEQ ID NO: 18. Hybridization conditions are known to one of skill in the art (see, e.g., U.S. Patent Application No. 2005/0048549 at, e.g., paragraphs 72 and 73). In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between NP and P transcription units, or between the NDV HN and L transcription units). In specific embodiments, the NDV F protein transmembrane and cytoplasmic domains of the chimeric F protein are from the same NDV strain as the transcription units of the NDV genome. In other embodiments, the NDV F protein transmembrane and cytoplasmic domains of the chimeric F protein are from a different NDV strain than the transcription units of the NDV genome. In a specific embodiment, the NDV genome is of the LaSota strain.

In another embodiment, provided herein is a transgene that comprises a nucleotide sequence encoding a chimeric F protein, wherein the chimeric F protein comprises a derivative of a SARS-CoV-2 delta variant spike protein ectodomain and an NDV F protein transmembrane and cytoplasmic domains, wherein the derivative comprises the amino acid sequence set forth in SEQ ID NO:12, 13, 16 or 17. In a specific embodiment, provided herein is a transgene that comprises a nucleotide sequence encoding a chimeric F protein, wherein the chimeric F protein comprises a derivative of a SARS-CoV-2 delta variant spike protein ectodomain and an NDV F protein transmembrane and cytoplasmic domains, wherein the derivative comprises the amino acid sequence set forth in SEQ ID NO:13 or 17. In another embodiment, provided herein is a transgene that comprises a nucleotide sequence encoding a chimeric F protein, wherein the chimeric F protein comprises a derivative of a SARS-CoV-2 delta variant spike protein ectodomain and an NDV F protein transmembrane and cytoplasmic domains, wherein the derivative comprises an amino acid sequence that is at least 85%, at least 90%, or at least 95%, identical to the amino acid sequence set forth in SEQ ID NO:12, 13, 16, or 17. In a specific embodiment, provided herein is a transgene that comprises a nucleotide sequence encoding a chimeric F protein, wherein the chimeric F protein comprises a derivative of a SARS-CoV-2 delta variant spike protein ectodomain and an NDV F protein transmembrane and cytoplasmic domains, wherein the derivative comprises an amino acid sequence that is at least 85%, at least 90%, or at least 95%, identical to the amino acid sequence set forth in SEQ ID NO: 13 or 17. In another embodiment, provided herein is a transgene that comprises a nucleotide sequence encoding a chimeric F protein, wherein the chimeric F protein comprises a derivative of a SARS-CoV-2 delta variant spike protein ectodomain and an NDV F protein transmembrane and cytoplasmic domains, wherein the derivative comprises the amino acid sequence set forth in SEQ ID NO:13. In another embodiment, provided herein is a transgene that comprises a nucleotide sequence encoding a chimeric F protein, wherein the chimeric F protein comprises a derivative of a SARS-CoV-2 delta variant spike protein ectodomain and an NDV F protein transmembrane and cytoplasmic domains, wherein the derivative comprises an amino acid sequence that is at least 96%, at least 97%, or at least 98%, at least 99%, or at least 99.5% identical to the amino acid sequence set forth in SEQ ID NO:13. In another embodiment, provided herein is a transgene that comprises a nucleotide sequence encoding a chimeric F protein, wherein the chimeric F protein comprises a derivative of a SARS-CoV-2 delta variant spike protein ectodomain and an NDV F protein transmembrane and cytoplasmic domains, wherein the derivative comprises the amino acid sequence set forth in SEQ ID NO:17. In another embodiment, provided herein is a transgene that comprises a nucleotide sequence encoding a chimeric F protein, wherein the chimeric F protein comprises a derivative of a SARS-CoV-2 delta variant spike protein ectodomain and an NDV F protein transmembrane and cytoplasmic domains, wherein the derivative comprises an amino acid sequence that is at least 85%, at least 90%, or at least 95%, identical to the amino acid sequence set forth in SEQ ID NO:17. In another embodiment, provided herein is a transgene that comprises a nucleotide sequence encoding a chimeric F protein, wherein the chimeric F protein comprises a derivative of a SARS-CoV-2 delta variant spike protein ectodomain and an NDV F protein transmembrane and cytoplasmic domains, wherein the derivative comprises an amino acid sequence that is at least 96%, at least 97%, or at least 98%, at least 99%, or at least 99.5% identical to the amino acid sequence set forth in SEQ ID NO:17. See, e.g., Table 2, infra, with the transmembrane and cytoplasmic domains of an NDV F protein indicated. In specific embodiments, the derivative of the SARS-CoV-2 delta variant spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:7). In some embodiments, the NDV F protein transmembrane and cytoplasmic domains are fused to directly to the derivative of the SARS-CoV-2 delta variant spike protein ectodomain. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units).

In another embodiment, provided herein is a transgene that comprises a nucleotide sequence encoding a chimeric F protein, wherein the chimeric F protein comprises a derivative of a SARS-CoV-2 delta variant spike protein ectodomain and an NDV F protein transmembrane and cytoplasmic domains, wherein the derivative comprises an amino acid sequence that is at least 85%, at least 90%, at least 95%, at least 96%, at least 97%, at least 98%, at least 99%, or at least 99.5% identical to the amino acid sequence set forth in SEQ ID NO:13 or 17, and wherein the derivative comprises: (1) amino acid substitutions to proline at two, three, four, five or all of the following amino acid positions 817, 892, 899, 942, 986, and 987 (as counted based on the SARS-CoV-2 spike protein found at GenBank Accession MN908947.3); and (2) RRAR to A amino acid substitution at amino acid positions 682 to 685 (as counted based on the SARS-CoV-2 spike protein found at GenBank Accession MN908947.3). In another embodiment, provided herein is a transgene that comprises a nucleotide sequence encoding a chimeric F protein, wherein the chimeric F protein comprises a derivative of a SARS-CoV-2 delta variant spike protein ectodomain and an NDV F protein transmembrane and cytoplasmic domains, wherein the derivative comprises an amino acid sequence that is at least 85%, at least 90%, at least 95%, at least 96%, at least 97%, at least 98%, at least 99%, or at least 99.5% identical to the amino acid sequence set forth in SEQ ID NO:13 or 17, and wherein the derivative comprises: (1) amino acid substitutions to proline at two, three, four, five or all of the following amino acid positions 817, 892, 899, 942, 986, and 987 (as counted based on the SARS-CoV-2 spike protein found at GenBank Accession MN908947.3); (2) RRAR to A amino acid substitution at amino acid positions 682 to 685 (as counted based on the SARS-CoV-2 spike protein found at GenBank Accession MN908947.3); and (3) amino acid substitutions at two, three, four, five, six, seven, eight, or all of the following amino acid positions 19, 142, 156, 157, 158, 452, 478, 614, and 950 (as counted based on the SARS-CoV-2 spike protein found at GenBank Accession MN908947.3), wherein the substitutions are selected from T19R, G142D, E156G, delF157, delR158, L452R, T478K, D614G, and D950N. In another embodiment, provided herein is a transgene that comprises a nucleotide sequence encoding a chimeric F protein, wherein the chimeric F protein comprises a derivative of a SARS-CoV-2 delta variant spike protein ectodomain and an NDV F protein transmembrane and cytoplasmic domains, wherein the derivative comprises an amino acid sequence that is at least 85%, at least 90%, at least 95%, at least 96%, at least 97%, at least 98%, at least 99%, or at least 99.5% identical to the amino acid sequence set forth in SEQ ID NO:13 or 17, and wherein the derivative comprises: (1) amino acid substitutions to proline at amino acid positions 817, 892, 899, 942, 986, and 987 (as counted based on the SARS-CoV-2 spike protein found at GenBank Accession MN908947.3); (2) RRAR to A amino acid substitution at amino acid positions 682 to 685 (as counted based on the SARS-CoV-2 spike protein found at GenBank Accession MN908947.3); and (3) amino acid substitutions at the following amino acid positions 19, 142, 156, 157, 158, 452, 478, 614, and 950 (as counted based on the SARS-CoV-2 spike protein found at GenBank Accession MN908947.3), wherein the substitutions are T19R, G142D, E156G, delF157, delR158, L452R, T478K, D614G, and D950N. In specific embodiments, the derivative comprises a proline at amino acid position 681 (as counted based on the SARS-CoV-2 spike protein found at GenBank Accession MN908947.3). In some specific embodiments, the derivative comprises an amino acid sequence that is at least 95%, at least 96%, at least 97%, at least 98%, or at least 99% identical to the amino acid sequence set forth in SEQ ID NO:13 or 17. In some specific embodiments, the derivative comprises at least 98% or at least 99% identical to the amino acid sequence set forth in SEQ ID NO:13 or 17. In some specific embodiments, the derivative comprises at least 99% or at least 99.5% identical to the amino acid sequence set forth in SEQ ID NO:13 or 17.

In another embodiment, provided herein is a transgene that comprises a nucleotide sequence encoding a chimeric F protein, wherein the chimeric F protein comprises a derivative of a SARS-CoV-2 spike protein ectodomain and an NDV F protein transmembrane and cytoplasmic domains, wherein the derivative is encoded by a nucleotide sequence that is at least 80%, at least 85%, or at least 90% identical to the nucleotide sequence of SEQ ID NO:19 or 20. In another embodiment, provided herein is a transgene that comprises a nucleotide sequence encoding a chimeric F protein, wherein the chimeric F protein comprises a derivative of a SARS-CoV-2 spike protein ectodomain and an NDV F protein transmembrane and cytoplasmic domains, wherein the derivative is encoded by a nucleotide sequence that is at least 95%, at least 98%, or at least 99% identical to the nucleotide sequence of SEQ ID NO:19 or 20. In another embodiment, provided herein is a transgene that comprises a nucleotide sequence encoding a chimeric F protein, wherein the chimeric F protein comprises a derivative of a SARS-CoV-2 spike protein ectodomain and an NDV F protein transmembrane and cytoplasmic domains, wherein the derivative is encoded by the nucleotide sequence of SEQ ID NO:19 or 20. See, e.g., Table 2, infra, with the transmembrane and cytoplasmic domains of an NDV F protein indicated. In specific embodiments, the derivative of the SARS-CoV-2 spike protein ectodomain is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)n (SEQ ID NO:24), wherein n is 1, 2, 3, 4, 5 or more. In another example, the linker may comprise (G)n, wherein n is 3, 4, 5, 6, 7, 8 or more. In a specific embodiment, the linker comprises the sequence GGGGS (SEQ ID NO:7). In some embodiments, the NDV F protein transmembrane and cytoplasmic domains are fused directly to the derivative of the SARS-CoV-2 spike protein ectodomain. In certain embodiments, the transgene encoding the chimeric F protein is codon optimized. See, e.g., Section 5.1.4, infra, for a discussion regarding codon optimization. In a specific embodiment, a transgene encoding a chimeric F protein is incorporated into the genome of any NDV type or strain (e.g., NDV LaSota strain). See, e.g., Section 5.1.1, supra, for types and strains of NDV that may be used. The transgene encoding a chimeric F protein may be incorporated between any two NDV transcription units (e.g., between the NDV P and M transcription units, between the NDV NP and P transcription units, or between the NDV HN and L transcription units).

In another embodiment, provided herein is a transgene that comprises a nucleotide sequence encoding a chimeric F protein, wherein the chimeric F protein comprises the amino acid sequence of SEQ ID NO:6, 11, 18, or 23. In a specific embodiment, provided herein is a transgene that comprises a nucleotide sequence encoding a chimeric F protein, wherein the chimeric F protein comprises the amino acid sequence of SEQ ID NO:6 or 23. In another embodiment, provided herein is a transgene that comprises a nucleotide sequence encoding a chimeric F protein, wherein the chimeric F protein comprises an amino acid sequence that is at least 85%, at least 90%, or at least 95%, identical to the amino acid sequence set forth in SEQ ID NO: 6, 11, 18, or 23. In a specific embodiment, provided herein is a transgene that comprises a nucleotide sequence encoding a chimeric F protein, wherein the chimeric F protein comprises an amino acid sequence that is at least 85%, at least 90%, or at least 95%, identical to the amino acid sequence set forth in SEQ ID NO: 6 or 18. In another embodiment, provided herein is a transgene that comprises a nucleotide sequence encoding a chimeric F protein, wherein the chimeric F protein comprises an amino acid sequence that is at least 96%, at least 97%, or at least 98%, at least 99%, or at least 99.5% identical to the amino acid sequence set forth in SEQ ID NO: 6, 11, 18, or 23. In a specific embodiment, provided herein is a transgene that comprises a nucleotide sequence encoding a chimeric F protein, wherein the chimeric F protein comprises an amino acid sequence that is at least 96%, at least 97%, or at least 98%, at least 99%, or at least 99.5% identical to the amino acid sequence set forth in SEQ ID NO: 6 or 18.

In another embodiment, provided herein is a transgene that comprises a nucleotide sequence of SEQ ID NO:5 or 21. In another embodiment, provided herein is a transgene that comprises a nucleotide sequence that is at least 85%, at least 90%, or at least 95%, identical to the nucleotide sequence of SEQ ID NO: 5 or 21. In another embodiment, provided herein is a transgene that comprises a nucleotide sequence comprises an nucleotide sequence that is at least 96%, at least 97%, or at least 98%, at least 99%, or at least 99.5% identical to the nucleotide sequence set forth in SEQ ID NO: 5 or 21.

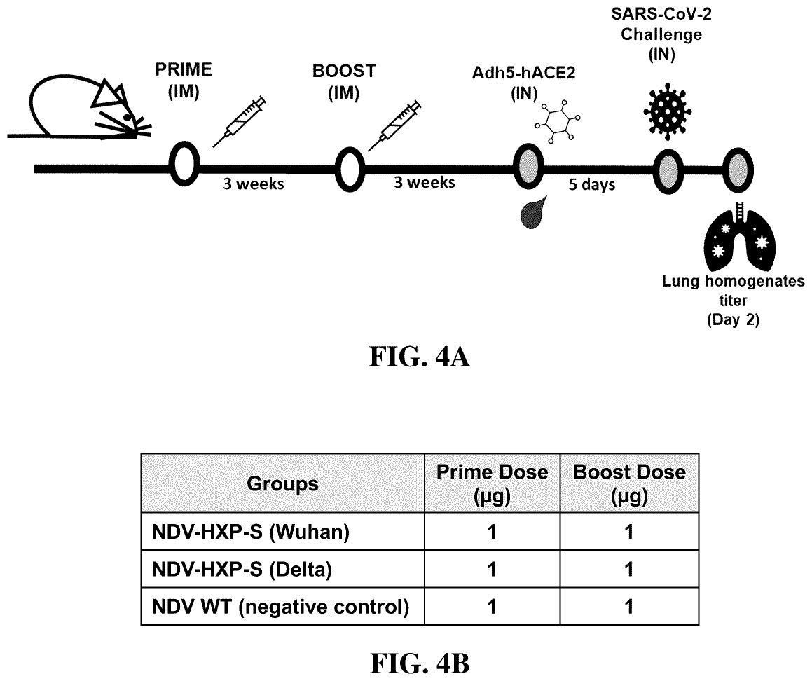

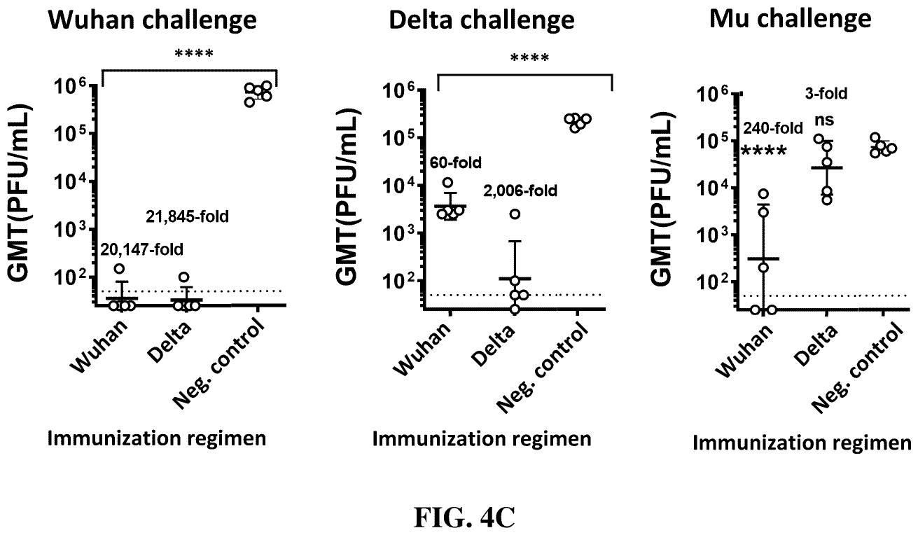

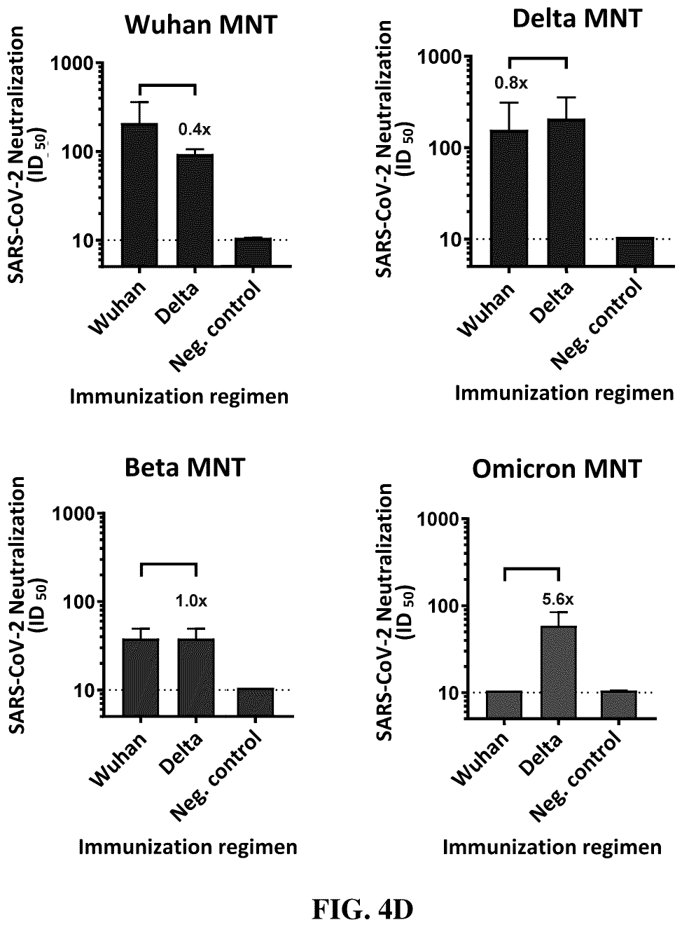

In a specific embodiment, a transgene comprises a nucleotide sequence described in Table 3, infra. In a specific embodiment, a transgene encodes a protein comprising an amino acid sequence described in Table 3, infra. In a specific embodiment, a transgene encoding a chimeric F protein comprises an amino acid sequence described in Table 3, infra. In a specific embodiment, a chimeric F protein is one described in Section 6, infra (e.g., in FIG. 4A). In a specific embodiment, a chimeric F protein comprises an amino acid sequence described in Table 3, infra.

In a specific embodiment, a transgene encoding a chimeric F protein is one described in the Example (Section 6), infra. For example, a chimeric F protein for SARS-CoV-2 delta variant. In specific embodiments, a chimeric F protein is not one known in the art.

In specific embodiments, NDV F protein transmembrane and cytoplasmic domains of a chimeric F protein may be from any NDV strain known in the art or described herein. For example, NDV F protein transmembrane and cytoplasmic domains of a chimeric F protein may be from the NDV F protein of LaSota strain, Hitchner B1 strain, Fuller strain, Ulster strain, Roakin strain, or Komarov strain. In some embodiments, the NDV F protein transmembrane and cytoplasmic domains comprise the amino acid sequence of SEQ ID NO:22.

In certain embodiments, a transgene encoding a protein comprising (or consisting of) a SARS-CoV-2 delta variant spike protein or portion thereof (e.g., the ectodomain, S1 domain, S2 domain, or receptor binding domain of SARS-CoV-2 spike protein) comprises NDV regulatory signals (e.g., gene end, intergenic, and gene start sequences) and Kozak sequences. In some embodiments, a transgene encoding a protein comprising (or consisting of) the ectodomain of a SARS-CoV-2 delta spike protein comprises NDV regulatory signals (e.g., gene end, intergenic, and gene start sequences) and Kozak sequences. In certain embodiments, a transgene encoding a protein comprising (or consisting of) a derivative of SARS-CoV-2 delta variant spike protein or portion thereof (e.g., the ectodomain, S1 domain, S2 domain, or receptor binding domain of SARS-CoV-2 spike protein) comprises NDV regulatory signals (e.g., gene end, intergenic, and gene start sequences) and Kozak sequences. In certain embodiments, a transgene encoding a protein comprising (or consisting of) a derivative of SARS-CoV-2 delta variant spike protein or portion thereof (e.g., the ectodomain, S1 domain, S2 domain, or receptor binding domain of SARS-CoV-2 spike protein) comprises NDV regulatory signals (e.g., gene end, intergenic, and gene start sequences) and Kozak sequences. In some embodiments, a transgene encoding a protein comprising (or consisting of) a derivative of the ectodomain of a SARS-CoV-2 delta variant spike protein comprises NDV regulatory signals (e.g., gene end, intergenic, and gene start sequences) and Kozak sequences. In certain embodiments, a transgene encoding a protein comprising (or consisting of) a chimeric F protein comprises NDV regulatory signals (e.g., gene end, intergenic, and gene start sequences) and Kozak sequences. In some embodiments, a transgene encoding a protein comprising (or consisting of) a SARS-CoV-2 delta variant spike protein or portion thereof (e.g., the ectodomain, S1 domain, S2 domain, or receptor binding domain of SARS-CoV-2 spike protein), or a chimeric F protein comprises NDV regulatory signals (e.g., gene end, intergenic, and gene start sequences), Kozak sequences and restriction sites to facilitate cloning. In some embodiments, a transgene encoding a protein comprising (or consisting of) a derivative of a SARS-CoV-2 delta variant spike protein or portion thereof (e.g., the ectodomain, S1 domain, S2 domain, or receptor binding domain of SARS-CoV-2 spike protein) comprises NDV regulatory signals (e.g., gene end, intergenic, and gene start sequences), Kozak sequences and restriction sites to facilitate cloning. In some embodiments, a transgene encoding a protein comprising (or consisting of) a derivative of a SARS-CoV-2 delta variant spike protein or portion thereof (e.g., the ectodomain, S1 domain, S2 domain, or receptor binding domain of SARS-CoV-2 spike protein) comprises NDV regulatory signals (e.g., gene end, intergenic, and gene start sequences), Kozak sequences and restriction sites to facilitate cloning. In some embodiments, a transgene encoding a protein comprising (or consisting of) a chimeric F protein comprises NDV regulatory signals (e.g., gene end, intergenic, and gene start sequences), Kozak sequences and restriction sites to facilitate cloning. In certain embodiments, a transgene encoding a protein comprising (or consisting of) a SARS-CoV-2 spike protein or portion thereof (e.g., the ectodomain, S1 domain, S2 domain, or receptor binding domain of SARS-CoV-2 spike protein) comprises NDV regulatory signals (gene end, intergenic and gene start sequences), Kozak sequences, restriction sites to facilitate cloning, and additional nucleotides in the non-coding region to ensure compliance with the rule of six. In certain embodiments, a transgene encoding a protein comprising (or consisting of) a derivative of a SARS-CoV-2 spike protein or portion thereof (e.g., the ectodomain, S1 domain, S2 domain, or receptor binding domain of SARS-CoV-2 spike protein) comprises NDV regulatory signals (gene end, intergenic and gene start sequences), Kozak sequences, restriction sites to facilitate cloning, and additional nucleotides in the non-coding region to ensure compliance with the rule of six. In some embodiments, a transgene encoding a protein comprising (or consisting of) a derivative of the ectodomain of a SARS-CoV-2 delta spike protein comprises NDV regulatory signals (gene end, intergenic and gene start sequences), Kozak sequences, restriction sites to facilitate cloning, and additional nucleotides in the non-coding region to ensure compliance with the rule of six. In certain embodiments, a transgene encoding a protein comprising (or consisting of) a chimeric F protein comprises NDV regulatory signals (gene end, intergenic and gene start sequences), Kozak sequences, restriction sites to facilitate cloning, and additional nucleotides in the non-coding region to ensure compliance with the rule of six. See, e.g., SEQ ID NOS: 8-10 and 14 for examples of a restriction sequence (SacII), a gene end sequence, a gene start sequence and a Kozak sequence that may be used. In a preferred embodiment, the transgene complies with the rule of six.

In a specific embodiment, a transgene described herein is isolated.

In one embodiment, provided herein is a nucleic acid sequence encoding a protein comprising (or consisting of) a SARS-CoV-2 delta variant spike protein or a portion thereof (e.g., ectodomain, S1, S2, or receptor binding domain). In another embodiment, provided herein is a nucleic acid sequence encoding a protein comprising (or consisting of) a derivative of a SARS-CoV-2 delta variant spike protein or a portion thereof (e.g., ectodomain, S1, S2, or receptor binding domain). The nucleic acid sequence may be RNA or DNA, and may comprise nucleotide analogs. In some embodiments, the nucleic acid sequence is isolated. In some embodiments, provided herein is a vector comprising the nucleic acid sequence. In some embodiments, the vector is a viral vector or a plasmid. In a specific embodiment, the viral vector is a recombinant NDV.

In a specific embodiment, provided herein is a nucleic acid sequence comprising a nucleotide sequence encoding a protein comprising (or consisting of) the amino acid sequence of SEQ ID NO: 13 or 17. In another specific embodiment, provided herein is a nucleic acid sequence comprising a nucleotide sequence encoding a protein comprising (or consisting of) an amino acid sequence that is at least 99% or at least 99.5% identical to SEQ ID NO: 13 or 17. In some embodiments, the nucleic acid sequence comprises (or consists of) the nucleotide sequence of SEQ ID NO:19 or 20. The nucleic acid sequence may be RNA or DNA, and may comprise nucleotide analogs. In some embodiments, the protein further comprises a trimerization domain (e.g., a T4 foldon domain). In some embodiments, the protein further comprises C-terminus a thrombin cleavage site and a T4 foldon trimerization domain. In some embodiments, the protein further comprises C-terminus a thrombin cleavage site, a T4 foldon trimerization domain, and a hexahistidine tag. In some embodiments, the nucleic acid sequence is isolated. In some embodiments, provided herein is a vector comprising the nucleic acid sequence. In some embodiments, the vector is a viral vector or a plasmid (e.g., a plasmid in Section 6). In a specific embodiment, the viral vector is a recombinant NDV.

In a specific embodiment, provided herein is a nucleic acid sequence comprising the nucleotide sequence encoding a protein comprising (or consisting of) the amino acid sequence of SEQ ID NO: 6 or 18. In another specific embodiment, provided herein is a nucleic acid sequence comprising a nucleotide sequence encoding a protein comprising (or consisting of) an amino acid sequence that is at least 99% or at least 99.5% identical to SEQ ID NO: 6 or 18. In some embodiments, the nucleic acid sequence comprises (or consists of) the nucleotide sequence of SEQ ID NO:5 or 21. The nucleic acid sequence may be RNA or DNA, and may comprise nucleotide analogs. In some embodiments, the nucleic acid sequence is isolated. In some embodiments, provided herein is a vector comprising the nucleic acid sequence. In some embodiments, the vector is a viral vector or a plasmid. In a specific embodiment, the viral vector is a recombinant NDV.

### 5.1.3 Recombinant NDV Encoding a SARS-Cov-2 Spike Protein or a Chimeric F Protein with a SARS-Cov-2 Spike Protein Ectodomain

In one aspect, presented herein are recombinant Newcastle disease virus (“NDV”) comprising a packaged genome, wherein the packaged genome comprises a transgene described herein. In one embodiment, a recombinant NDV comprises a packaged genome, wherein the packaged genome comprises a transgene encoding a SARS-CoV-2 delta variant spike protein or portion thereof (e.g., the ectodomain, S1 domain, S2 domain, or receptor binding domain of SARS-CoV-2 delta variant spike protein). See, e.g., Sections 5.1.2 and 6 for transgenes encoding a SARS-CoV-2 delta variant spike protein or portion thereof (e.g., the ectodomain, S1 domain, S2 domain, or receptor binding domain of SARS-CoV-2 delta variant spike protein) which the packaged genome may comprise. In a specific embodiment, the SARS-CoV-2 spike protein or portion thereof (e.g., the ectodomain, S1 domain, S2 domain, or receptor binding domain of SARS-CoV-2 delta variant spike protein) is expressed by cells infected with the recombinant NDV. In certain embodiments, the SARS-CoV-2 delta variant spike protein or portion thereof (e.g., the ectodomain, S1 domain, S2 domain, or receptor binding domain of SARS-CoV-2 spike protein) is incorporated into the NDV virion.

In another embodiment, a recombinant NDV comprises a packaged genome, wherein the packaged genome comprises a transgene encoding a protein comprising a SARS-CoV-2 delta variant spike protein or portion thereof (e.g., the ectodomain, S1 domain, S2 domain, or receptor binding domain of SARS-CoV-2 delta variant spike protein). See, e.g., Sections 5.1.2 and 6 for transgenes encoding a SARS-CoV-2 delta variant spike protein or portion thereof (e.g., the ectodomain, S1 domain, S2 domain, or receptor binding domain of SARS-CoV-2 delta variant spike protein) which the packaged genome may comprise. In a specific embodiment, the portion of the SARS-CoV-2 spike protein is the ectodomain. In a specific embodiment, the transgene is one described in Section 5.1.2 or 6. In a specific embodiment, the SARS-CoV-2 spike protein or portion thereof (e.g., the ectodomain, S1 domain, S2 domain, or receptor binding domain of SARS-CoV-2 delta variant spike protein) is expressed by cells infected with the recombinant NDV. In certain embodiments, the SARS-CoV-2 delta variant spike protein or portion thereof (e.g., the ectodomain, S1 domain, S2 domain, or receptor binding domain of SARS-CoV-2 spike protein) is incorporated into the NDV virion.

In another embodiment, a recombinant NDV comprises a packaged genome, wherein the packaged genome comprises a transgene encoding a protein comprising a derivative of a SARS-CoV-2 delta variant spike protein or portion thereof (e.g., the ectodomain, S1 domain, S2 domain, or receptor binding domain of SARS-CoV-2 delta variant spike protein). See, e.g., Sections 5.1.2 and 6 for transgenes encoding a derivative of a SARS-CoV-2 delta variant spike protein or portion thereof (e.g., the ectodomain, S1 domain, S2 domain, or receptor binding domain of SARS-CoV-2 delta variant spike protein) which the packaged genome may comprise. In a specific embodiment, the portion of the SARS-CoV-2 spike protein is the ectodomain. In a specific embodiment, the transgene is one described in Section 5.1.2 or 6. In a specific embodiment, the derivative of the SARS-CoV-2 spike protein or portion thereof (e.g., the ectodomain, S1 domain, S2 domain, or receptor binding domain of SARS-CoV-2 delta variant spike protein) is expressed by cells infected with the recombinant NDV. In certain embodiments, the derivative of the SARS-CoV-2 delta variant spike protein or portion thereof (e.g., the ectodomain, S1 domain, S2 domain, or receptor binding domain of SARS-CoV-2 spike protein) is incorporated into the NDV virion.

In another embodiment, described herein are recombinant NDV comprising a packaged genome, wherein the packaged genome comprises a transgene encoding a chimeric F protein described herein. In a specific embodiment, the chimeric F protein is expressed by cells infected with the recombinant NDV. In another specific embodiment, the chimeric F protein is incorporated into the NDV virion. In another specific embodiment, the chimeric F protein is expressed by cells infected with the recombinant NDV and the chimeric F protein is incorporated into the NDV virion.

In a specific embodiment, a recombinant NDV is one described in the Example (Section 6), infra. In specific embodiments, a recombinant NDV described herein is replication competent. In other embodiments, a recombinant NDV described herein has been inactivated.

In certain embodiments, the genome of the recombinant NDV does not comprise a heterologous sequence encoding a heterologous protein other than a SARS-CoV-2 delta variant spike protein or portion thereof (e.g., the ectodomain, S1 domain, S2 domain, or receptor binding domain of SARS-CoV-2 delta variant spike protein). In some embodiments, the genome of the recombinant NDV does not comprise a heterologous sequence encoding a heterologous protein other than a derivative of a SARS-CoV-2 delta variant spike protein or portion thereof (e.g., the ectodomain, S1 domain, S2 domain, or receptor binding domain of SARS-CoV-2 delta variant spike protein). In some embodiments, the genome of the recombinant NDV does not comprise a heterologous sequence encoding a heterologous protein other than a derivative of a SARS-CoV-2 delta variant spike protein ectodomain. In some embodiments, the genome of the recombinant NDV does not comprise a transgene other than a transgene encoding a SARS-CoV-2 delta variant spike protein or portion thereof (e.g., the ectodomain, S1 domain, S2 domain, or receptor binding domain of SARS-CoV-2 spike protein). In some embodiments, the genome of the recombinant NDV does not comprise a transgene other than a transgene encoding a derivative of a SARS-CoV-2 delta variant spike protein or portion thereof (e.g., the ectodomain, S1 domain, S2 domain, or receptor binding domain of SARS-CoV-2 spike protein). In some embodiments, the genome of the recombinant NDV does not comprise a transgene other than a transgene encoding a derivative of the ectodomain of a SARS-CoV-2 delta spike protein or fragment thereof. The fragment may comprise 200, 250, 300, 350, 400, 450, 500, 550, 600, 650, 700, 750, 800, 850, 900, 950, 1000, 1100, 1200 or more contiguous amino acid residues. In certain embodiments, a recombinant NDV described herein comprises a packaged genome, wherein the genome comprises the genes found in NDV and a transgene encoding a SARS-CoV-2 delta variant spike protein or portion thereof (e.g., the ectodomain, S1 domain, S2 domain, or receptor binding domain of SARS-CoV-2 spike protein). In other words, the recombinant NDV encodes for both NDV F protein and the SARS-CoV-2 delta variant spike protein or portion thereof (e.g., the ectodomain, S1 domain, S2 domain, or receptor binding domain of SARS-CoV-2 delta variant spike protein). In some embodiments, a recombinant NDV described herein comprises a packaged genome, wherein the genome comprises the genes found in NDV and a transgene encoding a SARS-CoV-2 delta variant spike protein or portion thereof (e.g., the ectodomain, S1 domain, S2 domain or receptor binding domain of SARS-CoV-2 delta variant spike protein) but does not include any other transgenes. In some embodiments, a recombinant NDV described herein comprises a packaged genome, wherein the genome comprises the genes found in NDV and a transgene encoding a derivative of the ectodomain of the SARS-CoV-2 delta variant spike protein or fragment thereof but does not include any other transgenes.

In certain embodiments, the genome of the recombinant NDV does not comprise a heterologous sequence encoding a heterologous protein other than a protein comprising (or consisting of) a SARS-CoV-2 delta variant spike protein or portion thereof (e.g., the ectodomain, S1 domain, S2 domain, or receptor binding domain of SARS-CoV-2 delta variant spike protein). In some embodiments, the genome of the recombinant NDV does not comprise a transgene other than a transgene encoding a protein comprising (or consisting of) a SARS-CoV-2 delta variant spike protein or portion thereof (e.g., the ectodomain, S1 domain, S2 domain, or receptor binding domain of SARS-CoV-2 delta variant spike protein). In certain embodiments, a recombinant NDV described herein comprises a packaged genome, wherein the genome comprises the genes found in NDV and a transgene encoding a protein comprising (or consisting of) a SARS-CoV-2 delta variant spike protein or portion thereof (e.g., the ectodomain, S1 domain, S2 domain, or receptor binding domain of SARS-CoV-2 delta variant spike protein). In other words, the recombinant NDV encodes for both NDV F protein and the protein comprising (or consisting of) the SARS-CoV-2 delta variant spike protein or portion thereof (e.g., the ectodomain, S1 domain, S2 domain, or receptor binding domain of SARS-CoV-2 delta variant spike protein). In some embodiments, a recombinant NDV described herein comprises a packaged genome, wherein the genome comprises the genes found in NDV and a transgene encoding a protein comprising (or consisting of) a SARS-CoV-2 delta variant spike protein or portion thereof (e.g., the ectodomain, S1 domain, S2 domain or receptor binding domain of SARS-CoV-2 delta variant spike protein) but does not include any other transgenes.

In some embodiments, the packaged genome of NDV encodes a chimeric F protein described herein. In certain embodiment, the genome of the recombinant NDV does not comprise a heterologous sequence encoding a heterologous protein other than the chimeric F protein. In some embodiments, the genome of the recombinant NDV does not comprise a transgene other than a transgene encoding a chimeric F protein described herein. In preferred embodiments, a recombinant NDV described herein comprises a packaged genome, wherein the genome comprises the genes found in NDV and a transgene encoding a chimeric F protein. In other words, the recombinant NDV encodes for both NDV F protein and the chimeric F protein. In some embodiments, a recombinant NDV described herein comprises a packaged genome, wherein the genome comprises the genes found in NDV and a transgene encoding a chimeric F protein, but does not include any other transgenes.

In a specific embodiment, provided herein is a NDV virion comprising a protein comprising (or consisting of) a SARS-CoV-2 delta variant spike protein or a portion thereof (e.g., ectodomain, S1 domain, S2 domain, or receptor binding domain thereof) described herein (e.g., a SARS-CoV-2 delta variant spike protein or portion thereof encoded by a transgene described herein). See, e.g., Section 5.1.2 for examples of such a protein that may incorporated into the virion of a recombinant NDV. In a specific embodiment, the protein is one described in Section 5.1.2, supra. In specific embodiments, the NDV virion is recombinantly produced.

In a specific embodiment, provided herein is a NDV virion comprising a protein comprising (or consisting of) a derivative of a SARS-CoV-2 delta variant spike protein or a portion thereof (e.g., ectodomain, S1 domain, S2 domain, or receptor binding domain thereof) described herein (e.g., a derivative of a SARS-CoV-2 delta variant spike protein or portion thereof encoded by a transgene described herein). See, e.g., Section 5.1.2 for examples of such a protein that may incorporated into the virion of a recombinant NDV. In a specific embodiment, the derivative is a derivative of the SARS-CoV-2 spike protein ectodomain. In a specific embodiment, the protein is one described in Section 5.1.2, supra. In a specific embodiment, the protein is one encoded by a transgene described herein (e.g., in Section 5.1.2 or 6). In specific embodiments, the NDV virion is recombinantly produced.

In a specific embodiment, a SARS-CoV-2 spike protein or portion thereof (e.g., ectodomain) described herein is in a pre-fusion conformation. In some embodiments, a SARS-CoV-2 spike protein or portion thereof (e.g., ectodomain) described herein is in a post-fusion conformation.

In a specific embodiment, a derivative of a SARS-CoV-2 spike protein or portion thereof (e.g., ectodomain) described herein is in a pre-fusion conformation. In some embodiments, a derivative of a SARS-CoV-2 spike protein or portion thereof (e.g., ectodomain) described herein is in a post-fusion conformation.

In a specific embodiment, provided herein is a NDV virion comprising a chimeric F protein described herein (e.g., a chimeric F protein encoded by a transgene described herein). See, e.g., Section 5.1.2 and the Example (e.g., Section 6) for examples of a chimeric F protein that may incorporated into the virion of a recombinant NDV. In a specific embodiment, the chimeric F protein comprises an amino acid sequence that is at least 80%, at least 85%, or at least 90% identical to the amino acid sequence of SEQ ID NO: 6 or 18. In a specific embodiment, the chimeric F protein comprises an amino acid sequence that is at least 95%, at least 98%, or at least 99% identical to the amino acid sequence of SEQ ID NO: 6 or 18. In a specific embodiment, the chimeric F protein comprises the amino acid sequence of SEQ ID NO: 6 or 18. In a specific embodiment, the chimeric F protein is one encoded by a transgene described in Section 5.1.2 or 6. In a specific embodiment, the chimeric F protein is one described in Section 5.1.2 or 6. In specific embodiments, the NDV virion is recombinantly produced.

In a specific embodiment, a chimeric F protein described herein is in a pre-fusion conformation. In some embodiments, a chimeric F protein described herein is in a post-fusion conformation.

### 5.1.4 Codon Optimization

Any codon optimization technique known to one of skill in the art may be used to codon optimize a nucleic acid sequence encoding a SARS-CoV-2 delta variant spike protein or a domain thereof (e.g., the ectodomain or receptor binding domain thereof), or a chimeric F protein. In a specific embodiment, any codon optimization technique known to one of skill in the art may be used to codon optimize a nucleic acid sequence encoding a derivative of a SARS-CoV-2 spike protein or a portion thereof (e.g., ectodomain, S1 domain, S2 domain, or receptor binding domain thereof). Methods of codon optimization are known in the art, e.g., the OptimumGene™ (GenScript®) protocol and Genewiz® protocol, which are incorporated by reference herein in its entirety. See also U.S. Pat. No. 8,326,547 for methods for codon optimization, which is incorporated herein by reference in its entirety.

As an exemplary method for codon optimization, each codon in the open frame of the nucleic acid sequence encoding a SARS-CoV-2 delta variant spike protein or a domain thereof (e.g., the ectodomain or receptor binding protein thereof), or a chimeric F protein is replaced by the codon most frequently used in mammalian proteins. This may be done using a web-based program (www.encorbio.com/protocols/Codon.htm) that uses the Codon Usage Database, maintained by the Department of Plant Gene Research in Kazusa, Japan. This nucleic acid sequence optimized for mammalian expression may be inspected for: (1) the presence of stretches of 5xA or more that may act as transcription terminators; (2) the presence of restriction sites that may interfere with subcloning; (3) compliance with the rule of six. Following inspection, (1) stretches of 5xA or more that may act as transcription terminators may be replaced by synonymous mutations; (2) restriction sites that may interfere with subcloning may be replaced by synonymous mutations; (3) NDV regulatory signals (gene end, intergenic and gene start sequences), and Kozak sequences for optimal protein expression may be added; and (4) nucleotides may be added in the non-coding region to ensure compliance with the rule of six. Synonymous mutations are typically nucleotide changes that do not change the amino acid encoded. For example, in the case of a stretch of 6 As (AAAAAA), which sequence encodes Lys-Lys, a synonymous sequence would be AAGAAG, which sequence also encodes Lys-Lys.

### 5.2 Construction of NDVs

The recombinant NDVs described herein (see, e.g., Sections 5.1 and 6) can be generated using the reverse genetics technique. The reverse genetics technique involves the preparation of synthetic recombinant viral RNAs that contain the non-coding regions of the negative-strand, viral RNA which are essential for the recognition by viral polymerases and for packaging signals necessary to generate a mature virion. The recombinant RNAs are synthesized from a recombinant DNA template and reconstituted in vitro with purified viral polymerase complex to form recombinant ribonucleoproteins (RNPs) which can be used to transfect cells. A more efficient transfection is achieved if the viral polymerase proteins are present during transcription of the synthetic RNAs either in vitro or in vivo. The synthetic recombinant RNPs can be rescued into infectious virus particles. The foregoing techniques are described in U.S. Pat. No. 5,166,057 issued Nov. 24, 1992; in U.S. Pat. No. 5,854,037 issued Dec. 29, 1998; in U.S. Pat. No. 6,146,642 issued Nov. 14, 2000; in European Patent Publication EP 0702085A1, published Feb. 20, 1996; in U.S. patent application Ser. No. 09/152,845; in International Patent Publications PCT WO 97/12032 published Apr. 3, 1997; WO 96/34625 published Nov. 7, 1996; in European Patent Publication EP A780475; WO 99/02657 published Jan. 21, 1999; WO 98/53078 published Nov. 26, 1998; WO 98/02530 published Jan. 22, 1998; WO 99/15672 published Apr. 1, 1999; WO 98/13501 published Apr. 2, 1998; WO 97/06270 published Feb. 20, 1997; and EPO 780 475A1 published Jun. 25, 1997, each of which is incorporated by reference herein in its entirety.

The helper-free plasmid technology can also be utilized to engineer a NDV described herein. Briefly, a complete cDNA of a NDV (e.g., the Hitchner B1 strain or LaSota strain) is constructed, inserted into a plasmid vector and engineered to contain a unique restriction site between two transcription units (e.g., the NDV P and M genes; the NDV NP and P genes; or the NDV HN and L genes). A nucleotide sequence encoding a heterologous amino acid sequence (e.g., a transgene or other sequence described herein such as, e.g., a nucleotide sequence encoding a SARS-CoV-2 delta variant spike protein or portion thereof (e.g., ectodomain or receptor binding domain of the SARS-CoV-2 delta variant spike protein), or a chimeric F protein) may be inserted into the viral genome at the unique restriction site. Alternatively, a nucleotide sequence encoding a heterologous amino acid sequence (e.g., a transgene or other sequence described herein such as, e.g., a nucleotide sequence encoding SARS-CoV-2 delta variant spike protein or portion thereof (e.g., ectodomain or receptor binding domain of the SARS-CoV-2 delta variant spike protein), or a chimeric F protein) may be engineered into a NDV transcription unit so long as the insertion does not affect the ability of the virus to infect and replicate. The single segment is positioned between a T7 promoter and the hepatitis delta virus ribozyme to produce an exact negative or positive transcript from the T7 polymerase. The plasmid vector and expression vectors comprising the necessary viral proteins are transfected into cells leading to production of recombinant viral particles (see, e.g., International Publication No. WO 01/04333; U.S. Pat. Nos. 7,442,379, 6,146,642, 6,649,372, 6,544,785 and 7,384,774; Swayne et al. (2003). Avian Dis. 47:1047-1050; and Swayne et al. (2001). J. Virol. 11868-11873, each of which is incorporated by reference in its entirety).

Bicistronic techniques to produce multiple proteins from a single mRNA are known to one of skill in the art. Bicistronic techniques allow the engineering of coding sequences of multiple proteins into a single mRNA through the use of IRES sequences. IRES sequences direct the internal recruitment of ribosomes to the RNA molecule and allow downstream translation in a cap independent manner. Briefly, a coding region of one protein is inserted downstream of the open reading frame (ORF) of a second protein. The insertion is flanked by an IRES and any untranslated signal sequences necessary for proper expression and/or function. The insertion must not disrupt the open reading frame, polyadenylation or transcriptional promoters of the second protein (see, e.g., Garcia-Sastre et al., 1994, J. Virol. 68:6254-6261 and Garcia-Sastre et al., 1994 Dev. Biol. Stand. 82:237-246, each of which are incorporated by reference herein in their entirety).

Methods for cloning recombinant NDV to encode a transgene and express a heterologous protein encoded by the transgene (e.g., a transgene comprises a nucleotide sequence encoding SARS-CoV-2 delta variant spike protein or portion thereof (e.g., ectodomain, S1 domain, S2 domain, or receptor binding domain of the SARS-CoV-2 delta variant spike protein), or a derivative thereof, or a chimeric F protein) are known to one skilled in the art, such as, e.g., insertion of the transgene into a restriction site that has been engineered into the NDV genome, inclusion an appropriate signals in the transgene for recognition by the NDV RNA-dependent-RNA polymerase (e.g., sequences upstream of the open reading frame of the transgene that allow for the NDV polymerase to recognize the end of the previous gene and the beginning of the transgene, which may be, e.g., spaced by a single nucleotide intergenic sequence), inclusion of a valid Kozak sequence (e.g., to improve eukaryotic ribosomal translation); incorporation of a transgene that satisfies the “rule of six” for NDV cloning; and inclusion of silent mutations to remove extraneous gene end and/or gene start sequences within the transgene. See, e.g., SEQ ID NO:8-10 and 14 for examples of a restriction site sequence, gene end sequence, gene start sequence, and Kozak sequence. Regarding the rule of six, one skilled in the art will understand that efficient replication of NDV (and more generally, most members of the paramyxoviridae family) is dependent on the genome length being a multiple of six, known as the “rule of six” (see, e.g., Calain, P. & Roux, L. The rule of six, a basic feature of efficient replication of Sendai virus defective interfering RNA. J. Virol. 67, 4822-4830 (1993)). Thus, when constructing a recombinant NDV described herein, care should be taken to satisfy the “Rule of Six” for NDV cloning. Methods known to one skilled in the art to satisfy the Rule of Six for NDV cloning may be used, such as, e.g., addition of nucleotides downstream of the transgene. See, e.g., Ayllon et al., Rescue of Recombinant Newcastle Disease Virus from cDNA. J. Vis. Exp. (80), e50830, doi:10.3791/50830 (2013) for a discussion of methods for cloning and rescuing of NDV (e.g., recombinant NDV), which is incorporated by reference herein in its entirety.

In a specific embodiment, an NDV described herein (see, e.g., Section 5.1, and 6) may be generated according to a method described in Section 6, infra.

In a specific embodiment, a recombinant NDV comprising a packaged genome comprising a transgene that comprises a nucleotide sequence encoding a SARS-CoV-2 delta variant spike protein or portion thereof (e.g., ectodomain or receptor binding domain of the SARS-CoV-2 delta variant spike protein) described herein comprises a LaSota strain backbone. In another specific embodiment, a recombinant NDV comprising a packaged genome comprising a transgene that comprises a nucleotide sequence encoding a SARS-CoV-2 delta variant spike protein or portion thereof (e.g., ectodomain, S1 domain, S2 domain, or receptor binding domain of the SARS-CoV-2 delta variant spike protein) described herein comprises a LaSota strain backbone. In a specific embodiment, the genomic sequence of the LaSota strain backbone (i.e., without the transgene) is as set forth in SEQ ID NO:1. In a specific embodiment, the genomic sequence of the LaSota strain backbone (i.e., without the transgene) is as set forth in SEQ ID NO:3. As the skilled person will appreciate, the genome of NDV is negative-sense and single stranded. SEQ ID NOS:1 and 3 provide cDNA sequences.

In a specific embodiment, a recombinant NDV comprising a packaged genome comprising a transgene that comprises a nucleotide sequence encoding a derivative of a SARS-CoV-2 delta variant spike protein or portion thereof (e.g., ectodomain or receptor binding domain of the SARS-CoV-2 delta variant spike protein) described herein comprises a LaSota strain backbone. In another specific embodiment, a recombinant NDV comprising a packaged genome comprising a transgene that comprises a nucleotide sequence encoding a derivative of a SARS-CoV-2 delta variant spike protein or portion thereof (e.g., ectodomain, S1 domain, S2 domain, or receptor binding domain of the SARS-CoV-2 delta variant spike protein) described herein comprises a LaSota strain backbone. In a specific embodiment, the genomic sequence of the LaSota strain backbone (i.e., without the transgene) is as set forth in SEQ ID NO:1. In a specific embodiment, the genomic sequence of the LaSota strain backbone (i.e., without the transgene) is as set forth in SEQ ID NO:3. As the skilled person will appreciate, the genome of NDV is negative-sense and single stranded. SEQ ID NOS:1 and 3 provide cDNA sequences.

In a specific embodiment, a recombinant NDV comprising a packaged genome comprising a transgene encoding a chimeric F protein described herein comprises a LaSota strain backbone. In a specific embodiment, a recombinant NDV comprising a packaged genome comprising a transgene encoding a chimeric F protein described herein comprises a LaSota strain backbone. In a specific embodiment, the genomic sequence of the LaSota strain backbone (i.e., without the transgene) is as set forth in SEQ ID NO:1. In another specific embodiment, the genomic sequence of the LaSota strain backbone (i.e., without the transgene) is as set forth in SEQ ID NO:3. As the skilled person will appreciate, the genome of NDV is negative-sense and single stranded. SEQ ID NOS:1 and 3 provide cDNA sequences.

### 5.3 Propagation of NDVs

The recombinant NDVs described herein (e.g., Sections 5.1 and 6) can be propagated in any substrate that allows the virus to grow to titers that permit the uses of the viruses described herein. In one embodiment, the substrate allows the recombinant NDVs described herein to grow to titers comparable to those determined for the corresponding wild-type viruses.

The recombinant NDVs described herein (e.g., Sections 5.1 and 6) may be grown in cells (e.g., avian cells, chicken cells, etc.) that are susceptible to infection by the viruses, embryonated eggs (e.g., chicken eggs or quail eggs) or animals (e.g., birds). Such methods are well known to those skilled in the art. In a specific embodiment, the recombinant NDVs described herein may be propagated in cancer cells, e.g., carcinoma cells (e.g., breast cancer cells and prostate cancer cells), sarcoma cells, leukemia cells, lymphoma cells, and germ cell tumor cells (e.g., testicular cancer cells and ovarian cancer cells). In another specific embodiment, the recombinant NDVs described herein may be propagated in cell lines, e.g., cancer cell lines such as HeLa cells, MCF7 cells, THP-1 cells, U87 cells, DU145 cells, Lncap cells, and T47D cells. In certain embodiments, the cells or cell lines (e.g., cancer cells or cancer cell lines) are obtained, derived, or obtained and derived from a human(s). In another embodiment, the recombinant NDVs described herein are propagated in interferon deficient systems or interferon (IFN) deficient substrates, such as, e.g., IFN deficient cells (e.g., IFN deficient cell lines) or IFN deficient embryonated eggs. In another embodiment, the recombinant NDVs described herein are propagated in chicken cells or embryonated chicken eggs. Representative chicken cells include, but are not limited to, chicken embryo fibroblasts and chicken embryo kidney cells. In a specific embodiment, the recombinant NDVs described herein are propagated in Vero cells. In another specific embodiment, the recombinant NDVs described herein are propagated in chicken eggs or quail eggs. In certain embodiments, a recombinant NDV virus described herein is first propagated in embryonated eggs and then propagated in cells (e.g., a cell line). In another specific embodiment, the recombinant NDVs described herein are propagated as described in Section 6, infra.

The recombinant NDVs described herein may be propagated in embryonated eggs (e.g. chicken embryonated eggs), e.g., from 6 to 14 days old, 6 to 12 days old, 6 to 10 days old, 6 to 9 days old, 6 to 8 days old, 8 to 10 day old, 9 to 11 days old, or 10 to 12 days old. In a specific embodiment, 10 day old embryonated chicken eggs are used to propagate the recombinant NDVs described herein. Young or immature embryonated eggs (e.g. chicken embryonated eggs) can be used to propagate the recombinant NDVs described herein. Immature embryonated eggs encompass eggs which are less than ten day old eggs, e.g., eggs 6 to 9 days old or 6 to 8 days old that are IFN-deficient. Immature embryonated eggs also encompass eggs which artificially mimic immature eggs up to, but less than ten day old, as a result of alterations to the growth conditions, e.g., changes in incubation temperatures; treating with drugs; or any other alteration which results in an egg with a retarded development, such that the IFN system is not fully developed as compared with ten to twelve day old eggs. The recombinant NDVs described herein can be propagated in different locations of the embryonated egg, e.g., the allantoic cavity (such as, e.g., the allantoic cavity of chicken embryonated eggs). For a detailed discussion on the growth and propagation viruses, see, e.g., U.S. Pat. Nos. 6,852,522 and 7,494,808, both of which are hereby incorporated by reference in their entireties.

In a specific embodiment, a virus is propagated as described in the Example below (e.g., Section 6).

For virus isolation, the recombinant NDVs described herein can be removed from embryonated eggs or cell culture and separated from cellular components, typically by well-known clarification procedures, e.g., such as centrifugation, depth filtration, and microfiltration, and may be further purified as desired using procedures well known to those skilled in the art, e.g., tangential flow filtration (TFF), density gradient centrifugation, differential extraction, or chromatography. In a specific embodiment, a virus is isolated as described in the Example below (e.g., Section 6).

In a specific embodiment, virus isolation from allantoic fluid of an infected egg (e.g., a chicken egg) begins with harvesting allantoic fluid, which is clarified using a filtration system to remove cells and other large debris.

In a specific embodiment, provided herein is a cell (e.g., a cell line) or embryonated egg (e.g., a chicken embryonated egg) comprising a recombinant NDV described herein. In another specific embodiment, provided herein is a method for propagating a recombinant NDV described herein, the method comprising culturing a cell (e.g., a cell line) or embryonated egg (e.g., a chicken embryonated egg) infected with the recombinant NDV. In some embodiments, the method may further comprise isolating or purifying the recombinant NDV from the cell or embryonated egg. In a specific embodiment, provided herein is a method for propagating a recombinant NDV described herein, the method comprising (a) culturing a cell (e.g., a cell line) or embryonated egg infected with a recombinant NDV described herein; and (b) isolating the recombinant NDV from the cell or embryonated egg. In specific embodiments, the cell (e.g., cell line) or embryonated egg (e.g., a chicken embryonated egg) is cultured in vitro or ex vivo. The cell or embryonated egg may be one described herein (e.g., in Section 5.3 or 6) or known to one of skill in the art. In some embodiments, the cell or embryonated egg is IFN deficient.

In a specific embodiment, provided herein is a method for producing a pharmaceutical composition (e.g., an immunogenic composition) comprising a recombinant NDV described herein, the method comprising (a) propagating a recombinant NDV described herein a cell (e.g., a cell line) or embryonated egg (e.g., a chicken embryonated egg); and (b) isolating the recombinant NDV from the cell or embryonated egg. In specific embodiments, the cell (e.g., cell line) or embryonated egg (e.g., a chicken embryonated egg) is cultured in vitro or ex vivo. The method may further comprise adding the recombinant NDV to a container along with a pharmaceutically acceptable carrier.

### 5.4 Compositions and Routes of Administration

Provided herein are compositions comprising a recombinant NDV described herein (e.g., Section 5.1, or 6). In a specific embodiment, the compositions are pharmaceutical compositions, such as immunogenic compositions (e.g., vaccine compositions). In a specific embodiment, provided herein are immunogenic compositions comprising a recombinant NDV described herein (e.g., Section 5.1, or 6). The compositions may be used in methods of inducing an immune response to SARS-CoV-2 delta variant spike protein. The compositions may be used in methods for inducing an immune response to SARS-CoV-2 delta variant or immunizing against SARS-CoV-2 delta variant. The compositions may be used in methods for immunizing against COVID-19. The compositions may be used in methods for preventing COVID-19.

In one embodiments, a pharmaceutical composition comprises a recombinant NDV described herein (e.g., Section 5.1, or 6), in an admixture with a pharmaceutically acceptable carrier. In some embodiments, the pharmaceutical composition further comprises one or more additional prophylactic or therapeutic agents. In a specific embodiment, a pharmaceutical composition comprises an effective amount of a recombinant NDV described herein (e.g., Section 5.1, or 6), and optionally one or more additional prophylactic or therapeutic agents, in a pharmaceutically acceptable carrier. In some embodiments, the recombinant NDV (e.g., Section 5.1, or 6) is the only active ingredient included in the pharmaceutical composition. In specific embodiments, two or more recombinant NDV are included in the pharmaceutical composition. In a particular embodiment, the pharmaceutical composition is an immunogenic composition. In a specific embodiment, administration of an immunogenic composition described herein to a subject (e.g., a human) generates neutralizing antibody (e.g., anti-SARS-CoV-2 delta variant spike protein IgG).

In a specific embodiment, an immunogenic composition is one described in Section 6, infra.

In a specific embodiments, provided herein are compositions comprising a nucleic acid sequence described herein or nucleotide sequence described herein. In a specific embodiment, the compositions are pharmaceutical compositions, such as immunogenic compositions (e.g., vaccine compositions). In a specific embodiments, provided herein is a pharmaceutical composition (e.g., an immunogenic composition) comprising a nucleic acid sequence described or nucleotide sequence described herein in an admixture with a pharmaceutically acceptable carrier. In some embodiments, the composition further comprises one or more additional prophylactic or therapeutic agents. In some embodiments, the nucleic acid sequence or nucleotide sequence is the only active ingredient included in the pharmaceutical composition. The compositions may be used in methods of inducing an immune response to SARS-CoV-2 delta variant spike protein. The compositions may be used in methods for inducing an immune response to SARS-CoV-2 delta variant or immunizing against SARS-CoV-2 delta variant. The compositions may be used in methods for immunizing against COVID-19. The compositions may be used in methods for preventing COVID-19.

In specific embodiments, provided herein are compositions comprising a transgene described herein. In a specific embodiment, the compositions are pharmaceutical compositions, such as immunogenic compositions (e.g., vaccine compositions). In a specific embodiments, provided herein is a pharmaceutical composition (e.g., an immunogenic composition) comprising a transgene described herein in an admixture with a pharmaceutically acceptable carrier. In some embodiments, the composition further comprises one or more additional prophylactic or therapeutic agents. In some embodiments, the transgene is the only active ingredient included in the pharmaceutical composition. The compositions may be used in methods of inducing an immune response to SARS-CoV-2 delta variant spike protein. The compositions may be used in methods for inducing an immune response to SARS-CoV-2 delta variant or immunizing against SARS-CoV-2 delta variant. The compositions may be used in methods for immunizing against COVID-19. The compositions may be used in methods for preventing COVID-19.

In specific embodiments, provided herein are compositions comprising a protein described herein. In a specific embodiment, the compositions are pharmaceutical compositions, such as immunogenic compositions (e.g., vaccine compositions). In a specific embodiment, the compositions are pharmaceutical compositions, such as immunogenic compositions (e.g., vaccine compositions). In a specific embodiments, provided herein is a pharmaceutical composition (e.g., an immunogenic composition) comprising a protein described herein in an admixture with a pharmaceutically acceptable carrier. In some embodiments, the composition further comprises one or more additional prophylactic or therapeutic agents. In some embodiments, the protein is the only active ingredient included in the pharmaceutical composition. The compositions may be used in methods of inducing an immune response to SARS-CoV-2 delta variant spike protein. The compositions may be used in methods for inducing an immune response to SARS-CoV-2 delta variant or immunizing against SARS-CoV-2 delta variant. The compositions may be used in methods for immunizing against COVID-19. The compositions may be used in methods for preventing COVID-19.

In certain embodiments, administration of an immunogenic composition described herein to a subject (e.g., a human) generates an immune response that provides some level of protection against developing COVID-19. In some embodiments, administration of an immunogenic composition to a subject (e.g., human) generates an immune response in the subject that reduces the likelihood of developing COVID-19 by at least 25%, at least 50%, at least 75%, at least 80%, at least 85%, at least 90%, or at least 95% relative a subject of the same species not administered the immunogenic composition.

In a specific embodiment, the recombinant NDV included in a pharmaceutical composition (e.g., an immunogenic composition) described herein is a live virus. In a particular embodiment, the recombinant NDV included in a pharmaceutical composition described herein is an attenuated live virus. In some embodiments, the recombinant NDV included in a pharmaceutical composition (e.g., an immunogenic composition) described herein is inactivated. Any technique known to one of skill in the art may be used to inactivate a recombinant NDV described herein. For example, formalin or beta-propiolactone may be used to inactivate a recombinant NDV described herein. In a specific embodiment, the recombinant NDV included in a composition described herein is inactivated using 0.05% to 2% (e.g., 0.05%, 0.1%, 0.5%, 1%, or 2%) beta-Propiolactone, or another technique known to one of skill in the art. In a specific embodiment, the recombinant NDV included in a pharmaceutical described herein is inactivated using 2% beta-Propiolactone, or another technique known to one of skill in the art. For example, in certain embodiments, to prepare inactivated concentrated recombinant NDV, 1 part of 0.5 M disodium phosphate (DSP) may be mixed with 38 parts of the allantoic fluid of an embryonated egg infected with the virus to stabilize the pH, one part of 2% beta-Propiolactone (BPL) is added dropwise to the mixture during shaking, and incubated on ice for 30 min, the mixture is then placed in a 37° C. water bath for approximately 1 to 3 hours shaken every 5-30 min. The inactivated allantoic fluid is clarified by centrifugation at 4,000 rpm for 20-40 minutes. In another example, recombinant NDV in allantoic fluid is inactivated in 0.05% beta-propiolactone. The inactivated allantoic fluid may be clarified by centrifugation at 4,000 rpm for 20-40 minutes (e.g., about 30 minutes). The clarified allantoic fluids may be laid on top of a 20% sucrose cushion in PBS and ultracentrifuged at 25,000 rpm for about 2 hours at 4° C. using, e.g., a Beckman L7-65 ultracentrifuge with a Beckman SW28 rotor, to pellet the virus through the sucrose cushion to remove soluble egg protein. The virus may then be resuspended in PBS at, e.g., about pH 7 to about 7.6 (such as, e.g., pH 7.4). In specific embodiments, the total protein is determined using the bicinchoninic acid (BCA) assay, or another assay known to one of skill in the art. In a specific embodiment, the recombinant NDV is inactivated as described in Section 6, infra. In a specific embodiment, a chimeric F protein is stable in an inactivated recombinant NDV described herein for a period of time (e.g., for 1 month, 2 months, 3 months, 4 months, 5 months, 6 months, or longer), as assessed by the ability of the inactivated recombinant NDV to induce anti-SARS-CoV-2 delta variant spike protein antibodies.

In specific embodiments, a pharmaceutical composition (e.g., an immunogenic composition) described herein or a recombinant NDV described herein does not require frozen storage, which makes it difficult to transport and store in low-income countries. In specific embodiments, a pharmaceutical composition (e.g., an immunogenic composition) described herein or a recombinant NDV described herein may be stored at about 2° C. to about SoC (e.g., 4° C.).

The pharmaceutical compositions provided herein can be in any form that allows for the composition to be administered to a subject. In a specific embodiment, the pharmaceutical compositions are suitable for veterinary administration, human administration, or both. As used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the pharmaceutical composition is administered. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Examples of suitable pharmaceutical carriers are described in “Remington's Pharmaceutical Sciences” by E. W. Martin. The formulation should suit the mode of administration.

In a specific embodiment, the pharmaceutical compositions are formulated to be suitable for the intended route of administration to a subject. For example, the pharmaceutical composition may be formulated to be suitable for parenteral, intravenous, intraarterial, intrapleural, inhalation, intranasal, intraperitoneal, oral, intradermal, colorectal, intraperitoneal, and intracranial administration. In one embodiment, the pharmaceutical composition may be formulated for intravenous, intraarterial, oral, intraperitoneal, intranasal, intratracheal, intrapleural, intracranial, subcutaneous, intramuscular, topical, or pulmonary administration. In a specific embodiment, the pharmaceutical composition may be formulated for intranasal administration. In certain embodiments, the pharmaceutical composition is formulated for a nasal spray. In another embodiment, the pharmaceutical composition may be formulated for intramuscular administration.

In a specific embodiment, the pharmaceutical composition comprising a recombinant NDV described herein (see, e.g., Sections 5.1 and 6) is formulated to be suitable for intranasal administration to the subject (e.g., human subject). In a particular embodiment, the pharmaceutical composition is an immunogenic composition.

In a specific embodiment, a pharmaceutical composition described herein (e.g., a pharmaceutical composition comprising an inactivated recombinant NDV described herein) may comprise an adjuvant. In certain embodiments, the compositions described herein comprise, or are administered in combination with, an adjuvant. The adjuvant for administration in combination with a composition described herein may be administered before, concomitantly with, or after administration of the composition. In specific embodiments, an inactivated virus immunogenic composition described herein comprises one or more adjuvants. In some embodiments, the term “adjuvant” refers to a compound that when administered in conjunction with or as part of a composition described herein augments, enhances and/or boosts the immune response to a recombinant NDV, but when the compound is administered alone does not generate an immune response to the virus. In some embodiments, the adjuvant generates an immune response to a recombinant NDV and does not produce an allergy or other adverse reaction. Adjuvants can enhance an immune response by several mechanisms including, e.g., lymphocyte recruitment, stimulation of B and/or T cells, and stimulation of macrophages. Specific examples of adjuvants include, but are not limited to, aluminum salts (alum) (such as aluminum hydroxide, aluminum phosphate, and aluminum sulfate), 3 De-O-acylated monophosphoryl lipid A (MPL) (see GB 2220211), MF59 (Novartis), AS03 (GlaxoSmithKline), AS04 (GlaxoSmithKline), polysorbate 80 (Tween 80; ICL Americas, Inc.), imidazopyridine compounds (see International Application No. PCT/US2007/064857, published as International Publication No. WO2007/109812), imidazoquinoxaline compounds (see International Application No. PCT/US2007/064858, published as International Publication No. WO2007/109813) and saponins, such as QS21 (see Kensil et al., in Vaccine Design: The Subunit and Adjuvant Approach (eds. Powell & Newman, Plenum Press, NY, 1995); U.S. Pat. No. 5,057,540). In some embodiments, the adjuvant is Freund's adjuvant (complete or incomplete). Other adjuvants are oil in water emulsions (such as squalene or peanut oil), optionally in combination with immune stimulants, such as monophosphoryl lipid A (see Stoute et al, N. Engl. J. Med. 336, 86-91 (1997)). Another adjuvant is CpG (Bioworld Today, Nov. 15, 1998). Such adjuvants can be used with or without other specific immunostimulating agents such as MPL or 3-DMP, QS21, polymeric or monomeric amino acids such as poly glutamic acid or polylysine. In certain embodiments, the adjuvant is a liposomal suspension adjuvant (R-enantiomer of the cationic lipid DOTAP, R-DOTAP) or an MF-59 like oil-in-water emulsion adjuvant (AddaVax). The adjuvant may be a toll-like receptor (TLR) agonist (e.g., a TLR7 agonist, TLR8 agonist, TLR7/8 agonist, or TLR9 agonist). In some embodiments, the adjuvant is a toll-like receptor 9 (TLR9) agonist adjuvant. In certain embodiments, the adjuvant is CpG 1018. In some embodiments, a composition described herein (e.g., a live recombinant NDV composition) does not contain an adjuvant.

In certain embodiments, a pharmaceutical composition (e.g., an immunogenic composition) described herein comprises an effective amount of a recombinant NDV described herein. In specific embodiments, an effective amount of a recombinant NDV described herein is an amount of recombinant NDV to generate an immune response in a subject or a population of subjects. In specific embodiments, an effective amount of a recombinant NDV described herein is 10⁴ to 10¹² PFU or EID50. In some embodiments, an effective amount comprises 1 to 15 micrograms of a SARS-CoV-2 delta variant spike protein or a portion thereof (e.g., an ectodomain), a derivative of a SARS-CoV-2 delta variant spike protein or a portion thereof (e.g., an ectodomain), or a chimeric F protein expressed by a recombinant NDV described herein.

In certain embodiments, a pharmaceutical composition (e.g., an immunogenic composition) described herein comprises 10⁴ to 10¹² EID50 of a recombinant NDV described herein. In some embodiments, a pharmaceutical composition (e.g., an immunogenic composition) described herein comprises 1 to 15 micrograms of a SARS-CoV-2 delta variant spike protein or a portion thereof (e.g., an ectodomain), a derivative of a SARS-CoV-2 delta variant spike protein or a portion thereof (e.g., an ectodomain), or a chimeric F protein expressed by a recombinant NDV described herein. In some embodiments, a pharmaceutical composition (e.g., an immunogenic composition) described herein comprises 1 to 15 micrograms of a SARS-CoV-2 delta variant spike protein or a portion thereof (e.g., an ectodomain), a derivative of a SARS-CoV-2 delta variant spike protein or a portion thereof (e.g., an ectodomain), or a chimeric F protein expressed by a recombinant NDV described herein. In some embodiments, a pharmaceutical composition (e.g., an immunogenic composition) described herein comprises 1 to 15 micrograms per ml of a SARS-CoV-2 delta variant spike protein or a portion thereof (e.g., an ectodomain), a derivative of a SARS-CoV-2 delta variant spike protein or a portion thereof (e.g., an ectodomain), or a chimeric F protein expressed by a recombinant NDV described herein.

In some embodiments, an immunogenic composition described herein comprises 1 to 15 micrograms of inactivated recombinant NDV described herein.

In a specific embodiment, a pharmaceutical composition (e.g., immunogenic composition) described herein may be stored at 2° to 8° C. (e.g., 4° C.). In certain embodiments, a pharmaceutical composition (e.g., immunogenic composition) described herein is stable for at least 1 month, at least 2 months, at least 3 months, at least 4 months, at least 5 months, at least 6 months, at least 9 months or at least 1 year at 2° to 8° C. In some embodiments, a pharmaceutical composition (e.g., immunogenic composition) described herein is stable for 3-6 months, 3-9 months, 6-12 months, or 9-12 months at 2° to 8° C. (e.g., 4° C.). In certain embodiments, the stability is assessed by protein denaturation assays, immunoassays or a combination thereof.

### 5.5 Uses of a Recombinant NDV

### 5.5.1 Prevention of COVID-19

The recombinant NDV described herein may be used to immunize a subject against SARS-CoV-2, induce an immune response to SARS-CoV-2 spike protein, or prevent COVID-19. In a specific aspect, the recombinant NDV described herein may be used to immunize a subject against a SARS-CoV-2 delta variant, induce an immune response to a SARS-CoV-2 delta variant spike protein, or prevent COVID-19 caused by or associated with a SARS-CoV-2 delta variant.

In a specific embodiment, presented herein is a method for inducing an immune response to a SARS-CoV-2 delta variant spike protein in a subject (e.g., a human subject), comprising administering the subject (e.g., a human subject) a recombinant NDV described herein or an immunogenic composition described herein. In another specific embodiment, presented herein is a method for inducing an immune response to a SARS-CoV-2 delta variant spike protein in a subject (e.g., a human subject), comprising administering the subject (e.g., a human subject) an effective amount of a recombinant NDV described herein or an immunogenic composition described herein. In a specific embodiment, the immunogenic composition is one described in Section 5.4 or 6.

In one aspect, presented herein are methods for inducing an immune response in a subject (e.g., a human subject), comprising administering the subject (e.g., a human subject) a recombinant NDV described herein or a composition comprising a recombinant NDV described herein. In one embodiment, presented herein is a method for inducing an immune response to a SARS-CoV-2 spike protein in a subject (e.g., a human subject), comprising administering the subject (e.g., a human subject) a recombinant NDV described herein or a composition described herein, such as described in Section 5.4. In a specific embodiment, presented herein is a method for inducing an immune response to a SARS-CoV-2 delta variant spike protein in a subject (e.g., a human subject), comprising administering the subject (e.g., a human subject) a recombinant NDV described herein or a composition described herein. In another embodiment, presented herein is a method for inducing an immune response to a SARS-CoV-2 spike protein in a subject (e.g., a human subject), comprising administering the subject (e.g., a human subject) an effective amount of a recombinant NDV described herein or a composition described herein. In a specific embodiment, presented herein is a method for inducing an immune response to a SARS-CoV-2 delta variant spike protein in a subject (e.g., a human subject), comprising administering the subject (e.g., a human subject) an effective amount of a recombinant NDV described herein or a composition described herein. See, e.g., Section 5.1 and the Examples for recombinant NDV and Section 5.4 for compositions. In a specific embodiment, the recombinant NDV is one described in Section 5.1 or 6, and the immunogenic composition is one described in Section 5.4 or 6.

In another aspect, presented herein are methods for immunizing a subject (e.g., a human subject) against SARS-CoV-2 (e.g., a SARS-CoV-2 delta variant) comprising administering the subject (e.g., a human subject) a recombinant NDV described herein or a composition comprising a recombinant NDV described herein. In one embodiment, presented herein is a method for immunizing a subject (e.g., a human subject) against SARS-CoV-2, comprising administering the subject (e.g., a human subject) a recombinant NDV described herein or a composition described herein. In another embodiment, presented herein is a method for immunizing a subject (e.g., a human subject) against SARS-CoV-2, comprising administering the subject (e.g., a human subject) an effective amount of a recombinant NDV described herein or a composition described herein. See, e.g., Section 5.1 and 6 for recombinant NDV and Section 5.4 and 6 for compositions. In a specific embodiment, the recombinant NDV is one described in Section 5.1 or 6, and the immunogenic composition is one described in Section 5.4 or 6.

In a specific embodiment, presented herein is a method for immunizing a subject (e.g., a human subject) against SARS-CoV-2 delta variant, comprising administering the subject (e.g., a human subject) a recombinant NDV described herein or a composition described herein. In another specific embodiment, presented herein is a method for immunizing a subject (e.g., a human subject) against SARS-CoV-2 delta variant, comprising administering the subject (e.g., a human subject) an effective amount of a recombinant NDV described herein or a composition described herein. See, e.g., Section 5.1 and the Examples for recombinant NDV and Section 5.4 for compositions. In a specific embodiment, the recombinant NDV is one described in Section 5.1 or 6, and the immunogenic composition is one described in Section 5.4 or 6.

In another aspect, presented herein are methods for preventing COVID-19 in a subject (e.g., a human subject), comprising administering the subject (e.g., a human subject) a recombinant NDV described herein or a composition comprising a recombinant NDV described herein. In one embodiment, presented herein is a method for preventing COVID-19 in a subject (e.g., a human subject), comprising administering the subject (e.g., a human subject) a recombinant NDV described herein or a composition described herein. In another embodiment, presented herein is a method for preventing COVID-19 in a subject (e.g., a human subject), comprising administering the subject (e.g., a human subject) an effective amount of a recombinant NDV described herein or a composition described herein. See, e.g., Section 5.1 and the Example for recombinant NDV and Section 5.4 for compositions. The COVID-19 may be caused by or associated with a SARS-CoV-2 delta variant. Alternatively, the COVID-19 may be caused or associated with SARS-CoV-2 that is not a delta variant.

The recombinant NDV described herein may be administered to a subject in combination with one or more other therapies. The recombinant NDV and one or more other therapies may be administered by the same or different routes of administration to the subject. In a specific embodiment, the recombinant NDV is administered to a subject intranasally. See, e.g., Sections 5.1, and 6, infra for information regarding recombinant NDV, Section 5.5.3 for information regarding other therapies, and Section 5.4, infra, for information regarding compositions and routes of administration.

The recombinant NDV and one or more additional therapies may be administered concurrently or sequentially to the subject. In certain embodiments, the recombinant NDV and one or more additional therapies are administered in the same composition. In other embodiments, the recombinant NDV and one or more additional therapies are administered in different compositions. The recombinant NDV and one or more other therapies may be administered by the same or different routes of administration to the subject. Any route known to one of skill in the art or described herein may be used to administer the recombinant NDV and one or more other therapies. In a specific embodiment, the recombinant NDV is administered intranasally or intramuscularly and the one or more other therapies are administered by the same or a different route. In a specific embodiment, the recombinant NDV is administered intranasally and the one or more other therapies is administered intravenously.

In certain embodiments, a recombinant NDV described herein or a composition described herein is administered to a subject previously vaccinated with a COVID-19 vaccine. In some embodiments, a recombinant NDV described herein or a composition described herein is administered to a subject previously vaccinated with a COVID-19 vaccine other than a NDV-based COVID-19 vaccine. The COVID-19 vaccine may be Pfizer's COVID-19 vaccine, Moderna's COVID-19 vaccine, AstraZeneca's COVID-19 vaccine, Johnson & Johnson's COVID-19, or another COVID-19 vaccine. In certain embodiments, a recombinant NDV described herein or a composition described herein is administered to a subject previously vaccinated with an immunogenic composition other than one described herein. In certain embodiments, a recombinant NDV described herein or a composition described herein is administered to a subject previously infected with SARS-CoV-2 (e.g., a SARS-CoV-2 delta variant or another SARS-CoV-2). In some embodiments, a recombinant NDV described herein or a composition described herein is administered to a subject previously diagnosed with a SARS-CoV-2 infection (e.g., a SARS-CoV-2 delta variant infection or another SARS-CoV-2 infection). In some embodiments, a recombinant NDV described herein or a composition described herein is administered to a subject previously experiencing symptoms of COVID-19. In certain embodiments, a recombinant NDV described herein or a composition described herein is administered to a subject previously diagnosed with COVID-19.

In a specific embodiment, provided herein is a method for boosting a subject's (e.g., a human's) immune response to SARS-CoV-2 (e.g., SARS-CoV-2 delta variant), comprising administering to the subject a recombinant NDV described herein or a composition described herein. In another specific embodiment, provided herein is a method for boosting a subject's (e.g., a human's) immune response to SARS-CoV-2 (e.g., SARS-CoV-2 delta variant), comprising administering to the subject an effective amount of a recombinant NDV described herein or a composition described herein. In specific embodiments, the subject administered the recombinant NDV or composition was previously vaccinated with a COVID-19 vaccine. In specific embodiments, the subject administered the recombinant NDV or composition was previously infected with SARS-CoV-2, as detected by techniques known in the art, such as, e.g., a rapid antigen test or PCR test. In specific embodiments, the subject administered the recombinant NDV or composition previously experienced COVID-19 symptoms.

In a specific embodiment, provided herein is a method for boosting a subject's (e.g., a human's) immune response to SARS-CoV-2 spike protein (e.g., SARS-CoV-2 delta variant spike protein), comprising administering to the subject a recombinant NDV described herein or a composition described herein. In another specific embodiment, provided herein is a method for boosting a subject's (e.g., a human's) immune response to SARS-CoV-2 spike protein (e.g., SARS-CoV-2 delta variant spike protein), comprising administering to the subject an effective amount of a recombinant NDV described herein or a composition described herein. In specific embodiments, the subject administered the recombinant NDV or composition was previously vaccinated with a COVID-19 vaccine. In specific embodiments, the subject administered the recombinant NDV or composition was previously infected with SARS-CoV-2, as detected by techniques known in the art, such as, e.g., a rapid antigen test or PCR test. In specific embodiments, the subject administered the recombinant NDV or composition previously experienced COVID-19 symptoms.

In another specific embodiment, provided herein is a method for boosting immunity to SARS-CoV-2 in a subject (e.g., a human), comprising administering to the subject the recombinant NDV described herein or the immunogenic composition described herein. In specific embodiments, the subject administered the recombinant NDV or composition was previously vaccinated with a COVID-19 vaccine. In specific embodiments, the subject administered the recombinant NDV or composition was previously infected with SARS-CoV-2, as detected by techniques known in the art, such as, e.g., a rapid antigen test or PCR test. In specific embodiments, the subject administered the recombinant NDV or composition previously experienced COVID-19 symptoms.

In a specific embodiment, provided herein is a method for increasing the titer of antibody(ies) that binds to SARS-CoV-2 spike protein (e.g., SARS-CoV-2 delta variant spike protein) in a subject (e.g., a human), comprising administering to the subject a recombinant NDV described herein or a composition described herein. In another specific embodiment, provided herein is a method for increasing the titer of antibody(ies) that binds to SARS-CoV-2 spike protein (e.g., SARS-CoV-2 delta variant spike protein) in a subject (e.g., a human), comprising administering to the subject an effective amount of a recombinant NDV described herein or a composition described herein. In specific embodiments, the subject administered the recombinant NDV or composition was previously vaccinated with a COVID-19 vaccine. In specific embodiments, the subject administered the recombinant NDV or composition was previously infected with SARS-CoV-2, as detected by techniques known in the art, such as, e.g., a rapid antigen test or PCR test. In specific embodiments, the subject administered the recombinant NDV or composition previously experienced COVID-19 symptoms.

In a specific embodiment, provided herein is a method for increasing the titer of neutralizing antibody(ies) that binds to SARS-CoV-2 spike protein (e.g., SARS-CoV-2 delta variant spike protein) in a subject (e.g., a human), comprising administering to the subject a recombinant NDV described herein or a composition described herein. In another specific embodiment, provided herein is a method for increasing the titer of neutralizing antibody(ies) that binds to SARS-CoV-2 spike protein (e.g., SARS-CoV-2 delta variant spike protein) in a subject (e.g., a human), comprising administering to the subject an effective amount of a recombinant NDV described herein or a composition described herein. In specific embodiments, the subject administered the recombinant NDV or composition was previously vaccinated with a COVID-19 vaccine. In specific embodiments, the subject administered the recombinant NDV or composition was previously infected with SARS-CoV-2, as detected by techniques known in the art, such as, e.g., a rapid antigen test or PCR test. In specific embodiments, the subject administered the recombinant NDV or composition previously experienced COVID-19 symptoms.

In a specific embodiment, the immune response resulting from administration of a recombinant NDV described herein provides some protection against COVID-19 caused by or associated with a SARS-CoV-2 delta variant. In another specific embodiment, an antibody induced by a recombinant NDV described herein binds to a SARS-CoV-2 spike protein delta variant. In another specific embodiment, an antibody induced by a recombinant NDV described herein may neutralize a SARS-CoV-2 delta variant, as assessed by an assay described herein or known to one of skill in the art. In some embodiments, the immune response resulting from administration of a recombinant NDV described herein provides some protection against COVID-19 that is not caused by or associated a SARS-CoV-2 delta variant, as assessed by an assay described herein or known to one of skill in the art. In some embodiments, an antibody induced by a recombinant NDV described herein cross-reacts with a SARS-CoV-2 spike protein that is not a SARS-CoV-2 spike protein delta variant, as assessed by an assay described herein or known to one of skill in the art. In certain embodiments, an antibody induced by a recombinant NDV described herein neutralizes a SARS-CoV-2 that is not a SARS-CoV-2 delta variant, as assessed by an assay described herein or known to one of skill in the art.

In some embodiments, an antibody induced by a recombinant NDV described herein, or an immunogenic composition described herein cross-reacts with a SARS-CoV-2 spike protein that is not a SARS-CoV-2 spike protein delta variant, as assessed by an assay described herein or known to one of skill in the art. In certain embodiments, an antibody induced by a recombinant NDV described herein, or an immunogenic composition described herein neutralizes a SARS-CoV-2 that is not a SARS-CoV-2 delta variant, as assessed by an assay described herein or known to one of skill in the art.

In some embodiments, a recombinant NDV described herein or a composition thereof, or a combination therapy described herein is administered to a patient to prevent the onset of one, two or more symptoms of COVID-19. In a specific embodiment, the administration of a recombinant NDV described herein or a composition thereof, or a combination therapy described herein to a subject prevents the onset or development of one, two or more symptoms of COVID-19, reduces the severity of one, two or more symptoms of COVID-19, or prevents the onset or development of one, two or more symptoms of COVID-19 and reduces the severity of one, two or more symptoms of COVID-19. Symptoms of COVID-19 include congested or runny nose, cough, fever, sore throat, headache, wheezing, rapid or shallow breathing or difficulty breathing, bluish color the skin due to lack of oxygen, chills, muscle pain, loss of taste and/or smell, nausea, vomiting, and diarrhea.

In one embodiment, the administration of a recombinant NDV described herein or a composition thereof, or a combination therapy described herein to a subject prevents the spread of SARS-CoV-2 infection. In a specific embodiment, the administration of a recombinant NDV described herein or a composition thereof, or a combination therapy described herein to a subject prevents the spread of SARS-CoV-2 delta virus infection. In another specific embodiment, the administration of a recombinant NDV described herein or a composition thereof, or a combination therapy described herein to a subject prevents hospitalization. In another specific embodiment, the administration of a recombinant NDV described herein or a composition thereof, or a combination therapy described herein to a subject prevents COVID-19. In another embodiment, the administration of a recombinant NDV described herein or a composition thereof, or a combination therapy described herein to a subject reduces the length of hospitalization. In another embodiment, the administration of a recombinant NDV described herein or a composition thereof, or a combination therapy described herein to a subject reduces the likelihood of intubation. In another specific embodiment, the administration of a recombinant NDV described herein or a composition thereof, or a combination therapy described herein to a subject prevents recurring SARS-CoV-2 infections. In another specific embodiment, the administration of a recombinant NDV described herein or a composition thereof, or a combination therapy described herein to a subject prevents recurring SARS-CoV-2 delta virus infections. In another specific embodiment, the administration of a recombinant NDV described herein or a composition thereof, or a combination therapy described herein to a subject prevents asymptomatic SARS-CoV-2 infection. In another specific embodiment, the administration of a recombinant NDV described herein or a composition thereof, or a combination therapy described herein to a subject prevents asymptomatic SARS-CoV-2 delta virus infection.

In another specific embodiment, the administration of a recombinant NDV described herein or a composition thereof induces antibodies to SARS-CoV-2 spike protein. In another specific embodiment, the administration of a recombinant NDV described herein or a composition thereof induces antibodies specific to SARS-CoV-2 spike protein. An antibody(ies) may specifically bind to a SARS-CoV-2 delta virus spike protein if it binds to the SARS-CoV-2 spike protein with a higher affinity than a spike protein that is not a SARS-CoV-2 spike protein, or other unrelated protein. For example, an antibody(ies) specific for SARS-CoV-2 delta virus spike protein may bind to a SARS-CoV-2 spike protein with a 10 fold higher for affinity than the antibody(ies) binds to a spike protein that is not a SARS-CoV-2 spike protein, or other unrelated protein. In another specific embodiment, the administration of a recombinant NDV described herein or a composition thereof induces antibodies specific to SARS-CoV-2 delta virus spike protein. An antibody(ies) may specifically bind to a SARS-CoV-2 delta virus spike protein if it binds to the SARS-CoV-2 delta virus spike protein with a higher affinity than a SARS-CoV-2 spike protein that is not a SARS-CoV-2 delta variant spike protein. For example, an antibody(ies) specific for SARS-CoV-2 delta virus spike protein may bind to a SARS-CoV-2 delta virus spike protein with a 10 fold higher for affinity than the antibody(ies) binds to a SARS-CoV-2 spike protein that is not a delta variant. In another specific embodiment, the administration of a recombinant NDV described herein or a composition thereof induces both mucosal and systemic antibodies to SARS-CoV-2 spike protein (e.g., neutralizing antibodies). In another specific embodiment, the administration of a recombinant NDV described herein or a composition thereof induces both mucosal and systemic antibodies to SARS-CoV-2 delta variant spike protein (e.g., neutralizing antibodies). In another specific embodiment, the administration of a recombinant NDV described herein or a composition thereof, or a combination therapy described herein to a subject induces neutralizing IgG antibody to SARS-CoV-2 spike protein. In another specific embodiment, the administration of a recombinant NDV described herein or a composition thereof, or a combination therapy described herein to a subject induces neutralizing IgG antibody to SARS-CoV-2 delta virus spike protein. In another specific embodiment, the administration of a recombinant NDV described herein or a composition thereof, or a combination therapy described herein to a subject induces IgG antibody to SARS-CoV-2 spike protein at a level that is considered moderate to high in an ELISA approved by the FDA for measuring antibody in a patient specimen. In another specific embodiment, the administration of a recombinant NDV described herein or a composition thereof, or a combination therapy described herein to a subject induces IgG antibody to SARS-CoV-2 delta virus spike protein at a level that is considered moderate to high in an ELISA approved by the FDA for measuring antibody in a patient specimen. In another specific embodiment, the administration of a recombinant NDV described herein or a composition thereof, or a combination therapy described herein to a subject induces neutralizing antibody to SARS-CoV-2 spike protein. In another specific embodiment, the administration of a recombinant NDV described herein or a composition thereof, or a combination therapy described herein to a subject induces neutralizing antibody to SARS-CoV-2 delta virus spike protein.

In some embodiments, antibody induced by a recombinant NDV described herein, or an immunogenic composition described herein cross-reacts with one, two, three, four, or more, or all of the following: a SARS-CoV-2 delta variant spike protein, a SARS-CoV-2 gamma variant spike protein, a SARS-CoV-2 beta variant spike protein, a SARS-CoV-2 Mu variant spike protein, a SARS-CoV-2 Omicron variant spike protein, and a SARS-CoV-2 Wuhan strain spike protein, as assessed by an assay described herein or known to one of skill in the art. In some embodiments, antibody induced by a recombinant NDV described herein, or an immunogenic composition described herein cross-reacts with a SARS-CoV-2 delta variant spike protein, a SARS-CoV-2 gamma variant spike protein, a SARS-CoV-2 beta variant spike protein, and a SARS-CoV-2 Wuhan strain spike protein, as assessed by an assay described herein or known to one of skill in the art. In some embodiments, antibody induced by a recombinant NDV described herein, or an immunogenic composition described herein cross-reacts with a SARS-CoV-2 delta variant spike protein, a SARS-CoV-2 gamma variant spike protein, a SARS-CoV-2 beta variant spike protein, a SARS-CoV-2 Mu variant spike protein, and a SARS-CoV-2 Wuhan strain spike protein, as assessed by an assay described herein or known to one of skill in the art.

In a specific embodiment, administration of a recombinant NDV described herein or a composition described herein induces an immune response, such as described in Section 6. In a specific embodiment, administration of a recombinant NDV described herein or a composition described herein induces antibody(ies) with one, two, or more of the characteristics of the antibody(ies) described in Section 6.

In another specific embodiment, the administration of a recombinant NDV described herein or a composition thereof, or a combination therapy described herein to a subject induces robust, long-lived (e.g., 6 months, 1 year, 2 years, 3 years or more), antigen-specific humoral immunity. In another specific embodiment, the administration of a recombinant NDV described herein or a composition thereof, or a combination therapy described herein to a subject induces T cell immunity. In another specific embodiment, the administration of a recombinant NDV described herein or a composition thereof, or a combination therapy described herein (e.g., by intranasal administration) to a subject induces mucosal immunity. In another specific embodiment, the administration of a recombinant NDV described herein or a composition thereof, or a combination therapy described herein (e.g., by intranasal administration) to a subject induces mucosal immunity which can lead to sterilizing immunity blocking infection and transmission. In another specific embodiment, the administration of a recombinant NDV described herein or a composition thereof, or a combination therapy described herein (e.g., by intranasal administration) to a subject induces sterilizing immunity and complete prevention of onward transmission of SARS-CoV-2, as assessed by a method known to one of skill in the art. In another specific embodiment, the administration of a recombinant NDV described herein or a composition thereof, or a combination therapy described herein (e.g., by intranasal administration) to a subject induces sterilizing immunity and complete prevention of onward transmission of SARS-CoV-2 delta variant, as assessed by a method known to one of skill in the art. In another specific embodiment, the administration of a recombinant NDV described herein or a composition thereof, or a combination therapy described herein (e.g., by intranasal administration) to a subject induces sterilizing immunity and reduces onward transmission of SARS-CoV-2 by at least 25%, at least 50%, at least 75%, at least 80%, at least 85%, at least 90%, or at least 95%, as assessed by a method known to one of skill in the art. In another specific embodiment, the administration of a recombinant NDV described herein or a composition thereof, or a combination therapy described herein (e.g., by intranasal administration) to a subject induces sterilizing immunity and reduces onward transmission of SARS-CoV-2 delta virus by at least 25%, at least 50%, at least 75%, at least 80%, at least 85%, at least 90%, or at least 95%, as assessed by a method known to one of skill in the art.

In some embodiments, the administration of an immunogenic composition described herein (e.g., by intranasal administration) to a subject induces a (partially or completely) protective immune response against a homologous SARS-CoV-2, such as, e.g., provided in Section 6, infra. In some embodiments, the protective immune response is complete. In some embodiments, the protective immune response is partial.

In some embodiments, the administration of an immunogenic composition described herein (e.g., by intranasal administration) to a subject induces a (partially or completely) protective immune response against a heterologous SARS-CoV-2, such as, e.g., provided in Section 6, infra. In some embodiments, the administration of an immunogenic composition described herein (e.g., by intranasal administration) to a subject induces a (partially or completely) protective immune response against one, two, three, or more heterologous SARS-CoV-2, such as, e.g., provided in Section 6, infra. In some embodiments, the protective immune response is complete. In some embodiments, the protective immune response is partial.

In another specific embodiment, the administration of an immunogenic composition described herein (e.g., by intranasal administration) to a subject induces a (partially or completely) protective immune response against one, two, three, four, or more, or all of the following: SARS-CoV-2 delta variant, SARS-CoV-2 gamma variant, SARS-CoV-2 beta variant, SARS-CoV-2 Mu variant, SARS-CoV-2 Omicron variant, and SARS-CoV-2 Wuhan strain. In another specific embodiment, the administration of an immunogenic composition described herein (e.g., by intranasal administration) to a subject induces a (partially or completely) protective immune response against SARS-CoV-2 delta variant, SARS-CoV-2 gamma variant, SARS-CoV-2 beta variant, and SARS-CoV-2 Wuhan strain. In another specific embodiment, the administration of an immunogenic composition described herein (e.g., by intranasal administration) to a subject induces a (partially or completely) protective immune response against SARS-CoV-2 delta variant, SARS-CoV-2 gamma variant, SARS-CoV-2 beta variant, SARS-CoV-2 Mu variant, and SARS-CoV-2 Wuhan strain. In some embodiments, the protective immune response is complete. In some embodiments, the protective immune response is partial.

In specific embodiments, the administration of a recombinant NDV described herein or a composition thereof, or a combination therapy described herein exhibits very good immunogenicity. In specific embodiments, the administration of a recombinant NDV described herein or a composition thereof, or a combination therapy described herein is safe and exhibits very low reactogenicity, as assessed by a method described herein or known in the art. For example, the administration of a recombinant NDV described herein or a composition thereof, or a combination therapy described herein to a subject may not result in injection site pain and/or transient influenza-like symptoms in the subject. In another example, the administration of a recombinant NDV described herein or a composition thereof, or a combination therapy described herein to a subject may result in less injection site pain and/or fewer transient influenza-like symptoms than one, two, or more authorized COVID-19 vaccines (e.g., Pfizer's COVID-19 vaccine (BNT162b2), Moderna's COVID-19 vaccine (mRNA-1273), Johnson & Johnson's COVID-19 vaccine (Ad26.COV2.S), AstraZeneca's COVID-19 vaccine, SinoVac's COVID-19 vaccine, SinoPharm's COVID-19 vaccine, Bharat's COVID-19 vaccine, or Cansino's COVID-19 vaccine).

In a specific embodiment, the administration of a recombinant NDV described herein or a composition thereof, or a combination therapy described herein induces protective immunity in a subject (e.g., a human subject or non-human subject). In a particular embodiment, the administration of a recombinant NDV described herein or a composition thereof, or a combination therapy described herein induces immunity in a subject (e.g., a human subject or non-human subject) that protects (partially or completely) the subject from disease (e.g., COVID-19) due to subsequent infection by SARS-CoV-2 (e.g., SARS-CoV-2 delta virus infection).

In some embodiments, a recombinant NDV described herein or a composition thereof, or a combination therapy described herein is administered to a subject predisposed or susceptible to COVID-19. In some embodiments, a recombinant NDV described herein or a composition thereof, or a combination therapy described herein is administered to a subject that has not been previously vaccinated for COVID-19. In some embodiments, a recombinant NDV described herein or a composition thereof, or a combination therapy described herein is administered to a subject that is not aware of previously being infected by SARS-CoV-2. In some embodiments, a recombinant NDV described herein or a composition thereof, or a combination therapy described herein is administered to a subject previously vaccinated for COVID-19. In some embodiments, a recombinant NDV described herein or a composition thereof, or a combination therapy described herein is administered to a subject previously infected with SARS-CoV-2, as assessed by a technique known in the art (e.g., a rapid antigen test or PCR). In some embodiments, a recombinant NDV described herein or a composition thereof, or a combination therapy described herein is administered to a subject that has previously experienced COVID-19 symptoms.

In certain embodiments, a recombinant NDV described herein or a composition thereof, or a combination therapy described herein is administered to a human. In some embodiments, a recombinant NDV described herein or a composition thereof, or a combination therapy described herein is administered to a human infant. In another specific embodiment, the subject is a human infant six months old or older. In other embodiments, a recombinant NDV described herein or a composition thereof, or a combination therapy described herein is administered to a human toddler. In other embodiments, a recombinant NDV described herein or a composition thereof, or a combination therapy described herein is administered to a human child. In other embodiments, a recombinant NDV described herein or a composition thereof, or a combination therapy described herein is administered to a human adult. In yet other embodiments, a recombinant NDV described herein or a composition thereof, or a combination therapy described herein is administered to an elderly human.

In a specific embodiment, a recombinant NDV described herein or a composition thereof, or a combination therapy described herein is administered a subject (e.g., a human subject) in close contact with an individual with increased risk of COVID-19 or SARS-CoV-2 infection (e.g., a SARS-CoV-2 delta virus infection). In some embodiments, a recombinant NDV described herein or a composition thereof, or a combination therapy described herein is administered a subject (e.g., a human subject) with a condition that increases susceptibility to SARS-CoV-2 complications or for which SARS-CoV-2 increases complications associated with the condition. Examples of conditions that increase susceptibility to SARS-CoV-2 complications or for which SARS-CoV-2 increases complications associated with the condition include conditions that affect the lung, such as cystic fibrosis, chronic obstructive pulmonary disease (COPD), emphysema, asthma, or bacterial infections (e.g., infections caused by Haemophilus influenzae, Streptococcus pneumoniae, Legionella pneumophila, and Chlamydia trachomatus); cardiovascular disease (e.g., congenital heart disease, congestive heart failure, and coronary artery disease); and endocrine disorders (e.g., diabetes).

In some embodiments, a recombinant NDV described herein or a composition thereof, or a combination therapy described herein is administered a subject (e.g., a human subject) that resides in a group home, such as a nursing home. In some embodiments, a recombinant NDV described herein or a composition thereof, or a combination therapy described herein is administered a subject (e.g., a human subject) that works in, or spends a significant amount of time in, a group home, e.g., a nursing home. In some embodiments, a recombinant NDV described herein or a composition thereof, or a combination therapy described herein is administered a subject (e.g., a human subject) that is a health care worker (e.g., a doctor or nurse). In some embodiments, a recombinant NDV described herein or a composition thereof, or a combination therapy described herein is administered a subject (e.g., a human subject) that is a smoker.

In some embodiments, a recombinant NDV described herein or a composition thereof, or a combination therapy described herein is administered to: (1) a subject (e.g., a human subject) who can transmit SARS-CoV-2 to those at high risk for complications, such as, e.g., members of households with high-risk subjects, including households that will include human infants (e.g., infants younger than 6 months), (2) a subject coming into contact with human infants (e.g., infants less than 6 months of age), (3) a subject who will come into contact with subjects who live in nursing homes or other long-term care facilities, (4) a subject who is or will come into contact with an elderly human, or (5) a subject who will come into contact with subjects with long-term disorders of the lungs, heart, or circulation; individuals with metabolic diseases (e.g., diabetes) or subjects with weakened immune systems (including immunosuppression caused by medications, malignancies such as cancer, organ transplant, or HIV infection).

In a specific embodiment, provided herein is a method of inducing an immune response to SARS-CoV-2 (e.g., SARS-CoV-2 delta variant) subject (e.g., a human subject), comprising administering the subject (e.g., a human subject) a nucleic acid sequence described herein, a nucleotide sequence described herein, or a composition thereof. In another specific embodiment, provided herein is a method of inducing an immune response to SARS-CoV-2 (e.g., SARS-CoV-2 delta variant) subject (e.g., a human subject), comprising administering the subject (e.g., a human subject) an effective amount of a nucleic acid sequence described herein or a nucleotide sequence described herein, or a composition thereof. The method may further the administration of one or more additional therapies, such as described herein in Section 5.5.3, infra. The subject administered the nucleic acid sequence, the nucleotide sequence, or composition is a subject described herein.

In a specific embodiment, provided herein is a method of immunizing a subject (e.g., human subject) against SARS-CoV-2 (e.g., SARS-CoV-2 delta variant), comprising administering the subject (e.g., a human subject) a nucleic acid sequence described herein or nucleotide sequence described herein, or a composition thereof. In another specific embodiment, provided herein is a method of immunizing a subject (e.g., human subject) against SARS-CoV-2 (e.g., SARS-CoV-2 delta variant), comprising administering the subject (e.g., a human subject) an effective amount of a nucleic acid sequence described herein or a nucleotide sequence described herein, or a composition thereof. The method may further the administration of one or more additional therapies, such as described herein in Section 5.5.3, infra. The subject administered the nucleic acid sequence, the nucleotide, or composition is a subject described herein.

In a specific embodiment, provided herein is a method of preventing COVID-19 in a subject (e.g., human subject), comprising administering the subject (e.g., a human subject) a nucleic acid sequence described herein or nucleotide sequence described herein, or a composition thereof. In another specific embodiment, provided herein is a method of preventing COVID-19 in a subject (e.g., human subject), comprising administering the subject (e.g., a human subject) an effective amount of a nucleic acid sequence described herein or a nucleotide sequence described herein, or a composition thereof. The method may further the administration of one or more additional therapies, such as described herein in Section 5.5.3, infra. The subject administered the nucleic acid sequence, the nucleotide sequence, or composition is a subject described herein.

In a specific embodiment, provided herein is a method of inducing an immune response to SARS-CoV-2 (e.g., SARS-CoV-2 delta variant) subject (e.g., a human subject), comprising administering the subject (e.g., a human subject) a protein described herein, or a composition thereof. In another specific embodiment, provided herein is a method of inducing an immune response to SARS-CoV-2 (e.g., SARS-CoV-2 delta variant) subject (e.g., a human subject), comprising administering the subject (e.g., a human subject) an effective amount of a protein described herein, or a composition thereof. The method may further the administration of one or more additional therapies, such as described herein in Section 5.5.3, infra. The subject administered the protein or composition is a subject described herein.

In a specific embodiment, provided herein is a method of immunizing a subject (e.g., human subject) against SARS-CoV-2 (e.g., SARS-CoV-2 delta variant), comprising administering the subject (e.g., a human subject) a protein described herein, or a composition thereof. In another specific embodiment, provided herein is a method of immunizing a subject (e.g., human subject) against SARS-CoV-2 (e.g., SARS-CoV-2 delta variant), comprising administering the subject (e.g., a human subject) an effective amount of a protein described herein, or a composition thereof. The method may further the administration of one or more additional therapies, such as described herein in Section 5.5.3, infra. The subject administered the protein or composition is a subject described herein.

In a specific embodiment, provided herein is a method of preventing COVID-19 in a subject (e.g., human subject), comprising administering the subject (e.g., a human subject) a protein described herein, or a composition thereof. In another specific embodiment, provided herein is a method of preventing COVID-19 in a subject (e.g., human subject), comprising administering the subject (e.g., a human subject) an effective amount of a protein described herein, or a composition thereof. The method may further the administration of one or more additional therapies, such as described herein in Section 5.5.3, infra. The subject administered the protein or composition is a subject described herein.

### 5.5.2 Dosage and Frequency

The amount of a recombinant NDV or a composition thereof, which will be effective in the prevention of COVID-19, or immunization against SARS-CoV-2 delta variant will depend on the route of administration, the general health of the subject, etc. Suitable dosage ranges of a recombinant NDV for administration are generally about 10⁴ to about 10¹² EID50, and can be administered to a subject once, twice, three, four or more times with intervals as often as needed. In some embodiments, a recombinant NDV described herein is administered to a subject (e.g., human) at a dose of 10⁴ to about 10¹² EID50. In some embodiments, a dose of about 10⁴ to about 10¹² EID50 of a composition comprising live recombinant NDV is administered to a subject (e.g., human). In a specific embodiment, a live recombinant NDV described herein is administered to a subject (e.g., human) at a dose of 10⁷ to 10⁹ EID50. In another specific embodiment, a dose of 10⁷ to 10⁹ EID50 of a composition comprising a live recombinant NDV described herein is administered to a subject (e.g., a human). In a specific embodiment, a live recombinant NDV described herein is administered to a subject (e.g., human) at a dose of about 10⁸ to about 10⁹ EID50. In a specific embodiment, a live recombinant NDV described herein is administered to a subject (e.g., human) at a dose of about 10⁷ to about 10⁸ EID50.

In certain embodiments, a recombinant NDV described herein is administered to a subject (e.g., human) at a dose of 1 to 15 micrograms of SARS-CoV-2 delta variant spike protein or a portion thereof, a derivative of SARS-CoV-2 delta variant spike protein or a portion thereof, or a chimeric F protein. In some embodiments, a recombinant NDV described herein is administered to a subject (e.g., human) at a dose of 1 to 10 micrograms of SARS-CoV-2 delta variant spike protein or a portion thereof, a derivative of a SARS-CoV-2 delta variant spike protein or a portion thereof, or a chimeric F protein. In a specific embodiment, a recombinant NDV described herein is administered to a subject (e.g., human) at a dose of 1 microgram, 3 micrograms, or 10 micrograms of SARS-CoV-2 delta variant spike protein or a portion thereof, a derivative of SARS-CoV-2 delta variant spike protein or a portion thereof, or a chimeric F protein. In another specific embodiment, a recombinant NDV described herein is administered to a subject (e.g., human) at a dose of 4 micrograms, 5 micrograms, 6 micrograms, 7 micrograms, 8 micrograms or 9 micrograms of SARS-CoV-2 delta variant spike protein or a portion thereof, a derivative of SARS-CoV-2 delta variant spike protein or a portion thereof, or a chimeric F protein.

In certain embodiments, a composition described herein is administered to a subject (e.g., human) at a dose of 1 to 15 micrograms of SARS-CoV-2 delta variant spike protein or a portion thereof, a derivative of SARS-CoV-2 delta variant spike protein or a portion thereof, or a chimeric F protein. In some embodiments, a composition described herein is administered to a subject (e.g., human) at a dose of 1 to 10 micrograms of SARS-CoV-2 delta variant spike protein or a portion thereof, a derivative of SARS-CoV-2 delta variant spike protein or a portion thereof, or a chimeric F protein. In a specific embodiment, a composition NDV described herein is administered to a subject (e.g., human) at a dose of 1 microgram, 3 micrograms, or 10 micrograms of SARS-CoV-2 delta variant spike protein or a portion thereof, a derivative of SARS-CoV-2 delta variant spike protein or a portion thereof, or a chimeric F protein. In another specific embodiment, a composition described herein is administered to a subject (e.g., human) at a dose of 4 micrograms, 5 micrograms, 6 micrograms, 7 micrograms, 8 micrograms or 9 micrograms of SARS-CoV-2 delta variant spike protein or a portion thereof, a derivative of SARS-CoV-2 delta variant spike protein or a portion thereof, or a chimeric F protein.

In some embodiments, a composition described herein is administered to a subject (e.g., human) at a dose of 1 to 15 micrograms of inactivated recombinant NDV described herein. In some embodiments, a composition described herein is administered to a subject (e.g., human) at a dose of 1 to 10 micrograms of inactivated recombinant NDV described herein. In specific embodiments, a composition described herein is administered to a subject (e.g., human) at a dose of 1 micrograms, 3 micrograms, or 10 micrograms of inactivated recombinant NDV described herein.

In certain embodiments, dosages of a recombinant NDV described herein, or a composition described herein similar to those currently being used in clinical trials for NDV are administered to a subject.

In some embodiments, a recombinant NDV described herein or a composition described herein is administered to a subject (e.g., a human) once. In other embodiments, a recombinant NDV described herein or a composition described herein is administered to a subject (e.g., a human) more than once.

In certain embodiments, a recombinant NDV or a composition thereof is administered to a subject as a single dose followed by a second dose 1 to 6 weeks, 1 to 5 weeks, 1 to 4 weeks, 1 to 3 weeks, 1 to 2 weeks, 6 to 12 weeks, 3 to 6 months, 6 to 9 months, 6 to 12 months, or 6 to 9 months later. In some embodiments, a recombinant NDV or a composition thereof is administered to a subject as a single dose followed by a second dose about 3 to about 6 months, about 6 to about 9 months, or about 6 to about 12 months later. In specific embodiments, a recombinant NDV or a composition thereof is administered to a subject as a single dose followed by a second dose about 6 months later. In accordance with these embodiments, booster inoculations may be administered to the subject at 3 to 6 month or 6 to 12 month intervals following the second inoculation. In accordance with these embodiments, booster inoculations may be administered to the subject at about 6 months following the second inoculation. In certain embodiments, a subject is administered one or more boosters. The recombinant NDV used for each booster may be the same or different. The two or more recombinant NDVs or compositions thereof administered to the subject may administered by the same or different routes. For example, one recombinant NDV or a composition thereof may be administered to the subject intranasally and another recombinant NDV or a composition thereof may be administered to the subject intramuscularly. In another example, one recombinant NDV or a composition thereof may be administered to the subject intramuscularly and another recombinant NDV or a composition thereof may be administered to the subject intranasally. In another example, one recombinant NDV or a composition thereof may be administered to the subject intranasally or intramuscularly and another recombinant NDV or a composition thereof may be administered to the subject by the same route of administration.

In certain embodiments, administration of the same recombinant NDV or a composition thereof may be repeated and the administrations may be separated by at least 7 days, 10 days, 14 days, 15 days, 21 days, 28 days, 30 days, 45 days, 2 months, 75 days, 3 months, or at least 6 months. In other embodiments, administration of the same recombinant NDV or a composition thereof may be repeated and the administrations may be separated by 1 to 14 days, 1 to 7 days, 7 to 14 days, 1 to 30 days, 15 to 30 days, 15 to 45 days, 15 to 75 days, 15 to 90 days, 1 to 3 months, 3 to 6 months, 3 to 12 months, or 6 to 12 months. In some embodiments, a first recombinant NDV or a composition thereof is administered to a subject followed by the administration of a second recombinant NDV or a composition thereof. In some embodiments, the first and second recombinant NDV are different from each other. In certain embodiments, a first pharmaceutical composition is administered to a subject as a priming dose and after a certain period (e.g., 1 month, 2 months, 3 months, 4 months, 5 months, 6 months, or 1-6 months) a booster dose of a second pharmaceutical composition is administered. For example, the first recombinant NDV may comprise a packaged genome comprising a transgene that comprises a nucleotide sequence encoding a SARS-CoV-2 spike protein or portion thereof (e.g., ectodomain or receptor binding domain of the SARS-CoV-2 spike protein), and the second recombinant NDV may comprise a package genome comprising a transgene that comprises a nucleotide sequence encoding a chimeric F protein, wherein the chimeric F protein comprises a SARS-CoV-2 delta variant spike protein ectodomain or a derivative thereof and NDV F protein transmembrane and cytoplasmic domains. In specific embodiments, the derivative of the SARS-CoV-2 spike protein ectodomain lacks the polybasic cleavage site (e.g., amino acid residues of the polybasic cleavage site (RRAR) are substituted with a single alanine), and amino acid residues corresponding to amino acid residues 817, 892, 899, 942, 986, and 987 of the spike protein found at GenBank Accession No. MN908947 are substituted with prolines. In specific embodiments, the derivative of the SARS-CoV-2 delta variant spike protein ectodomain does not comprise the amino acid substitution of P681R. In specific embodiments, the derivative of the SARS-CoV-2 spike protein ectodomain lacks the polybasic cleavage site (e.g., amino acid residues of the polybasic cleavage site (RRAR) are substituted with a single alanine), amino acid residues corresponding to amino acid residues 817, 892, 899, 942, 986, and 987 of the spike protein found at GenBank Accession No. MN908947.3 are substituted with prolines, and amino acid residues corresponding to the following amino acid residues of the SARS-CoV-2 spike protein found at GenBank Accession No. MN908947.3 are modified as follows: T19R, G142D, delE156, delF157, L452R, T478K, D614G, and D950N. In specific embodiments, the derivative of the SARS-CoV-2 spike protein ectodomain comprises the amino acid sequence of SEQ ID NO:12 with amino acid residues corresponding to the following amino acid residues of the SARS-CoV-2 spike protein found at GenBank Accession No. MN908947.3 are modified as follows: T19R, G142D, delE156, delF157, R158G, L452R, T478K, D614G, and D950N. In specific embodiments, the derivative of the SARS-CoV-2 spike protein ectodomain comprises the amino acid sequence of SEQ ID NO:12 with amino acid residue modifications for Delta shown in FIG. 3A. In specific embodiments, the SARS-CoV-2 delta variant spike protein ectodomain or derivative thereof is fused to the NDV F protein transmembrane and cytoplasmic domains via a linker (e.g., GGGGS (SEQ ID NO:7)). The linker may be any linker that does not interfere with folding of the ectodomain, function of the ectodomain or both. In some embodiments, the linker is an amino acid sequence (e.g., a peptide) that is 1, 2, 3, 4, 5, 6, 7, 8, 9, 10, 11, 12, 13, 14, 15, 16, 17, 18, 19, 20 or more amino acids long. In some embodiments, the linker is a glycine (G) linker or glycine and serine (GS) linker. For example, the linker may comprise the sequence of (GGGGS)ₙ (SEQ ID NO:24), wherein n is 1, 2, 3, 4, 5 or more. In another example, the linker may comprise (G)ₙ, wherein n is 3, 4, 5, 6, 7, 8 or more. In a specific embodiment, the linker comprises the sequence GGGGS (SEQ ID NO:7). In some embodiments, the NDV F protein transmembrane and cytoplasmic domains are fused to directly to the SARS-CoV-2 delta variant spike protein ectodomain or derivative thereof. See, e.g., Sections 5.1 and 6 for examples of recombinant NDVs. In specific embodiments, the chimeric F protein comprises the amino acid sequence of SEQ ID NO: 6 or 21. In certain embodiments, the first and second recombinant NDVs or compositions thereof may be separated by at least 7 days, 10 days, 14 days, 15 days, 21 days, 28 days, 30 days, 45 days, 2 months, 75 days, 3 months, or at least 6 months. In other embodiments, the first and second recombinant NDVs or compositions thereof may be separated by 1 to 14 days, 1 to 7 days, 7 to 14 days, 1 to 30 days, 15 to 30 days, 15 to 45 days, 15 to 75 days, 15 to 90 days, 1 to 3 months, 3 to 6 months, 3 to 12 months, or 6 to 12 months.

In certain embodiments, a first dose of a recombinant NDV described herein or composition described herein may be administered to a subject (e.g., a human) and a second dose of the recombinant NDV or composition may be administered to the subject 3 to 6 weeks later. In some embodiments, the subject is administered two or more boosters of the recombinant NDV.

In some embodiments, a recombinant NDV described herein or composition thereof is administered as a booster to a subject previously vaccinated with a COVID-19 vaccine. The COVID-19 vaccine may be Pfizer's COVID-19 vaccine (BNT162b2), Moderna's COVID-19 vaccine (mRNA-1273), AstraZeneca's COVID-19 vaccine, Johnson & Johnson's COVID-19 (Ad26.COV2.S), SinoVac's COVID-19 vaccine, SinoPharm's COVID-19 vaccine, Bharat's COVID-19 vaccine, Cansino's COVID-19 vaccine, or another COVID-19 vaccine. In a specific embodiment, the subject was previously vaccinated with a COVID-19 other than an immunogenic composition described herein. In a specific embodiment, the subject was previously vaccinated with a COVID-19 other than a recombinant NDV-based COVID-19 vaccine.

In some embodiments, a recombinant NDV described herein or composition thereof is administered as a booster to a subject previously infected with SARS-CoV-2. In certain embodiments, a recombinant NDV described herein or composition thereof is administered as a booster to a subject previously diagnosed with a SARS-CoV-2 infection (e.g., a SARS-CoV-2 delta variant infection or another SARS-CoV-2 infection).

In certain embodiments, a recombinant NDV or composition thereof is administered to a subject in combination with one or more additional therapies, such as a therapy described in Section 5.5.3, infra. The dosage of the other one or more additional therapies will depend upon various factors including, e.g., the therapy, the route of administration, the general health of the subject, etc. and should be decided according to the judgment of a medical practitioner. In specific embodiments, the dose of the other therapy is the dose and/or frequency of administration of the therapy recommended for the therapy for use as a single agent is used in accordance with the methods disclosed herein. Recommended doses for approved therapies can be found in the Physician's Desk Reference.

In certain embodiments, a recombinant NDV or composition thereof is administered to a subject concurrently with the administration of one or more additional therapies. In some embodiments, a first pharmaceutical composition comprising recombinant NDV and a second pharmaceutical composition comprising one or more additional therapies may be administered concurrently, or before or after each other. In certain embodiments, the first and second pharmaceutical compositions are administered concurrently to the subject, or within 1 minute, 2 minutes, 5 minutes, 10 minutes, 15 minutes, 20 minutes, 30 minutes, 45 minutes, 60 minutes, 1.5 hours, 2 hours, 3 hours, 4 hours, 5 hours, or 6 hours of each other. In certain embodiments, the first and second pharmaceutical compositions are administered to the subject within 7 days, 2 weeks, 3 weeks, 4 weeks, 5 weeks, 6 weeks, 7 weeks, 8 weeks or 12 weeks of each other. In certain embodiments, the first and second pharmaceutical compositions are administered to the subject within 3-6 months, 6-9 months, 6-12 months, or 3 months, 4 months, 6 months, 9 months, or 12 months of each other.

### 5.5.3 Additional Therapies

Additional therapies that can be used in a combination with a recombinant NDV described herein or a composition thereof include, but are not limited to, acetaminophen, ibuprofen, throat lozenges, cough suppressants, inhalers, antibiotics, monoclonal antibodies, and oxygen. In a specific embodiment, the additional therapy is a second recombinant NDV described herein. In a specific embodiment, the additional therapy is a monoclonal antibody, such as sotrovimab. In another specific embodiment, the additional therapy(ies) may include remdesivir, sotrovimab, bamlanivimab plus etesevimab (AIIa), casirivimab plus imdevimab (AIIa), dexamethasone, tocilizumab, oxygen, or a combination thereof.

### 5.5.4 Other Uses of Recombinant NDV

In some embodiments, a recombinant NDV described herein is administered to a non-human subject (e.g., a mouse, rat, etc.) and the antibodies generated in response to the polypeptide are isolated. Hybridomas may be made and monoclonal antibodies produced as known to one of skill in the art. The antibodies may also be optimized. In some embodiments, the antibodies produced are humanized or chimerized. In certain embodiments, the non-human subject produces human antibodies. The antibodies produced using a recombinant NDV described herein may be optimized, using techniques known to one of skill in the art. In a specific embodiment, antibodies generated using a recombinant NDV described herein may be used to prevent, treat, or prevent and treat COVID-19. In some embodiments, antibodies generated using a recombinant NDV described herein may be used in an immunoassay to detect SARS-CoV-2 (e.g., SARS-CoV-2 delta variant). In some embodiments, antibodies generated using a recombinant NDV described herein may be used in an immunoassay to detect SARS-CoV-2 spike protein (e.g., SARS-CoV-2 delta variant spike protein).

In some embodiments, a recombinant NDV described herein is used in an immunoassay (e.g., an ELISA assay) known to one of skill in the art or described herein to detect antibody specific for SARS-CoV-2 spike protein (e.g., SARS-CoV-2 delta virus spike protein). In one embodiment, provided herein is a method for detecting the presence of antibody specific to SARS-CoV-2 spike protein (e.g., SARS-CoV-2 delta virus spike protein), comprising contacting a specimen with a recombinant NDV described herein in an immunoassay (e.g., an ELISA). In some embodiments, the specimen is a biological specimen. In a specific embodiment, the biological specimen is blood, plasma or sera from a subject (e.g., a human subject). In other embodiments, the specimen is an antibody or antisera.

In some embodiments, a nucleic acid sequence described herein, a nucleotide sequence described herein, a protein described herein, or a composition described herein is administered to a non-human subject (e.g., a mouse, rat, etc.) and the antibodies generated in response to the polypeptide are isolated. Hybridomas may be made and monoclonal antibodies produced as known to one of skill in the art. The antibodies may also be optimized. In some embodiments, the antibodies produced are humanized or chimerized. In certain embodiments, the non-human subject produces human antibodies. The antibodies produced using a nucleic acid sequence described herein, a nucleotide sequence described herein, a protein described herein, or a composition described herein may be optimized, using techniques known to one of skill in the art. In a specific embodiment, antibodies generated using a nucleic acid sequence described herein, a nucleotide sequence described herein, a protein described herein, or a composition described herein may be used to prevent, treat, or prevent and treat COVID-19. In some embodiments, antibodies generated using a nucleic acid sequence described herein, a nucleotide sequence described herein, a protein described herein, or a composition described herein may be used in an immunoassay to detect SARS-CoV-2 (e.g., SARS-CoV-2 delta variant). In some embodiments, antibodies generated using a nucleic acid sequence described herein, a nucleotide sequence described herein, a protein described herein, or a composition described herein may be used in an immunoassay to detect SARS-CoV-2 spike protein (e.g., SARS-CoV-2 delta variant spike protein).

In some embodiments, a protein described herein, or a composition described herein is used in an immunoassay (e.g., an ELISA assay) known to one of skill in the art or described herein to detect antibody specific for SARS-CoV-2 spike protein (e.g., SARS-CoV-2 delta virus spike protein). In one embodiment, provided herein is a method for detecting the presence of antibody specific to SARS-CoV-2 spike protein (e.g., SARS-CoV-2 delta virus spike protein), comprising contacting a specimen with a protein described herein, or a composition described herein in an immunoassay (e.g., an ELISA). In some embodiments, the specimen is a biological specimen. In a specific embodiment, the biological specimen is blood, plasma or sera from a subject (e.g., a human subject). In other embodiments, the specimen is an antibody or antisera.

### 5.6 Biological Assays

In a specific embodiment, one, two or more of the assays described in Section 6 may be used to characterize a recombinant NDV described herein, or a SARS-CoV-2 delta variant spike protein or portion thereof (e.g., the ectodomain or receptor binding domain of the SARS-CoV-2 spike protein), or a chimeric F protein. In another specific embodiment, one, two or more of the assays described in Section 6 may be used to characterize immunoglobulin samples from a subject (e.g., a human subject) administered a recombinant NDV described herein or a composition described herein, such as, e.g., described in the Example, infra (e.g., Section 6). For example, the IgG titer and microneutralization of IgG may be assessed as described herein or known to one of skill in the art. In some embodiments, a subject administered a recombinant NDV described herein or a composition described herein is assessed for anti-NDV antibodies as well as anti-SARS-CoV-2 spike protein (e.g., SARS-CoV-2 delta virus spike protein) antibodies.

### 5.6.1 In Vitro Viral Assays

Viral assays include those that indirectly measure viral replication (as determined, e.g., by plaque formation) or the production of viral proteins (as determined, e.g., by western blot analysis) or viral RNAs (as determined, e.g., by RT-PCR or northern blot analysis) in cultured cells in vitro using methods which are well known in the art.

Growth of the recombinant NDVs described herein can be assessed by any method known in the art or described herein (e.g., in cell culture (e.g., cultures of BSTT7 or embryonated chicken cells) (see, e.g., Section 6). Viral titer may be determined by inoculating serial dilutions of a recombinant NDV described herein into cell cultures (e.g., BSTT7 or embryonated chicken cells), chick embryos (e.g., 9 to 11 day old embryonated eggs), or live non-human animals. After incubation of the virus for a specified time, the virus is isolated using standard methods. Physical quantitation of the virus titer can be performed using PCR applied to viral supernatants (Quinn & Trevor, 1997; Morgan et al., 1990), hemagglutination assays, tissue culture infectious doses (TCID50) or egg infectious doses (EID50).

Incorporation of nucleotide sequences encoding a heterologous peptide or protein (e.g., a transgene into the genome of a recombinant NDV described herein can be assessed by any method known in the art or described herein (e.g., in cell culture, an animal model or viral culture in embryonated eggs)). For example, viral particles from cell culture of the allantoic fluid of embryonated eggs can be purified by centrifugation through a sucrose cushion and subsequently analyzed for protein expression by Western blotting using methods well known in the art. In a specific embodiment, a method described in Section 6, infra, is used to assess the incorporation of a transgene into the genome of a recombinant NDV.

Immunofluorescence-based approaches may also be used to detect virus and assess viral growth. Such approaches are well known to those of skill in the art, e.g., fluorescence microscopy and flow cytometry. Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

### 5.6.2 Interferon Assays

IFN induction and release by a recombinant NDV described herein may be determined using techniques known to one of skill in the art. For example, the amount of IFN induced in cells following infection with a recombinant NDV described herein may be determined using an immunoassay (e.g., an ELISA or Western blot assay) to measure IFN expression or to measure the expression of a protein whose expression is induced by IFN. Alternatively, the amount of IFN induced may be measured at the RNA level by assays, such as Northern blots and quantitative RT-PCR, known to one of skill in the art. In specific embodiments, the amount of IFN released may be measured using an ELISPOT assay. The IFN may be any one, two or all of: IFN-α, IFN-β, and IFN-γ. Further, the induction and release of cytokines and/or interferon-stimulated genes may be determined by, e.g., an immunoassay or ELISPOT assay at the protein level and/or quantitative RT-PCR or northern blots at the RNA level.

### 5.6.3 Toxicity Studies

In some embodiments, the recombinant NDVs described herein or compositions thereof, or combination therapies described herein are tested for cytotoxicity in mammalian, preferably human, cell lines. In certain embodiments, cytotoxicity is assessed in one or more of the following non-limiting examples of cell lines: U937, a human monocyte cell line; primary peripheral blood mononuclear cells (PBMC); Huh7, a human hepatoblastoma cell line; HL60 cells, HT1080, HEK 293T and 293H, MLPC cells, human embryonic kidney cell lines; human melanoma cell lines, such as SkMel2, SkMel-119 and SkMel-197; THP-1, monocytic cells; a HeLa cell line; and neuroblastoma cells lines, such as MC-IXC, SK-N-MC, SK-N-MC, SK-N-DZ, SH-SY5Y, and BE(2)-C. In some embodiments, the ToxLite assay is used to assess cytotoxicity.

Many assays well known in the art can be used to assess viability of cells or cell lines following infection with a recombinant NDV described herein or composition thereof, and, thus, determine the cytotoxicity of the recombinant NDV or composition thereof. For example, cell proliferation can be assayed by measuring Bromodeoxyuridine (BrdU) incorporation, (³H) thymidine incorporation, by direct cell count, or by detecting changes in transcription, translation or activity of known genes such as proto-oncogenes (e.g., fos, myc) or cell cycle markers (Rb, cdc2, cyclin A, D1, D2, D3, E, etc.). The levels of such protein and mRNA and activity can be determined by any method well known in the art. For example, protein can be quantitated by known immunodiagnostic methods such as ELISA, Western blotting or immunoprecipitation using antibodies, including commercially available antibodies. mRNA can be quantitated using methods that are well known and routine in the art, for example, using northern analysis, RNase protection, or polymerase chain reaction in connection with reverse transcription. Cell viability can be assessed by using trypan-blue staining or other cell death or viability markers known in the art. In a specific embodiment, the level of cellular ATP is measured to determined cell viability. In preferred embodiments, a recombinant NDV described herein or composition thereof does not kill healthy (i.e., non-cancerous) cells.

In specific embodiments, cell viability may be measured in three-day and seven-day periods using an assay standard in the art, such as the CellTiter-Glo Assay Kit (Promega) which measures levels of intracellular ATP. A reduction in cellular ATP is indicative of a cytotoxic effect. In another specific embodiment, cell viability can be measured in the neutral red uptake assay. In other embodiments, visual observation for morphological changes may include enlargement, granularity, cells with ragged edges, a filmy appearance, rounding, detachment from the surface of the well, or other changes.

The recombinant NDVs described herein or compositions thereof, or combination therapies can be tested for in vivo toxicity in animal models. For example, animals are administered a range of pfu of a recombinant NDV described herein, and subsequently, the animals are monitored over time for various parameters, such as one, two or more of the following: lethality, weight loss or failure to gain weight, and levels of serum markers that may be indicative of tissue damage (e.g., creatine phosphokinase level as an indicator of general tissue damage, level of glutamic oxalic acid transaminase or pyruvic acid transaminase as indicators for possible liver damage). These in vivo assays may also be adapted to test the toxicity of various administration mode and regimen in addition to dosages. See, e.g., the Examples, infra, for assays that may be used to assess toxicity.

The toxicity, efficacy or both of a recombinant NDV described herein or a composition thereof, or a combination therapy described herein can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

The data obtained from the cell culture assays and animal studies can be used in formulating a range of dosage of the therapies for use in subjects.

### 5.6.4 Biological Activity Assays

The recombinant NDVs described herein or compositions thereof, or combination therapies described herein can be tested for biological activity using animal models for inhibiting COVID-19, antibody response to the recombinant NDVs, etc. (see, e.g., Section 6). Such animal model systems include, but are not limited to, rats, mice, hamsters, cotton rats, chicken, cows, monkeys (e.g., African green monkey), pigs, dogs, rabbits, etc.

In a specific embodiment, the recombinant NDVs described herein or compositions thereof, or combination therapies described herein may be tested using animal models for the ability to induce a certain geometric mean titer of antibody(ies) that binds to the SARS-CoV-2 spike protein. An immunoassay, such as an ELISA, or known to one of skill in the art may be used to measure antibody titer. In another specific embodiment, the recombinant NDVs described herein or compositions thereof, or combination therapies described herein may be tested using animal models for the ability to induce antibodies that have neutralizing activity against SARS-CoV-2 spike protein (e.g., SARS-CoV-2 delta virus spike protein) in a microneutralization assay. In some embodiments, the recombinant NDVs described herein or compositions thereof, or combination therapies described herein may be tested using animal models for the ability to induce antibodies that neutralize SARS-CoV-2 (e.g., SARS-CoV-2 delta virus) in a microneutralization assay. In some embodiments, the recombinant NDVs described herein or compositions thereof, or combination therapies described herein may be tested using animal models for the ability to induce a certain geometric mean titer of antibody(ies) that binds to the SARS-CoV-2 spike protein (e.g., SARS-CoV-2 delta virus spike protein) and neutralizes SARS-CoV-2 (e.g., SARS-CoV-2 delta virus) in a microneutralization assay. In some embodiments, the recombinant NDVs described herein or compositions thereof, or combination therapies described herein may be tested using animal models for the ability to induce a certain geometric mean titer of antibody(ies) that binds to the SARS-CoV-2 spike protein (e.g., SARS-CoV-2 delta virus spike protein) and neutralizes SARS-CoV-2 (e.g., SARS-CoV-2 delta virus) in a microneutralization assay such as described herein. In certain embodiments, the recombinant NDVs described herein or compositions thereof, or combination therapies described herein may be tested using animal models for the ability to induce a protective immune response.

In a specific embodiment, a recombinant NDV described herein or a composition thereof, or a combination therapy described herein may be tested in a clinical trial study. In certain embodiments, a recombinant NDV described herein or a composition thereof, or a combination therapy described herein is administered to a human subject. In some embodiments, a human subject administered a recombinant NDV described herein or a composition thereof, or a combination therapy described herein may be assessed for one, two or more, or all of the following may be assessed following administration of a recombinant NDV described herein or a composition thereof, or a combination therapy described herein: GMT, anti-SARS-CoV-2 spike protein Ig (e.g., IgG, IgA, IgM, etc.), T cell response, NT50 seropositive response, NT80 seropositive response, T cell response, anti-NDV HN antibody, and anti-NDV F antibody.

### 5.6.5 Expression of Transgene

Assays for testing the expression of SARS-CoV-2 delta variant spike protein or portion thereof (e.g., SARS-CoV-2 delta variant spike protein ectodomain or receptor binding domain), or a chimeric F protein in cells infected with a recombinant NDV comprising a packaged genome comprising a transgene that comprises a nucleotide sequence encoding SARS-CoV-2 delta variant spike protein or portion thereof (e.g., SARS-CoV-2 delta variant spike protein ectodomain or receptor binding domain), or chimeric F protein, respectively may be conducted using any assay known in the art, such as, e.g., western blot, immunofluorescence, and ELISA, or any assay described herein.

In a specific aspect, ELISA is utilized to detect expression of a SARS-CoV-2 delta variant spike protein or portion thereof (e.g., SARS-CoV-2 delta variant spike protein ectodomain or receptor binding domain), or a chimeric F protein in cells infected with a recombinant NDV comprising a packaged genome comprising a transgene that comprises a nucleotide sequence encoding of a SARS-CoV-2 delta variant spike protein or portion thereof (e.g., SARS-CoV-2 delta variant spike protein ectodomain or receptor binding domain), a chimeric F protein.

In one embodiment, a SARS-CoV-2 delta variant spike protein or portion thereof (e.g., SARS-CoV-2 delta variant spike protein ectodomain or receptor binding domain), or a chimeric F protein encoded by a packaged genome of a recombinant NDV described herein is assayed for proper folding by testing its ability to bind specifically to an anti-SARS-CoV-2 spike protein (e.g., anti-SARS-CoV-2 delta variant spike protein antibody) using any assay for antibody-antigen interaction known in the art. In another embodiment, a SARS-CoV-2 delta variant spike protein or portion thereof (e.g., SARS-CoV-2 delta variant spike protein ectodomain or receptor binding domain), or a chimeric F protein encoded by a packaged genome of a recombinant NDV described herein is assayed for proper folding by determination of the structure or conformation of the SARS-CoV-2 delta variant spike protein or portion thereof (e.g., SARS-CoV-2 delta variant spike protein ectodomain or receptor binding domain), or chimeric F protein, respectively using any method known in the art such as, e.g., NMR, X-ray crystallographic methods, or secondary structure prediction methods, e.g., circular dichroism. Additional assays assessing the conformation and antigenicity of SARS-CoV-2 delta variant spike protein or portion thereof (e.g., SARS-CoV-2 delta variant spike protein ectodomain or receptor binding domain), or a chimeric F protein may include, e.g., immunofluorescence microscopy, flow cytometry, western blot, and ELISA may be used.

### 5.7 Kits

In one aspect, provided herein is a pharmaceutical pack or kit comprising one or more containers filled with one or more of the ingredients of a composition (e.g., a pharmaceutical compositions) described herein. In a specific embodiment, provided herein is a pharmaceutical pack or kit comprising a container, wherein the container comprises a recombinant NDV described herein. In a specific embodiment, provided herein is a pharmaceutical pack or kit comprising a container, wherein the container comprises an immunogenic composition described herein. Optionally associated with such container(s) can be a notice in the form prescribed by a governmental agency regulating the manufacture, use or sale of pharmaceuticals or biological products, which notice reflects approval by the agency of manufacture, use or sale for human administration.

In another embodiment, provided herein is a kit comprising in one or more containers filled with one or more recombinant NDVs described herein. In another embodiment, provided herein is a kit comprising in one or more containers containing one or more transgenes described herein. In another embodiment, provided herein is a kit comprising in one or more containers containing one or more nucleic acid sequences described herein. In another embodiment, provided herein is a kit comprising in one or more containers containing one or more nucleotide sequences comprising the genome of NDV and a transgene described herein. In another embodiment, provided herein is a kit comprising, in a container, a vector comprising a transgene described herein. In another embodiment, provided herein is a kit comprising, in a container, a vector comprising a nucleic acid sequence described herein.

In a specific embodiment, provided herein is a kit comprising, in a container, a nucleotide sequence comprising a transgene described herein and (1) a NDV F transcription unit, (2) a NDV NP transcription unit, (3) a NDV M transcription unit, (4) a NDV L transcription unit, (5) a NDV P transcription unit, and (6) a NDV HN transcription unit. In some embodiments, the NDV F transcription unit encodes a NDV F protein comprising a leucine to alanine amino acid substitution at the amino residue corresponding to amino acid residue 289 of the F protein of the LaSota NDV strain.

In a specific embodiment, provided herein is a kit comprising, in a container, a vector comprising a nucleotide sequence, wherein the nucleotide sequence comprises a transgene described herein and (1) a NDV F transcription unit, (2) a NDV NP transcription unit, (3) a NDV M transcription unit, (4) a NDV L transcription unit, (5) a NDV P transcription unit, and (6) a NDV HN transcription unit. In some embodiments, the NDV F transcription unit encodes a NDV F protein comprising a leucine to alanine amino acid substitution at the amino residue corresponding to amino acid residue 289 of the F protein of the LaSota NDV strain.

In another embodiment, provided herein is a kit comprising in one or more containers filled with one or more recombinant proteins described herein. In a specific embodiment, provided herein is a pharmaceutical pack or kit comprising a container, wherein the container comprises a recombinant protein described herein.

### 5.8 Sequences

TABLE 1
cDNA of genome of NDV Strains

| | | |
|---|---|---|
| cDNA of | accaaacagagaatccgtgagttacgataaaaggcgaaggagcaattgaagtcgcacggg | SEQ ID |
| genomic | tagaaggtgtgaatctcgagtgcgagcccgaagcacaaactcgagaaagccttctgccaac | NO: 1 |
| sequence of | atgtcttccgtatttgatgagtacgaacagctcctcgcggctcagactcgccccaatggagct | |
| NDV strain | catggagggggagaaaaagggagtaccttaaaagtagacgtcccggtattcactcttaaca | |
| LaSota | gtgatgacccagaagatagatggagctttgtggtattctgcctccggattgctgttagcgaag | |
| | atgccaacaaaccactcaggcaaggtgctctcatatctcttttatgctcccactcacaggtaat | |
| | gaggaaccatgttgccCttgcagggaaacagaatgaagccacattggccgtgcttgagatt | |
| | gatggctttgccaacggcacgccccagttcaacaataggagtggagtgtctgaagagagag | |
| | cacagagatttgcgatgatagcaggatctctccctcgggcatgcagcaacggaaccccgttc | |
| | gtcacagccggggcCgaagatgatgcaccagaagacatcaccgataccctggagaggat | |
| | cctctctatccaggctcaagtatgggtcacagtagcaaaagccatgactgcgtatgagactg | |
| | cagatgagtcggaaacaaggcgaatcaataagtatatgcagcaaggcagggtccaaaaga | |
| | aatacatcctctaccccgtatgcaggagcacaatccaactcacgatcagacagtctcttgcag | |
| | tccgcatctttttggttagcgagctcaagagaggccgcaacacggcaggtggtacctctactt | |
| | attataacctggtaggggacgtagactcatacatcaggaataccgggcttactgcattcttctt | |
| | gacactcaagtacggaatcaacaccaagacatcagcccttgcacttagtagcctctcaggcg | |
| | acatccagaagatgaagcagctcatgcgtttgtatcggatgaaaggagataatgcgccgtac | |
| | atgacattacttggtgatagtgaccagatgagctttgcgcctgccgagtatgcacaactttact | |
| | cctttgccatgggtatggcatcagtcctagataaaggtactgggaaataccaatttgccaggg | |
| | actttatgagcacatcattctggagacttggagtagagtacgctcaggctcagggaagtagc | |
| | attaacgaggatatggctgccgagctaaagctaaccccagcagcaaGgaGgggcctggc | |
| | agctgctgcccaacgggtctccgaGgaGaccagcagcataGacatgcctactcaacaag | |
| | tcggagtcctcactgggcttagcgagggggggtcccaagctctacaaggcggatcgaata | |
| | gatcgcaagggcaaccagaagccggggatggggagacccaattcctggatctgatgaga | |
| | gcggtagcaaatagcatgagggaggcgccaaactctgcacagggcactccccaatcggg | |
| | gcctcccccaactcctgggccatcccaagataacgacaccgactgggggtattgatggaca | |
| | aaacccagcctgcttccacaaaaacatcccaatgccctcacccgtagtcgacccctcgatttg | |
| | cggctctatatgaccacaccctcaaacaaacatccccctctttcctccctccccctgctgtaca | |
| | actAcgTacgccctagataccacaggcacaatgcggctcactaacaatcaaaacagagc | |
| | cgagggaattagaaaaaagtacgggtagaagagggatattcagagatcagggcaagtctc | |
| | ccgagtctctgctctctcctctacctgatagaccaggacaaacatggccacctttacagatgc | |
| | agagatcgacgagctatttgagacaagtggaactgtcattgacaacataattacagcccagg | |
| | gtaaaccagcagagactgttggaaggagtgcaatcccacaaggcaagaccaaggtgctga | |
| | gcgcagcatgggagaagcatgggagcatccagccaccggccagtcaagacaaccccgat | |
| | cgacaggacagatctgacaaacaaccatccacacccgagcaaacgaccccgcatgacag | |
| | cccgccggccacatccgccgaccagccccccacccaggccacagacgaagccgtcgac | |
| | acacagCtcaggaccggagcaagcaactctctgctgttgatgcttgacaagctcagcaata | |
| | aatcgtccaatgctaaaaagggcccatggtcgagcccccaagaggggaatcaccaacgtc | |
| | cgactcaacagcaggggagtcaacccagtcgcggaaacagtcaggaaagaccgcagaa | |
| | ccaagtcaaggccgcccctggaaaccagggcacagacgtgaacacagcatatcatggac | |
| | aatgggaggagtcacaactatcagctggtgcaacccctcatgctctccgatcaaggcagag | |
| | ccaagacaatacccttgtatctgcggatcatgtccagccacctgtagactttgtgcaagcgat | |
| | gatgtctatgatggaggcgatatcacagagagtaagtaaggttgactatcagctagatcttgtc | |
| | ttgaaacagacatcctccatccctatgatgcggtccgaaatccaacagctgaaaacatctgtt | |
| | gcagtcatggaagccaacttgggaatgatgaagattctggatcccggttgtgccaacatttca | |
| | tctctgagtgatctacgggcagttgcccgatctcacccggttttagtttcaggccctggagacc | |
| | cctctccctatgtgacacaaggaggcgaaatggcacttaataaactttcgcaaccagtgcca | |
| | catccatctgaattgattaaacccgccactgcatgcgggcctgatataggagtggaaaagga | |
| | cactgtccgtgcattgatcatgtcacgcccaatgcacccgagttcttcagccaagctcctaag | |
| | caagttagatgcagccgggtcgatcgaggaaatcaggaaaatcaagcgccttgctctaaat | |
| | ggctaattactactgccacacgtagcgggtccctgtccactcggcatcacacggaatctgca | |
| | ccgagttcccccccgcGgacccaaggtccaactctccaagcggcaatcctctctcgcttcct | |
| | cagccccactgaatgAtcgcgtaaccgtaattaatctagctacatttaagattaagaaaaaat | |
| | acgggtagaattggagtgccccaattgtgccaagatggactcatctaggacaattgggctgt | |
| | actttgattctgcccattcttctagcaacctgttagcatttccgatcgtcctacaagAcacagga | |
| | gatgggaagaagcaaatcgccccgcaatataggatccagcgccttgacttgtggactgata | |
| | gtaaggaggactcagtattcatcaccacctatggattcatctttcaagttgggaatgaagaagc | |
| | cacCgtcggcatgatcgatgataaacccaagcgcgagttactttccgctgcgatgctctgcc | |
| | taggaagcgtcccaaataccggagaccttattgagctggcaagggcctgtctcactatgata | |
| | gtcacatgcaagaagagtgcaactaatactgagagaatggttttctcagtagtgcaggcacc | |
| | ccaagtgctgcaaagctgtagggttgtggcaaacaaatactcatcagtgaatgcagtcaagc | |
| | acgtgaaagcgccagagaagattcccgggagtggaaccctagaatacaaggtgaactttgt | |
| | ctccttgactgtggtaccgaagaGggatgtctacaagatcccagctgcagtattgaaggtttc | |
| | tggctcgagtctgtacaatcttgcgctcaatgtcactattaatgtggaggtagacccgaggagt | |
| | cctttggttaaatctCtgtctaagtctgacagcggatactatgctaacctcttcttgcatattgga | |
| | cttatgaccacTgtagataggaaggggaagaaagtgacatttgacaagctggaaaagaaa | |
| | ataaggagccttgatctatctgtcgggctcagtgatgtgctcgggccttccgtgttggtaaaag | |
| | caagaggtgcacggactaagcttttggcacctttcttctctagcagtgggacagcctgctatc | |
| | ccatagcaaatgcttctcctcaggtggccaagatactctggagtcaaaccgcgtgcctgcgg | |
| | agcgttaaaatcattatccaagcaggtacccaacgcgctgtcgcagtgaccgccgaccacg | |
| | aggttacctctactaagctggagaaggggcacacccttgccaaatacaatccttttaagaaat | |
| | aagctgcgtctctgagattgcgctccgcccactcacccagatcatcatgacacaaaaaacta | |
| | atctgtcttgattatttacagttagtttacctgtctatcaagttagaaaaaacacgggtagaagatt | |
| | ctggatcccggttggcgccctccaggtgcaagatgggctccagaccttctaccaagaaccc | |
| | agcacctatgatgctgactatccgggttgcgctggtactgagttgcatctgtccggcaaactc | |
| | cattgatggcaggcctcttgcagctgcaggaattgtggttacaggagacaaagccgtcaaca | |
| | tatacacctcatcccagacaggatcaatcatagttaagctcctcccgaatctgcccaaggata | |
| | aggaggcatgtgcgaaagcccccttggatgcatacaacaggacattgaccactttgctcacc | |
| | ccccttggtgactctatccgtaggatacaagagtctgtgactacatctggaggggggagaca | |
| | ggggcgccttataggcgccattattggcggtgtggctcttggggttgcaactgccgcacaaa | |
| | taacagcggccgcagctctgatacaagccaaacaaaatgctgccaacatcctccgacttaaa | |
| | gagagcattgccgcaaccaatgaggctgtgcatgaggtcactgacggattatcgcaactag | |
| | cagtggcagttgggaagatgcagcagtttgttaatgaccaatttaataaaacagctcaggaat | |
| | tagactgcatcaaaattgcacagcaagttggtgtagagctcaacctgtacctaaccgaattga | |
| | ctacagtattcggaccacaaatcacttcacctgctttaaacaagctgactattcaggcactttac | |
| | aatctagctggtggaaatatggattacttattgactaagttaggtgtagggaacaatcaactca | |
| | gctcattaatcggtagcggcttaatcaccggtaaccctattctatacgactcacagactcaact | |
| | cttgggtatacaggtaactctaccttcagtcgggaacctaaataatatgcgtgccacctacttg | |
| | gaaaccttatccgtaagcacaaccaggggatttgcctcggcacttgtcccAaaagtggtga | |
| | cacaggtcggttctgtgatagaagaacttgacacctcatactgtatagaaactgacttagattta | |
| | tattgtacaagaatagtaacgttccctatgtcccctggtatttattcctgcttgagcggcaatacg | |
| | tcggcctgtatgtactcaaagaccgaaggcgcacttactacaccatacatgactatcaaaggt | |
| | tcagtcatcgccaactgcaagatgacaacatgtagatgtgtaaaccccccgggtatcatatcg | |
| | caaaactatggagaagccgtgtctctaatagataaacaatcatgcaatgttttatccttaggcg | |
| | ggataactttaaggctcagtggggaattcgatgtaacttatcagaagaatatctcaatacaaga | |
| | ttctcaagtaataataacaggcaatcttgatatctcaactgagcttgggaatgtcaacaactcg | |
| | atcagtaatgctttgaataagttagaggaaagcaacagaaaactagacaaagtcaatgtcaa | |
| | actgactagcacatctgctctcattacctatatcgttttgactatcatatctcttgtttttggtatactt | |
| | agcctgattctagcatgctacctaatgtacaagcaaaaggcgcaacaaaagaccttattatgg | |
| | cttgggaataatactctagatcagatgagagccactacaaaaatgtgaacacagatgaggaa | |
| | cgaaggtttccctaatagtaatttgtgtgaaagttctggtagtctgtcagttcagagagttaaga | |
| | aaaaactaccggttgtagatgaccaaaggacgatatacgggtagaacggtaagagaggcc | |
| | gcccctcaattgcgagccaggcttcacaacctccgttctaccgcttcaccgacaacagtcctc | |
| | aatcatggaccgcgccgttagccaagttgcgttagagaatgatgaaagagaggcaaaaaat | |
| | acatggcgcttgatattccggattgcaatcttattcttaacagtagtgaccttggctatatctgta | |
| | gcctcccttttatatagcatgggggctagcacacctagcgatcttgtaggcataccgactagg | |
| | atttccagggcagaagaaaagattacatctacacttggttccaatcaagatgtagtagatagg | |
| | atatataagcaagtggcccttgagtctccgttggcattgttaaatactgagaccacaattatgaa | |
| | cgcaataacatctctctcttatcagattaatggagctgcaaacaacagtgggtggggggcac | |
| | ctatccatgacccagattatataggggggataggcaaagaactcattgtagatgatgctagtg | |
| | atgtcacatcattctatccctctgcatttcaagaacatctgaattttatcccggcgcctactacag | |
| | gatcaggttgcactcgaataccctcatttgacatgagtgctacccattactgctacacccataat | |
| | gtaatattgtctggatgcagagatcactcacattcatatcagtatttagcacttggtgtgctccg | |
| | gacatctgcaacagggagggtattcttttctactctgcgttccatcaacctggacgacaccca | |
| | aaatcggaagtcttgcagtgtgagtgcaactcccctgggttgtgatatgctgtgctcgaaagt | |
| | cacggagacagaggaagaagattataactcagctgtccctacgcggatggtacatgggag | |
| | gttagggttcgacggccagtaccacgaaaaggacctagatgtcacaacattattcggggact | |
| | gggtggccaactacccaggagtagggggtggatcttttattgacagccgcgtatggttctca | |
| | gtctacggagggttaaaacccaattcacccagtgacactgtacaggaagggaaatatgtgat | |
| | atacaagcgatacaatgacacatgcccagatgagcaagactaccagattcgaatggccaag | |
| | tcttcgtataagcctggacggtttggtgggaaacgcatacagcaggctatcttatctatcaagg | |
| | tgtcaacatccttaggcgaagacccggtactgactgtaccgcccaacacagtcacactcatg | |
| | ggggccgaaggcagaattctcacagtagggacatctcatttcttgtatcaacgagggtcatca | |
| | tacttctctcccgcgttattatatcctatgacagtcagcaacaaaacagccactcttcatagtcct | |
| | tatacattcaatgccttcactcggccaggtagtatcccttgccaggcttcagcaagatgcccca | |
| | actcgtgtgttactggagtctatacagatccatatcccctaatcttctatagaaaccacaccttg | |
| | cgaggggtattcgggacaatgcttgatggtgtacaagcaagacttaaccctgcgtctgcagt | |
| | attcgatagcacatcccgcagtcgcattactcgagtgagttcaagcagtaccaaagcagcat | |
| | acacaacatcaacttgttttaaagtggtcaagactaataagacctattgtctcagcattgctgaa | |
| | atatctaatactctcttcggagaattcagaatcgtcccgttactagttgagatcctcaaagatga | |
| | cggggttagagaagccaggtctggctagttgagtcaattataaaggagttggaaagatggca | |
| | ttgtatcacctatcttctgcgacatcaagaatcaaaccgaatgccggcgcgtgctcgaattcca | |
| | tgttgccagttgaccacaatcagccagtgctcatgcgatcagattaagccttgtcaAtaGtct | |
| | cttgattaagaaaaaatgtaagtggcaatgagatacaaggcaaaacagctcatggtTaaCa | |
| | atacgggtaggacatggcgagctccggtcctgaaagggcagagcatcagattatcctacca | |
| | gagTcacacctgtcttcaccattggtcaagcacaaactactctattactggaaattaactggg | |
| | ctaccgcttcctgatgaatgtgacttcgaccacctcattctcagccgacaatggaaaaaaata | |
| | cttgaatcggcctctcctgatactgagagaatgataaaactcggaagggcagtacaccaaac | |
| | tcttaaccacaattccagaataaccggagtgctccaccccaggtgtttagaaGaactggcta | |
| | atattgaggtcccagattcaaccaacaaatttcggaagattgagaagaagatccaaattcaca | |
| | acacgagatatggagaactgttcacaaggctgtgtacgcatatagagaagaaactgctggg | |
| | gtcatcttggtctaacaatgtcccccggtcagaggagttcagcagcattcgtacggatccggc | |
| | attctggtttcactcaaaatggtccacagccaagtttgcatggctccatataaaacagatccag | |
| | aggcatctgatggtggcagctaGgacaaggtctgcggccaacaaattggtgatgctaaccc | |
| | ataaggtaggccaagtctttgtcactcctgaacttgtcgttgtgacgcatacgaatgagaacaa | |
| | gttcacatgtcttacccaggaacttgtattgatgtatgcagatatgatggagggcagagatatg | |
| | gtcaacataatatcaaccacggcggtgcatctcagaagcttatcagagaaaattgatgacattt | |
| | tgcggttaatagacgctctggcaaaagacttgggtaatcaagtctacgatgttgtatcactaat | |
| | ggagggatttgcatacggagctgtccagctactcgagccgtcaggtacatttgcaggagattt | |
| | cttcgcattcaacctgcaggagcttaaagacattctaattggcctcctccccaatgatatagca | |
| | gaatccgtgactcatgcaatcgctactgtattctctggtttagaacagaatcaagcagctgaga | |
| | tgttgtgtctgttgcgtctgtggggtcacccactgcttgagtcccgtattgcagcaaaggcagt | |
| | caggagccaaatgtgcgcaccgaaaatggtagactttgatatgatccttcaggtactgtctttc | |
| | ttcaagggaacaatcatcaacgggtacagaaagaagaatgcaggtgtgtggccgcgagtc | |
| | aaagtggatacaatatatgggaaggtcattgggcaactacatgcagattcagcagagatttca | |
| | cacgatatcatgttgagagagtataagagtttatctgcacttgaatttgagccatgtatagaatat | |
| | gaccctgtcaccaacctgagcatgttcctaaaagacaaggcaatcgcacaccccaacgata | |
| | attggcttgcctcgtttaggcggaaccttctctccgaagaccagaagaaacatgtaaaagaa | |
| | gcaacttcgactaatcgcctcttgatagagtttttagagtcaaatgattttgatccatataaagag | |
| | atggaatatctgacgacccttgagtaccttagagatgacaatgtggcagtatcatactcgctca | |
| | aggagaaggaagtgaaagttaatggacggatcttcgctaagctgacaaagaagttaaggaa | |
| | ctgtcaggtgatggcggaagggatcctagccgatcagattgcacctttctttcagggaaatgg | |
| | agtcattcaggatagcatatccttgaccaagagtatgctagcgatgagtcaactgtcttttaaca | |
| | gcaataagaaacgtatcactgactgtaaagaaagagtatcttcaaaccgcaatcatgatccga | |
| | aaagcaagaaccgtcggagagttgcaaccttcataacaactgacctgcaaaagtactgtctt | |
| | aattggagatatcagacaatcaaattgttcgctcatgccatcaatcagttgatgggcctacctc | |
| | acttcttcgaatggattcacctaagactgatggacactacgatgttcgtaggagaccctttcaat | |
| | cctccaagtgaccctactgactgtgacctctcaagagtccctaatgatgacatatatattgtca | |
| | gtgccagagggggtatcgaaggattatgccagaagctatggacaatgatctcaattgctgca | |
| | atccaacttgctgcagctagatcgcattgtcgtgttgcctgtatggtacagggtgataatcaag | |
| | taatagcagtaacgagagaggtaagatcagacgactctccggagatggtgttgacacagttg | |
| | catcaagccagtgataatttcttcaaggaattaattcatgtcaatcatttgattggccataatttga | |
| | aggatcgtgaaaccatcaggtcagacacattcttcatatacagcaaacgaatcttcaaagatg | |
| | gagcaatcctcagtcaagtcctcaaaaattcatctaaattagtgctagtgtcaggtgatctcagt | |
| | gaaaacaccgtaatgtcctgtgccaacattgcctctactgtagcacggctatgcgagaacgg | |
| | gcttcccaaagacttctgttactatttaaactatataatgagttgtgtgcagacatactttgactct | |
| | gagttctccatcaccaacaattcgcaccccgatcttaatcagtcgtggattgaggacatctcttt | |
| | tgtgcactcatatgttctgactcctgcccaattagggggactgagtaaccttcaatactcaagg | |
| | ctctacactagaaatatcggtgacccggggactactgcttttgcagagatcaagcgactaga | |
| | agcagtgggattactgagtcctaacattatgactaatatcttaactaggccgcctgggaatgg | |
| | agattgggccagtctgtgcaacgacccatactctttcaattttgagactgttgcaagcccaaat | |
| | attgttcttaagaaacatacgcaaagagtcctatttgaaacttgttcaaatcccttattgtctgga | |
| | gtgcacacagaggataatgaggcagaagagaaggcattggctgaattcttgcttaatcaaga | |
| | ggtgattcatccccgcgttgcgcatgccatcatggaggcaagctctgtaggtaggagaaag | |
| | caaattcaagggcttgttgacacaacaaacaccgtaattaagattgcgcttactaggaggcca | |
| | ttaggcatcaagaggctgatgcggatagtcaattattctagcatgcatgcaatgtgtttagag | |
| | acgatgttttttcctccagtagatccaaccaccccttagtctcttctaatatgtgttctctgacact | |
| | ggcagactatgcacggaatagaagctggtcacctttgacgggaggcaggaaaatactgggt | |
| | gtatctaatcctgatacgatagaactcgtagagggtgagattcttagtgtaagcggagggtgt | |
| | acaagatgtgacagcggagatgaacaatttacttggttccatcttccaagcaatatagaattga | |
| | ccgatgacaccagcaagaatcctccgatgagggtaccatatctcgggtcaaagacacagga | |
| | gaggagagctgcctcacttgcaaaaatagctcatatgtcgccacatgtaaaggctgccctaa | |
| | gggcatcatccgtgttgatctgggcttatggggataatgaagtaaattggactgctgctcttac | |
| | gattgcaaaatctcggtgtaatgtaaacttagagtatcttcggttactgtcccctttacccacgg | |
| | ctgggaatcttcaacatagactagatgatggtataactcagatgacattcacccctgcatctct | |
| | ctacaggGtgtcaccttacattcacatatccaatgattctcaaaggctgttcactgaagaagg | |
| | agtcaaagaggggaatgtggtttaccaacagatcatgctcttgggtttatctctaatcgaatcg | |
| | atctttccaatgacaacaaccaggacatatgatgagatcacactgcacctacatagtaaattta | |
| | gttgctgtatcagagaagcacctgttgcggttcctttcgagctacttggggtggtaccggaact | |
| | gaggacagtgacctcaaataagtttatgtatgatcctagccctgtatcggagggagactttgc | |
| | gagacttgacttagctatcttcaagagttatgagcttaatctggagtcatatcccacgatagag | |
| | ctaatgaacattctttcaatatccagcgggaagttgattggccagtctgtggtttcttatgatgaa | |
| | gatacctccataaagaatgacgccataatagtgtatgacaatacccgaaattggatcagtgaa | |
| | gctcagaattcagatgtggtccgcctatttgaatatgcagcacttgaagtgctcctcgactgttc | |
| | ttaccaactctattacctgagagtaagaggcctGgacaatattgtcttatatatgggtgatttata | |
| | caagaatatgccaggaattctactttccaacattgcagctacaatatctcatcccgtcattcattc | |
| | aaggttacatgcagtgggcctggtcaaccatgacggatcacaccaacttgcagatacggatt | |
| | ttatcgaaatgtctgcaaaactattagtatcttgcacccgacgtgtgatctccggcttatattcag | |
| | gaaataagtatgatctgctgttcccatctgtcttagatgataacctgaatgagaagatgcttcag | |
| | ctgatatcccggttatgtgtctgtacacggtactctttgctacaacaagagaaatcccgaaaa | |
| | taagaggcttaactgcagaagagaaatgttcaatactcactgagtatttactgtcggatgctgt | |
| | gaaaccattacttagccccgatcaagtgagctctatcatgtctcctaacataattacattcccag | |
| | ctaatctgtactacatgtctcggaagagcctcaatttgatcagggaaagggaggacagggat | |
| | actatcctggcgttgttgttcccccaagagccattattagagttcccttctgtgcaagatattggt | |
| | gctcgagtgaaagatccattcacccgacaacctgcggcatttttgcaagagttagatttgagt | |
| | gctccagcaaggtatgacgcattcacacttagtcagattcatcctgaactcacatctccaaatc | |
| | cggaggaagactacttagtacgatacttgttcagagggatagggactgcatcttcctcttggta | |
| | taaggcatctcatctcctttctgtacccgaggtaagatgtgcaagacacgggaactccttatac | |
| | ttagctgaagggagcggagccatcatgagtcttctcgaactgcatgtaccacatgaaactatc | |
| | tattacaatacgctcttttcaaatgagatgaaccccccgcaacgacatttcgggccgacccca | |
| | actcagtttttgaattcggttgtttataggaatctacaggcggaggtaacatgcaaagatggatt | |
| | tgtccaagagttccgtccattatggagagaaaatacagaggaaagCgacctgacctcagat | |
| | aaagTagtggggtatattacatctgcagtgccctacagatctgtatcattgctgcattgtgaca | |
| | ttgaaattcctccagggtccaatcaaagcttactagatcaactagctatcaatttatctctgattg | |
| | ccatgcattctgtaagggagggcggggtagtaatcatcaaagtgttgtatgcaatgggatact | |
| | actttcatctactcatgaacttgtttgctccgtgttccacaaaaggatatattctctctaatggttat | |
| | gcatgtcgaggagatatggagtgttacctggtatttgtcatgggttacctgggcgggcctaca | |
| | tttgtacatgaggtggtgaggatggcGaaaactctggtgcagcggcacggtacgctTttgt | |
| | ctaaatcagatgagatcacactgaccaggttattcacctcacagcggcagcgtgtgacagac | |
| | atcctatccagtcctttaccaagattaataaagtacttgaggaagaatattgacactgcgctgat | |
| | tgaagccgggggacagcccgtccgtccattctgtgcggagagtctggtgagcacgctagc | |
| | gaacataactcagataacccagatCatcgctagtcacattgacacagttatccggtctgtgat | |
| | atatatggaagctgagggtgatctcgctgacacagtatttctatttaccccttacaatctctctac | |
| | tgacgggaaaaagaggacatcacttaAacagtgcacgagacagatcctagaggttacaat | |
| | actaggtcttagagtcgaaaatctcaataaaataggcgatataatcagcctagtgcttaaagg | |
| | catgatctccatggaggaccttatcccactaaggacatacttgaagcatagtacctgccctaa | |
| | atatttgaaggctgtcctaggtattaccaaactcaaagaaatgtttacagacacttctgtaCtgt | |
| | acttgactcgtgctcaacaaaaattctacatgaaaactataggcaatgcagtcaaaggatatta | |
| | cagtaactgtgactcttaacgaaaatcacatattaataggctccttttttggccaattgtattcttgt | |
| | tgatttaatcatattatgttagaaaaaagttgaaccctgactccttaggactcgaattcgaactca | |
| | aataaatgtcttaaaaaaaggttgcgcacaattattcttgagtgtagtctcgtcattcaccaaatc | |
| | tttgtttggt | |
| cDNA of | ACCAAACAGAGAATCCGTAAGTTACGATAAAAGGCGA | SEQ ID |
| genomic | AGGAGCAATTGAAGTCGCACGGGTAGAAGGTGTGAATC | NO: 2 |
| sequence of | TCGAGTGCGAGCCCGAAGCACAAACTCGAGGAAGCCTT | |
| NDV strain | CTGCCAACATGTCTTCCGTATTCGACGAGTACGAACAG | |
| Hitchner B1 | CTCCTCGCGGCTCAGACTCGCCCCAATGGAGCTCATGG | |
| | AGGGGGGGAGAAAGGGAGTACCTTAAAAGTAGACGTC | |
| | CCGGTATTCACTCTTAACAGTGATGACCCAGAAGATAG | |
| | GTGGAGCTTTGTGGTATTCTGCCTCCGGATTGCTGTTAG | |
| | CGAAGATGCCAACAAACCACTCAGGCAAGGTGCTCTCA | |
| | TATCTCTTTTATGCTCCCACTCACAGGTAATGAGGAACC | |
| | ATGTTGCCCTTGCAGGGAAACAGAATGAAGCCACATTG | |
| | GCCGTGCTTGAGATTGATGGCTTTGCCAACGGCACGCC | |
| | CCAGTTCAACAATAGGAGTGGAGTGTCTGAAGAGAGAG | |
| | CACAGAGATTTGCGATGATAGCAGGATCTCTCCCTCGG | |
| | GCATGCAGCAACGGCACCCCGTTCGTCACAGCCGGGGC | |
| | TGAAGATGATGCACCAGAAGACATCACCGATACCCTGG | |
| | AGAGGATCCTCTCTATCCAGGCTCAAGTATGGGTCACA | |
| | GTAGCAAAAGCCATGACTGCGTATGAGACTGCAGATGA | |
| | GTCGGAAACAAGGCGAATCAATAAGTATATGCAGCAAG | |
| | GCAGGGTCCAAAAGAAATACATCCTCTACCCCGTATGC | |
| | AGGAGCACAATCCAACTCACGATCAGACAGTCTCTTGC | |
| | AGTCCGCATCTTTTTGGTTAGCGAGCTCAAGAGAGGCC | |
| | GCAACACGGCAGGTGGTACCTCTACTTATTATAACCTA | |
| | GTAGGGGACGTAGACTCATATATCAGGAATACCGGGCT | |
| | TACTGCATTCTTCTTGACACTCAAGTACGGAATCAACAC | |
| | CAAGACATCAGCCCTTGCACTTAGTAGCCTCTCAGGCG | |
| | ACATCCAGAAGATGAAGCAGCTCATGCGTTTGTATCGG | |
| | ATGAAAGGAGATAATGCGCCGTACATGACATTACTTGG | |
| | TGATAGTGACCAGATGAGCTTTGCGCCTGCCGAGTATG | |
| | CACAACTTTACTCCTTTGCCATGGGTATGGCATCAGTCC | |
| | TAGATAAAGGTACTGGGAAATACCAATTTGCCAGGGAC | |
| | TTTATGAGCACATCATTCTGGAGACTTGGAGTAGAGTA | |
| | CGCTCAGGCTCAGGGAAGTAGCATTAACGAGGATATGG | |
| | CTGCCGAGCTAAAGCTAACCCCGGCAGCAAGGAGGGGC | |
| | CTGGCAGCTGCTGCCCAACGAGTCTCCGAGGTGACCAG | |
| | CAGCATAGACATGCCTACTCAACAAGTCGGAGTCCTCA | |
| | CTGGGCTTAGCGAGGGGGGATCCCAAGCCCTACAAGGC | |
| | GGATCGAATAGATCGCAAGGGCAACCAGAAGCCGGGG | |
| | ATGGGGAGACCCAATTCCTGGATCTGATGAGAGCGGTA | |
| | GCAAATAGCATGAGGGAGGCGCCAAACTCTGCACAGG | |
| | GCACTCCCCAATCGGGGCCTCCCCCAACTCCTGGGCCAT | |
| | CCCAAGATAACGACACCGACTGGGGGTATTGATTGACA | |
| | AAACCCAGCCTGCTTCTACAAGAACATCCCAATGCTCTC | |
| | ACCCGTAGTCGACCCCTCGATTTGCGGCTCTATATGACC | |
| | ACACCCTCAAACAAACATCCCCCTCTTTCCTCCCTCCCC | |
| | CTGCTGTACAACTCCGCACGCCCTAGATACCACAGGCA | |
| | CACCGCGGCTCACTAACAATCAAAACAGAGCCGAGGGA | |
| | ATTAGAAAAAAGTACGGGTAGAAGAGGGATATTCAGA | |
| | GATCAGGGCAAGTCTCCCGAGTCTCTGCTCTCTCCTCTA | |
| | CCTGATAGACCAGGACAAACATGGCCACCTTTACAGAT | |
| | GCAGAGATCGACGAGCTATTTGAGACAAGTGGAACTGT | |
| | CATTGACAACATAATTACAGCCCAGGGTAAACCAGCAG | |
| | AGACTGTTGGAAGGAGTGCAATCCCACAGGGCAAGACC | |
| | AAGGTGCTGAGCGCAGCATGGGAGAAGCATGGGAGCA | |
| | TCCAGCCACCGGCCAGTCAAGACAACCTCGATCGACAG | |
| | GACAGATCTGACAAACAACCATCCACACCCGAGCAAAC | |
| | GACCCCGCACGACAGCCCGCCGGCCACATCCGCTGACC | |
| | AGCCCCCCACCCAGGCCACAGACGAAGCCGTCGACACA | |
| | CAGCTCAGGACCGGAGCAAGCAACTCTCTGCTGTTGAT | |
| | GCTTGACAAGCTCAGCAATAAATCGTCCAATGCTAAAA | |
| | AGGGCCCATGGTCGAGCCCCCAAGAGGGGAATCACCAA | |
| | CGTCCGACTCAACAGCAGGGGAGTCAACCCAGTCGCGG | |
| | AAACAGCCAGGAAAGACTGCAGAACCAAGTCAAGGCC | |
| | GCCCCTGGAAACCAGGGCACAGACGTGAACACAGCATA | |
| | TCATGGACAATGGGAGGAGTCACAACTATCAGCTGGTG | |
| | CAACCCCTCATGCTCTCCGATCAAGGCAGAGCCAAGAC | |
| | AATACCCTTGTATCTGCGGATCATGTCCAGCCACCTGTA | |
| | GACTTTGTGCAAGCGATGATGTCTATGATGGGGGCGAT | |
| | ATCACAGAGAGTAAGTAAGGTTGACTATCAGCTAGATC | |
| | TTGTCTTGAAACAGACATCCTCCATCCCTATGATGCGGT | |
| | CCGAAATCCAACAGCTGAAAACATCTGTTGCAGTCATG | |
| | GAAGCCAACTTGGGAATGATGAAGATTCTGGATCCCGG | |
| | TTGTGCCAACATTTCATCTCTGAGTGATCTACGGGCAGT | |
| | TGCCCGATCTCACCCGGTTTTAGTTTCAGGCCCTGGAGA | |
| | CCCATCTCCCTATGTGATACAAGGAGGCGAAATGGCAC | |
| | TTAATAAACTTTCGCAACCAGTGCCACATCCATCTGAAT | |
| | TGATTAAACCCGCCACTGCATGCGGGCCTGATATAGGA | |
| | GTGGAGAGGGACACTGTCCGTGCATTGATCATGTCACG | |
| | CCCAATGCACCCGAGTTCTTCAGCCAAGCTCCTAAGCA | |
| | AGTTAGATGCAGCCGGGTCGATCGAGGAAATCAGGAAA | |
| | ATCAAGCGCCTTGCTCTAAATGGCTAATTACTACTGCCA | |
| | CACGTAGCGGGTCCCTGTCCACTCGGCATCACACGGAA | |
| | TCTGCACCGAGTTCCCCCCCGCAGACCCAAGGTCCAAC | |
| | TCTAGAAGCGGCAATCCTCTCTCGCTTCCTCAGCCCCAC | |
| | TGAATGATCGCGTAACCGTAATTAATCTAGCTACATTAA | |
| | GGATTAAGAAAAAATACGGGTAGAATTGGAGTGCCCCA | |
| | ATTGTGCCAAGATGGACTCATCTAGGACAATTGGGCTG | |
| | TACTTTGATTCTGCCCATTCTTCTAGCAACCTGTTAGCA | |
| | TTTCCGATCGTCCTACAAGACACAGGAGATGGGAAGAA | |
| | GCAAATCGCCCCGCAATATAGGATCCAGCGCCTTGACT | |
| | CGTGGACTGATAGTAAGGAAGACTCAGTATTCATCACC | |
| | ACCTATGGATTCATCTTTCAAGTTGGGAATGAGGAAGC | |
| | CACTGTCGGCATGATCGATGATAAACCCAAGCGCGAGT | |
| | TACTTTCCGCTGCGATGCTCTGCCTAGGAAGCGTCCCAA | |
| | ATACCGGAGACCTTGTTGAGCTGGCAAGGGCCTGTCTC | |
| | ACTATGATGGTCACATGCAAGAAGAGTGCAACTAATAC | |
| | TGAGAGAATGGTTTTCTCAGTAGTGCAGGCACCCCAAG | |
| | TGCTGCAAAGCTGTAGGGTTGTGGCAAATAAATACTCA | |
| | TCAGTGAATGCAGTCAAGCACGTGAAAGCGCCAGAGAA | |
| | GATCCCCGGGAGTGGAACCCTAGAATACAAGGTGAACT | |
| | TTGTCTCCTTGACTGTGGTACCGAAGAAGGATGTCTACA | |
| | AGATCCCAGCTGCAGTATTGAAGATTTCTGGCTCGAGTC | |
| | TGTACAATCTTGCGCTCAATGTCACTATTAATGTGGAGG | |
| | TAGACCCGAGGAGTCCTTTGGTTAAATCTCTGTCTAAGT | |
| | CTGACAGCGGATACTATGCTAACCTCTTCTTGCATATTG | |
| | GACTTATGACCACCGTAGATAGGAAGGGGAAGAAAGT | |
| | GACATTTGACAAGCTGGAAAAGAAAATAAGGAGCCTTG | |
| | ATCTATCTGTCGGGCTCAGTGATGTGCTCGGGCCTTCCG | |
| | TGTTGGTAAAAGCAAGAGGTGCACGGACTAAGCTTTTG | |
| | GCACCTTTCTTCTCTAGCAGTGGGACAGCCTGCTATCCC | |
| | ATAGCAAATGCTTCTCCTCAGGTGGCCAAGATACTCTG | |
| | GAGTCAAACCGCGTGCCTGCGGAGCGTTAAAATCATTA | |
| | TCCAAGCAGGTACCCAACGCGCTGTCGCAGTGACCGCT | |
| | GACCACGAGGTTACCTCTACTAAGCTGGAGAAGGGGCA | |
| | CACCCTTGCCAAATACAATCCTTTTAAGAAATAAGCTGC | |
| | GTCTCTGAGATTGCGCTCCGCCCACTCACCCAGATCATC | |
| | ATGACACAAAAAACTAATCTGTCTTGATTATTTACAGTT | |
| | AGTTTACCTGTCCATCAAGTTAGAAAAAACACGGGTAG | |
| | AAGATTCTGGATCCCGGTTGGCGCCCTCCAGGTGCAGG | |
| | ATGGGCTCCAGACCTTCTACCAAGAACCCAGCACCTAT | |
| | GATGCTGACTATCCGGGTCGCGCTGGTACTGAGTTGCAT | |
| | CTGCCCGGCAAACTCCATTGATGGCAGGCCTCTTGCAG | |
| | CTGCAGGAATTGTGGTTACAGGAGACAAAGCAGTCAAC | |
| | ATATACACCTCATCCCAGACAGGATCAATCATAGTTAA | |
| | GCTCCTCCCGAATCTGCCCAAGGATAAGGAGGCATGTG | |
| | CGAAAGCCCCCTTGGATGCATACAACAGGACATTGACC | |
| | ACTTTGCTCACCCCCCTTGGTGACTCTATCCGTAGGATA | |
| | CAAGAGTCTGTGACTACATCTGGAGGGGGGAGACAGGG | |
| | GCGCCTTATAGGCGCCATTATTGGCGGTGTGGCTCTTGG | |
| | GGTTGCAACTGCCGCACAAATAACAGCGGCCGCAGCTC | |
| | TGATACAAGCCAAACAAAATGCTGCCAACATCCTCCGA | |
| | CTTAAAGAGAGCATTGCCGCAACCAATGAGGCTGTGCA | |
| | TGAGGTCACTGACGGATTATCGCAACTAGCAGTGGCAG | |
| | TTGGGAAGATGCAGCAGTTTGTTAATGACCAATTTAAT | |
| | AAAACAGCTCAGGAATTAGACTGCATCAAAATTGCACA | |
| | GCAAGTTGGTGTAGAGCTCAACCTGTACCTAACCGAAT | |
| | TGACTACAGTATTCGGACCACAAATCACTTCACCTGCCT | |
| | TAAACAAGCTGACTATTCAGGCACTTTACAATCTAGCTG | |
| | GTGGGAATATGGATTACTTATTGACTAAGTTAGGTATA | |
| | GGGAACAATCAACTCAGCTCATTAATCGGTAGCGGCTT | |
| | AATCACCGGTAACCCTATTCTATACGACTCACAGACTCA | |
| | ACTCTTGGGTATACAGGTAACTCTACCTTCAGTCGGGAA | |
| | CCTAAATAATATGCGTGCCACCTACTTGGAAACCTTATC | |
| | CGTAAGCACAACCAGGGGATTTGCCTCGGCACTTGTCC | |
| | CAAAAGTGGTGACACAGGTCGGTTCTGTGATAGAAGAA | |
| | CTTGACACCTCATACTGTATAGAAACTGACTTAGATTTA | |
| | TATTGTACAAGAATAGTAACGTTCCCTATGTCCCCTGGT | |
| | ATTTACTCCTGCTTGAGCGGCAATACATCGGCCTGTATG | |
| | TACTCAAAGACCGAAGGCGCACTTACTACACCATATAT | |
| | GACTATCAAAGGCTCAGTCATCGCTAACTGCAAGATGA | |
| | CAACATGTAGATGTGTAAACCCCCCGGGTATCATATCG | |
| | CAAAACTATGGAGAAGCCGTGTCTCTAATAGATAAACA | |
| | ATCATGCAATGTTTTATCCTTAGGCGGGATAACTTTAAG | |
| | GCTCAGTGGGGAATTCGATGTAACTTATCAGAAGAATA | |
| | TCTCAATACAAGATTCTCAAGTAATAATAACAGGCAAT | |
| | CTTGATATCTCAACTGAGCTTGGGAATGTCAACAACTCG | |
| | ATCAGTAATGCTTTGAATAAGTTAGAGGAAAGCAACAG | |
| | AAAACTAGACAAAGTCAATGTCAAACTGACCAGCACAT | |
| | CTGCTCTCATTACCTATATCGTTTTGACTATCATATCTCT | |
| | TGTTTTTGGTATACTTAGCCTGATTCTAGCATGCTACCT | |
| | AATGTACAAGCAAAAGGCGCAACAAAAGACCTTATTAT | |
| | GGCTTGGGAATAATACCCTAGATCAGATGAGAGCCACT | |
| | ACAAAAATGTGAACACAGATGAGGAACGAAGGTTTCCC | |
| | TAATAGTAATTTGTGTGAAAGTTCTGGTAGTCTGTCAGT | |
| | TCGGAGAGTTAAGAAAAAACTACCGGTTGTAGATGACC | |
| | AAAGGACGATATACGGGTAGAACGGTAAGAGAGGCCG | |
| | CCCCTCAATTGCGAGCCAGACTTCACAACCTCCGTTCTA | |
| | CCGCTTCACCGACAACAGTCCTCAATCATGGACCGCGC | |
| | CGTTAGCCAAGTTGCGTTAGAGAATGATGAAAGAGAGG | |
| | CAAAAAATACATGGCGCTTGATATTCCGGATTGCAATC | |
| | TTATTCTTAACAGTAGTGACCTTGGCTATATCTGTAGCC | |
| | TCCCTTTTATATAGCATGGGGGCTAGCACACCTAGCGAT | |
| | CTTGTAGGCATACCGACTAGGATTTCCAGGGCAGAAGA | |
| | AAAGATTACATCTACACTTGGTTCCAATCAAGATGTAGT | |
| | AGATAGGATATATAAGCAAGTGGCCCTTGAGTCTCCAT | |
| | TGGCATTGTTAAATACTGAGACCACAATTATGAACGCA | |
| | ATAACATCTCTCTCTTATCAGATTAATGGAGCTGCAAAC | |
| | AACAGCGGGTGGGGGGCACCTATTCATGACCCAGATTA | |
| | TATAGGGGGGATAGGCAAAGAACTCATTGTAGATGATG | |
| | CTAGTGATGTCACATCATTCTATCCCTCTGCATTTCAAG | |
| | AACATCTGAATTTTATCCCGGCGCCTACTACAGGATCAG | |
| | GTTGCACTCGAATACCCTCATTTGACATGAGTGCTACCC | |
| | ATTACTGCTACACCCATAATGTAATATTGTCTGGATGCA | |
| | GAGATCACTCACACTCACATCAGTATTTAGCACTTGGTG | |
| | TGCTCCGGACATCTGCAACAGGGAGGGTATTCTTTTCTA | |
| | CTCTGCGTTCCATCAACCTGGACGACACCCAAAATCGG | |
| | AAGTCTTGCAGTGTGAGTGCAACTCCCCTGGGTTGTGAT | |
| | ATGCTGTGCTCGAAAGCCACGGAGACAGAGGAAGAAG | |
| | ATTATAACTCAGCTGTCCCTACGCGGATGGTACATGGG | |
| | AGGTTAGGGTTCGACGGCCAATATCACGAAAAGGACCT | |
| | AGATGTCACAACATTATTCGGGGACTGGGTGGCCAACT | |
| | ACCCAGGAGTAGGGGGTGGATCTTTTATTGACAGCCGC | |
| | GTATGGTTCTCAGTCTACGGAGGGTTAAAACCCAATAC | |
| | ACCCAGTGACACTGTACAGGAAGGGAAATATGTGATAT | |
| | ACAAGCGATACAATGACACATGCCCAGATGAGCAAGAC | |
| | TACCAGATTCGAATGGCCAAGTCTTCGTATAAGCCTGG | |
| | ACGGTTTGGTGGGAAACGCATACAGCAGGCTATCTTAT | |
| | CTATCAAAGTGTCAACATCCTTAGGCGAAGACCCGGTA | |
| | CTGACTGTACCGCCCAACACAGTCACACTCATGGGGGC | |
| | CGAAGGCAGAATTCTCACAGTAGGGACATCCCATTTCT | |
| | TGTATCAGCGAGGGTCATCATACTTCTCTCCCGCGTTAT | |
| | TATATCCTATGACAGTCAGCGACAAAACAGCCACTCTT | |
| | CATAGTCCTTATACATTCAATGCCTTCACTCGGCCAGGT | |
| | AGTATCCCTTGCCAGGCTTCAGCAAGATGCCCCAACTC | |
| | GTGTGTTACTGGAGTCTATACAGATCCATATCCCCTAAT | |
| | CTTCTATAGAAACCACACCTTGCGAGGGGTATTCGGGA | |
| | CAATGCTTGATGGTGAACAAGCAAGACTTAACCCTGCG | |
| | TCTGCAGTATTCGATAGCACATCCCGCAGTCGCATAACT | |
| | CGAGTGAGTTCAAGCAGCATCAAAGCAGCATACACAAC | |
| | ATCAACTTGTTTTAAAGTGGTCAAGACCAATAAGACCT | |
| | ATTGTCTCAGCATTGCTGAAATATCTAATACTCTCTTCG | |
| | GAGAATTCAGAATCGTCCCGTTACTAGTTGAGATCCTCA | |
| | AAGATGACGGGGTTAGAGAAGCCAGGTCTGGCTAGTTG | |
| | AGTCAACTATGAAAGAGTTGGAAAGATGGCATTGTATC | |
| | ACCTATCTTCTGCGACATCAAGAATCAAACCGAATGCC | |
| | GGCGCGTGCTCGAATTCCATGTCGCCAGTTGACCACAA | |
| | TCAGCCAGTGCTCATGCGATCAGATTAAGCCTTGTCAAT | |
| | AGTCTCTTGATTAAGAAAAAATGTAAGTGGCAATGAGA | |
| | TACAAGGCAAAACAGCTCACGGTAAATAATACGGGTAG | |
| | GACATGGCGAGCTCCGGTCCTGAAAGGGCAGAGCATCA | |
| | GATTATCCTACCAGAGTCACACCTGTCTTCACCATTGGT | |
| | CAAGCACAAACTACTCTATTATTGGAAATTAACTGGGC | |
| | TACCGCTTCCTGATGAATGTGACTTCGACCACCTCATTC | |
| | TCAGCCGACAATGGAAAAAAATACTTGAATCGGCCTCT | |
| | CCTGATACTGAGAGAATGATAAAACTCGGAAGGGCAGT | |
| | ACACCAAACTCTTAACCACAATTCCAGAATAACCGGAG | |
| | TACTCCACCCCAGGTGTTTAGAAGAACTGGCTAATATTG | |
| | AGGTCCCTGATTCAACCAACAAATTTCGGAAGATTGAG | |
| | AAGAAGATCCAAATTCACAACACGAGATATGGAGAACT | |
| | GTTCACAAGGCTGTGTACGCATATAGAGAAGAAACTGC | |
| | TGGGGTCATCTTGGTCTAACAATGTCCCCCGGTCAGAG | |
| | GAGTTCAGCAGCATTCGTACGGATCCGGCATTCTGGTTT | |
| | CACTCAAAATGGTCCACAGCCAAGTTTGCATGGCTCCA | |
| | TATAAAACAGATCCAGAGGCATCTGATTGTGGCAGCTA | |
| | GGACAAGGTCTGCGGCCAACAAATTGGTGATGCTAACC | |
| | CATAAGGTAGGCCAAGTCTTTGTCACTCCTGAACTTGTT | |
| | GTTGTGACGCATACGAATGAGAACAAGTTCACATGTCT | |
| | TACCCAGGAACTTGTATTGATGTATGCAGATATGATGG | |
| | AGGGCAGAGATATGGTCAACATAATATCAACCACGGCG | |
| | GTGCATCTCAGAAGCTTATCAGAGAAAATTGATGACAT | |
| | TTTGCGGTTAATAGACGCTCTGGCAAAAGACTTGGGTA | |
| | ATCAAGTCTACGATGTTGTATCACTAATGGAGGGATTTG | |
| | CATACGGAGCTGTCCAGCTACTCGAGCCGTCAGGTACA | |
| | TTTGCGGGAGATTTCTTCGCATTCAACCTGCAGGAGCTT | |
| | AAAGACATTCTAATTGGCCTCCTCCCCAATGATATAGCA | |
| | GAATCCGTGACTCATGCAATCGCTACTGTATTCTCTGGT | |
| | TTAGAACAGAATCAAGCAGCTGAGATGTTGTGCCTGTT | |
| | GCGTCTGTGGGGTCACCCACTGCTTGAGTCCCGTATTGC | |
| | AGCAAAGGCAGTCAGGAGCCAAATGTGCGCACCGAAA | |
| | ATGGTAGACTTTGATATGATCCTTCAGGTACTGTCTTTC | |
| | TTCAAGGGAACAATCATCAACGGATACAGAAAGAAGA | |
| | ATGCAGGTGTGTGGCCGCGAGTCAAAGTGGATACAATA | |
| | TATGGGAAGGTCATTGGGCAACTACATGCAGATTCAGC | |
| | AGAGATTTCACACGATATCATGTTGAGAGAGTATAAGA | |
| | GTTTATCTGCACTTGAATTTGAGCCATGTATAGAATACG | |
| | ACCCTGTCACTAACCTGAGCATGTTCCTAAAAGACAAG | |
| | GCAATCGCACACCCCAACGATAATTGGCTTGCCTCGTTT | |
| | AGGCGGAACCTTCTCTCCGAAGACCAGAAGAAACATGT | |
| | AAAGGAAGCGACTTCGACTAACCGCCTCTTGATAGAGT | |
| | TTTTAGAGTCAAATGATTTTGATCCATATAAAGAGATGG | |
| | AATATCTGACGACCCTTGAGTACCTTAGAGATGACAAT | |
| | GTGGCAGTATCATACTCGCTCAAAGAGAAGGAAGTGAA | |
| | AGTTAATGGACGGATCTTCGCTAAGCTGACAAAGAAGT | |
| | TAAGGAACTGTCAGGTGATGGCGGAAGGGATCCTAGCC | |
| | GATCAGATTGCACCTTTCTTTCAGGGAAATGGAGTCATT | |
| | CAGGATAGCATATCCTTGACCAAGAGTATGCTAGCGAT | |
| | GAGTCAACTGTCTTTTAACAGCAATAAGAAACGTATCA | |
| | CTGACTGTAAAGAAAGAGTATGTTCAAACCGCAATCAT | |
| | GATCCGAAAAGCAAGAACCGTCGGAGAGTTGCAACCTT | |
| | CATAACAACTGACCTGCAAAAGTACTGTCTTAATTGGA | |
| | GATATCAGACGATCAAATTGTTCGCTCATGCCATCAATC | |
| | AGTTGATGGGCCTACCTCATTTCTTCGAGTGGATTCACC | |
| | TAAGACTGATGGACACTACGATGTTCGTAGGAGACCCT | |
| | TTCAATCCTCCAAGTGACCCTACTGACTGTGACCTCTCA | |
| | AGAGTCCCTAATGATGACATATATATTGTCAGTGCCAG | |
| | AGGGGGTATCGAAGGATTATGCCAGAAGCTATGGACAA | |
| | TGATCTCAATTGCTGCAATCCAACTTGCTGCAGCTAGAT | |
| | CGCATTGTCGTGTTGCCTGTATGGTACAGGGTGATAATC | |
| | AAGTAATAGCAGTAACGAGAGAGGTAAGATCAGATGA | |
| | CTCTCCGGAGATGGTGTTGACACAGTTGCATCAAGCCA | |
| | GTGATAATTTCTTCAAGGAATTAATCCATGTCAATCATT | |
| | TGATTGGCCATAATTTGAAGGATCGTGAAACCATCAGG | |
| | TCAGACACATTCTTCATATACAGCAAACGAATCTTCAA | |
| | AGATGGAGCAATCCTCAGTCAAGTCCTCAAAAATTCAT | |
| | CTAAATTAGTGCTAGTGTCAGGTGATCTCAGTGAAAAC | |
| | ACCGTAATGTCCTGTGCCAACATTGCCTCTACTGTAGCA | |
| | CGGCTATGCGAGAACGGGCTTCCCAAAGACTTCTGTTA | |
| | CTATTTAAACTATATAATGAGTTGTGTGCAGACATACTT | |
| | TGACTCTGAGTTCTCCATCACCAACAATTCGCACCCCGA | |
| | TCTTAATCAGTCGTGGATTGAGGACATCTCTTTTGTGCA | |
| | CTCATATGTTCTGACTCCTGCCCAATTAGGGGGACTGAG | |
| | TAACCTTCAATACTCAAGGCTCTACACTAGAAATATCG | |
| | GTGACCCGGGGACTACTGCTTTTGCAGAGATCAAGCGA | |
| | CTAGAAGCAGTGGGACTACTGAGTCCTAACATTAGGAC | |
| | TAATATCTTAACTAGGCCGCCTGGGAATGGAGATTGGG | |
| | CCAGTCTGTGCAACGACCCATACTCTTTCAATTTTGAGA | |
| | CTGTTGCAAGCCCAAACATTGTTCTTAAGAAACATACG | |
| | CAAAGAGTCCTATTTGAAACTTGTTCAAATCCCTTATTG | |
| | TCTGGAGTGCACACAGAGGATAATGAGGCAGAAGAGA | |
| | AGGCATTGGCTGAATTCTTGCTTAATCAAGAGGTGATTC | |
| | ATCCCCGCGTTGCGCATGCCATCATGGAGGCAAGCTCT | |
| | GTAGGTAGGAGAAAGCAAATTCAAGGGCTTGTTGACAC | |
| | AACAAACACTGTAATTAAGATTGCGCTTACTAGGAGGC | |
| | CATTAGGCATCAAGAGGCTGATGCGGATAGTCAATTAT | |
| | TCTAGCATGCATGCAATGCTGTTTAGAGACGATGTTTTT | |
| | TCCTCTAGTAGATCCAACCACCCCTTAGTCTCTTCTAAT | |
| | ATGTGTTCTCTGACACTGGCAGACTATGCACGGAATAG | |
| | AAGCTGGTCACCTTTGACGGGAGGCAGGAAAATACTGG | |
| | GTGTATCTAATCCTGATACGATAGAACTCGTAGAGGGT | |
| | GAGATTCTTAGTGTAAGCGGAGGGTGTACAAGATGTGA | |
| | CAGCGGAGATGAACAATTTACTTGGTTCCATCTTCCAAG | |
| | CAATATAGAATTGACCGATGACACCAGCAAGAATCCTC | |
| | CGATGAGGGTACCATATCTCGGGTCAAAGACACAGGAG | |
| | AGGAGAGCTGCCTCACTTGCGAAAATAGCTCATATGTC | |
| | GCCACATGTGAAGGCTGCCCTAAGGGCATCATCCGTGT | |
| | TGATCTGGGCTTATGGGGATAATGAAGTAAATTGGACT | |
| | GCTGCTCTTACGATTGCAAAATCTCGGTGTAATGTAAAC | |
| | TTAGAGTATCTTCGGTTACTGTCCCCTTTACCCACGGCT | |
| | GGGAATCTTCAACATAGACTAGATGATGGTATAACTCA | |
| | GATGACATTCACCCCTGCATCTCTCTACAGGGTGTCACC | |
| | TTACATTCACATATCCAATGATTCTCAAAGGCTGTTCAC | |
| | TGAAGAAGGAGTCAAAGAGGGGAATGTGGTTTACCAAC | |
| | AGATCATGCTCTTGGGTTTATCTCTAATCGAATCGATCT | |
| | TTCCAATGACAACAACCAGAACATATGATGAGATCACA | |
| | CTGCACCTACATAGTAAATTTAGTTGCTGTATCAGGGAA | |
| | GCACCTGTTGCGGTTCCTTTCGAGCTACTTGGGGTGGCA | |
| | CCGGAACTGAGGACAGTGACCTCAAATAAGTTTATGTA | |
| | TGATCCTAGCCCTGTATCGGAGGGAGACTTTGCGAGAC | |
| | TTGACTTAGCTATCTTCAAGAGTTATGAGCTTAATCTGG | |
| | AGTCATATCCCACGATAGAGCTAATGAACATTCTTTCAA | |
| | TATCCAGCGGGAAGTTGATTGGCCAGTCTGTGGTTTCTT | |
| | ATGATGAAGATACCTCCATAAAGAATGATGCCATAATA | |
| | GTGTATGACAATACCCGAAATTGGATCAGTGAAGCTCA | |
| | GAATTCAGATGTGGTCCGCCTATTTGAATATGCAGCACT | |
| | TGAAGTGCTCCTCGACTGTTCTTACCAACTCTATTACCT | |
| | GAGAGTAAGAGACCTAGACAATATTGTCTTATATATGG | |
| | GTGATTTATACAAGAATATGCCAGGAATTCTACTTTCCA | |
| | ACATTGCAGCTACAATATCTCATCCTGTCATTCATTCAA | |
| | GGTTACATGCAGTGGGCCTGGTCAACCATGACGGATCA | |
| | CACCAACTTGCAGATACGGATTTTATCGAAATGTCTGCA | |
| | AAACTGTTAGTATCTTGCACCCGACGTGTGATCTCCGGC | |
| | TTATATTCAGGAAATAAGTATGATCTGCTGTTCCCATCT | |
| | GTCTTAGATGATAACCTGAATGAGAAGATGCTTCAGCT | |
| | GATATCCCGGTTATGCTGTCTGTACACGGTACTCTTTGC | |
| | TACAACAAGAGAAATCCCGAAAATAAGAGGCTTAACTG | |
| | CAGAAGAGAAATGTTCAATACTCACTGAGTATTTACTG | |
| | TCGGATGCTGTGAAACCATTACTTAGCCCCGATCAAGT | |
| | GAGCTCTATCATGTCTCCTAACATAATTACATTCCCAGC | |
| | TAATCTGTACTACATGTCTCGGAAGAGCCTCAATTTGAT | |
| | CAGGGAAAGGGAGGACAGGGATACTATCCTGGCGTTGT | |
| | TGTTCCCCCAAGAGCCATTATTAGAGTTCCCTTCTGTGC | |
| | AAGATATTGGTGCTCGAGTGAAAGATCCATTCACCCGA | |
| | CAACCTGCGGCATTTTTGCAAGAGTTAGATTTGAGTGCT | |
| | CCAGCAAGGTATGACGCATTCACACTTAGTCAGATTCA | |
| | TCCTGAACTCACATCTCCAAATCCGGAGGAAGACTACT | |
| | TAGTACGATACTTGTTCAGAGGGATAGGGACTGCATCT | |
| | TCCTCTTGGTATAAGGCATCCCATCTCCTTTCTGTACCC | |
| | GAGGTAAGATGTGCAAGACACGGGAACTCCTTATACTT | |
| | GGCTGAAGGAAGCGGAGCCATCATGAGTCTTCTTGAAC | |
| | TGCATGTACCACATGAAACTATCTATTACAATACGCTCT | |
| | TTTCAAATGAGATGAACCCCCCGCAACGACATTTCGGG | |
| | CCGACCCCAACTCAGTTTTTGAATTCGGTTGTTTATAGG | |
| | AATCTACAGGCGGAGGTAACATGCAAGGATGGATTTGT | |
| | CCAAGAGTTCCGTCCATTATGGAGAGAAAATACAGAGG | |
| | AAAGTGACCTGACCTCAGATAAAGCAGTGGGGTATATT | |
| | ACATCTGCAGTACCCTACAGATCTGTATCATTGCTGCAT | |
| | TGTGACATTGAAATTCCTCCAGGGTCCAATCAAAGCTTA | |
| | CTAGATCAACTAGCTATCAATTTATCTCTGATTGCCATG | |
| | CATTCTGTAAGGGAGGGCGGGGTAGTAATCATCAAAGT | |
| | GTTGTATGCAATGGGATACTACTTTCATCTACTCATGAA | |
| | CTTGTTTGCTCCGTGTTCCACAAAAGGATATATTCTCTC | |
| | TAATGGTTATGCATGTCGAGGGGATATGGAGTGTTACC | |
| | TGGTATTTGTCATGGGTTACCTGGGCGGGCCTACATTTG | |
| | TACATGAGGTGGTGAGGATGGCAAAAACTCTGGTGCAG | |
| | CGGCACGGTACGCTTTTGTCTAAATCAGATGAGATCAC | |
| | ACTGACCAGGTTATTCACCTCACAGCGGCAGCGTGTGA | |
| | CAGACATCCTATCCAGTCCTTTACCAAGATTAATAAAGT | |
| | ACTTGAGGAAGAATATTGACACTGCGCTGATTGAAGCC | |
| | GGGGGACAGCCCGTCCGTCCATTCTGTGCGGAGAGTCT | |
| | GGTGAGCACGCTAGCGAACATAACTCAGATAACCCAGA | |
| | TCATCGCTAGTCACATTGACACAGTCATCCGGTCTGTGA | |
| | TATATATGGAAGCTGAGGGTGATCTCGCTGACACAGTA | |
| | TTTCTATTTACCCCTTACAATCTCTCTACTGACGGGAAA | |
| | AAGAGGACATCACTTAAACAGTGCACGAGACAGATCCT | |
| | AGAGGTTACAATACTAGGTCTTAGAGTCGAAAATCTCA | |
| | ATAAAATAGGCGATATAATCAGCCTAGTGCTTAAAGGC | |
| | ATGATCTCCATGGAGGACCTTATCCCACTAAGGACATA | |
| | CTTGAAGCATAGTACCTGCCCTAAATATTTGAAGGCTGT | |
| | CCTAGGTATTACCAAACTCAAAGAAATGTTTACAGACA | |
| | CTTCTGTACTGTACTTGACTCGTGCTCAACAAAAATTCT | |
| | ACATGAAAACTATAGGCAATGCAGTCAAAGGATATTAC | |
| | AGTAACTGTGACTCCTAACGAAAATCACATATTAATAG | |
| | GCTCCTTTTTTGGCCAATTGTATTCTTGTTGATTTAATTA | |
| | TATTATGTTAGAAAAAAGTTGAACTCTGACTCCTTAGGA | |
| | CTCGAATTCGAACTCAAATAAATGTCTTTAAAAAAGGT | |
| | TGCGCACAATTATTCTTGAGTGTAGTCTCGTCATTCACC | |
| | AAATCTTTGTTTGGT | |
| cDNA of | accaaacagagaatccgtgagttacgataaaaggcgaaggagcaattgaagtcgcacggg | SEQ ID |
| genomic | tagaaggtgtgaatctcgagtgcgagcccgaagcacaaactcgagaaagccttctgccaac | NO:3 |
| sequence of | atgtcttccgtatttgatgagtacgaacagctcctcgcggctcagactcgccccaatggagct | |
| NDV strain | catggagggggagaaaaagggagtaccttaaaagtagacgtcccggtattcactcttaaca | |
| LaSota | gtgatgacccagaagatagatggagctttgtggtattctgcctccggattgctgttagcgaag | |
| (L289A | atgccaacaaaccactcaggcaaggtgctctcatatctcttttatgctcccactcacaggtaat | |
| mutation) | gaggaaccatgttgcccttgcagggaaacagaatgaagccacattggccgtgcttgagattg | |
| | atggctttgccaacggcacgccccagttcaacaataggagtggagtgtctgaagagagagc | |
| | acagagatttgcgatgatagcaggatctctccctcgggcatgcagcaacggaaccccgttc | |
| | gtcacagccggggccgaagatgatgcaccagaagacatcaccgataccctggagaggat | |
| | cctctctatccaggctcaagtatgggtcacagtagcaaaagccatgactgcgtatgagactg | |
| | cagatgagtcggaaacaaggcgaatcaataagtatatgcagcaaggcagggtccaaaaga | |
| | aatacatcctctaccccgtatgcaggagcacaatccaactcacgatcagacagtctcttgcag | |
| | tccgcatctttttggttagcgagctcaagagaggccgcaacacggcaggtggtacctctactt | |
| | attataacctggtaggggacgtagactcatacatcaggaataccgggcttactgcattcttctt | |
| | gacactcaagtacggaatcaacaccaagacatcagcccttgcacttagtagcctctcaggcg | |
| | acatccagaagatgaagcagctcatgcgtttgtatcggatgaaaggagataatgcgccgtac | |
| | atgacattacttggtgatagtgaccagatgagctttgcgcctgccgagtatgcacaactttact | |
| | cctttgccatgggtatggcatcagtcctagataaaggtactgggaaataccaatttgccaggg | |
| | actttatgagcacatcattctggagacttggagtagagtacgctcaggctcagggaagtagc | |
| | attaacgaggatatggctgccgagctaaagctaaccccagcagcaaggaggggcctggca | |
| | gctgctgcccaacgggtctccgaggagaccagcagcatagacatgcctactcaacaagtc | |
| | ggagtcctcactgggcttagcgagggggggtcccaagctctacaaggcggatcgaataga | |
| | tcgcaagggcaaccagaagccggggatggggagacccaattcctggatctgatgagagc | |
| | ggtagcaaatagcatgagggaggcgccaaactctgcacagggcactccccaatcggggc | |
| | ctcccccaactcctgggccatcccaagataacgacaccgactgggggtattgatggacaaa | |
| | acccagcctgcttccacaaaaacatcccaatgccctcacccgtagtcgacccctcgatttgc | |
| | ggctctatatgaccacaccctcaaacaaacatccccctctttcctccctccccctgctgtacaa | |
| | ctacgtacgccctagataccacaggcacaatgcggctcactaacaatcaaaacagagccga | |
| | gggaattagaaaaaagtacgggtagaagagggatattcagagatcagggcaagtctcccg | |
| | agtctctgctctctcctctacctgatagaccaggacaaacatggccacctttacagatgcaga | |
| | gatcgacgagctatttgagacaagtggaactgtcattgacaacataattacagcccagggta | |
| | aaccagcagagactgttggaaggagtgcaatcccacaaggcaagaccaaggtgctgagc | |
| | gcagcatgggagaagcatgggagcatccagccaccggccagtcaagacaaccccgatcg | |
| | acaggacagatctgacaaacaaccatccacacccgagcaaacgaccccgcatgacagcc | |
| | cgccggccacatccgccgaccagccccccacccaggccacagacgaagccgtcgacac | |
| | acagctcaggaccggagcaagcaactctctgctgttgatgcttgacaagctcagcaataaat | |
| | cgtccaatgctaaaaagggcccatggtcgagcccccaagaggggaatcaccaacgtccga | |
| | ctcaacagcaggggagtcaacccagtcgcggaaacagtcaggaaagaccgcagaaccaa | |
| | gtcaaggccgcccctggaaaccagggcacagacgtgaacacagcatatcatggacaatgg | |
| | gaggagtcacaactatcagctggtgcaacccctcatgctctccgatcaaggcagagccaag | |
| | acaatacccttgtatctgcggatcatgtccagccacctgtagactttgtgcaagcgatgatgtc | |
| | tatgatggaggcgatatcacagagagtaagtaaggttgactatcagctagatcttgtcttgaaa | |
| | cagacatcctccatccctatgatgcggtccgaaatccaacagctgaaaacatctgttgcagtc | |
| | atggaagccaacttgggaatgatgaagattctggatcccggttgtgccaacatttcatctctga | |
| | gtgatctacgggcagttgcccgatctcacccggttttagtttcaggccctggagacccctctc | |
| | cctatgtgacacaaggaggcgaaatggcacttaataaactttcgcaaccagtgccacatcca | |
| | tctgaattgattaaacccgccactgcatgcgggcctgatataggagtggaaaaggacactgt | |
| | ccgtgcattgatcatgtcacgcccaatgcacccgagttcttcagccaagctcctaagcaagtt | |
| | agatgcagccgggtcgatcgaggaaatcaggaaaatcaagcgccttgctctaaatggctaa | |
| | ttactactgccacacgtagcgggtccctgtccactcggcatcacacggaatctgcaccgagtt | |
| | cccccccgcggacccaaggtccaactctccaagcggcaatcctctctcgcttcctcagcccc | |
| | actgaatgatcgcgtaaccgtaattaatctagctacatttaagattaagaaaaaatacgggtag | |
| | aattggagtgccccaattgtgccaagatggactcatctaggacaattgggctgtactttgattct | |
| | gcccattcttctagcaacctgttagcatttccgatcgtcctacaagacacaggagatgggaag | |
| | aagcaaatcgccccgcaatataggatccagcgccttgacttgtggactgatagtaaggagg | |
| | actcagtattcatcaccacctatggattcatctttcaagttgggaatgaagaagccaccgtcgg | |
| | catgatcgatgataaacccaagcgcgagttactttccgctgcgatgctctgcctaggaagcgt | |
| | cccaaataccggagaccttattgagctggcaagggcctgtctcactatgatagtcacatgcaa | |
| | gaagagtgcaactaatactgagagaatggttttctcagtagtgcaggcaccccaagtgctgc | |
| | aaagctgtagggttgtggcaaacaaatactcatcagtgaatgcagtcaagcacgtgaaagc | |
| | gccagagaagattcccgggagtggaaccctagaatacaaggtgaactttgtctccttgactgt | |
| | ggtaccgaagagggatgtctacaagatcccagctgcagtattgaaggtttctggctcgagtct | |
| | gtacaatcttgcgctcaatgtcactattaatgtggaggtagacccgaggagtcctttggttaaat | |
| | ctctgtctaagtctgacagcggatactatgctaacctcttcttgcatattggacttatgaccactg | |
| | tagataggaaggggaagaaagtgacatttgacaagctggaaaagaaaataaggagccttg | |
| | atctatctgtcgggctcagtgatgtgctcgggccttccgtgttggtaaaagcaagaggtgcac | |
| | ggactaagcttttggcacctttcttctctagcagtgggacagcctgctatcccatagcaaatgc | |
| | ttctcctcaggtggccaagatactctggagtcaaaccgcgtgcctgcggagcgttaaaatcat | |
| | tatccaagcaggtacccaacgcgctgtcgcagtgaccgccgaccacgaggttacctctact | |
| | aagctggagaaggggcacacccttgccaaatacaatccttttaagaaataagctgcgtctctg | |
| | agattgcgctccgcccactcacccagatcatcatgacacaaaaaactaatctgtcttgattattt | |
| | acagttagtttacctgtctatcaagttagaaaaaacacgggtagaagattctggatcccggttg | |
| | gcgccctccaggtgcaagatgggctccagaccttctaccaagaacccagcacctatgatgc | |
| | tgactatccgggttgcgctggtactgagttgcatctgtccggcaaactccattgatggcaggc | |
| | ctcttgcagctgcaggaattgtggttacaggagacaaagccgtcaacatatacacctcatccc | |
| | agacaggatcaatcatagttaagctcctcccgaatctgcccaaggataaggaggcatgtgcg | |
| | aaagcccccttggatgcatacaacaggacattgaccactttgctcaccccccttggtgactct | |
| | atccgtaggatacaagagtctgtgactacatctggaggggggagacaggggcgccttatag | |
| | gcgccattattggcggtgtggctcttggggttgcaactgccgcacaaataacagcggccgc | |
| | agctctgatacaagccaaacaaaatgctgccaacatcctccgacttaaagagagcattgccg | |
| | caaccaatgaggctgtgcatgaggtcactgacggattatcgcaactagcagtggcagttgg | |
| | gaagatgcagcagtttgttaatgaccaatttaataaaacagctcaggaattagactgcatcaaa | |
| | attgcacagcaagttggtgtagagctcaacctgtacctaaccgaattgactacagtattcgga | |
| | ccacaaatcacttcacctgctttaaacaagctgactattcaggcactttacaatctagctggtgg | |
| | aaatatggattacttattgactaagttaggtgtagggaacaatcaactcagctcattaatcggta | |
| | gcggcttaatcaccggtaaccctattctatacgactcacagactcaactcttgggtatacaggt | |
| | aactgccccttcagtcgggaacctaaataatatgcgtgccacctacttggaaaccttatccgta | |
| | agcacaaccaggggatttgcctcggcacttgtcccaaaagtggtgacacaggtcggttctgt | |
| | gatagaagaacttgacacctcatactgtatagaaactgacttagatttatattgtacaagaatag | |
| | taacgttccctatgtcccctggtatttattcctgcttgagcggcaatacgtcggcctgtatgtact | |
| | caaagaccgaaggcgcacttactacaccatacatgactatcaaaggttcagtcatcgccaac | |
| | tgcaagatgacaacatgtagatgtgtaaaccccccgggtatcatatcgcaaaactatggaga | |
| | agccgtgtctctaatagataaacaatcatgcaatgttttatccttaggcgggataactttaaggc | |
| | tcagtggggaattcgatgtaacttatcagaagaatatctcaatacaagattctcaagtaataata | |
| | acaggcaatcttgatatctcaactgagcttgggaatgtcaacaactcgatcagtaatgctttga | |
| | ataagttagaggaaagcaacagaaaactagacaaagtcaatgtcaaactgactagcacatct | |
| | gctctcattacctatatcgttttgactatcatatctcttgtttttggtatacttagcctgattctagcat | |
| | gctacctaatgtacaagcaaaaggcgcaacaaaagaccttattatggcttgggaataatactc | |
| | tagatcagatgagagccactacaaaaatgtgaacacagatgaggaacgaaggtttccctaat | |
| | agtaatttgtgtgaaagttctggtagtctgtcagttcagagagttaagaaaaaactaccggttgt | |
| | agatgaccaaaggacgatatacgggtagaacggtaagagaggccgcccctcaattgcgag | |
| | ccaggcttcacaacctccgttctaccgcttcaccgacaacagtcctcaatcatggaccgcgc | |
| | cgttagccaagttgcgttagagaatgatgaaagagaggcaaaaaatacatggcgcttgatatt | |
| | ccggattgcaatcttattcttaacagtagtgaccttggctatatctgtagcctcccttttatatagc | |
| | atgggggctagcacacctagcgatcttgtaggcataccgactaggatttccagggcagaag | |
| | aaaagattacatctacacttggttccaatcaagatgtagtagataggatatataagcaagtggc | |
| | ccttgagtctccgttggcattgttaaatactgagaccacaattatgaacgcaataacatctctct | |
| | cttatcagattaatggagctgcaaacaacagtgggggggggcacctatccatgacccagat | |
| | tatataggggggataggcaaagaactcattgtagatgatgctagtgatgtcacatcattctatc | |
| | cctctgcatttcaagaacatctgaattttatcccggcgcctactacaggatcaggttgcactcg | |
| | aataccctcatttgacatgagtgctacccattactgctacacccataatgtaatattgtctggatg | |
| | cagagatcactcacattcatatcagtatttagcacttggtgtgctccggacatctgcaacaggg | |
| | agggtattcttttctactctgcgttccatcaacctggacgacacccaaaatcggaagtcttgca | |
| | gtgtgagtgcaactcccctgggttgtgatatgctgtgctcgaaagtcacggagacagaggaa | |
| | gaagattataactcagctgtccctacgcggatggtacatgggaggttagggttcgacggcca | |
| | gtaccacgaaaaggacctagatgtcacaacattattcggggactgggtggccaactaccca | |
| | ggagtagggggtggatcttttattgacagccgcgtatggttctcagtctacggagggttaaaa | |
| | cccaattcacccagtgacactgtacaggaagggaaatatgtgatatacaagcgatacaatga | |
| | cacatgcccagatgagcaagactaccagattcgaatggccaagtcttcgtataagcctggac | |
| | ggtttggtgggaaacgcatacagcaggctatcttatctatcaaggtgtcaacatccttaggcg | |
| | aagacccggtactgactgtaccgcccaacacagtcacactcatgggggccgaaggcagaa | |
| | ttctcacagtagggacatctcatttcttgtatcaacgagggtcatcatacttctctcccgcgttatt | |
| | atatcctatgacagtcagcaacaaaacagccactcttcatagtccttatacattcaatgccttca | |
| | ctcggccaggtagtatcccttgccaggcttcagcaagatgccccaactcgtgtgttactggag | |
| | tctatacagatccatatcccctaatcttctatagaaaccacaccttgcgaggggtattcgggac | |
| | aatgcttgatggtgtacaagcaagacttaaccctgcgtctgcagtattcgatagcacatcccg | |
| | cagtcgcattactcgagtgagttcaagcagtaccaaagcagcatacacaacatcaacttgtttt | |
| | aaagtggtcaagactaataagacctattgtctcagcattgctgaaatatctaatactctcttcgg | |
| | agaattcagaatcgtcccgttactagttgagatcctcaaagatgacggggttagagaagcca | |
| | ggtctggctagttgagtcaattataaaggagttggaaagatggcattgtatcacctatcttctgc | |
| | gacatcaagaatcaaaccgaatgccggcgcgtgctcgaattccatgttgccagttgaccaca | |
| | atcagccagtgctcatgcgatcagattaagccttgtcaatagtctcttgattaagaaaaaatgta | |
| | agtggcaatgagatacaaggcaaaacagctcatggttaacaatacgggtaggacatggcga | |
| | gctccggtcctgaaagggcagagcatcagattatcctaccagagtcacacctgtcttcaccat | |
| | tggtcaagcacaaactactctattactggaaattaactgggctaccgcttcctgatgaatgtga | |
| | cttcgaccacctcattctcagccgacaatggaaaaaaatacttgaatcggcctctcctgatact | |
| | gagagaatgataaaactcggaagggcagtacaccaaactcttaaccacaattccagaataa | |
| | ccggagtgctccaccccaggtgtttagaagaactggctaatattgaggtcccagattcaacc | |
| | aacaaatttcggaagattgagaagaagatccaaattcacaacacgagatatggagaactgtt | |
| | cacaaggctgtgtacgcatatagagaagaaactgctggggtcatcttggtctaacaatgtccc | |
| | ccggtcagaggagttcagcagcattcgtacggatccggcattctggtttcactcaaaatggtc | |
| | cacagccaagtttgcatggctccatataaaacagatccagaggcatctgatggtggcagcta | |
| | ggacaaggtctgcggccaacaaattggtgatgctaacccataaggtaggccaagtctttgtc | |
| | actcctgaacttgtcgttgtgacgcatacgaatgagaacaagttcacatgtcttacccaggaa | |
| | cttgtattgatgtatgcagatatgatggagggcagagatatggtcaacataatatcaaccacg | |
| | gcggtgcatctcagaagcttatcagagaaaattgatgacattttgcggttaatagacgctctgg | |
| | caaaagacttgggtaatcaagtctacgatgttgtatcactaatggagggatttgcatacggag | |
| | ctgtccagctactcgagccgtcaggtacatttgcaggagatttcttcgcattcaacctgcagga | |
| | gcttaaagacattctaattggcctcctccccaatgatatagcagaatccgtgactcatgcaatc | |
| | gctactgtattctctggtttagaacagaatcaagcagctgagatgttgtgtctgttgcgtctgtg | |
| | gggtcacccactgcttgagtcccgtattgcagcaaaggcagtcaggagccaaatgtgcgca | |
| | ccgaaaatggtagactttgatatgatccttcaggtactgtctttcttcaagggaacaatcatcaa | |
| | cgggtacagaaagaagaatgcaggtgtgtggccgcgagtcaaagtggatacaatatatgg | |
| | gaaggtcattgggcaactacatgcagattcagcagagatttcacacgatatcatgttgagaga | |
| | gtataagagtttatctgcacttgaatttgagccatgtatagaatatgaccctgtcaccaacctga | |
| | gcatgttcctaaaagacaaggcaatcgcacaccccaacgataattggcttgcctcgtttaggc | |
| | ggaaccttctctccgaagaccagaagaaacatgtaaaagaagcaacttcgactaatcgcctc | |
| | ttgatagagtttttagagtcaaatgattttgatccatataaagagatggaatatctgacgaccctt | |
| | gagtaccttagagatgacaatgtggcagtatcatactcgctcaaggagaaggaagtgaaagt | |
| | taatggacggatcttcgctaagctgacaaagaagttaaggaactgtcaggtgatggcggaa | |
| | gggatcctagccgatcagattgcacctttctttcagggaaatggagtcattcaggatagcatat | |
| | ccttgaccaagagtatgctagcgatgagtcaactgtcttttaacagcaataagaaacgtatcac | |
| | tgactgtaaagaaagagtatcttcaaaccgcaatcatgatccgaaaagcaagaaccgtcgga | |
| | gagttgcaaccttcataacaactgacctgcaaaagtactgtcttaattggagatatcagacaat | |
| | caaattgttcgctcatgccatcaatcagttgatgggcctacctcacttcttcgaatggattcacct | |
| | aagactgatggacactacgatgttcgtaggagaccctttcaatcctccaagtgaccctactga | |
| | ctgtgacctctcaagagtccctaatgatgacatatatattgtcagtgccagagggggtatcga | |
| | aggattatgccagaagctatggacaatgatctcaattgctgcaatccaacttgctgcagctag | |
| | atcgcattgtcgtgttgcctgtatggtacagggtgataatcaagtaatagcagtaacgagaga | |
| | ggtaagatcagacgactctccggagatggtgttgacacagttgcatcaagccagtgataattt | |
| | cttcaaggaattaattcatgtcaatcatttgattggccataatttgaaggatcgtgaaaccatca | |
| | ggtcagacacattcttcatatacagcaaacgaatcttcaaagatggagcaatcctcagtcaag | |
| | tcctcaaaaattcatctaaattagtgctagtgtcaggtgatctcagtgaaaacaccgtaatgtcc | |
| | tgtgccaacattgcctctactgtagcacggctatgcgagaacgggcttcccaaagacttctgtt | |
| | actatttaaactatataatgagttgtgtgcagacatactttgactctgagttctccatcaccaaca | |
| | attcgcaccccgatcttaatcagtcgtggattgaggacatctcttttgtgcactcatatgttctga | |
| | ctcctgcccaattagggggactgagtaaccttcaatactcaaggctctacactagaaatatcg | |
| | gtgacccggggactactgcttttgcagagatcaagcgactagaagcagtgggattactgagt | |
| | cctaacattatgactaatatcttaactaggccgcctgggaatggagattgggccagtctgtgc | |
| | aacgacccatactctttcaattttgagactgttgcaagcccaaatattgttcttaagaaacatac | |
| | gcaaagagtcctatttgaaacttgttcaaatcccttattgtctggagtgcacacagaggataat | |
| | gaggcagaagagaaggcattggctgaattcttgcttaatcaagaggtgattcatccccgcgtt | |
| | gcgcatgccatcatggaggcaagctctgtaggtaggagaaagcaaattcaagggcttgttg | |
| | acacaacaaacaccgtaattaagattgcgcttactaggaggccattaggcatcaagaggctg | |
| | atgcggatagtcaattattctagcatgcatgcaatgctgtttagagacgatgttttttcctccagt | |
| | agatccaaccaccccttagtctcttctaatatgtgttctctgacactggcagactatgcacgga | |
| | atagaagctggtcacctttgacgggaggcaggaaaatactgggtgtatctaatcctgatacg | |
| | atagaactcgtagagggtgagattcttagtgtaagcggagggtgtacaagatgtgacagcg | |
| | gagatgaacaatttacttggttccatcttccaagcaatatagaattgaccgatgacaccagcaa | |
| | gaatcctccgatgagggtaccatatctcgggtcaaagacacaggagaggagagctgcctca | |
| | cttgcaaaaatagctcatatgtcgccacatgtaaaggctgccctaagggcatcatccgtgttg | |
| | atctgggcttatggggataatgaagtaaattggactgctgctcttacgattgcaaaatctcggt | |
| | gtaatgtaaacttagagtatcttcggttactgtcccctttacccacggctgggaatcttcaacat | |
| | agactagatgatggtataactcagatgacattcacccctgcatctctctacagggtgtcacctt | |
| | acattcacatatccaatgattctcaaaggctgttcactgaagaaggagtcaaagaggggaat | |
| | gtggtttaccaacagatcatgctcttgggtttatctctaatcgaatcgatctttccaatgacaaca | |
| | accaggacatatgatgagatcacactgcacctacatagtaaatttagttgctgtatcagagaa | |
| | gcacctgttgcggttcctttcgagctacttggggggtaccggaactgaggacagtgacctca | |
| | aataagtttatgtatgatcctagccctgtatcggagggagactttgcgagacttgacttagctat | |
| | cttcaagagttatgagcttaatctggagtcatatcccacgatagagctaatgaacattctttcaat | |
| | atccagcgggaagttgattggccagtctgtggtttcttatgatgaagatacctccataaagaat | |
| | gacgccataatagtgtatgacaatacccgaaattggatcagtgaagctcagaattcagatgtg | |
| | gtccgcctatttgaatatgcagcacttgaagtgctcctcgactgttcttaccaactctattacctg | |
| | agagtaagaggcctggacaatattgtcttatatatgggtgatttatacaagaatatgccaggaa | |
| | ttctactttccaacattgcagctacaatatctcatcccgtcattcattcaaggttacatgcagtgg | |
| | gcctggtcaaccatgacggatcacaccaacttgcagatacggattttatcgaaatgtctgcaa | |
| | aactattagtatcttgcacccgacgtgtgatctccggcttatattcaggaaataagtatgatctg | |
| | ctgttcccatctgtcttagatgataacctgaatgagaagatgcttcagctgatatcccggttatg | |
| | ctgtctgtacacggtactctttgctacaacaagagaaatcccgaaaataagaggcttaactgc | |
| | agaagagaaatgttcaatactcactgagtatttactgtcggatgctgtgaaaccattacttagcc | |
| | ccgatcaagtgagctctatcatgtctcctaacataattacattcccagctaatctgtactacatgt | |
| | ctcggaagagcctcaatttgatcagggaaagggaggacagggatactatcctggcgttgttg | |
| | ttcccccaagagccattattagagttcccttctgtgcaagatattggtgctcgagtgaaagatc | |
| | cattcacccgacaacctgcggcatttttgcaagagttagatttgagtgctccagcaaggtatga | |
| | cgcattcacacttagtcagattcatcctgaactcacatctccaaatccggaggaagactactta | |
| | gtacgatacttgttcagagggatagggactgcatcttcctcttggtataaggcatctcatctcct | |
| | ttctgtacccgaggtaagatgtgcaagacacgggaactccttatacttagctgaagggagcg | |
| | gagccatcatgagtcttctcgaactgcatgtaccacatgaaactatctattacaatacgctctttt | |
| | caaatgagatgaaccccccgcaacgacatttcgggccgaccccaactcagtttttgaattcg | |
| | gttgtttataggaatctacaggcggaggtaacatgcaaagatggatttgtccaagagttccgtc | |
| | cattatggagagaaaatacagaggaaagcgacctgacctcagataaagtagtggggtatatt | |
| | acatctgcagtgccctacagatctgtatcattgctgcattgtgacattgaaattcctccagggtc | |
| | caatcaaagcttactagatcaactagctatcaatttatctctgattgccatgcattctgtaaggga | |
| | gggcggggtagtaatcatcaaagtgttgtatgcaatgggatactactttcatctactcatgaact | |
| | tgtttgctccgtgttccacaaaaggatatattctctctaatggttatgcatgtcgaggagatatgg | |
| | agtgttacctggtatttgtcatgggttacctgggcgggcctacatttgtacatgaggtggtgag | |
| | gatggcgaaaactctggtgcagcggcacggtacgcttttgtctaaatcagatgagatcacact | |
| | gaccaggttattcacctcacagcggcagcgtgtgacagacatcctatccagtcctttaccaag | |
| | attaataaagtacttgaggaagaatattgacactgcgctgattgaagccgggggacagcccg | |
| | tccgtccattctgtgcggagagtctggtgagcacgctagcgaacataactcagataacccag | |
| | atcatcgctagtcacattgacacagttatccggtctgtgatatatatggaagctgagggtgatct | |
| | cgctgacacagtatttctatttaccccttacaatctctctactgacgggaaaaagaggacatca | |
| | cttaaacagtgcacgagacagatcctagaggttacaatactaggtcttagagtcgaaaatctc | |
| | aataaaataggcgatataatcagcctagtgcttaaaggcatgatctccatggaggaccttatc | |
| | ccactaaggacatacttgaagcatagtacctgccctaaatatttgaaggctgtcctaggtatta | |
| | ccaaactcaaagaaatgtttacagacacttctgtactgtacttgactcgtgctcaacaaaaattc | |
| | tacatgaaaactataggcaatgcagtcaaaggatattacagtaactgtgactcttaacgaaaat | |
| | cacatattaataggctccttttttggccaattgtattcttgttgatttaatcatattatgttagaaaaa | |
| | agttgaaccctgactccttaggactcgaattcgaactcaaataaatgtcttaaaaaaaggttgc | |
| | gcacaattattcttgagtgtagtctcgtcattcaccaaatctttgtttggt | |

TABLE 2
NDV LaSota F protein

| | | |
|---|---|---|
| Amino acid | MGSRPSTKNPAPMTLTIRVALVLSCICPANSIDGRPLAAAG | SEQ ID |
| sequence of F | IVVTGDKAVNIYTSSQTGSIIVKLLPNLPKDKEACAKAPLD | NO: 4 |
| protein of NDV | AYNRTLTTLLTPLGDSIRRIQESVTTSGGGRQGRLIGAIIGG | |
| strain LaSota | VALGVATAAQITAAAALIQAKQNAANILRLKESIAATNEA | |
| (transmembrane | VHEVTDGLSQLAVAVGKMQQFVNDQFNKTAQELDCIKIA | |
| domain is | QQVGVELNLYLTELTTVFGPQITSPALNKLTIQALYNLAG | |
| underlined and | GNMDYLLTKLGVGNNQLSSLIGSGLITGNPILYDSQTQLLG | |
| cytoplasmic | IQVTLPSVGNLNNMRATYLETLSVSTTRGFASALVPKVVT | |
| domain is in | QVGSVIEELDTSYCIETDLDLYCTRIVTFPMSPGIYSCLSGN | |
| bold) | TSACMYSKTEGALTTPYMTIKGSVIANCKMTTCRCVNPPG | |
| | IISQNYGEAVSLIDKQSCNVLSLGGITLRLSGEFDVTYQKNI | |
| | SIQDSQVIITGNLDISTELGNVNNSISNALNKLEESNRKLDK | |
| | VNVKLTSTSALITYIVLTIISLVFGILSLILACYLMYKQKAQ | |
| | QKTLLWLGNNTLDQMRATTKM | |
| Amino acid | LITYIVLTIISLVFGILSLILACYLMYKQKAQQKTLLWLGNN | SEQ ID |
| sequence of | TLDQMRATTKM | NO: 22 |
| transmembrane | | |
| and cytoplasmic | | |
| domains of F | | |
| protein of NDV | | |
| strain LaSota | | |

TABLE 3
SARS-CoV-2 Spike Protein Sequences

Nucleotide Sequence of the spike protein of NDV-HXP-S (Delta, P681): SEQ ID NO: 5
ATGTTCGTGTTTCTGGTGCTGCTGCCTCTGGTGTCCAGCCAGTGTGTGAACCTGaga
ACAAGAACCCAGCTGCCTCCAGCCTACACCAACAGCTTTACCAGAGGCGTGTAC
TACCCCGACAAGGTGTTCAGATCCAGCGTGCTGCACTCTACCCAGGACCTGTTCC
TGCCTTTCTTCAGCAACGTGACCTGGTTCCACGCCATCCACGTGTCCGGCACCAA
TGGCACCAAGAGATTCGACAACCCCGTGCTGCCCTTCAACGACGGGGTGTACTTT
GCCAGCACCGAGAAGTCCAACATCATCAGAGGCTGGATCTTCGGCACCACACTG
GACAGCAAGACCCAGAGCCTGCTGATCGTGAACAACGCCACCAACGTGGTCATC
AAAGTGTGCGAGTTCCAGTTCTGCAACGACCCCTTCCTGgacGTCTACTATCACAA
GAACAACAAGAGCTGGATGGAAAGCggcGTGTACAGCAGCGCCAACAACTGCAC
CTTCGAGTACGTGTCCCAGCCTTTCCTGATGGACCTGGAAGGCAAGCAGGGCAA
CTTCAAGAACCTGCGCGAGTTCGTGTTCAAGAACATCGACGGCTACTTCAAGATC
TACAGCAAGCACACCCCTATCAACCTCGTGCGGGATCTGCCTCAGGGCTTCTCTG
CTCTGGAACCCCTGGTGGATCTGCCCATCGGCATCAACATCACCCGGTTTCAGAC
ACTGCTGGCCCTGCACAGAAGCTACCTGACACCTGGCGATAGCAGCAGCGGATG
GACAGCTGGTGCCGCCGCTTACTATGTGGGCTACCTGCAGCCTAGAACCTTTCTG
CTGAAGTACAACGAGAACGGCACCATCACCGACGCCGTGGATTGTGCTCTGGAT
CCTCTGAGCGAGACAAAGTGCACCCTGAAGTCCTTCACCGTGGAgAAGGGCATCT
ACCAGACCAGCAACTTCCGGGTGCAGCCCACCGAATCCATCGTGCGGTTCCCCA
ATATCACCAATCTGTGCCCCTTCGGCGAGGTGTTCAATGCCACCAGATTCGCCTC
TGTGTACGCCTGGAACCGGAAGCGGATCAGCAATTGCGTGGCCGACTACTCCGT
GCTGTACAACTCCGCCAGCTTCAGCACCTTCAAGTGCTACGGCGTGTCCCCTACC
AAGCTGAACGACCTGTGCTTCACAAACGTGTACGCCGACAGCTTCGTGATCCGG
GGAGATGAAGTGCGGCAGATTGCCCCTGGACAGACAGGCAAGATCGCCGACTAC
AACTACAAGCTGCCCGACGACTTCACCGGCTGTGTGATTGCCTGGAACAGCAAC
AACCTGGACTCCAAAGTCGGCGGCAACTACAATTACeggTACCGGCTGTTCCGGA
AGTCCAATCTGAAGCCCTTCGAGCGGGACATCTCCACCGAGATCTATCAGGCCG
GCAGCaagCCTTGTAACGGCGTGGAAGGCTTCAACTGCTACTTCCCACTGCAGTCC
TACGGCTTTCAGCCCACAAATGGCGTGGGCTATCAGCCCTACAGAGTGGTGGTGC
TGAGCTTCGAACTGCTGCATGCCCCTGCCACAGTGTGCGGCCCTAAGAAgAGCAC
CAATCTCGTGAAGAACAAATGCGTGAACTTCAACTTCAACGGCCTGACCGGCAC
CGGCGTGCTGACAGAGAGCAACAAGAAGTTCCTGCCATTCCAGCAGTTTGGCCG
GGATATCGCCGATACCACAGACGCCGTTAGAGATCCCCAGACACTGGAAATCCT
GGACATCACCCCTTGCAGCTTCGGCGGAGTGTCTGTGATCACCCCTGGCACCAAC
ACCAGCAATCAGGTGGCAGTGCTGTACCAGggcGTGAACTGTACCGAAGTGCCCG
TGGCCATTCACGCCGATCAGCTGACACCTACATGGCGGGTGTACTCCACCGGCAG
CAATGTGTTTCAGACCAGAGCCGGCTGTCTGATCGGAGCCGAGCACGTGAACAA
TAGCTACGAGTGCGACATCCCCATCGGCGCTGGCATCTGTGCCAGCTACCAGACA
CAGACAAACAGCagaGCCTCTGTGGCCAGCCAGAGCATCATTGCCTACACAATGT
CTCTGGGCGCCGAGAACAGCGTGGCCTACTCCAACAACTCTATCGCTATCCCCAC
CAACTTCACCATCAGCGTGACCACAGAGATCCTGCCTGTGTCCATGACCAAGACC
AGCGTGGACTGCACCATGTACATCTGCGGCGATTCCACCGAGTGCTCCAACCTGC
TGCTGCAGTACGGCAGCTTCTGCACCCAGCTGAATAGAGCCCTGACAGGGATCG
CCGTGGAACAGGACAAGAACACCCAAGAGGTGTTCGCCCAAGTGAAGCAGATCT
ACAAGACCCCTCCTATCAAGGACTTCGGCGGCTTCAATTTCAGCCAGATTCTGCC
CGATCCTAGCAAGCCCAGCAAGCGGAGCcctATCGAGGACCTGCTGTTCAACAAA
GTGACACTGGCCGACGCCGGCTTCATCAAGCAGTATGGCGATTGTCTGGGCGAC
ATTGCCGCCAGGGATCTGATTTGCGCCCAGAAGTTTAACGGACTGACAGTGCTGC
CTCCTCTGCTGACCGATGAGATGATCGCCCAGTACACATCTGCCCTGCTGGCCGG
CACAATCACAAGCGGCTGGACATTTGGAGCTGGCcctGCTCTGCAGATCCCCTTTcc
aATGCAGATGGCCTACCGGTTCAACGGCATCGGAGTGACCCAGAATGTGCTGTAC
GAGAACCAGAAGCTGATCGCCAACCAGTTCAACAGCGCCATCGGCAAGATCCAG
GACAGCCTGAGCAGCACAcccAGCGCCCTGGGAAAGCTGCAGaacGTGGTCAACCA
GAATGCCCAGGCACTGAACACCCTGGTCAAGCAGCTGTCCTCCAACTTCGGCGCC
ATCAGCTCTGTGCTGAACGATATCCTGAGCAGACTGGACccccctGAAGCCGAGGTG
CAGATCGACAGACTGATCACCGGAAGGCTGCAGTCCCTGCAGACCTACGTTACC
CAGCAGCTGATCAGAGCCGCCGAGATTAGAGCCTCTGCCAATCTGGCCGCCACC
AAGATGTCTGAGTGTGTGCTGGGCCAGAGCAAGAGAGTGGACTTTTGCGGCAAG
GGCTACCACCTGATGAGCTTCCCTCAGTCTGCCCCTCACGGCGTGGTGTTTCTGC
ACGTGACATACGTGCCCGCTCAAGAGAAGAATTTCACCACCGCTCCAGCCATCTG
CCACGACGGCAAAGCCCACTTTCCTAGAGAAGGCGTGTTCGTGTCCAACGGCAC
CCATTGGTTCGTGACCCAGCGGAACTTCTACGAGCCCCAGATCATCACCACCGAC
AACACCTTCGTGTCTGGCAACTGCGACGTCGTGATCGGCATTGTGAACAATACCG
TGTACGACCCTCTGCAGCCCGAGCTGGACAGCTTCAAAGAGGAACTGGATAAGT
ACTTTAAGAACCACACAAGCCCCGACGTGGACCTGGGCGATATCAGCGGAATCA
ATGCCAGCGTCGTGAACATCCAGAAAGAGATCGACCGGCTGAACGAGGTGGCCA
AGAATCTGAACGAGAGCCTGATCGACCTGCAAGAACTGGGGAAGTACGAGCAGT
ACATCAAGTGGCCCggcggaggtgggtcgCTCATAACATACATCGTCCTGACTATAATCA
GCTTGGTATTTGGTATTTTGTCTTTGATTCTTGCATGCTATTTGATGTATAAACAG
AAAGCTCAGCAGAAGACTCTCCTGTGGCTCGGTAACAACACACTCGACCAGATG
AGAGCAACTACAAAGATGTGA
Amino Acid Sequence of the spike protein of NDV-HXP-S (Delta, P681): SEQ ID NO: 6
MFVFLVLLPLVSSQCVNLRTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLDVYYHKNNKSWMESGVYSSANNCTFEYVSQP
FLMDLEGKQGNFKNLREFVFKNIDGYFKIYSKHTPINLVRDLPQGFSALEPLVDLPIGI
NITRFQTLLALHRSYLTPGDSSSGWTAGAAAYYVGYLQPRTFLLKYNENGTITDAVD
CALDPLSETKCTLKSFTVEKGIYQTSNFRVQPTESIVRFPNITNLCPFGEVENATRFAS
VYAWNRKRISNCVADYSVLYNSASFSTFKCYGVSPTKLNDLCFTNVYADSFVIRGDE
VRQIAPGQTGKIADYNYKLPDDFTGCVIAWNSNNLDSKVGGNYNYRYRLFRKSNLK
PFERDISTEIYQAGSKPCNGVEGFNCYFPLQSYGFQPTNGVGYQPYRVVVLSFELLHA
PATVCGPKKSTNLVKNKCVNFNFNGLTGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEHVNNSYECDIPIGAGICASYQTQTNSRASVASQSIIAYT
MSLGAENSVAYSNNSIAIPTNFTISVTTEILPVSMTKTSVDCTMYICGDSTECSNLLLQ
YGSFCTQLNRALTGIAVEQDKNTQEVFAQVKQIYKTPPIKDFGGFNFSQILPDPSKPS
KRSPIEDLLFNKVTLADAGFIKQYGDCLGDIAARDLICAQKFNGLTVLPPLLTDEMIA
QYTSALLAGTITSGWTFGAGPALQIPFPMQMAYRENGIGVTQNVLYENQKLIANQFN
SAIGKIQDSLSSTPSALGKLQNVVNQNAQALNTLVKQLSSNFGAISSVLNDILSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWPGGG
GSLITYIVLTIISLVFGILSLILACYLMYKQKAQQKTLLWLGNNTLDQMRATTKM*
S-F chimera HexaPro (Amino acid sequence): SEQ ID NO: 11
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLP
IGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGFNCYFPLQSYGFQPTNGVGYQPYRVVVLSFELL
HAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSPASVASQSII
AYTMSLGAENSVAYSNNSIAIPTNFTISVTTEILPVSMTKTSVDCTMYICGDSTECSNL
LLQYGSFCTQLNRALTGIAVEQDKNTQEVFAQVKQIYKTPPIKDFGGFNFSQILPDPS
KPSKRSPIEDLLFNKVTLADAGFIKQYGDCLGDIAARDLICAQKFNGLTVLPPLLTDE
MIAQYTSALLAGTITSGWTFGAGPALQIPFPMQMAYRENGIGVTQNVLYENQKLIAN
QFNSAIGKIQDSLSSTPSALGKLQDVVNQNAQALNTLVKQLSSNFGAISSVLNDILSR
LDPPEAEVQIDRLITGRLQSLQTYVTQQLIRAAEIRASANLAATKMSECVLGQSKRVD
FCGKGYHLMSFPQSAPHGVVFLHVTYVPAQEKNFTTAPAICHDGKAHFPREGVFVS
NGTHWFVTQRNFYEPQIITTDNTFVSGNCDVVIGIVNNTVYDPLQPELDSFKEELDKY
FKNHTSPDVDLGDISGINASVVNIQKEIDRLNEVAKNLNESLIDLQELGKYEQYIKWP
GGGGSLITYIVLTIISLVFGILSLILACYLMYKQKAQQKTLLWLGNNTLDQMRATTKM*
Ectodomain Portion of SEQ ID NO: 11 (Amino Acid Sequence): SEQ ID NO: 12
MFVFLVLLPLVSSQCVNLTTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLGVYYHKNNKSWMESEFRVYSSANNCTFEYVS
QPFLMDLEGKQGNFKNLREFVFKNIDGYFKIYSKHTPINLVRDLPQGFSALEPLVDLP
IGINITRFQTLLALHRSYLTPGDSSSGWTAGAAAYYVGYLQPRTFLLKYNENGTITDA
VDCALDPLSETKCTLKSFTVEKGIYQTSNFRVQPTESIVRFPNITNLCPFGEVFNATRF
ASVYAWNRKRISNCVADYSVLYNSASFSTFKCYGVSPTKLNDLCFTNVYADSFVIRG
DEVRQIAPGQTGKIADYNYKLPDDFTGCVIAWNSNNLDSKVGGNYNYLYRLFRKSN
LKPFERDISTEIYQAGSTPCNGVEGENCYFPLQSYGFQPTNGVGYQPYRVVVLSFELL
HAPATVCGPKKSTNLVKNKCVNFNFNGLTGTGVLTESNKKFLPFQQFGRDIADTTD
AVRDPQTLEILDITPCSFGGVSVITPGTNTSNQVAVLYQDVNCTEVPVAIHADQLTPT
WRVYSTGSNVFQTRAGCLIGAEHVNNSYECDIPIGAGICASYQTQTNSPASVASQSII
AYTMSLGAENSVAYSNNSIAIPTNFTISVTTEILPVSMTKTSVDCTMYICGDSTECSNL
LLQYGSFCTQLNRALTGIAVEQDKNTQEVFAQVKQIYKTPPIKDFGGFNFSQILPDPS
KPSKRSPIEDLLFNKVTLADAGFIKQYGDCLGDIAARDLICAQKFNGLTVLPPLLTDE
MIAQYTSALLAGTITSGWTFGAGPALQIPFPMQMAYRENGIGVTQNVLYENQKLIAN
QFNSAIGKIQDSLSSTPSALGKLQDVVNQNAQALNTLVKQLSSNFGAISSVLNDILSR
LDPPEAEVQIDRLITGRLQSLQTYVTQQLIRAAEIRASANLAATKMSECVLGQSKRVD
FCGKGYHLMSFPQSAPHGVVFLHVTYVPAQEKNFTTAPAICHDGKAHFPREGVFVS
NGTHWFVTQRNFYEPQIITTDNTFVSGNCDVVIGIVNNTVYDPLQPELDSFKEELDKY
FKNHTSPDVDLGDISGINASVVNIQKEIDRLNEVAKNLNESLIDLQELGKYEQYIKWP
Ectodomain Portion of SEQ ID NO: 6 (Amino Acid Sequence): SEQ ID NO: 13
MFVFLVLLPLVSSQCVNLRTRTQLPPAYTNSFTRGVYYPDKVFRSSVLHSTQDLFLPF
FSNVTWFHAIHVSGTNGTKRFDNPVLPFNDGVYFASTEKSNIIRGWIFGTTLDSKTQS
LLIVNNATNVVIKVCEFQFCNDPFLDVYYHKNNKSWMESGVYSSANNCTFEYVSQP
FLMDLEGKQGNFKNLREFVFKNIDGYFKIYSKHTPINLVRDLPQGFSALEPLVDLPIGI
NITRFQTLLALHRSYLTPGDSSSGWTAGAAAYYVGYLQPRTFLLKYNENGTITDAVD
CALDPLSETKCTLKSFTVEKGIYQTSNFRVQPTESIVRFPNITNLCPFGEVENATRFAS
VYAWNRKRISNCVADYSVLYNSASFSTFKCYGVSPTKLNDLCFTNVYADSFVIRGDE
VRQIAPGQTGKIADYNYKLPDDFTGCVIAWNSNNLDSKVGGNYNYRYRLFRKSNLK
PFERDISTEIYQAGSKPCNGVEGENCYFPLQSYGFQPTNGVGYQPYRVVVLSFELLHA
PATVCGPKKSTNLVKNKCVNFNFNGLTGTGVLTESNKKFLPFQQFGRDIADTTDAVR
DPQTLEILDITPCSFGGVSVITPGTNTSNQVAVLYQGVNCTEVPVAIHADQLTPTWRV
YSTGSNVFQTRAGCLIGAEHVNNSYECDIPIGAGICASYQTQTNSRASVASQSIIAYT
MSLGAENSVAYSNNSIAIPTNFTISVTTEILPVSMTKTSVDCTMYICGDSTECSNLLLQ
YGSFCTQLNRALTGIAVEQDKNTQEVFAQVKQIYKTPPIKDFGGFNFSQILPDPSKPS
KRSPIEDLLFNKVTLADAGFIKQYGDCLGDIAARDLICAQKFNGLTVLPPLLTDEMIA
QYTSALLAGTITSGWTFGAGPALQIPFPMQMAYRFNGIGVTQNVLYENQKLIANQFN
SAIGKIQDSLSSTPSALGKLQNVVNQNAQALNTLVKQLSSNFGAISSVLNDILSRLDPP
EAEVQIDRLITGRLQSLQTYVTQQLIRAAEIRASANLAATKMSECVLGQSKRVDFCG
KGYHLMSFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQYIKWP
Ectodomain Portion of SEQ ID NO: 11 Without the Signal Sequence (Amino Acid
Sequence): SEQ ID NO: 16
QCVNLTTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIK
VCEFQFCNDPFLGVYYHKNNKSWMESEFRVYSSANNCTFEYVSQPFLMDLEGKQG
NFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASVYAWNRKRIS
NCVADYSVLYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFERDISTEIY
QAGSTPCNGVEGENCYFPLQSYGFQPTNGVGYQPYRVVVLSFELLHAPATVCGPKK
STNLVKNKCVNFNFNGLTGTGVLTESNKKFLPFQQFGRDIADTTDAVRDPQTLEILDI
TPCSFGGVSVITPGTNTSNQVAVLYQDVNCTEVPVAIHADQLTPTWRVYSTGSNVFQ
TRAGCLIGAEHVNNSYECDIPIGAGICASYQTQTNSPASVASQSIIAYTMSLGAENSVA
YSNNSIAIPTNFTISVTTEILPVSMTKTSVDCTMYICGDSTECSNLLLQYGSFCTQLNR
ALTGIAVEQDKNTQEVFAQVKQIYKTPPIKDFGGFNFSQILPDPSKPSKRSPIEDLLEN
KVTLADAGFIKQYGDCLGDIAARDLICAQKFNGLTVLPPLLTDEMIAQYTSALLAGTI
TSGWTFGAGPALQIPFPMQMAYRENGIGVTQNVLYENQKLIANQFNSAIGKIQDSLSS
TPSALGKLQDVVNQNAQALNTLVKQLSSNFGAISSVLNDILSRLDPPEAEVQIDRLIT
GRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFY
EPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWP
Ectodomain Portion of SEQ ID NO: 6 Without the Signal Sequence (Amino Acid
Sequence): SEQ ID NO: 17
QCVNLRTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIK
VCEFQFCNDPFLDVYYHKNNKSWMESGVYSSANNCTFEYVSQPFLMDLEGKQGNF
KNLREFVFKNIDGYFKIYSKHTPINLVRDLPQGFSALEPLVDLPIGINITRFQTLLALHR
SYLTPGDSSSGWTAGAAAYYVGYLQPRTFLLKYNENGTITDAVDCALDPLSETKCTL
KSFTVEKGIYQTSNFRVQPTESIVRFPNITNLCPFGEVENATRFASVYAWNRKRISNCV
ADYSVLYNSASFSTFKCYGVSPTKLNDLCFTNVYADSFVIRGDEVRQIAPGQTGKIA
DYNYKLPDDFTGCVIAWNSNNLDSKVGGNYNYRYRLFRKSNLKPFERDISTEIYQAG
SKPCNGVEGFNCYFPLQSYGFQPTNGVGYQPYRVVVLSFELLHAPATVCGPKKSTNL
VKNKCVNFNFNGLTGTGVLTESNKKFLPFQQFGRDIADTTDAVRDPQTLEILDITPCS
FGGVSVITPGTNTSNQVAVLYQGVNCTEVPVAIHADQLTPTWRVYSTGSNVFQTRA
GCLIGAEHVNNSYECDIPIGAGICASYQTQTNSRASVASQSIIAYTMSLGAENSVAYS
NNSIAIPTNFTISVTTEILPVSMTKTSVDCTMYICGDSTECSNLLLQYGSFCTQLNRAL
TGIAVEQDKNTQEVFAQVKQIYKTPPIKDFGGFNFSQILPDPSKPSKRSPIEDLLFNKV
TLADAGFIKQYGDCLGDIAARDLICAQKENGLTVLPPLLTDEMIAQYTSALLAGTITS
GWTFGAGPALQIPFPMQMAYRENGIGVTQNVLYENQKLIANQFNSAIGKIQDSLSSTP
SALGKLQNVVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQ
IITTDNTFVSGNCDVVIGIVNNTVYDPLQPELDSFKEELDKYFKNHTSPDVDLGDISGI
NASVVNIQKEIDRLNEVAKNLNESLIDLQELGKYEQYIKWP
Amino Acid Sequence of the spike protein of NDV-HXP-S (Delta, P681) Without the
Signal Sequence: SEQ ID NO: 18
QCVNLRTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIK
VCEFQFCNDPFLDVYYHKNNKSWMESGVYSSANNCTFEYVSQPFLMDLEGKQGNF
KNLREFVFKNIDGYFKIYSKHTPINLVRDLPQGFSALEPLVDLPIGINITRFQTLLALHR
SYLTPGDSSSGWTAGAAAYYVGYLQPRTFLLKYNENGTITDAVDCALDPLSETKCTL
KSFTVEKGIYQTSNFRVQPTESIVRFPNITNLCPFGEVENATRFASVYAWNRKRISNCV
ADYSVLYNSASFSTFKCYGVSPTKLNDLCFTNVYADSFVIRGDEVRQIAPGQTGKIA
DYNYKLPDDFTGCVIAWNSNNLDSKVGGNYNYRYRLFRKSNLKPFERDISTEIYQAG
SKPCNGVEGFNCYFPLQSYGFQPTNGVGYQPYRVVVLSFELLHAPATVCGPKKSTNL
VKNKCVNFNFNGLTGTGVLTESNKKFLPFQQFGRDIADTTDAVRDPQTLEILDITPCS
FGGVSVITPGTNTSNQVAVLYQGVNCTEVPVAIHADQLTPTWRVYSTGSNVFQTRA
GCLIGAEHVNNSYECDIPIGAGICASYQTQTNSRASVASQSIIAYTMSLGAENSVAYS
NNSIAIPTNFTISVTTEILPVSMTKTSVDCTMYICGDSTECSNLLLQYGSFCTQLNRAL
TGIAVEQDKNTQEVFAQVKQIYKTPPIKDFGGFNFSQILPDPSKPSKRSPIEDLLFNKV
TLADAGFIKQYGDCLGDIAARDLICAQKFNGLTVLPPLLTDEMIAQYTSALLAGTITS
GWTFGAGPALQIPFPMQMAYRFNGIGVTQNVLYENQKLIANQFNSAIGKIQDSLSSTP
SALGKLQNVVNQNAQALNTLVKQLSSNFGAISSVLNDILSRLDPPEAEVQIDRLITGR
LQSLQTYVTQQLIRAAEIRASANLAATKMSECVLGQSKRVDFCGKGYHLMSFPQSAP
HGVVFLHVTYVPAQEKNFTTAPAICHDGKAHFPREGVFVSNGTHWFVTQRNFYEPQ
IITTDNTFVSGNCDVVIGIVNNTVYDPLQPELDSFKEELDKYFKNHTSPDVDLGDISGI
NASVVNIQKEIDRLNEVAKNLNESLIDLQELGKYEQYIKWPGGGGSLITYIVLTIISLV
FGILSLILACYLMYKQKAQQKTLLWLGNNTLDQMRATTKM*
Nucleotide Sequence of Ectodomain Portion of NDV-HXP-S (Delta, P681) (SEQ ID
NO: 5) With Signal Sequence: SEQ ID NO: 19
ATGTTCGTGTTTCTGGTGCTGCTGCCTCTGGTGTCCAGCCAGTGTGTGAACCTGaga
ACAAGAACCCAGCTGCCTCCAGCCTACACCAACAGCTTTACCAGAGGCGTGTAC
TACCCCGACAAGGTGTTCAGATCCAGCGTGCTGCACTCTACCCAGGACCTGTTCC
TGCCTTTCTTCAGCAACGTGACCTGGTTCCACGCCATCCACGTGTCCGGCACCAA
TGGCACCAAGAGATTCGACAACCCCGTGCTGCCCTTCAACGACGGGGTGTACTTT
GCCAGCACCGAGAAGTCCAACATCATCAGAGGCTGGATCTTCGGCACCACACTG
GACAGCAAGACCCAGAGCCTGCTGATCGTGAACAACGCCACCAACGTGGTCATC
AAAGTGTGCGAGTTCCAGTTCTGCAACGACCCCTTCCTGgacGTCTACTATCACAA
GAACAACAAGAGCTGGATGGAAAGCggcGTGTACAGCAGCGCCAACAACTGCAC
CTTCGAGTACGTGTCCCAGCCTTTCCTGATGGACCTGGAAGGCAAGCAGGGCAA
CTTCAAGAACCTGCGCGAGTTCGTGTTCAAGAACATCGACGGCTACTTCAAGATC
TACAGCAAGCACACCCCTATCAACCTCGTGCGGGATCTGCCTCAGGGCTTCTCTG
CTCTGGAACCCCTGGTGGATCTGCCCATCGGCATCAACATCACCCGGTTTCAGAC
ACTGCTGGCCCTGCACAGAAGCTACCTGACACCTGGCGATAGCAGCAGCGGATG
GACAGCTGGTGCCGCCGCTTACTATGTGGGCTACCTGCAGCCTAGAACCTTTCTG
CTGAAGTACAACGAGAACGGCACCATCACCGACGCCGTGGATTGTGCTCTGGAT
CCTCTGAGCGAGACAAAGTGCACCCTGAAGTCCTTCACCGTGGAgAAGGGCATCT
ACCAGACCAGCAACTTCCGGGTGCAGCCCACCGAATCCATCGTGCGGTTCCCCA
ATATCACCAATCTGTGCCCCTTCGGCGAGGTGTTCAATGCCACCAGATTCGCCTC
TGTGTACGCCTGGAACCGGAAGCGGATCAGCAATTGCGTGGCCGACTACTCCGT
GCTGTACAACTCCGCCAGCTTCAGCACCTTCAAGTGCTACGGCGTGTCCCCTACC
AAGCTGAACGACCTGTGCTTCACAAACGTGTACGCCGACAGCTTCGTGATCCGG
GGAGATGAAGTGCGGCAGATTGCCCCTGGACAGACAGGCAAGATCGCCGACTAC
AACTACAAGCTGCCCGACGACTTCACCGGCTGTGTGATTGCCTGGAACAGCAAC
AACCTGGACTCCAAAGTCGGCGGCAACTACAATTACcggTACCGGCTGTTCCGGA
AGTCCAATCTGAAGCCCTTCGAGCGGGACATCTCCACCGAGATCTATCAGGCCG
GCAGCaagCCTTGTAACGGCGTGGAAGGCTTCAACTGCTACTTCCCACTGCAGTCC
TACGGCTTTCAGCCCACAAATGGCGTGGGCTATCAGCCCTACAGAGTGGTGGTGC
TGAGCTTCGAACTGCTGCATGCCCCTGCCACAGTGTGCGGCCCTAAGAAgAGCAC
CAATCTCGTGAAGAACAAATGCGTGAACTTCAACTTCAACGGCCTGACCGGCAC
CGGCGTGCTGACAGAGAGCAACAAGAAGTTCCTGCCATTCCAGCAGTTTGGCCG
GGATATCGCCGATACCACAGACGCCGTTAGAGATCCCCAGACACTGGAAATCCT
GGACATCACCCCTTGCAGCTTCGGCGGAGTGTCTGTGATCACCCCTGGCACCAAC
ACCAGCAATCAGGTGGCAGTGCTGTACCAGggcGTGAACTGTACCGAAGTGCCCG
TGGCCATTCACGCCGATCAGCTGACACCTACATGGCGGGTGTACTCCACCGGCAG
CAATGTGTTTCAGACCAGAGCCGGCTGTCTGATCGGAGCCGAGCACGTGAACAA
TAGCTACGAGTGCGACATCCCCATCGGCGCTGGCATCTGTGCCAGCTACCAGACA
CAGACAAACAGCagaGCCTCTGTGGCCAGCCAGAGCATCATTGCCTACACAATGT
CTCTGGGCGCCGAGAACAGCGTGGCCTACTCCAACAACTCTATCGCTATCCCCAC
CAACTTCACCATCAGCGTGACCACAGAGATCCTGCCTGTGTCCATGACCAAGACC
AGCGTGGACTGCACCATGTACATCTGCGGCGATTCCACCGAGTGCTCCAACCTGC
TGCTGCAGTACGGCAGCTTCTGCACCCAGCTGAATAGAGCCCTGACAGGGATCG
CCGTGGAACAGGACAAGAACACCCAAGAGGTGTTCGCCCAAGTGAAGCAGATCT
ACAAGACCCCTCCTATCAAGGACTTCGGCGGCTTCAATTTCAGCCAGATTCTGCC
CGATCCTAGCAAGCCCAGCAAGCGGAGCcctATCGAGGACCTGCTGTTCAACAAA
GTGACACTGGCCGACGCCGGCTTCATCAAGCAGTATGGCGATTGTCTGGGCGAC
ATTGCCGCCAGGGATCTGATTTGCGCCCAGAAGTTTAACGGACTGACAGTGCTGC
CTCCTCTGCTGACCGATGAGATGATCGCCCAGTACACATCTGCCCTGCTGGCCGG
CACAATCACAAGCGGCTGGACATTTGGAGCTGGCcctGCTCTGCAGATCCCCTTTcc
aATGCAGATGGCCTACCGGTTCAACGGCATCGGAGTGACCCAGAATGTGCTGTAC
GAGAACCAGAAGCTGATCGCCAACCAGTTCAACAGCGCCATCGGCAAGATCCAG
GACAGCCTGAGCAGCACAcccAGCGCCCTGGGAAAGCTGCAGaacGTGGTCAACCA
GAATGCCCAGGCACTGAACACCCTGGTCAAGCAGCTGTCCTCCAACTTCGGCGCC
ATCAGCTCTGTGCTGAACGATATCCTGAGCAGACTGGACccccctGAAGCCGAGGTG
CAGATCGACAGACTGATCACCGGAAGGCTGCAGTCCCTGCAGACCTACGTTACC
CAGCAGCTGATCAGAGCCGCCGAGATTAGAGCCTCTGCCAATCTGGCCGCCACC
AAGATGTCTGAGTGTGTGCTGGGCCAGAGCAAGAGAGTGGACTTTTGCGGCAAG
GGCTACCACCTGATGAGCTTCCCTCAGTCTGCCCCTCACGGCGTGGTGTTTCTGC
ACGTGACATACGTGCCCGCTCAAGAGAAGAATTTCACCACCGCTCCAGCCATCTG
CCACGACGGCAAAGCCCACTTTCCTAGAGAAGGCGTGTTCGTGTCCAACGGCAC
CCATTGGTTCGTGACCCAGCGGAACTTCTACGAGCCCCAGATCATCACCACCGAC
AACACCTTCGTGTCTGGCAACTGCGACGTCGTGATCGGCATTGTGAACAATACCG
TGTACGACCCTCTGCAGCCCGAGCTGGACAGCTTCAAAGAGGAACTGGATAAGT
ACTTTAAGAACCACACAAGCCCCGACGTGGACCTGGGCGATATCAGCGGAATCA
ATGCCAGCGTCGTGAACATCCAGAAAGAGATCGACCGGCTGAACGAGGTGGCCA
AGAATCTGAACGAGAGCCTGATCGACCTGCAAGAACTGGGGAAGTACGAGCAGT
ACATCAAGTGGCCC
Nucleotide Sequence of Ectodomain Portion of NDV-HXP-S (Delta, P681) (SEQ ID
NO: 5) Without Signal Sequence: SEQ ID NO: 20
CAGTGTGTGAACCTGagaACAAGAACCCAGCTGCCTCCAGCCTACACCAACAGCT
TTACCAGAGGCGTGTACTACCCCGACAAGGTGTTCAGATCCAGCGTGCTGCACTC
TACCCAGGACCTGTTCCTGCCTTTCTTCAGCAACGTGACCTGGTTCCACGCCATCC
ACGTGTCCGGCACCAATGGCACCAAGAGATTCGACAACCCCGTGCTGCCCTTCA
ACGACGGGGTGTACTTTGCCAGCACCGAGAAGTCCAACATCATCAGAGGCTGGA
TCTTCGGCACCACACTGGACAGCAAGACCCAGAGCCTGCTGATCGTGAACAACG
CCACCAACGTGGTCATCAAAGTGTGCGAGTTCCAGTTCTGCAACGACCCCTTCCT
GgacGTCTACTATCACAAGAACAACAAGAGCTGGATGGAAAGCggcGTGTACAGCA
GCGCCAACAACTGCACCTTCGAGTACGTGTCCCAGCCTTTCCTGATGGACCTGGA
AGGCAAGCAGGGCAACTTCAAGAACCTGCGCGAGTTCGTGTTCAAGAACATCGA
CGGCTACTTCAAGATCTACAGCAAGCACACCCCTATCAACCTCGTGCGGGATCTG
CCTCAGGGCTTCTCTGCTCTGGAACCCCTGGTGGATCTGCCCATCGGCATCAACA
TCACCCGGTTTCAGACACTGCTGGCCCTGCACAGAAGCTACCTGACACCTGGCGA
TAGCAGCAGCGGATGGACAGCTGGTGCCGCCGCTTACTATGTGGGCTACCTGCA
GCCTAGAACCTTTCTGCTGAAGTACAACGAGAACGGCACCATCACCGACGCCGT
GGATTGTGCTCTGGATCCTCTGAGCGAGACAAAGTGCACCCTGAAGTCCTTCACC
GTGGAgAAGGGCATCTACCAGACCAGCAACTTCCGGGTGCAGCCCACCGAATCC
ATCGTGCGGTTCCCCAATATCACCAATCTGTGCCCCTTCGGCGAGGTGTTCAATG
CCACCAGATTCGCCTCTGTGTACGCCTGGAACCGGAAGCGGATCAGCAATTGCGT
GGCCGACTACTCCGTGCTGTACAACTCCGCCAGCTTCAGCACCTTCAAGTGCTAC
GGCGTGTCCCCTACCAAGCTGAACGACCTGTGCTTCACAAACGTGTACGCCGACA
GCTTCGTGATCCGGGGAGATGAAGTGCGGCAGATTGCCCCTGGACAGACAGGCA
AGATCGCCGACTACAACTACAAGCTGCCCGACGACTTCACCGGCTGTGTGATTGC
CTGGAACAGCAACAACCTGGACTCCAAAGTCGGCGGCAACTACAATTACcggTAC
CGGCTGTTCCGGAAGTCCAATCTGAAGCCCTTCGAGCGGGACATCTCCACCGAG
ATCTATCAGGCCGGCAGCaagCCTTGTAACGGCGTGGAAGGCTTCAACTGCTACTT
CCCACTGCAGTCCTACGGCTTTCAGCCCACAAATGGCGTGGGCTATCAGCCCTAC
AGAGTGGTGGTGCTGAGCTTCGAACTGCTGCATGCCCCTGCCACAGTGTGCGGCC
CTAAGAAgAGCACCAATCTCGTGAAGAACAAATGCGTGAACTTCAACTTCAACG
GCCTGACCGGCACCGGCGTGCTGACAGAGAGCAACAAGAAGTTCCTGCCATTCC
AGCAGTTTGGCCGGGATATCGCCGATACCACAGACGCCGTTAGAGATCCCCAGA
CACTGGAAATCCTGGACATCACCCCTTGCAGCTTCGGCGGAGTGTCTGTGATCAC
CCCTGGCACCAACACCAGCAATCAGGTGGCAGTGCTGTACCAGggcGTGAACTGT
ACCGAAGTGCCCGTGGCCATTCACGCCGATCAGCTGACACCTACATGGCGGGTG
TACTCCACCGGCAGCAATGTGTTTCAGACCAGAGCCGGCTGTCTGATCGGAGCCG
AGCACGTGAACAATAGCTACGAGTGCGACATCCCCATCGGCGCTGGCATCTGTG
CCAGCTACCAGACACAGACAAACAGCagaGCCTCTGTGGCCAGCCAGAGCATCAT
TGCCTACACAATGTCTCTGGGCGCCGAGAACAGCGTGGCCTACTCCAACAACTCT
ATCGCTATCCCCACCAACTTCACCATCAGCGTGACCACAGAGATCCTGCCTGTGT
CCATGACCAAGACCAGCGTGGACTGCACCATGTACATCTGCGGCGATTCCACCG
AGTGCTCCAACCTGCTGCTGCAGTACGGCAGCTTCTGCACCCAGCTGAATAGAGC
CCTGACAGGGATCGCCGTGGAACAGGACAAGAACACCCAAGAGGTGTTCGCCCA
AGTGAAGCAGATCTACAAGACCCCTCCTATCAAGGACTTCGGCGGCTTCAATTTC
AGCCAGATTCTGCCCGATCCTAGCAAGCCCAGCAAGCGGAGCcctATCGAGGACC
TGCTGTTCAACAAAGTGACACTGGCCGACGCCGGCTTCATCAAGCAGTATGGCG
ATTGTCTGGGCGACATTGCCGCCAGGGATCTGATTTGCGCCCAGAAGTTTAACGG
ACTGACAGTGCTGCCTCCTCTGCTGACCGATGAGATGATCGCCCAGTACACATCT
GCCCTGCTGGCCGGCACAATCACAAGCGGCTGGACATTTGGAGCTGGCcctGCTCT
GCAGATCCCCTTTccaATGCAGATGGCCTACCGGTTCAACGGCATCGGAGTGACCC
AGAATGTGCTGTACGAGAACCAGAAGCTGATCGCCAACCAGTTCAACAGCGCCA
TCGGCAAGATCCAGGACAGCCTGAGCAGCACAcccAGCGCCCTGGGAAAGCTGCA
GaacGTGGTCAACCAGAATGCCCAGGCACTGAACACCCTGGTCAAGCAGCTGTCC
TCCAACTTCGGCGCCATCAGCTCTGTGCTGAACGATATCCTGAGCAGACTGGACc
cccctGAAGCCGAGGTGCAGATCGACAGACTGATCACCGGAAGGCTGCAGTCCCTG
CAGACCTACGTTACCCAGCAGCTGATCAGAGCCGCCGAGATTAGAGCCTCTGCC
AATCTGGCCGCCACCAAGATGTCTGAGTGTGTGCTGGGCCAGAGCAAGAGAGTG
GACTTTTGCGGCAAGGGCTACCACCTGATGAGCTTCCCTCAGTCTGCCCCTCACG
GCGTGGTGTTTCTGCACGTGACATACGTGCCCGCTCAAGAGAAGAATTTCACCAC
CGCTCCAGCCATCTGCCACGACGGCAAAGCCCACTTTCCTAGAGAAGGCGTGTTC
GTGTCCAACGGCACCCATTGGTTCGTGACCCAGCGGAACTTCTACGAGCCCCAGA
TCATCACCACCGACAACACCTTCGTGTCTGGCAACTGCGACGTCGTGATCGGCAT
TGTGAACAATACCGTGTACGACCCTCTGCAGCCCGAGCTGGACAGCTTCAAAGA
GGAACTGGATAAGTACTTTAAGAACCACACAAGCCCCGACGTGGACCTGGGCGA
TATCAGCGGAATCAATGCCAGCGTCGTGAACATCCAGAAAGAGATCGACCGGCT
GAACGAGGTGGCCAAGAATCTGAACGAGAGCCTGATCGACCTGCAAGAACTGGG
GAAGTACGAGCAGTACATCAAGTGGCCC
Nucleotide Sequence of NDV-HXP-S (Delta) (SEQ ID NO: 5) Without Signal Sequence:
SEQ ID NO: 21
CAGTGTGTGAACCTGagaACAAGAACCCAGCTGCCTCCAGCCTACACCAACAGCT
TTACCAGAGGCGTGTACTACCCCGACAAGGTGTTCAGATCCAGCGTGCTGCACTC
TACCCAGGACCTGTTCCTGCCTTTCTTCAGCAACGTGACCTGGTTCCACGCCATCC
ACGTGTCCGGCACCAATGGCACCAAGAGATTCGACAACCCCGTGCTGCCCTTCA
ACGACGGGGTGTACTTTGCCAGCACCGAGAAGTCCAACATCATCAGAGGCTGGA
TCTTCGGCACCACACTGGACAGCAAGACCCAGAGCCTGCTGATCGTGAACAACG
CCACCAACGTGGTCATCAAAGTGTGCGAGTTCCAGTTCTGCAACGACCCCTTCCT
GgacGTCTACTATCACAAGAACAACAAGAGCTGGATGGAAAGCggcGTGTACAGCA
GCGCCAACAACTGCACCTTCGAGTACGTGTCCCAGCCTTTCCTGATGGACCTGGA
AGGCAAGCAGGGCAACTTCAAGAACCTGCGCGAGTTCGTGTTCAAGAACATCGA
CGGCTACTTCAAGATCTACAGCAAGCACACCCCTATCAACCTCGTGCGGGATCTG
CCTCAGGGCTTCTCTGCTCTGGAACCCCTGGTGGATCTGCCCATCGGCATCAACA
TCACCCGGTTTCAGACACTGCTGGCCCTGCACAGAAGCTACCTGACACCTGGCGA
TAGCAGCAGCGGATGGACAGCTGGTGCCGCCGCTTACTATGTGGGCTACCTGCA
GCCTAGAACCTTTCTGCTGAAGTACAACGAGAACGGCACCATCACCGACGCCGT
GGATTGTGCTCTGGATCCTCTGAGCGAGACAAAGTGCACCCTGAAGTCCTTCACC
GTGGAgAAGGGCATCTACCAGACCAGCAACTTCCGGGTGCAGCCCACCGAATCC
ATCGTGCGGTTCCCCAATATCACCAATCTGTGCCCCTTCGGCGAGGTGTTCAATG
CCACCAGATTCGCCTCTGTGTACGCCTGGAACCGGAAGCGGATCAGCAATTGCGT
GGCCGACTACTCCGTGCTGTACAACTCCGCCAGCTTCAGCACCTTCAAGTGCTAC
GGCGTGTCCCCTACCAAGCTGAACGACCTGTGCTTCACAAACGTGTACGCCGACA
GCTTCGTGATCCGGGGAGATGAAGTGCGGCAGATTGCCCCTGGACAGACAGGCA
AGATCGCCGACTACAACTACAAGCTGCCCGACGACTTCACCGGCTGTGTGATTGC
CTGGAACAGCAACAACCTGGACTCCAAAGTCGGCGGCAACTACAATTACcggTAC
CGGCTGTTCCGGAAGTCCAATCTGAAGCCCTTCGAGCGGGACATCTCCACCGAG
ATCTATCAGGCCGGCAGCaagCCTTGTAACGGCGTGGAAGGCTTCAACTGCTACTT
CCCACTGCAGTCCTACGGCTTTCAGCCCACAAATGGCGTGGGCTATCAGCCCTAC
AGAGTGGTGGTGCTGAGCTTCGAACTGCTGCATGCCCCTGCCACAGTGTGCGGCC
CTAAGAAgAGCACCAATCTCGTGAAGAACAAATGCGTGAACTTCAACTTCAACG
GCCTGACCGGCACCGGCGTGCTGACAGAGAGCAACAAGAAGTTCCTGCCATTCC
AGCAGTTTGGCCGGGATATCGCCGATACCACAGACGCCGTTAGAGATCCCCAGA
CACTGGAAATCCTGGACATCACCCCTTGCAGCTTCGGCGGAGTGTCTGTGATCAC
CCCTGGCACCAACACCAGCAATCAGGTGGCAGTGCTGTACCAGggcGTGAACTGT
ACCGAAGTGCCCGTGGCCATTCACGCCGATCAGCTGACACCTACATGGCGGGTG
TACTCCACCGGCAGCAATGTGTTTCAGACCAGAGCCGGCTGTCTGATCGGAGCCG
AGCACGTGAACAATAGCTACGAGTGCGACATCCCCATCGGCGCTGGCATCTGTG
CCAGCTACCAGACACAGACAAACAGCagaGCCTCTGTGGCCAGCCAGAGCATCAT
TGCCTACACAATGTCTCTGGGCGCCGAGAACAGCGTGGCCTACTCCAACAACTCT
ATCGCTATCCCCACCAACTTCACCATCAGCGTGACCACAGAGATCCTGCCTGTGT
CCATGACCAAGACCAGCGTGGACTGCACCATGTACATCTGCGGCGATTCCACCG
AGTGCTCCAACCTGCTGCTGCAGTACGGCAGCTTCTGCACCCAGCTGAATAGAGC
CCTGACAGGGATCGCCGTGGAACAGGACAAGAACACCCAAGAGGTGTTCGCCCA
AGTGAAGCAGATCTACAAGACCCCTCCTATCAAGGACTTCGGCGGCTTCAATTTC
AGCCAGATTCTGCCCGATCCTAGCAAGCCCAGCAAGCGGAGCcctATCGAGGACC
TGCTGTTCAACAAAGTGACACTGGCCGACGCCGGCTTCATCAAGCAGTATGGCG
ATTGTCTGGGCGACATTGCCGCCAGGGATCTGATTTGCGCCCAGAAGTTTAACGG
ACTGACAGTGCTGCCTCCTCTGCTGACCGATGAGATGATCGCCCAGTACACATCT
GCCCTGCTGGCCGGCACAATCACAAGCGGCTGGACATTTGGAGCTGGCcctGCTCT
GCAGATCCCCTTTccaATGCAGATGGCCTACCGGTTCAACGGCATCGGAGTGACCC
AGAATGTGCTGTACGAGAACCAGAAGCTGATCGCCAACCAGTTCAACAGCGCCA
TCGGCAAGATCCAGGACAGCCTGAGCAGCACAcccAGCGCCCTGGGAAAGCTGCA
GaacGTGGTCAACCAGAATGCCCAGGCACTGAACACCCTGGTCAAGCAGCTGTCC
TCCAACTTCGGCGCCATCAGCTCTGTGCTGAACGATATCCTGAGCAGACTGGACc
cccctGAAGCCGAGGTGCAGATCGACAGACTGATCACCGGAAGGCTGCAGTCCCTG
CAGACCTACGTTACCCAGCAGCTGATCAGAGCCGCCGAGATTAGAGCCTCTGCC
AATCTGGCCGCCACCAAGATGTCTGAGTGTGTGCTGGGCCAGAGCAAGAGAGTG
GACTTTTGCGGCAAGGGCTACCACCTGATGAGCTTCCCTCAGTCTGCCCCTCACG
GCGTGGTGTTTCTGCACGTGACATACGTGCCCGCTCAAGAGAAGAATTTCACCAC
CGCTCCAGCCATCTGCCACGACGGCAAAGCCCACTTTCCTAGAGAAGGCGTGTTC
GTGTCCAACGGCACCCATTGGTTCGTGACCCAGCGGAACTTCTACGAGCCCCAGA
TCATCACCACCGACAACACCTTCGTGTCTGGCAACTGCGACGTCGTGATCGGCAT
TGTGAACAATACCGTGTACGACCCTCTGCAGCCCGAGCTGGACAGCTTCAAAGA
GGAACTGGATAAGTACTTTAAGAACCACACAAGCCCCGACGTGGACCTGGGCGA
TATCAGCGGAATCAATGCCAGCGTCGTGAACATCCAGAAAGAGATCGACCGGCT
GAACGAGGTGGCCAAGAATCTGAACGAGAGCCTGATCGACCTGCAAGAACTGGG
GAAGTACGAGCAGTACATCAAGTGGCCCggcggaggtgggtcgCTCATAACATACATCG
TCCTGACTATAATCAGCTTGGTATTTGGTATTTTGTCTTTGATTCTTGCATGCTATT
TGATGTATAAACAGAAAGCTCAGCAGAAGACTCTCCTGTGGCTCGGTAACAACA
CACTCGACCAGATGAGAGCAACTACAAAGATGTGA
Amino Acid Sequence of S-F chimera HexaPro (SEQ ID NO: 11) Without Signal
Sequence: SEQ ID NO: 23
QCVNLTTRTQLPPAYTNSFTRGVYYPDKVFRSSVLHSTQDLFLPFFSNVTWFHAIHVS
GTNGTKRFDNPVLPFNDGVYFASTEKSNIIRGWIFGTTLDSKTQSLLIVNNATNVVIK
VCEFQFCNDPFLGVYYHKNNKSWMESEFRVYSSANNCTFEYVSQPFLMDLEGKQG
NFKNLREFVFKNIDGYFKIYSKHTPINLVRDLPQGFSALEPLVDLPIGINITRFQTLLAL
HRSYLTPGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASVYAWNRKRIS
NCVADYSVLYNSASFSTFKCYGVSPTKLNDLCFTNVYADSFVIRGDEVRQIAPGQTG
KIADYNYKLPDDFTGCVIAWNSNNLDSKVGGNYNYLYRLFRKSNLKPFERDISTEIY
QAGSTPCNGVEGENCYFPLQSYGFQPTNGVGYQPYRVVVLSFELLHAPATVCGPKK
STNLVKNKCVNFNFNGLTGTGVLTESNKKFLPFQQFGRDIADTTDAVRDPQTLEILDI
TPCSFGGVSVITPGTNTSNQVAVLYQDVNCTEVPVAIHADQLTPTWRVYSTGSNVFQ
TRAGCLIGAEHVNNSYECDIPIGAGICASYQTQTNSPASVASQSIIAYTMSLGAENSVA
YSNNSIAIPTNFTISVTTEILPVSMTKTSVDCTMYICGDSTECSNLLLQYGSFCTQLNR
ALTGIAVEQDKNTQEVFAQVKQIYKTPPIKDFGGFNFSQILPDPSKPSKRSPIEDLLFN
KVTLADAGFIKQYGDCLGDIAARDLICAQKFNGLTVLPPLLTDEMIAQYTSALLAGTI
TSGWTFGAGPALQIPFPMQMAYRFNGIGVTQNVLYENQKLIANQFNSAIGKIQDSLSS
TPSALGKLQDVVNQNAQALNTLVKQLSSNFGAISSVLNDILSRLDPPEAEVQIDRLIT
GRLQSLQTYVTQQLIRAAEIRASANLAATKMSECVLGQSKRVDFCGKGYHLMSFPQ
SAPHGVVFLHVTYVPAQEKNFTTAPAICHDGKAHFPREGVFVSNGTHWFVTQRNFY
EPQIITTDNTFVSGNCDVVIGIVNNTVYDPLQPELDSFKEELDKYFKNHTSPDVDLGDI
SGINASVVNIQKEIDRLNEVAKNLNESLIDLQELGKYEQYIKWPGGGGSLITYIVLTIIS
LVFGILSLILACYLMYKQKAQQKTLLWLGNNTLDQMRATTKM*

TABLE 4
Other Sequences

| | | |
|---|---|---|
| Linker | GGGGS | SEQ ID NO: 7 |
| Sequence | | |
| SacII | CCGCGG | SEQ ID NO: 8 |
| Restriction | | |
| Sequence | | |
| NDV Gene | TTAGAAAAAA | SEQ ID NO: 9 |
| End Sequence | | |
| NDV Gene | ACGGGTAGAA | SEQ ID NO: 10 |
| Start Sequence | | |
| Kozak | CCGCCACC | SEQ ID NO: 14 |
| Sequence | | |
| Signal | MFVFLVLLPLVSS | SEQ ID NO: 15 |
| Sequence | | |
| Linker | (GGGGS)ₙ, wherein n is 1, 2, 3, 4, 5 | SEQ ID NO: 24 |
| | or more | |
| Linker | (His)ₙ, wherein n is 6; or His-His-His- | SEQ ID NO: 25 |
| | His-His-His | |

### 6. EXAMPLES

### 6.1 Example 1: Recombinant NDV Expressing Chimeric F Protein

The Delta variant (B.1.617.2) initially originated from India in the late 2020 and was later became the dominant variant of concern globally. Resistance of the Delta variant to antibody neutralization and breakthrough infections have been observed in vaccinated humans (1-3). Herein it is shown that the NDV platform could be rapidly adapted to make the Delta variant vaccine.

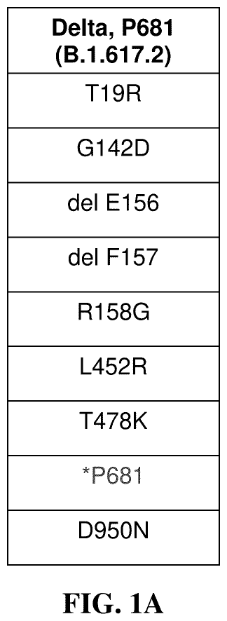

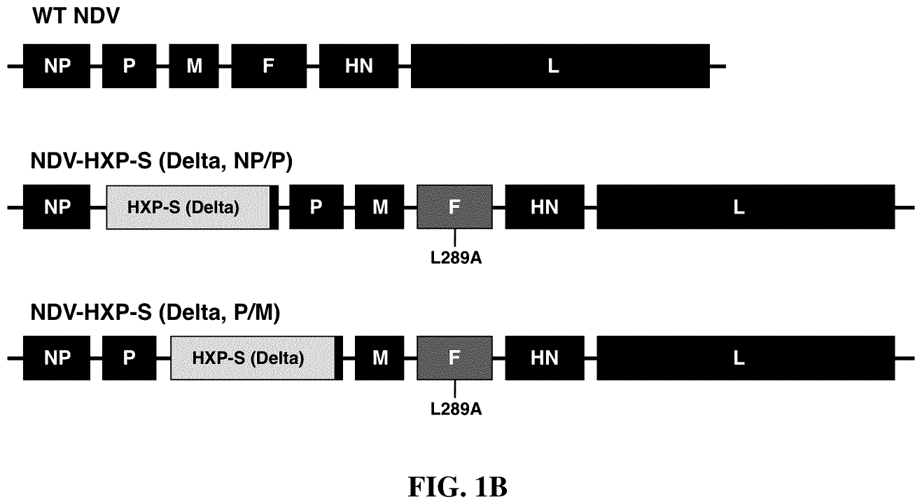

Briefly, Delta spike specific mutations were introduced into our prototype HXP-S backbone, in which the polybasic cleavage site was removed (⁶⁸²RRAR⁶⁸⁵ to A), HexaPro stabilizing mutations (8) are kept and the cytoplasmic tail and the transmembrane domain of the spike were replaced with those of the fusion (F) protein of NDV. The mutation representing the Delta variant spike is shown in FIG. 1A. Of note, one mutation in the Delta spike (P681R) was not included as our preliminary data (not shown) and additional studies (4-6) demonstrated that P681R enhanced the cleavage the of spike protein, therefore destabilizing the protein from pre-fusion conformation. As a vaccine, a prefusion conformation is preferred for the induction of protective neutralizing antibodies.

Two constructs of NDV-HXP-S to express the Delta variant spike protein were designed, aiming to explore whether one construct is superior than the other in terms of immunogenicity and stability. It is known that during replication of the NDV, a transcription gradient of mRNA of each gene is produced from 3′ to 5′ of the genome, giving the NP gene with the most abundant mRNA and L gene the least (7). This was due to a “stop-start” transcription model, in which the disassociated viral RNA dependent RNA polymerase (vRdRP) can only re-initiate transcription by binding to the leader sequence (Le) at the very 3′ end. By moving the independent transcription unit of the spike gene toward the 3′ end, the transcription of the spike could be improved, leading to possibly higher immunogenicity of the vaccine especially for the live vaccine. Therefore, through reverse genetics, the spike gene was inserted between the NP and P genes or the P and M genes. These two constructs were designated as NDV-HXP-S (Delta, NP/P) and NDV-HXP-S (Delta, P/M) respectively (FIG. 1B).

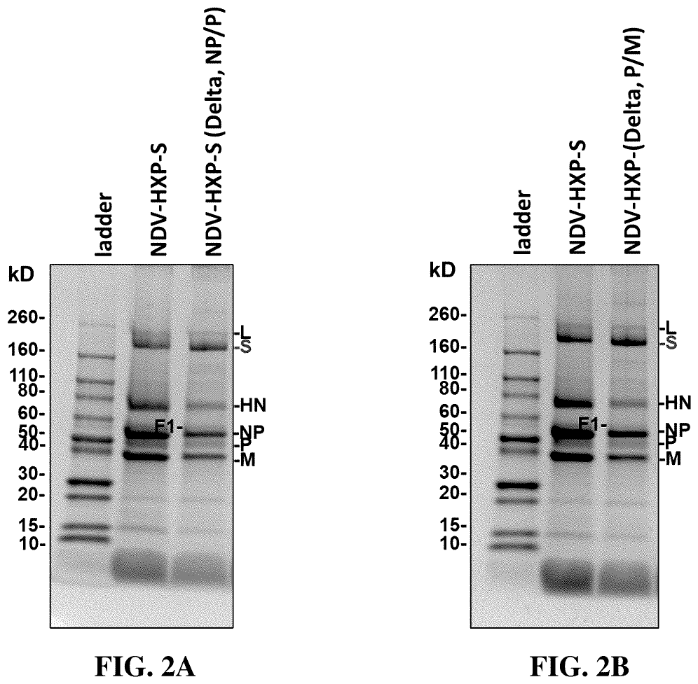

Despite of the different insertion sites, both constructs were designed to express the same Delta spike protein. Both viruses were rescued and limiting dilutions of the viruses in chicken embryonated eggs were performed to obtain homogenous clones of the pre-master virus seed (pre-MVS). To verify that the presence of the spike protein. The pre-MVS was propagated in eggs and virus was purified from allantoic fluid through a 20% sucrose cushion via ultracentrifugation. The purified viruses were resolved on 4-20% SDS-PAGE followed by Coomassie Blue staining with the prototype NDV-HXP-S as the control. Both NDV-HXP-S (Delta, NP/P) (FIG. 2A) and NDV-HXP-S (Delta, P/M) (FIG. 2B) were observed to express the spike protein very well.

The immunogenicity of the two constructs may be determined in animal studies.

### 6.1.1 References Cited in Example 1

1. Planas D, Veyer D, Baidaliuk A, Staropoli I, Guivel-Benhassine F, Rajah M M, Planchais C, Porrot F, Robillard N, Puech J, Prot M, Gallais F, Gantner P, Velay A, Le Guen J, Kassis-Chikhani N, Edriss D, Belec L, Seve A, Courtellemont L, Pere H, Hocqueloux L, Fafi-Kremer S, Prazuck T, Mouquet H, Bruel T, Simon-Loriere E, Rey F A, Schwartz O. 2021. Reduced sensitivity of SARS-CoV-2 variant Delta to antibody neutralization. Nature 596:276-280.
2. Liu C, Ginn H M, Dejnirattisai W, Supasa P, Wang B, Tuekprakhon A, Nutalai R, Zhou D, Mentzer A J, Zhao Y, Duyvesteyn H M E, Lopez-Camacho C, Slon-Campos J, Walter T S, Skelly D, Johnson S A, Ritter T G, Mason C, Costa Clemens S A, Gomes Naveca F, Nascimento V, Nascimento F, Fernandes da Costa C, Resende P C, Pauvolid-Correa A, Siqueira M M, Dold C, Temperton N, Dong T, Pollard A J, Knight J C, Crook D, Lambe T, Clutterbuck E, Bibi S, Flaxman A, Bittaye M, Belij-Rammerstorfer S, Gilbert S C, Malik T, Carroll M W, Klenerman P, Barnes E, Dunachie S J, Baillie V, Serafin N, Ditse Z, Da Silva K, Paterson N G, Williams M A, et al. 2021. Reduced neutralization of SARS-CoV-2 B.1.617 by vaccine and convalescent serum. Cell 184:4220-4236 e13.
3. Wang B, Goh Y S, Fong S W, Young B E, Ngoh E Z X, Chavatte J M, Salleh S N M, Yeo N K, Amrun S N, Hor P X, Loh C Y, Lee C Y, Chan Y H, Chang Z W, Tay M Z, Rouers A, Torres-Ruesta A, Carissimo G, Soh M K, Lee R T C, Xu Y, Pada S, Lin R T P, Leo Y S, Lye D C, Maurer-Stroh S, Ng L F P, Renia L, Wang C I. 2021. Resistance of SARS-CoV-2 Delta variant to neutralization by BNT162b2-elicited antibodies in Asians. Lancet Reg Health West Pac 15:100276.
4. Takeda M. 2021. Proteolytic activation of SARS-CoV-2 spike protein. Microbiol Immunol doi:10.1111/1348-0421.12945.
5. Liu Y, Liu J, Johnson B A, Xia H, Ku Z, Schindewolf C, Widen S G, An Z, Weaver S C, Menachery V D, Xie X, Shi P Y. 2021. Delta spike P681R mutation enhances SARS-CoV-2 fitness over Alpha variant. bioRxiv doi:10.1101/2021.08.12.456173.
6. Lubinski B, Frazier L E, M VTP, Bugembe D L, Tang T, Daniel S, Cotten M, Jaimes J A, Whittaker G R. 2021. Spike protein cleavage-activation mediated by the SARS-CoV-2 P681R mutation: a case-study from its first appearance in variant of interest (VOI) A.23.1 identified in Uganda. bioRxiv doi:10.1101/2021.06.30.450632.
7. Wignall-Fleming E B, Hughes D J, Vattipally S, Modha S, Goodbourn S, Davison A J, Randall R E. 2019. Analysis of Paramyxovirus Transcription and Replication by High-Throughput Sequencing. J Virol 93.
8. Ching-Lin Hsieh, Jory A. Goldsmith, Jeffrey M. Schaub, Andrea M. Divenerhung-Che, Kuokamyab Javanmardi, Kevin C. Le, Daniel Wrapp, Alison G. Lee, Jason S. McLellan. 2020. Structure-based design of prefusion-stabilized SARS-CoV-2 spikes. Science•369: 1501-1505

### 6.2 Example 2: NDV-HXP-S Vaccine Protects Against SARS-Cov-2

This example describes the development of NDV-HXP-S, which expresses the stabilized spike protein of SARS CoV 2 delta variant. This example describes the immunogenicity and protection triggered by vaccination with inactivated NDV-HXP-S Delta variant in mice. The data indicate that delta variant-specific vaccine induced a strong antibody response towards the homologous SARS-CoV-2 variant.

### 6.2.1 Materials and methods

### Cells

DF-1 (ATCC® CRL-12203), BSRT7 (1), and VERO-E6 (ATCC, CRL-1586) were maintained in Dulbecco's Modified Eagle's Medium (DMEM; Gibco, MA, USA) containing 10% (vol/vol) fetal bovine serum (FBS), 100 unit/mL of penicillin, 100 μg/mL of streptomycin (P/S; Gibco) and 10 mM 4-(2-hydroxyethyl)-1-piperazineethanesulfonic acid (HEPES) at 37° C. with 5% CO₂. VERO-TMPRSS2 cells (BPS Biosciences, #78081) were maintained in DMEM (Gibco) containing 10% (vol/vol) FBS, 100 unit/mL of penicillin, 100 μg/mL of streptomycin (P/S; Gibco), 5 mL of Nonessential Amino Acid Solution (NEAA, Corning™ MEM, NY, USA), 3 μg/mL puromycin (Invivogen, CA, USA) and 0.1 mg/mL Normocin (Invivogen) at 37° C. with 5% CO₂.

### Plasmids

Spike variant mutations were introduced into the HXP-S sequence in silico and the new constructs were obtained as synthetic double-stranded DNA fragments from Integrated DNA Technologies, using the gBlocks® Gene Fragments service and PCR (2). Briefly, the variant HXP-S were inserted into the pNDV_LS/L289A rescue plasmid (between P and M genes) by in-Fusion cloning (Clontech, CA, USA). The recombinant product was transformed into MAX Efficiency™ Stb12™ Competent Cells (Thermo Fisher Scientific, MA, USA) to generate the pNDV-HXP-S rescue plasmid. The plasmid was purified using PureLink™ HiPure Plasmid Maxiprep Kit (Thermo Fisher Scientific).

### Rescue of the NDV-HXP-S

As described in previous studies (3), BSRT7 cells stably expressing the T7 polymerase were seeded onto 6-well plates at 3×10⁵ cell per well in duplicate. The next day, cells were transfected with 2 μg of pNDV-HXP-S, 1 μg of pTM1-NP, 0.5 μg of pTM1-P, 0.5 μg of pTM1-L and 1 μg of pCI-T7opt and were re-suspended in 250 μl of Opti-MEM (Gibco). The plasmid cocktail was then gently mixed with 15 μL of TransIT LT1 transfection reagent (Mirus, GA, USA). The growth media was replaced with Opti-MEM during transfection. To increase rescue efficiency, BSRT7-DF-1 co-culture was established the next day as described previously (4). Specifically, transfected BSRT7 and DF-1 cells were washed with warm PBS and trypsinized. Trypsinized cells were neutralized with excessive amount of growth media. BSRT7 cells were mixed with DF-1 cells (˜1:2.5) in a 10-cm dish. The co-culture was incubated at 37° C. overnight. The next day, the media was removed and cells were gently washed with warm PBS, Opti-MEM supplemented with 1% P/S and 0.1 μg/ml of TPCK-trypsin. The co-cultures were incubated for 2 or 3 days before inoculation into 8 or 9 day-old specific pathogen free (SPF) embryonated chicken eggs (Charles River Laboratories, CT, USA). To inoculate eggs, cells and supernatants were harvested and homogenized by several syringe strokes. One or two hundred microliters of the mixture were injected into each egg. Eggs were incubated at 37° C. for 3-5 days and cooled at 4° C. overnight. Allantoic fluids (AF) were harvested from cooled eggs and the rescue of the viruses was determined by hemagglutination (HA) assays. RNA of the rescued virus was extracted, and RT-PCR was performed to amplify the cDNA segments of the viral genome. The cDNA segments were then sequenced by Sanger sequencing (Psomagen, MA, USA). The genetic stability of the recombinant viruses was evaluated across multiples passages in 10 days old-SPF embryonated chicken eggs.

### Virus Titration by EID₅₀ Assays

Fifty percent of embryo (egg) infectious dose (EID₅₀) assay was performed in 9 to 11-day old chicken embryonated eggs. Virus in allantoic fluid was 10-fold serially diluted in PBS, resulting in 10-5 to 1010 dilutions of the virus. One hundred microliters of each dilution were injected into each egg for a total of 5-10 egg per dilution. The eggs were incubated at 37° C. for 3 days and then cooled at 4° C. overnight, allantoic fluids were collected and analyzed by HA assay. The EID₅₀ titer of the NDV, determined by the number of HA-positive and HA-negative eggs in each dilution, was calculated using the Reed and Muench method.

### Preparation of Inactivated Concentrated Virus

The viruses in the allantoic fluid were first inactivated using 0.05% beta-propiolactone (BPL) as described previously (3). To concentrate the viruses, allantoic fluids were clarified by centrifugation at 4,000 rpm at 4° C. for 30 min using a Sorvall Legend RT Plus Refrigerated Benchtop Centrifuge (Thermo Fisher Scientific). Clarified allantoic fluids were laid on top of a 20% sucrose cushion in PBS (Gibco). Ultracentrifugation in a Beckman L7-65 ultracentrifuge at 25,000 rpm for 2 hours at 4° C. using a Beckman SW28 rotor (Beckman Coulter, CA, USA) was performed to pellet the viruses through the sucrose cushion while soluble egg proteins were removed. The virus pellets were re-suspended in PBS (pH 7.4). The total protein content was determined using the bicinchoninic acid (BCA) assay (Thermo Fisher Scientific).

### SDS-PAGE and Western Blot

The concentrated NDV-HXP-S or WT NDV was mixed with Novex™ Tris-Glycine SDS Sample Buffer (2×) (Thermo Fisher Scientific), NuPAGE™ Sample Reducing Agent (10×) (Thermo Fisher Scientific) and PBS at appropriate amounts to reach a total protein content. The mixture was heated at 90° C. for 5 min. The samples were mixed by pipetting and loaded to a 4-20% 10-well Mini-PROTEAN TGX™ precast gel. Ten microliters of the Novex™ Sharp Pre-stained Protein standard (Thermo Fisher Scientific) was used as the ladder. The electrophoresis was run in Tris/Glycine SDS/Buffer (Bio-Rad).

For Coomassie blue staining, the gel was washed with distilled water at room temperature several times until the dye front in the gel was no longer visible. The gel was stained with 20 mL of SimplyBlue™ SafeStain (Thermo Fisher Scientific) for a minimum of 1 h to overnight. The SimplyBlue™ SafeStain was decanted and the gel was washed with distilled water several times until the background was clear. Gels were imaged using the Bio-Rad Universal Hood IiIMolecular imager (Bio-Rad) and processed by Image Lab Software (Bio-Rad).

For Western Blot, proteins were transferred onto polyvinylidene difluoride (PVDF) membrane (GE Healthcare, IL, USA). The membrane was blocked with 5% dry milk in PBS containing 0.1% v/v Tween 20 (PBST) for 1 h at RT. The membrane was washed with PBST on a shaker 3 times (10 min at RT each time) and incubated with primary antibodies diluted in PBST containing 1% BSA overnight at 4° C. To detect the spike protein of SARS-CoV-2, a mouse monoclonal antibody 2B3E5 recognizing the S1 kindly provided by Dr. Thomas Moran at ISMMS was used. The HN protein was detected by a mouse monoclonal antibody 8H2 (MCA2822, Bio-Rad). The membranes were then washed with PBST on a shaker 3 times (10 min at RT each time) and incubated with sheep anti-mouse IgG linked with horseradish peroxidase (HRP) diluted (1:2,000) in PBST containing 5% dry milk for 1 h at RT. The secondary antibody was discarded and the membranes were washed with PBST on a shaker 3 times (10 min at RT each time). Pierce™ ECL Western Blotting Substrate (Thermo Fisher Scientific) was added to the membrane, the blots were imaged using the Bio-Rad Universal Hood Ii Molecular imager (Bio-Rad) and processed by Image Lab Software (Bio-Rad).

### Animal Experiments

All the animal experiments were performed in accordance with protocols approved by the Icahn School of Medicine at Mount Sinai (ISMMS) Institutional Animal Care and Use Committee (IACUC). All experiments with live SARS-CoV-2 were performed in the Centers for Disease Control and Prevention (CDC)/US Department of Agriculture (USDA)-approved biosafety level 3 (BSL-3) biocontainment facility of the Global Health and Emerging Pathogens Institute at the Icahn School of Medicine at Mount Sinai, in accordance with institutional biosafety requirements.

### Mouse Immunization and Challenge Studies

Female BALB/c mice were used in all studies. Intramuscular vaccination using 1 μg of total protein of inactivated NDV-HXP-S vaccine or negative control WT NDV was prepared in 100 μl total volume. Two immunizations were performed for all the mice with a 21-day interval. For SARS-CoV-2 infection, mice were intranasally infected with 2.5×10⁸ plaque forming units (PFU) of Ad5-hACE2 5 days prior to being challenged with 1×10⁵ PFU USA-WA1/2020 strain (Wuhan), 1.6×10⁵ PFU of the PV29995/2021 strain (Delta, kindly provided by Dr. Viviana Simon from ISMMS, Mount Sinai Pathogen Surveillance Program) and 5.0×10³ PFU of the hCoV-19/USA/WI-UW-4340/2021 strain (Mu, a kind gift from Dr. Yoshi Kawaoka lab from University of Wisconsin-Madison). Viral titers in the lung homogenates of mice 2 days post-infection were used as the readout for protection. Briefly, the lung lobes were harvested from a subset of animals per group and homogenized in 1 mL of sterile PBS. Viral titers in the lung homogenates were measured by plaque assay on Vero cells. Blood was collected by submandibular vein bleeding. Sera were isolated by low-speed centrifugation and stored at −80° C. before use.

### Recombinant Proteins

Recombinant WA1, Beta, and Alpha spike protein and recombinant WA1, Beta, Alpha and Omicron and Delta RBD were generated and expressed in Expi293F cells (Life Technologies, Thermo Fisher Scientific) as previously described (5, 6). Proteins were then purified after transient transfections with each respective plasmid. Briefly, the mammalian-cell codon-optimized nucleotide sequence of a soluble spike protein (amino acids 1-1,213) lacking the polybasic cleavage site, carrying two stabilizing mutations (K986P and V987P), a signal peptide, and at the C-terminus a thrombin cleavage site, a T4 foldon trimerization domain, and a hexahistidine tag was cloned into the mammalian expression vector pCAGGS. Protein was purified using gravity flow purification with Ni-nitrilotriacetic acid (NTA) agarose (Qiagen, Germany) and concentrated and buffer exchanged in Amicon centrifugal units (EMD Millipore, MA, USA). The purified recombinant proteins were analyzed via reducing sodium dodecyl sulfate-polyacrylamide gel electrophoresis (SDS-PAGE). The Omicron spike protein was provided by Dr. Noah Sather from Seattle Children's hospital. The desired protein folding was confirmed through ELISAs using the Receptor Binding Domain (RBD)-specific monoclonal antibody CR3022 (7). Recombinant Delta (10878-CV-10) spike protein was purchased from R&D Systems (R&D Systems, Bio-Techne, MN, USA). S2 (Catalogue #40590-V08B) recombinant protein was acquired from Sino Biological.

### Enzyme-Linked Immunosorbent Assay (ELISA)

Spike-specific IgG in mice sera vaccinated with NDV-HXP-S was measured by ELISA as described previously (3, 8). Proteins were coated onto Immulon® 4 HBX 96-well microtiter plates (Thermo Fisher Scientific) at 2 μg/mL in 1× coating buffer (SeraCare Life Sciences Inc., MA, USA) at 50 μL/well overnight at 4° C. All plates were washed 3 times with 225 μL PBS containing 0.1% (vol/vol) Tween-20 (PBST) and 220 μL blocking solution (3% goat serum, 0.5% non-fat dried milk powder, 96.5% PBST) was added to each well and incubated for 1 hour at RT. Individual serum samples or pooled sera were serially diluted 3-fold in blocking solution followed by a 2-hour incubation at RT at a starting dilution of 1:30. ELISA plates were afterwards washed 3 times with PBST and 50 μL of anti-mouse IgG-horseradish peroxidase (HRP) conjugated antibody (Cytiva, GE Healthcare) was added at a dilution of 1:3,000 in blocking solution. After 1 hour, plates were washed 3 times with PBST and developed using SigmaFast OPD (Sigma-Aldrich, MI, USA) for 10 minutes. Reactions were stopped by adding 50 μL 3M hydrochloric acid and absorbance at 492 nm was determined on a Synergy 4 plate reader (BioTek) or similar. For each ELISA plate, the blank average absorbance plus 3 standard deviations was used as a cutoff to determine endpoint titers and the area under the curve (AUC) using GraphPad Prism.

### Microneutralization Assays Using the Authentic SARS-CoV-2 Viruses

Microneutralization assays using the authentic SARS-CoV-2 viruses were performed as described previously in Vero TMPRSS2 (9). All procedures were performed in a biosafety level 3 (BSL-3) facility at the Icahn School of Medicine at Mount Sinai following standard safety guidelines. Vero-E6-TMPRSS2 cells were seeded in 96-well high binding cell culture plates (Costar, #07620009, Corning) at a density of 20,000 cells/well in complete Dulbecco's modified Eagle medium (cDMEM) one day prior to the infection. Heat inactivated serum samples (56° C. for 1 hour) were serially diluted (3-fold) in minimum essential media (MEM; Gibco, #11430-030) supplemented with 2 mM L-glutamine (Gibco, #25030081), 0.1% sodium bicarbonate (w/v, HyClone, #SH30033.01), 10 mM 4-(2-hydroxyethyl)-1-piperazineethanesulfonic acid (HEPES; Gibco, #15630080), 100 U/ml 242 penicillin, 100 μg/ml streptomycin (Gibco, #15140122) and 0.2% bovine serum albumin (BSA, M Biomedicals, Cat #. 810063) starting at 1:10. Remdesivir (Medkoo Bioscience Inc., #329511) was included to monitor assay variation. Serially diluted sera were incubated with 10,000 TCID50 of WT USA-WA1/2020 SARS-CoV-2, PV29995/2021 (B.1617.2, Delta), MSHSPSP-PV27007/2021 (B.1.351, Beta) and PV44488/2021 (B.1.1.529, Omicron) for one hour at RT, followed by the transfer of 120 μL of the virus-sera mix to Vero-E6-TMPRSS2 plates. Infection proceeded for one hour at 37° C. and inoculum was removed. 100 μL/well of the corresponding antibody dilutions plus 100 μL/well of infection media supplemented with 2% fetal bovine serum (FBS; Gibco, #10082-147) were added to the cells. Plates were incubated for 48 h at 37° C. followed by fixation 250 overnight at 4° C. in 200 μl/well of a 10% formaldehyde solution. For staining of the nucleoprotein, formaldehyde solution was removed, and cells were washed with PBS (pH 7.4) (Gibco, #10010-031) and permeabilized by adding 150 μl/well of PBS, 0.1% Triton X-100 (Fisher Bioreagents, #BP151-100) for 15 min at RT. Permeabilization solution was removed, plates were washed with 200 μl/well of PBS (Gibco, #10010-031) twice and blocked with PBS, 3% BSA for 1 hour at RT. During this time the primary antibody was biotinylated according to manufacturer protocol (Thermo Scientific EZ-Link NHS-PEG4-Biotin). Blocking solution was removed and 100 μl/well of biotinylated mAb 1C7C7, a mouse anti-SARS nucleoprotein monoclonal antibody generated at the Center for Therapeutic Antibody Development at The Icahn School of Medicine at Mount Sinai ISMMS (Millipore Sigma, Cat #ZMS1075) at a concentration of 1 μg/ml in PBS, 1% BSA was added for 1 hour at RT. Cells were washed with 200 μl/well of PBS twice and 100 μl/well of HRP-conjugated streptavidin (Thermo Fisher Scientific) diluted in PBS, 1% BSA were added at a 1:2,000 dilution for 1 hour at RT. Cells were washed twice with PBS, and 100 μl/well of o-phenylenediamine dihydrochloride (Sigmafast OPD; Sigma-Aldrich) were added for 10 min at RT, followed by addition of 50 μl/well of a 3 M HCl solution (Thermo Fisher Scientific). Optical density (OD) was measured (490 nm) using a microplate reader (Synergy H1; Biotek). Analysis was performed using Prism 7 software (GraphPad). After subtraction of background and calculation of the percentage of neutralization with respect to the “virus only” control, a nonlinear regression curve fit analysis was performed to calculate the 50% inhibitory dilution (ID50), with top and bottom constraints set to 100% and 0% respectively. All samples were analyzed in a blinded manner.

### SARS-CoV-2 Plaque Assay

Plaque assays with SARS-Cov-2 viruses were performed in the BSL3 facility. Vero E6 cells or Vero TMPRSS2 were seeded onto 12-well plates in growth media at 1:5 and cultured for two days. Tissue homogenates were 10-fold serially diluted in infection medium (DMEM containing 2% FBS, 100 unit/mL of penicillin, 100 μg/mL of streptomycin (P/S; Gibco) and 10 mM HEPES). Two hundred microliters of each dilution were inoculated onto each well starting with a 1:10 dilution of the sample. The plates were incubated at 37° C. for 1 h with occasional rocking every 10 min. The inoculum in each well was then removed and 1 mL of agar overlay containing 0.7% of agar in 2×MEM was placed onto each well. Once the agar was solidified, the plates were incubated at 37° C. with 5% CO₂. Two days later, the plates were fixed with 5% formaldehyde in PBS overnight before being taken out from BSL3 for subsequent staining under BSL2 conditions. The plaques were immuno-stained with an anti-SARS-CoV-2 NP primary mouse monoclonal antibody 1C7C7 kindly provided by Dr. Thomas Moran at ISMMS. An HRP-conjugated goat anti-mouse secondary antibody was used at 1:2000 and the plaques were visualized using TrueBlue™ Peroxidase Substrate (SeraCare Life Sciences Inc.).

### 6.2.2 Results

### Design and Production of NDV-HXP-S Delta Variant Vaccine

NDV-HXP-S variant vaccine based on Delta (B.1.617.2) was rescued using reverse genetics in mammalian cell cultures and further amplified in specific-pathogen free (SPF) embryonated chicken eggs as previously described (FIG. 3A-3D) (2). The nucleotide sequence of the construct was codon-optimized for mammalian host expression. The HXP-S sequence was inserted between the P and M genes of the NDV genome. The furin polybasic cleavage site (⁶⁸²RRAR⁶⁸⁵) was removed and the transmembrane domain (TM) and cytoplasmic tail (CT) of the spike were replaced with those from the fusion (F) protein of LaSota NDV. The HXP mutations were introduced into the spike S2 region of the variant S protein to improve stability as reported previously (FIGS. 3A and 3B) (10). In addition, a completely cleaved Delta spike was observed when natural P681R mutation was included. Therefore, the 681 was kept as proline to ensure the homogeneity of S0 conformation. (FIG. 3C).

Research-grade beta-propiolactone (BPL) inactivated NDV-HXP-S Delta variant vaccine preparation was produced. Virus was concentrated from the harvested allantoic fluid through a sucrose cushion and resolved on a sodium dodecyl-sulfate polyacrylamide gel (SDS-PAGE) with Coomassie Blue staining to evaluate the presence of NDV-HXP-S protein. NDV-HXP-S Delta variant showed an extra band between 160 kDa and 260 kDa below the L protein of the NDV that corresponds to the size of the uncleaved S0 (FIG. 3D) (2).

### NDV-HXP-S Delta Variant Vaccination Increases Protection In Vivo Against Phylogenetically Distant SARS-CoV-2 Variants

When the Delta vaccine construct was generated, a mouse immunization and challenge study was performed testing vaccine formulations containing the Delta component (FIG. 4A). The following groups were evaluated: monovalent Wuhan and monovalent Delta. In all cases, the vaccination dose was 1 μg of total protein (FIG. 4B).

In this study, the goal was not only to test the protection against homologous challenge, but also against a phylogenetically distant SARS-CoV-2 variant, which is unmatched to any vaccine component. To do so, animals in each group were divided into 3 subgroups and challenged with Wuhan, Delta and Mu variants (FIG. 4C). As expected, Wuhan and Delta showed protection against its homologous virus challenge.

Considering the new emergence of the Omicron variant, the neutralizing activity of post-boost serum antibodies against the homologous Wuhan, Delta, Beta and Omicron variants in authentic virus neutralization assays was tested (FIG. 4D). The ID₅₀ obtained against Wuhan for the Delta-vaccinated group was 0.4-fold compared to Wuhan. In the case of Delta microneutralization, the neutralizing activity for Wuhan alone showed a smaller neutralizing activity (0.8-fold) compared to Delta. A similar ID₅₀ was obtained against Beta with monovalent Wuhan and Delta vaccination. Little neutralizing activity was found against Omicron in the Wuhan vaccination, whereas Delta vaccine showed a 5.6-fold increase.

### NDV-HXP-S Delta Vaccination Increases Antibody Binding to the Spike and RBD of SARS-CoV-2 Variants

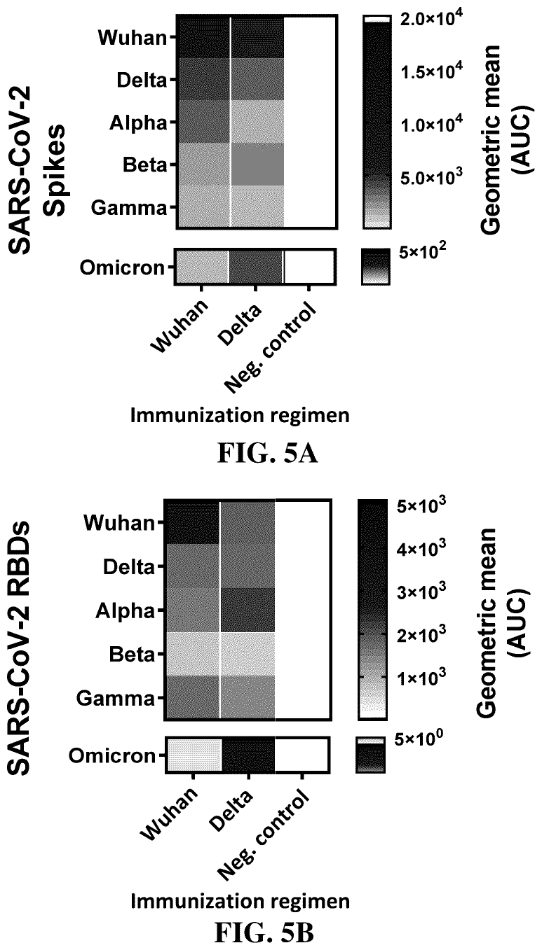

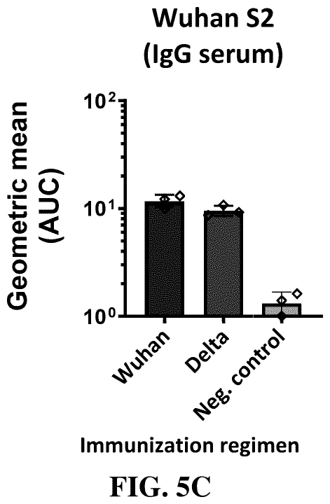

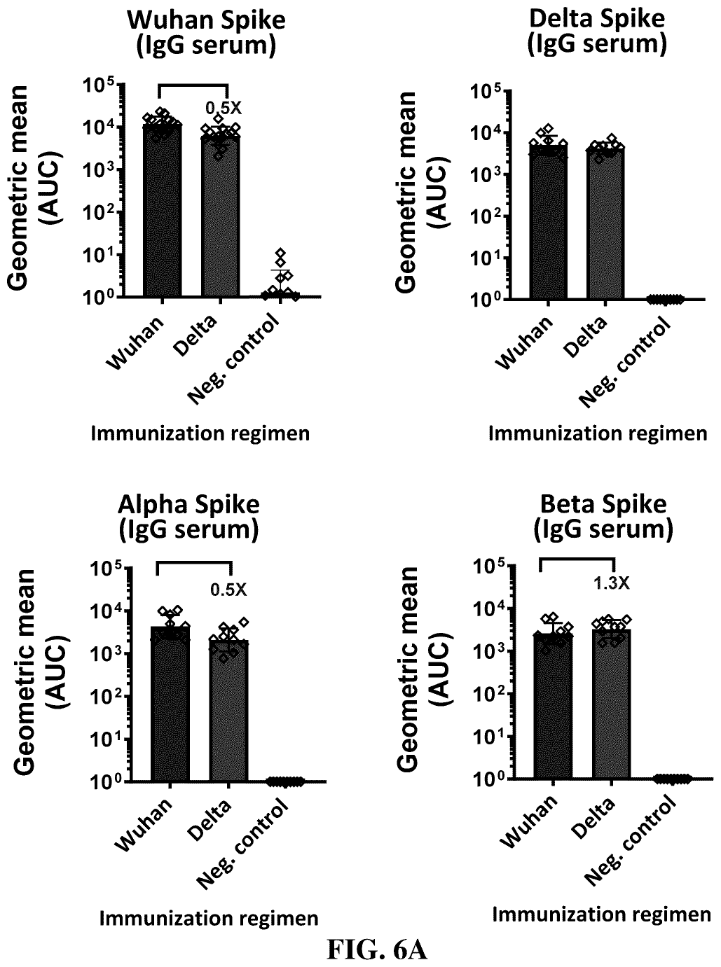

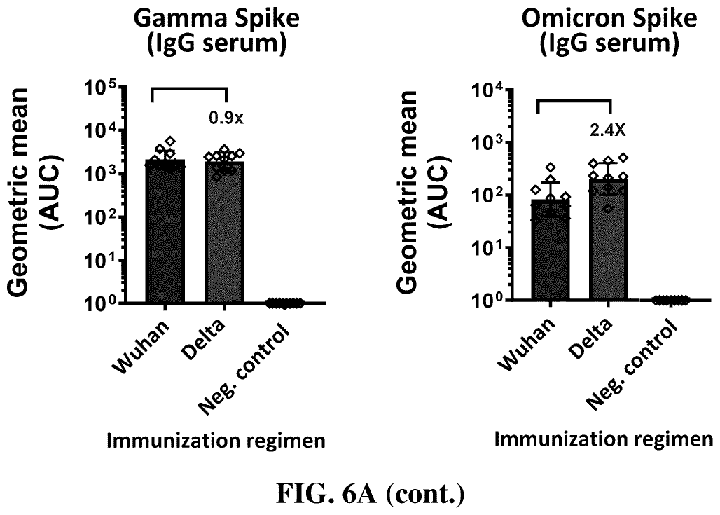

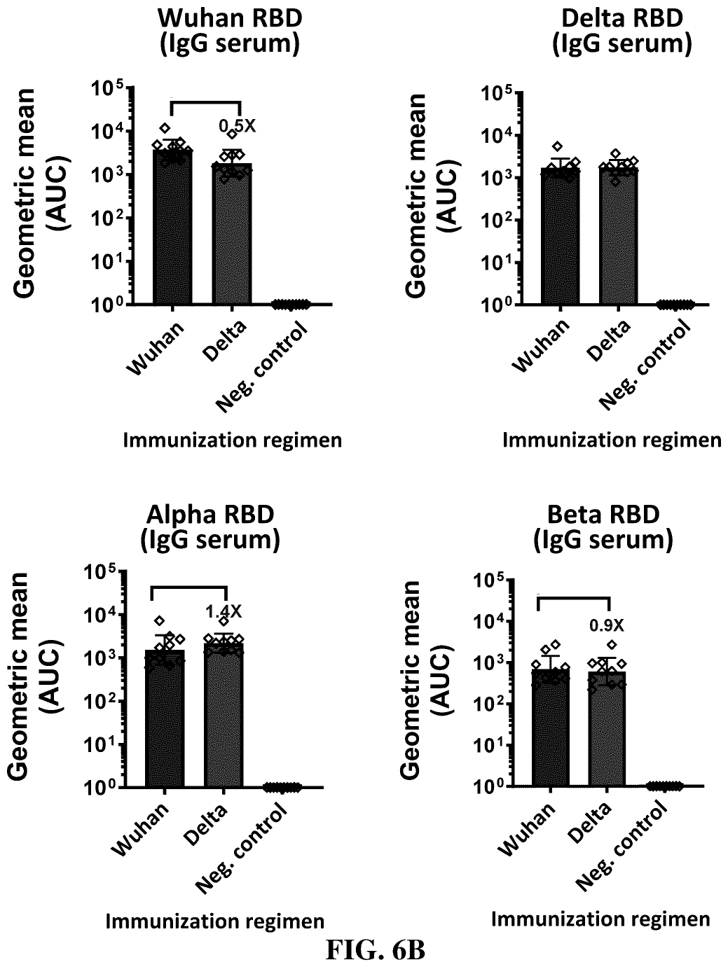

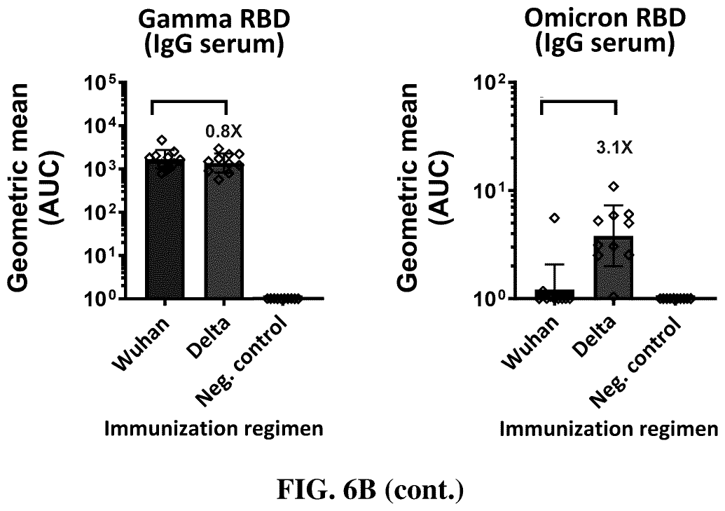

In the attempt to elucidate the mechanism of cross-protection induced by vaccine formulations, antibody-binding profiles were measured against a panel of SARS-CoV-2 spike proteins, including the Wuhan, Delta, Alpha, Beta, Gamma and Omicron (FIG. 5A and FIGS. 6A and 6B). Wuhan and Delta spike-specific IgG levels were comparable among all vaccination groups. Finally, vaccinated mice sera were also tested against Omicron spike. Omicron presents 30 different amino acid changes and deletions most of them occurring in the RBD. A general reduction in antibody titers was found in all groups compared to the other spikes tested (FIG. 5A, see legend axes). Despite this drop, Delta seemed to increase spike-specific IgG titers against Omicron: 2.4-fold in Delta alone compared to the Wuhan group, which correlates with the neutralization titers against Omicron (FIG. 4D). Immune sera against the RBDs of Wuhan, Delta, Beta and Omicron was also tested (FIG. 5B and FIGS. 6A and 6B). Again, monovalent Wuhan was shown to be the best against Wuhan RBD.

IgG antibody titers were further measured against S2 region (FIG. 5C) of the original Wuhan spike.

### 6.2.3 Discussion

The COVID-19 pandemic has seen an unprecedented race in the development of next generation vaccines. Compared to the traditionally slow vaccine development, novel vaccine platforms such as Moderna and Pfizer's mRNA vaccines or Janssen and AstraZeneca's adenovirus-based vaccines have been authorized for emergency use in one year (11, 12). Despite of this tremendous effort, only a 63.1% of the global population has been fully vaccinated largely due to inequal vaccine distribution (11). This fact, together with the continuing emergence of variants of concern (VOC), which present a higher transmissibility and resistance to vaccine-mediated protection, has caused thousands of deaths per day since the beginning of the pandemic. In the last months the situation has worsened with the arrival of Omicron, where the world has experienced the highest number of daily cases (11).

With the NDV-based vaccine platform, variant-specific vaccine for the Delta variant was successfully constructed. In mice, variant-specific vaccines effectively conferred protection against their homologous viruses with a limited breadth. To test if the combined presentation of prior variants could potentially induce cross-protective immune responses against unmatched and antigenically distinct lineages, a monovalent formulation of inactivated NDV-HXP-S Delta variant vaccine was examined. In vitro microneutralization assays demonstrated an increase number of cross-neutralizing antibodies against the Omicron variant (FIG. 4D).

In the case of the Mu variant, the Wuhan vaccine seems to be more beneficial than Delta vaccine (FIG. 4C). From the neutralization assays, it appears that the Delta vaccine contains neutralizing epitope(s) that are shared by the Omicron spike (FIG. 4D), since monovalent Delta vaccine substantially increased antibody neutralizing activity against the Omicron variant (5.6-fold compared to Wuhan group, FIG. 4D).

In previous work, it has been shown that inactivated NDV-HXP-S induced higher ratio of neutralizing antibodies over binding antibodies in vaccinated people than the Pfizer mRNA vaccine (13). The introduction of the HexaPro mutations, and the replacement of the furin cleavage site in the spike sequence may have contributed to increase the immunogenicity of NDV-HXP-S vaccine compared to the mRNA vaccine (2, 10). A second factor that may contribute is the antigen presentation in the surface of the NDV versus the protein alone generated by mRNA vaccines. NDV-HXP-S also expresses the cognate HN and F glycoproteins on the surface of the virion, which are known to have a size of ˜75 kDa and ˜55 kDa, respectively, meanwhile HXP-S trimer has a size of ˜200 kDa. Hence, there might be steric hindrance to the S2 region of the spike, focusing the immune response towards the more immunogenic but less conserved S1 region.

The NDV-HXP-S vaccine has proven its safety and immunogenicity in several preclinical (2, 14-16) and clinical studies (17-19). As an egg-based vaccine, NDV-HXP-S could be produced using the egg-based influenza manufacturing capacities located worldwide. Sparrow and colleagues estimated that influenza manufacturing capacities on its own can produce billions of doses annually (14).

In summary, this example describes the development of novel NDV-HXP-S Delta variant vaccine and demonstrates the generation of neutralizing antibodies against unmatched VOCs.

### 6.2.4 References Cited in Example 2

1. Ikegame S, Hashiguchi T, Hung C T, Dobrindt K, Brennand K J, Takeda M, Lee B. 2021. Fitness selection of hyperfusogenic measles virus F proteins associated with neuropathogenic phenotypes. Proc Natl Acad Sci USA 118.
2. Sun W, Liu Y, Amanat F, Gonzalez-Dominguez I, McCroskery S, Slamanig S, Coughlan L, Rosado V, Lemus N, Jangra S, Rathnasinghe R, Schotsaert M, Martinez J L, Sano K, Mena I, Innis B L, Wirachwong P, Thai D H, Oliveira R D N, Scharf R, Hjorth R, Raghunandan R, Krammer F, Garcia-Sastre A, Palese P. 2021. A Newcastle disease virus expressing a stabilized spike protein of SARS-CoV-2 induces protective immune responses. Nat Commun 12:6197.
3. Sun W, McCroskery S, Liu W C, Leist S R, Liu Y, Albrecht R A, Slamanig S, Oliva J, Amanat F, Schafer A, Dinnon K H, 3rd, Innis B L, Garcia-Sastre A, Krammer F, Baric R S, Palese P. 2020. A Newcastle Disease Virus (NDV) Expressing a Membrane-Anchored Spike as a Cost-Effective Inactivated SARS-CoV-2 Vaccine. Vaccines (Basel) 8.
4. Ayllon J, Garcia-Sastre A, Martinez-Sobrido L. 2013. Rescue of recombinant Newcastle disease virus from cDNA. J Vis Exp doi:10.3791/50830.
5. Amanat F, Stadlbauer D, Strohmeier S, Nguyen T H O, Chromikova V, McMahon M, Jiang K, Arunkumar G A, Jurczyszak D, Polanco J, Bermudez-Gonzalez M, Kleiner G, Aydillo T, Miorin L, Fierer D S, Lugo L A, Kojic E M, Stoever J, Liu S T H, Cunningham-Rundles C, Felgner P L, Moran T, Garcia-Sastre A, Caplivski D, Cheng A C, Kedzierska K, Vapalahti O, Hepojoki J M, Simon V, Krammer F. 2020. A serological assay to detect SARS-CoV-2 seroconversion in humans. Nat Med 26:1033-1036.
6. Stadlbauer D, Amanat F, Chromikova V, Jiang K, Strohmeier S, Arunkumar G A, Tan J, Bhavsar D, Capuano C, Kirkpatrick E, Meade P, Brito R N, Teo C, McMahon M, Simon V, Krammer F. 2020. SARS-CoV-2 Seroconversion in Humans: A Detailed Protocol for a Serological Assay, Antigen Production, and Test Setup. Curr Protoc Microbiol 57:e100.
7. ter Meulen J, van den Brink E N, Poon L L, Marissen W E, Leung C S, Cox F, Cheung C Y, Bakker A Q, Bogaards J A, van Deventer E, Preiser W, Doerr H W, Chow V T, de Kruif J, Peiris J S, Goudsmit J. 2006. Human monoclonal antibody combination against SARS coronavirus: synergy and coverage of escape mutants. PLoS Med 3:e237.
8. Sun W, Leist S R, McCroskery S, Liu Y, Slamanig S, Oliva J, Amanat F, Schafer A, Dinnon K H, 3rd, Garcia-Sastre A, Krammer F, Baric R S, Palese P. 2020. Newcastle disease virus (NDV) expressing the spike protein of SARS-CoV-2 as a live virus vaccine candidate. EBioMedicine 62:103132.
9. Carreno J M, Alshammary H, Tcheou J, Singh G, Raskin A, Kawabata H, Sominsky L, Clark J, Adelsberg D C, Bielak D, Gonzalez-Reiche A S, Dambrauskas N, Vigdorovich V, Group P P S, Srivastava K, Sather D N, Sordillo E M, Bajic G, van Bakel H, Simon V, Krammer F. 2021. Activity of convalescent and vaccine serum against SARS-CoV-2 Omicron. Nature doi:10.1038/d41586-021-03846-z.
10. Hsieh C L, Goldsmith J A, Schaub J M, DiVenere A M, Kuo H C, Javanmardi K, Le K C, Wrapp D, Lee A G, Liu Y, Chou C W, Byrne P O, Hjorth C K, Johnson N V, Ludes-Meyers J, Nguyen A W, Park J, Wang N, Amengor D, Lavinder J J, Ippolito G C, Maynard J A, Finkelstein I J, McLellan J S. 2020. Structure-based design of prefusion-stabilized SARS-CoV-2 spikes. Science 369:1501-1505.
11. Hannah Ritchie E M, Lucas Rodés-Guirao, Cameron Appel, Charlie Giattino, Esteban Ortiz-Ospina, Joe Hasell, Bobbie Macdonald, Diana Beltekian and Max Roser. 2020. Coronavirus Pandemic (COVID-19), on Our World Data. https://ourworldindata.org/covid-vaccinations. Accessed 5 Feb. 2022.
12. Krammer F. 2020. SARS-CoV-2 vaccines in development. Nature 586:516-527.
13. Carreno J M, Raskin A, Singh G, Tcheou J, Kawabata H, Gleason C, Srivastava K, Vigdorovich V, Dambrauskas N, Gupta S L, Gonzalez I, Martinez J L, Slamanig S, Sather D N, Raghunandan R, Wirachwong P, Muangnoicharoen S, Pitisuttithum P, Wrammert J, Suthar M S, Sun W, Palese P, Garcia-Sastre A, Simon V, Krammer F. 2022. The inactivated NDV-HXP-S COVID-19 vaccine induces a significantly higher ratio of neutralizing to non-neutralizing antibodies in humans as compared to mRNA vaccines. medRxiv doi:10.1101/2022.01.25.22269808.
14. Sparrow E, Wood J G, Chadwick C, Newall A T, Torvaldsen S, Moen A, Torelli G. 2021. Global production capacity of seasonal and pandemic influenza vaccines in 2019. Vaccine 39:512-520.
15. Tcheou J, Raskin A, Singh G, Kawabata H, Bielak D, Sun W, Gonzalez-Dominguez I, Sather D N, Garcia-Sastre A, Palese P, Krammer F, Carreno J M. 2021. Safety and Immunogenicity Analysis of a Newcastle Disease Virus (NDV-HXP-S) Expressing the Spike Protein of SARS-CoV-2 in Sprague Dawley Rats. Front Immunol 12:791764.
16. Lara-Puente J H, Carreno J M, Sun W, Suarez-Martinez A, Ramirez-Martinez L, Quezada-Monroy F, Paz-De la Rosa G, Vigueras-Moreno R, Singh G, Rojas-Martinez O, Chagoya-Cortes H E, Sarfati-Mizrahi D, Soto-Priante E, Lopez-Macias C, Krammer F, Castro-Peralta F, Palese P, Garcia-Sastre A, Lozano-Dubernard B. 2021. Safety and Immunogenicity of a Newcastle Disease Virus Vector-Based SARS-CoV-2 Vaccine Candidate, AVX/COVID-12-HEXAPRO (Patria), in Pigs. mBio 12:e0190821.
17. Pitisuttithum P, Luvira V, Lawpoolsri S, Muangnoicharoen S, Kamolratanakul S, Sivakorn C, Narakorn P, Surichan S, Prangpratanporn S, Puksuriwong S, Lamola S, Mercer L D, Raghunandan R, Sun W, Liu Y, Carreno J M, Scharf R, Phumratanaprapin W, Amanat F, Gagnon L, Hsieh C L, Kaweepornpoj R, Khan S, Lal M, McCroskery S, McLellan J, Mena I, Meseck M, Phonrat B, Sabmee Y, Singchareon R, Slamanig S, Suthepakul N, Tcheou J, Thantamnu N, Theerasurakarn S, Tran S, Vilasmongkolchai T, White J A, Garcia-Sastre A, Palese P, Krammer F, Poopipatpol K, Wirachwong P, Hjorth R, Innis B L. 2021. Safety and Immunogenicity of an Inactivated Recombinant Newcastle Disease Virus Vaccine Expressing SARS-CoV-2 Spike: Interim Results of a Randomised, Placebo-Controlled, Phase 1/2 Trial. medRxiv doi:10.1101/2021.09.17.21263758.
18. Samuel Ponce-de-León M T, Luis Enrique Soto-Ramirez, Juan Jos6 Calva, Patricio Santillan-Doherty, Dora Eugenia Carranza-Salazar, Juan Manuel Carreno, Claudia Carranza, Esmeralda Juárez, Laura E. Carreto-Binaghi, Luis Ramirez-Martinez, Georgina Paz-De la Rosa, Rosalia Vigueras-Moreno, Alejandro Ortiz-Stern, Yolanda López-Vidal, Alejandro E. Macias, Jesús Torres-Flores, Oscar Rojas-Martinez, Alejandro Suirez-Martinez, Gustavo Peralta-Sinchez, Hisaaki Kawabata, Irene González-Dominguez, José Luis Martinez-Guevara, Weina Sun, David Sarfati-Mizrahi, Ernesto Soto-Priante, Héctor Elias Chagoya-Cortés, Constantino López-Macias, Felipa Castro-Peralta, Peter Palese, Adolfo Garcia-Sastre, Florian Krammer, Bernardo Lozano-Dubernard. 2022. Safety and immunogenicity of a live recombinant Newcastle disease virus-based COVID-19 vaccine (Patria) administered via the intramuscular or intranasal route: Interim results of a non-randomized open label phase I trial in Mexico. medRxiv doi:doi.org/10.1101/2022.02.08.22270676.
19. Dang A D, Vu T D, Vu H H, Ta V T, Pham A T V, Dang M T N, Van Le B, Duong T H, Van Nguyen D, Lawpoolsri S, Chinwangso P, McLellan J S, Hsieh C-L, Garcia-Sastre A, Palese P, Sun W, Martinez J L, Gonzalez-Dominguez I, Slamanig S, Carreno J M, Tcheou J, Krammer F, Raskin A, Vu H M, Tran T C, Nguyen H M, Mercer L D, Raghunandan R, Lal M, White J A, Hjorth R, Innis B L, Scharf R. 2022. Safety and Immunogenicity of An Egg-Based Inactivated Newcastle Disease Virus Vaccine Expressing SARS-CoV-2 Spike: Interim Results of a Randomized, Placebo-Controlled, Phase 1/2 Trial in Vietnam. doi:10.1101/2022.02.01.22270253.

### 6.3 Example 3: NDV-HXP-S Delta Vaccine

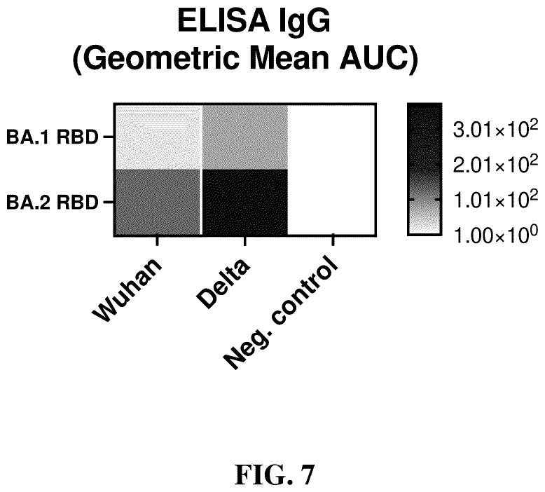

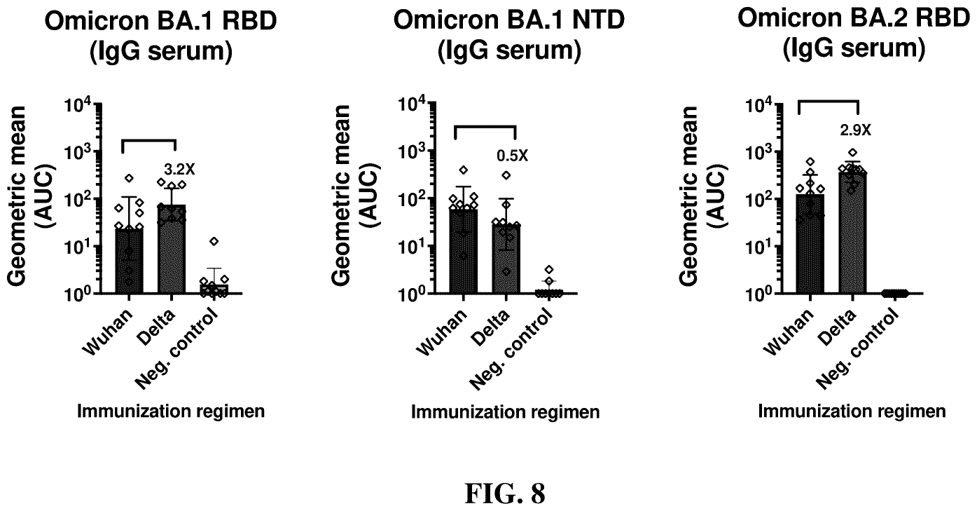

Mice were vaccinated with inactivated NDV-HXP-S vaccine or NDV-HXP-S Delta vaccine via the intramuscular route as described in Example 2, sera was obtained from those mice, and antibody in the sera was assessed for binding to recombinant proteins produced by techniques such as described in Example 2 in ELISAs, such as described in Example 2. Antibody-binding profiles were measured against SARS-CoV-2 spike protein receptor binding domains of Omicron BA.1 and BA.2 (FIG. 7). Sera from mice vaccinated with NDV-HXP-S vaccine or NDV-HXP-S Delta vaccine were tested for binding to the receptor binding domains of the spike protein of SARS-CoV-2 Omicron BA.1 and BA.2, and the N-terminal domain of the spike protein of Omicron BA.1. Sera from mice immunized with the NDV-HXP-S Delta vaccine increased RBD-specific IgG titers against Omicron BA.1 (about 3.2-fold) compared to RBD-specific IgG titers against Omicron BA.1 in sera from mice immunized with the NDV-HXP-S vaccine (FIG. 8). Similarly, sera from mice immunized with the NDV-HXP-S Delta vaccine increased RBD-specific IgG titers against Omicron BA.2 (about 2.9-fold) compared to RBD-specific IgG titers against Omicron BA.2 in sera from mice immunized with the NDV-HXP-S vaccine (FIG. 8).

### 7. EMBODIMENTS

The following are exemplary embodiments:
1. A recombinant Newcastle disease virus (NDV) comprising a packaged genome, wherein the package genome comprises a transgene, wherein the transgene comprises a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:12 with one, two, three, four, five, six, seven, eight, or all of the following amino acid modifications: T19R, G142D, delE156, delF157, R158G, L452R, T478K, D614G, and D950N.
2. A recombinant Newcastle disease virus (NDV) comprising a packaged genome, wherein the package genome comprises a transgene, wherein the transgene comprises a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:12 with the following amino acid modifications: T19R, G142D, delE156, delF157, R158G, L452R, T478K, D614G, and D950N.
3. A recombinant Newcastle disease virus (NDV) comprising a packaged genome, wherein the package genome comprises a transgene, wherein the transgene comprises a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:16 with one, two, three, four, five, six, seven, eight, or all of the following amino acid modifications: T6R, G129D, delE143, delF144, R145G, L439R, T465K, D601G, and D937N.
4. A recombinant NDV comprising a packaged genome, wherein the packaged genome comprises a transgene, wherein the transgene comprises a nucleotide sequence encoding a chimeric F protein, wherein the chimeric F protein comprises a derivative SARS-CoV-2 delta virus spike protein ectodomain and NDV F protein transmembrane and cytoplasmic domains, wherein the derivative comprises a SARS-CoV-2 delta spike protein ectodomain in which: (1) amino acid residues corresponding to amino acid residues 817, 892, 899, 942, 986, and 987 of SARS-CoV-2 spike protein found at GenBank Accession No. MN908947.3 are substituted with prolines, (2) amino acid residues corresponding to amino acid residues 682 to 685 of the polybasic cleavage site of SARS-CoV-2 spike protein found at GenBank Accession No. MN908947.3 are substituted such that the polybasic cleavage site is inactivated, and (3) two, three, four, five, six, seven, or more amino acid residues corresponding to two, three, four, five, six, seven, eight or more of amino acid residues 19, 142, 156, 157, 158, 452, 478, 614, and 950 of the spike protein found at GenBank Accession No. MN908947.3 are: 19R, 142D, del156, del157, 158G, 452R, 478K, 614G, and 950N.
5. A recombinant NDV comprising a packaged genome, wherein the packaged genome comprises a transgene, wherein the transgene comprises a nucleotide sequence encoding a chimeric F protein, wherein the chimeric F protein comprises a derivative SARS-CoV-2 delta virus spike protein ectodomain and NDV F protein transmembrane and cytoplasmic domains, wherein the derivative comprises a SARS-CoV-2 delta spike protein ectodomain in which: (1) amino acid residues corresponding to amino acid residues 817, 892, 899, 942, 986, and 987 of SARS-CoV-2 spike protein found at GenBank Accession No. MN908947.3 are substituted with prolines, (2) amino acid residues corresponding to amino acid residues 682 to 685 of the polybasic cleavage site of SARS-CoV-2 spike protein found at GenBank Accession No. MN908947.3 are substituted such that the polybasic cleavage site is inactivated, and (3) amino acid residues corresponding to amino acid residues 19, 142, 156, 157, 158, 452, 478, 614, and 950 of the spike protein found at GenBank Accession No. MN908947.3 are: R, D, deleted, deleted, G, R, K, G, and N, respectively.
6. The recombinant NDV of embodiment 4 or 5, wherein amino acid residues corresponding to amino acid residues 682 to 685 of the polybasic cleavage site of the SARS-CoV-2 spike protein found at GenBank Accession No. MN908947.3 are substituted with a single alanine.
7. A recombinant Newcastle disease virus (NDV) comprising a packaged genome, wherein the packaged genome comprises a transgene, wherein the transgene comprises a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises an amino acid sequence at least 99% identical to the amino acid sequence of SEQ ID NO:13 or 17.
8. A recombinant Newcastle disease virus (NDV) comprising a packaged genome, wherein the packaged genome comprises a transgene, wherein the transgene comprises a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises an amino acid sequence at least 99.5% identical to the amino acid sequence of SEQ ID NO:13 or 17.
9. A recombinant Newcastle disease virus (NDV) comprising a packaged genome, wherein the packaged genome comprises a transgene, wherein the transgene comprises a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:13 or 17.
10. The recombinant NDV of any one of embodiments 1 to 9, wherein the SARS-CoV-2 delta virus spike protein ectodomain is linked via a linker to the NDV F protein transmembrane and cytoplasmic domains.
11. A recombinant NDV comprising a packaged genome, wherein the packaged genome comprises a transgene, wherein the transgene comprises a nucleotide sequence encoding a chimeric F protein, and wherein the chimeric F protein comprises an amino acid sequence at least 99% identical to the amino acid sequence of SEQ ID NO:6 or 18.
12. A recombinant NDV comprising a packaged genome, wherein the packaged genome comprises a transgene, wherein the transgene comprises a nucleotide sequence encoding a chimeric F protein, and wherein the chimeric F protein comprises an amino acid sequence at least 99.5% identical to the amino acid sequence of SEQ ID NO:6 or 18.
13. A recombinant NDV comprising a packaged genome, wherein the packaged genome comprises a transgene, wherein the transgene comprises a nucleotide sequence encoding a chimeric F protein, and wherein the chimeric F protein comprises the amino acid sequence of SEQ ID NO:6 or 18.
14. A recombinant NDV comprising a packaged genome, wherein the packaged genome comprises a transgene, wherein the transgene comprises a nucleotide sequence encoding a chimeric F protein, wherein the transgene comprises an RNA sequence corresponding to the negative sense of the cDNA sequence of SEQ ID NO:5 or 21.
15. The recombinant NDV of any one of embodiments 1 to 14, wherein the NDV virion comprises the chimeric F protein.
16. The recombinant NDV of any one of embodiments 1 to 15, wherein the genome comprises a NDV F transcription unit, a NDV NP transcription unit, a NDV M transcription unit, a NDV L transcription unit, a NDV P transcription unit, and a NDV HN transcription unit.
17. The recombinant NDV of any one of embodiments 1 to 15, wherein the genome comprises a NDV F transcription unit, a NDV NP transcription unit, a NDV M transcription unit, a NDV L transcription unit, a NDV P transcription unit, and a NDV HN transcription unit, and wherein the NDV F transcription unit encodes a NDV F protein comprising a leucine to alanine amino acid substitution at the amino residue corresponding to amino acid residue 289 of the LaSota NDV strain.
18. The recombinant NDV of any one of embodiments 1 to 17, wherein the transgene is between two NDV transcription units of the packaged genome.
19. The recombinant NDV of embodiment 18, wherein the two transcription units of the packaged genome are the transcription units for the NDV P gene and the NDV M gene.
20. The recombinant NDV of embodiment 18, wherein the two transcription units of the packaged genome are the transcription units for the NDV NP gene and the NDV P gene
21. A recombinant Newcastle disease virus (NDV) comprising a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:12 with one, two, three, four, five, six, seven, or all of the following amino acid modifications: T19R, G142D, delE156, delF157, R158G, L452R, T478K, D614G, and D950N.
22. A recombinant Newcastle disease virus (NDV) comprising a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:16 with one, two, three, four, five, six, seven, or all of the following amino acid modifications: T6R, G129D, delE143, delF144, R145G, L439R, T465K, D601G, and D937N.
23. A recombinant Newcastle disease virus (NDV) comprising a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:12 with the following amino acid modifications: T19R, G142D, delE156, delF157, R158G, L452R, T478K, D614G, and D950N.
24. A recombinant NDV comprising a chimeric F protein, wherein the chimeric F protein comprises a derivative SARS-CoV-2 delta virus spike protein ectodomain and NDV F protein transmembrane and cytoplasmic domains, wherein the derivative comprises a SARS-CoV-2 delta spike protein ectodomain in which: (1) amino acid residues corresponding to amino acid residues 817, 892, 899, 942, 986, and 987 of SARS-CoV-2 spike protein found at GenBank Accession No. MN908947.3 are substituted with prolines, (2) amino acid residues corresponding to amino acid residues 682 to 685 of the polybasic cleavage site of SARS-CoV-2 spike protein found at GenBank Accession No. MN908947.3 are substituted such that the polybasic cleavage site is inactivated, and (3) two, three, four, five, six, seven, or more amino acid residues corresponding to two, three, four, five, six, seven, or more of amino acid residues 19, 142, 156, 157, 158, 452, 478, 614, and 950 of the spike protein found at GenBank Accession No. MN908947.3 are as follows: 19R, 142D, del156, del157, 158G, 452R, 478K, 614G, and 950N.
25. A recombinant NDV comprising a chimeric F protein, wherein the chimeric F protein comprises a derivative SARS-CoV-2 delta virus spike protein ectodomain and NDV F protein transmembrane and cytoplasmic domains, wherein the derivative comprises a SARS-CoV-2 delta spike protein ectodomain in which: (1) amino acid residues corresponding to amino acid residues 817, 892, 899, 942, 986, and 987 of SARS-CoV-2 spike protein found at GenBank Accession No. MN908947.3 are substituted with prolines, (2) amino acid residues corresponding to amino acid residues 682 to 685 of the polybasic cleavage site of SARS-CoV-2 spike protein found at GenBank Accession No. MN908947.3 are substituted such that the polybasic cleavage site is inactivated, and (3) amino acid residues corresponding to amino acid residues 19, 142, 156, 157, 158, 452, 478, 614, and 950 of the spike protein found at GenBank Accession No. MN908947.3 are: R, D, deleted, deleted, G, R, K, G, and N, respectively.
26. The recombinant NDV of embodiment 24 or 25, wherein amino acid residues corresponding to amino acid residues 682 to 685 of the polybasic cleavage site of the SARS-CoV-2 spike protein found at GenBank Accession No. MN908947.3 are substituted with a single alanine.
27. A recombinant Newcastle disease virus (NDV) comprising a chimeric F protein, wherein the chimeric F protein comprises a derivative of a SARS-CoV-2 delta virus spike protein ectodomain and NDV F protein transmembrane and cytoplasmic domains, and wherein the derivative comprises an amino acid sequence at least 99% identical to the amino acid sequence of SEQ ID NO:13 or 17.
28. A recombinant Newcastle disease virus (NDV) comprising a chimeric F protein, wherein the chimeric F protein comprises a derivative of a SARS-CoV-2 delta virus spike protein ectodomain and NDV F protein transmembrane and cytoplasmic domains, and wherein the derivative comprises an amino acid sequence at least 99.5% identical to the amino acid sequence of SEQ ID NO:13 or 17.
29. A recombinant Newcastle disease virus (NDV) comprising a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:13 or 17.
30. The recombinant NDV of any one of embodiments 21 to 29, wherein the SARS-CoV-2 delta virus spike protein ectodomain is linked via a linker to the NDV F protein transmembrane and cytoplasmic domains.
31. A recombinant NDV comprising a chimeric F protein, wherein the chimeric F protein comprises an amino acid sequence that is at least 99% identical to the amino acid sequence of SEQ ID NO:6 or 18.
32. A recombinant NDV comprising a chimeric F protein, wherein the chimeric F protein comprises an amino acid sequence that is at least 99.5% identical to the amino acid sequence of SEQ ID NO:6 or 18.
33. A recombinant NDV comprising a chimeric F protein, wherein the chimeric F protein comprises the amino acid sequence of SEQ ID NO:6 or 18.
34. A recombinant NDV comprising a chimeric F protein, wherein the chimeric F protein is encoded by a nucleotide sequence comprising the nucleotide sequence of SEQ ID NO:5 or 21.
35. The recombinant NDV of any one of embodiments 1 to 34, which comprises an NDV backbone which is lentogenic.
36. The recombinant NDV of any one of embodiments 1 to 34, which comprises an NDV backbone of LaSota strain.
37. The recombinant NDV of any one of embodiments 1 to 34, which comprises an NDV backbone of Hitchner B1 strain.
38. A composition comprising the recombinant NDV of any one of embodiments 1 to 37.
39. An immunogenic composition comprising the recombinant NDV of any one of embodiments 1 to 37.
40. The immunogenic composition of embodiment 39, wherein the recombinant NDV is inactivated.
41. The immunogenic composition of embodiment 39 or 40, further comprising an adjuvant.
42. A method for inducing an immune response to SARS-CoV-2 spike protein, comprising administering the immunogenic composition of any one of embodiments 39 to 41 to a subject.
43. A method for inducing an immune response to SARS-CoV-2 delta virus spike protein, comprising administering the immunogenic composition of any one of embodiments 39 to 41 to a subject.
44. A method for preventing COVID-19, comprising administering the immunogenic composition of any one of embodiments 39 to 41 to a subject.
45. A method for immunizing a subject against SARS-CoV-2, comprising administering the immunogenic composition of any one of embodiments 39 to 41 to a subject.
46. A method for immunizing a subject against SARS-CoV-2 delta variant, comprising administering the immunogenic composition of any one of embodiments 39 to 41 to a subject.
47. The method of any one of embodiments 42 to 46, wherein the composition is administered to the subject intranasally or intramuscularly.
48. The method of any one of embodiments 42 to 47, wherein the subject is a human.
49. The method of any one of embodiments 42 to 48, wherein the subject has been previously vaccinated with a COVID-19 vaccine.
50. A kit comprising the recombinant NDV of any one of embodiments 1 to 37.
51. A cell line or chicken embryonated egg comprising propagating the recombinant NDV of any one of embodiments 1 to 37.
52. A method for propagating the recombinant NDV of any one of embodiments 1 to 37, the method comprising culturing the cell or embryonated egg of embodiment 51.
53. The method of embodiment 52, wherein the method further comprises isolating the recombinant NDV from the cell or embryonated egg.
54. A method for detecting the presence of antibody specific to SARS-CoV-2 spike protein, comprising contacting a specimen with the recombinant NDV of any one of embodiments 1 to 37 in an immunoassay.
55. The method of embodiment 54, wherein the specimen is a biological specimen.
56. The method of embodiment 54, wherein the biological specimen is blood, plasma or sera from a subject.
57. The method of embodiment 56, wherein the subject is human.
58. The method of embodiment 54, wherein the specimen is an antibody or antisera
59. A transgene comprising a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:12 with one, two, three, four, five, six, seven, or all of the following amino acid modifications: T19R, G142D, delE156, delF157, R158G, L452R, T478K, D614G, and D950N.
60. A transgene comprising a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:12 with the following amino acid modifications: T19R, G142D, delE156, delF157, R158G, L452R, T478K, D614G, and D950N.
61. A transgene comprising a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:16 with one, two, three, four, five, six, seven, or all of the following amino acid modifications: T6R, G129D, delE143, delF144, R145G, L439R, T465K, D601G, and D937N.
62. A transgene comprising a nucleotide sequence encoding a chimeric F protein, wherein the chimeric F protein comprises a derivative SARS-CoV-2 delta virus spike protein ectodomain and NDV F protein transmembrane and cytoplasmic domains, wherein the derivative comprises a SARS-CoV-2 delta spike protein ectodomain in which: (1) amino acid residues corresponding to amino acid residues 817, 892, 899, 942, 986, and 987 of SARS-CoV-2 spike protein found at GenBank Accession No. MN908947.3 are substituted with prolines, (2) amino acid residues corresponding to amino acid residues 682 to 685 of the polybasic cleavage site of SARS-CoV-2 spike protein found at GenBank Accession No. MN908947.3 are substituted such that the polybasic cleavage site is inactivated, and (3) two, three, four, five, six, seven, or more amino acid residues corresponding to two, three, four, five, six, seven, or more of amino acid residues 19, 142, 156, 157, 158, 452, 478, 614, and 950 of the spike protein found at GenBank Accession No. MN908947.3 are as follows: 19R, 142D, del156, del157, 158G, 452R, 478K, 614G, and 950N.
63. A transgene comprising a nucleotide sequence encoding a chimeric F protein, wherein the chimeric F protein comprises a derivative SARS-CoV-2 delta virus spike protein ectodomain and NDV F protein transmembrane and cytoplasmic domains, wherein the derivative comprises a SARS-CoV-2 delta spike protein ectodomain in which: (1) amino acid residues corresponding to amino acid residues 817, 892, 899, 942, 986, and 987 of SARS-CoV-2 spike protein found at GenBank Accession No. MN908947.3 are substituted with prolines, (2) amino acid residues corresponding to amino acid residues 682 to 685 of the polybasic cleavage site of SARS-CoV-2 spike protein found at GenBank Accession No. MN908947.3 are substituted such that the polybasic cleavage site is inactivated, and (3) amino acid residues corresponding to amino acid residues 19, 142, 156, 157, 158, 452, 478, 614, and 950 of the spike protein found at GenBank Accession No. MN908947.3 are: R, D, deleted, deleted, G, R, K, G, and N, respectively.
64. The transgene of embodiment 62 or 63, wherein amino acid residues corresponding to amino acid residues 682 to 685 of the polybasic cleavage site of the SARS-CoV-2 spike protein found at GenBank Accession No. MN908947.3 are substituted with a single alanine.
65. A transgene comprising a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises an amino acid sequence at least 99% identical to the amino acid sequence of SEQ ID NO:13 or 17.
66. A transgene comprising a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises an amino acid sequence at least 99.5% identical to the amino acid sequence of SEQ ID NO:13 or 17.
67. A transgene comprising a nucleotide sequence encoding a chimeric F protein, wherein the chimeric F protein comprises a derivative of a SARS-CoV-2 delta virus spike protein ectodomain and NDV F protein transmembrane and cytoplasmic domains, and wherein the derivative comprises the amino acid sequence of SEQ ID NO:13 or 17.
68. The transgene of any one of embodiments 59 to 67, wherein the SARS-CoV-2 delta virus spike protein ectodomain is linked via a linker to the NDV F protein transmembrane and cytoplasmic domains.
69. A transgene comprising a nucleotide sequence encoding a chimeric F protein, wherein the chimeric F protein comprises an amino acid sequence that is at least 99% identical to the amino acid sequence of SEQ ID NO:6 or 18.
70. A transgene comprising a nucleotide sequence encoding a chimeric F protein, wherein the chimeric F protein comprises an amino acid sequence that is at least 99.5% identical to the amino acid sequence of SEQ ID NO:6 or 18.
71. A transgene comprising a nucleotide sequence encoding a chimeric F protein, wherein the chimeric F protein comprises the amino acid sequence of SEQ ID NO:6 or 18.
72. A transgene comprising a nucleotide sequence encoding a chimeric F protein, wherein the chimeric F protein is encoded by a nucleotide sequence comprising the nucleotide sequence of SEQ ID NO:5 or 21.
73. A vector comprising the transgene of any one of embodiments 59 to 72.
74. A nucleotide sequence comprising the transgene of any one of embodiments 59 to 72 and (1) a NDV F transcription unit, (2) a NDV NP transcription unit, (3) a NDV M transcription unit, (4) a NDV L transcription unit, (5) a NDV P transcription unit, and (6) a NDV HN transcription unit.
75. A nucleotide sequence comprising the transgene of any one of embodiments 59 to 72 and (1) a NDV F transcription unit, (2) a NDV NP transcription unit, (3) a NDV M transcription unit, (4) a NDV L transcription unit, (5) a NDV P transcription unit, and (6) a NDV HN transcription unit, wherein the NDV F transcription unit encodes a NDV F protein comprising a leucine to alanine amino acid substitution at the amino residue corresponding to amino acid residue 289 of the LaSota NDV strain.
76. A vector comprising the nucleotide sequence of embodiment 74 or 75.
77. A kit comprising the nucleotide sequence of embodiment 74 or 75, the transgene of any one of embodiments 59 to 72, the vector of embodiment 73 or 76, or the recombinant NDV of any one of embodiments 1 to 37.
78. A method for boosting antibody titer to SARS-CoV-2 spike protein in a subject previously vaccinated for COVID-19 or previously infected with SARS-CoV-2, comprising administering to the subject the recombinant NDV of any one of embodiments 1 to 37 or the immunogenic composition of any one of embodiments 39 to 41.
79. A method for boosting immunity to SARS-CoV-2 in a subject previously vaccinated for COVID-19 or previously infected with SARS-CoV-2, comprising administering to the subject the recombinant NDV of any one of embodiments 1 to 37 or the immunogenic composition of any one of embodiments 39 to 41.
80. The method of embodiments 78 or 79, wherein the subject is human.
81. A recombinant protein comprising a derivative of the ectodomain of SARS-CoV-2 delta variant, wherein the derivative comprises the amino acid sequence of SEQ ID NO:13 or 17.
82. An isolated nucleotide sequence encoding the recombinant protein of embodiment 81.
83. A vector comprising the nucleotide sequence of embodiment 82.
84. The vector of embodiment 83, which is a plasmid or a viral vector.
85. A cell line or ex vivo embryonated egg comprising the nucleotide sequence of embodiment 82, or the vector of embodiment 83 or 84.

The invention is not to be limited in scope by the specific embodiments described herein. Indeed, various modifications of the invention in addition to those described will become apparent to those skilled in the art from the foregoing description and accompanying Figures. Such modifications are intended to fall within the scope of the appended claims.

All references cited herein are incorporated herein by reference in their entirety and for all purposes to the same extent as if each individual publication or patent or patent application was specifically and individually indicated to be incorporated by reference in its entirety for all purposes.

What is claimed is:

1.-85. (canceled)

86. A transgene comprising a nucleotide sequence encoding a chimeric F protein, wherein the chimeric F protein comprises a modified SARS-CoV-2 delta virus spike protein ectodomain, and an NDV F protein transmembrane and cytoplasmic domains, and wherein
(a) the modified SARS-CoV-2 delta virus spike protein comprises the amino acid sequence of SEQ ID NO: 12 with one, two, three, four, five, six, seven, or all of the following amino acid modifications: T19R, G142D, delE156, delF157, R158G, L452R, T478K, D614G, and D950N, preferably the derivative comprises the amino acid sequence of SEQ ID NO: 12 with the following amino acid modifications: T19R, G142D, delE156, delF157, R158G, L452R, T478K, D614G, and D950N;
(b) the modified SARS-CoV-2 delta virus spike protein comprises the amino acid sequence of SEQ ID NO: 16 with one, two, three, four, five, six, seven, or all of the following amino acid modifications: T6R, G129D, delE143, delF144, R145G, L439R, T465K, D601G, and D937N;
(c) the modified SARS-CoV-2 delta virus spike protein ectodomain comprises:
(1) amino acid residues corresponding to amino acid residues 817, 892, 899, 942, 986, and 987 of SARS-CoV-2 spike protein according to GenBank Accession No. MN908947.3 are substituted with prolines,
(2) amino acid residues corresponding to amino acid residues 682 to 685 of the polybasic cleavage site of SARS-CoV-2 spike protein found at GenBank Accession No. MN908947.3 are substituted such that the polybasic cleavage site is inactivated, preferably the amino acid residues corresponding to amino acid residues 682 to 685 of the polybasic cleavage site of the SARS-CoV-2 spike protein according to GenBank Accession No. MN908947.3 are substituted with a single alanine, and
(3) two, three, four, five, six, seven, or more amino acid residues corresponding to two, three, four, five, six, seven, or more of amino acid residues 19, 142, 156, 157, 158, 452, 478, 614, and 950 of the spike protein according to GenBank Accession No. MN908947.3 are as follows: 19R, 142D, del156, del157, 158G, 452R, 478K, 614G, and 950N, preferably amino acid residues corresponding to amino acid residues 19, 142, 156, 157, 158, 452, 478, 614, and 950 of the spike protein according to GenBank Accession No. MN908947.3 are: R, D, deleted, deleted, G, R, K, G, and N, respectively; or
(d) the modified SARS-CoV-2 delta virus spike protein ectodomain comprises an amino acid sequence at least 99% or at least 99.5% identical to the amino acid sequence of SEQ ID NO: 13 or 17, preferably the modified SARS-CoV-2 delta virus spike protein ectodomain comprises the amino acid sequence of SEQ ID NO: 13 or 17.

87. The transgene of claim 86, wherein (a) the modified SARS-CoV-2 delta virus spike protein comprises the amino acid sequence of SEQ ID NO: 12 with one, two, three, four, five, six, seven, or all of the following amino acid modifications: T19R, G142D, delE156, delF157, R158G, L452R, T478K, D614G, and D950N, preferably the derivative comprises the amino acid sequence of SEQ ID NO: 12 with the following amino acid modifications: T19R, G142D, delE156, delF157, R158G, L452R, T478K, D614G, and D950N.

88. The transgene of claim 86, wherein (b) the modified SARS-CoV-2 delta virus spike protein comprises the amino acid sequence of SEQ ID NO: 16 with one, two, three, four, five, six, seven, or all of the following amino acid modifications: T6R, G129D, delE143, delF144, R145G, L439R, T465K, D601G, and D937N.

89. The transgene of claim 86, wherein (c) the modified SARS-CoV-2 delta virus spike protein ectodomain comprises:
(1) amino acid residues corresponding to amino acid residues 817, 892, 899, 942, 986, and 987 of SARS-CoV-2 spike protein according to GenBank Accession No. MN908947.3 are substituted with prolines,
(2) amino acid residues corresponding to amino acid residues 682 to 685 of the polybasic cleavage site of SARS-CoV-2 spike protein found at GenBank Accession No. MN908947.3 are substituted such that the polybasic cleavage site is inactivated, preferably the amino acid residues corresponding to amino acid residues 682 to 685 of the polybasic cleavage site of the SARS-CoV-2 spike protein according to GenBank Accession No. MN908947.3 are substituted with a single alanine, and
(3) two, three, four, five, six, seven, or more amino acid residues corresponding to two, three, four, five, six, seven, or more of amino acid residues 19, 142, 156, 157, 158, 452, 478, 614, and 950 of the spike protein according to GenBank Accession No. MN908947.3 are as follows: 19R, 142D, del156, del157, 158G, 452R, 478K, 614G, and 950N, preferably amino acid residues corresponding to amino acid residues 19, 142, 156, 157, 158, 452, 478, 614, and 950 of the spike protein according to GenBank Accession No. MN908947.3 are: R, D, deleted, deleted, G, R, K, G, and N, respectively.

90. The transgene of claim 86, wherein (d) the modified SARS-CoV-2 delta virus spike protein ectodomain comprises an amino acid sequence at least 99% or at least 99.5% identical to the amino acid sequence of SEQ ID NO: 13 or 17, preferably the modified SARS-CoV-2 delta virus spike protein ectodomain comprises the amino acid sequence of SEQ ID NO: 13 or 17.

91. The transgene of claim 86, wherein the modified SARS-CoV-2 delta virus spike protein ectodomain is linked via a linker to the NDV F protein transmembrane and cytoplasmic domains.

92. A transgene comprising a nucleotide sequence encoding a chimeric F protein, wherein
(1) the chimeric F protein comprises an amino acid sequence that is at least 99% or at least 99.5% identical to the amino acid sequence of SEQ ID NO: 6 or 18, preferably the chimeric F protein comprises the amino acid sequence of SEQ ID NO:6 or 18; or
(2) the chimeric F protein is encoded by a nucleotide sequence comprising the nucleotide sequence of SEQ ID NO:5 or 21.

93. A recombinant Newcastle disease virus (NDV) comprising a chimeric F protein as defined in claim 86.

94. The recombinant Newcastle disease virus (NDV) of claim 93, wherein
(a) the NDV virion comprises the chimeric F protein;
(b) the genome comprises:
(i) a NDV F transcription unit, a NDV NP transcription unit, a NDV M transcription unit, a NDV L transcription unit, a NDV P transcription unit, and a NDV HN transcription unit; or
(ii) a NDV F transcription unit, a NDV NP transcription unit, a NDV M transcription unit, a NDV L transcription unit, a NDV P transcription unit, and a NDV HN transcription unit, and
wherein the NDV F transcription unit encodes a NDV F protein comprising a leucine to alanine amino acid substitution at the amino residue corresponding to amino acid residue 289 of the LaSota NDV strain;
(c) the transgene is between two NDV transcription units of the packaged genome, preferably the two transcription units of the packaged genome are the transcription units for
(i) the NDV P gene and the NDV M gene, or
(ii) the NDV NP gene and the NDV P gene; or
(d) a combination thereof.

95. The recombinant Newcastle disease virus (NDV) of claim 93, which comprises
(a) a Newcastle disease virus (NDV) backbone which is lentogenic,
(b) a Newcastle disease virus (NDV) backbone of LaSota strain, or
(c) a Newcastle disease virus (NDV) backbone of Hitchner B1 strain.

96. The recombinant Newcastle disease virus (NDV) of claim 93, which comprises
(a) a Newcastle disease virus (NDV) backbone which is lentogenic,
(b) a Newcastle disease virus (NDV) backbone of LaSota strain, or
(c) a Newcastle disease virus (NDV) backbone of Hitchner B1 strain.

97. A recombinant Newcastle disease virus (NDV) comprising a packaged genome, wherein the package genome comprises a transgene from claim 86.

98. The recombinant Newcastle disease virus (NDV) of claim 97, wherein
(a) the NDV virion comprises the chimeric F protein;
(b) the genome comprises: (i) a NDV F transcription unit, a NDV NP transcription unit, a NDV M transcription unit, a NDV L transcription unit, a NDV P transcription unit, and a NDV HN transcription unit; or (ii) a NDV F transcription unit, a NDV NP transcription unit, a NDV M transcription unit, a NDV L transcription unit, a NDV P transcription unit, and a NDV HN transcription unit, and wherein the NDV F transcription unit encodes a NDV F protein comprising a leucine to alanine amino acid substitution at the amino residue corresponding to amino acid residue 289 of the LaSota NDV strain;
(c) the transgene is between two NDV transcription units of the packaged genome, preferably the two transcription units of the packaged genome are the transcription units for (i) the NDV P gene and the NDV M gene (ii) the NDV NP gene and the NDV P gene; or
(d) a combination thereof.

99. The recombinant Newcastle disease virus (NDV) of claim 97, which comprises (a) a Newcastle disease virus (NDV) backbone which is lentogenic, (b) a Newcastle disease virus (NDV) backbone of LaSota strain, or (c) a Newcastle disease virus (NDV) backbone of Hitchner B1 strain.

100. An immunogenic composition comprising the recombinant Newcastle disease virus (NDV) of claim 93.

101. The immunogenic composition of claim 100, wherein (a) the recombinant Newcastle disease virus (NDV) is inactivated, (b) the immunogenic composition further comprises an adjuvant, or (c) a combination thereof.

102. A method for (a) inducing an immune response to severe acute respiratory syndrome coronavirus 2 (SARS-CoV-2) spike protein, such as SARS-CoV-2 delta virus spike protein, (b) immunizing a subject against SARS-CoV-2, such as SARS-CoV-2 delta variant, (c) preventing Coronavirus Disease 2019 (COVID-19), or (d) boosting antibody titer or immunity to SARS-CoV-2 spike protein in a subject previously vaccinated for COVID-19 or previously infected with SARS-CoV-2, comprising administering the immunogenic composition of claim 100 to a subject.

103. The method of claim 102, wherein
(a) the composition is administered to the subject intranasally or intramuscularly;
(b) the subject is a human;
(c) the subject has been previously vaccinated with a COVID-19 vaccine; or
(d) a combination thereof.

104. A method for propagating the recombinant Newcastle disease virus (NDV) of claim 93, the method comprising culturing a cell or embryonated egg comprising the recombinant Newcastle disease virus (NDV), and optionally isolating the recombinant Newcastle disease virus (NDV) from the cell or embryonated egg.

105. A method for detecting the presence of antibody specific to SARS-CoV-2 spike protein, comprising contacting a specimen with the recombinant Newcastle disease virus (NDV) of claim 93 in an immunoassay.


SEQUENCE LISTING (25 sequences)

SEQ ID NO: 1 - DNA, 15186 bp, synthetic construct
  source 1..15186: mol_type = other DNA; note = cDNA of genomic sequence of NDV strain LaSota
accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 2 - DNA, 15186 bp, synthetic construct
  source 1..15186: mol_type = other DNA; note = cDNA of genomic sequence of NDV strain Hitchner B1
accaaacagagaatccgtaagttacgataaaaggcgaaggagcaattgaagtcgcacgggtagaaggtgtgaatctcgagtgcgagcccgaagcacaaactcgaggaagccttctgccaacatgtcttccgtattcgacgagtacgaacagctcctcgcggctcagactcgccccaatggagctcatggagggggggagaaagggagtaccttaaaagtagacgtcccggtattcactcttaacagtgatgacccagaagataggtggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggcaccccgttcgtcacagccggggctgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctagtaggggacgtagactcatatatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccggcagcaaggaggggcctggcagctgctgcccaacgagtctccgaggtgaccagcagcatagacatgcctactcaacaagtcggagtcctcactgggcttagcgaggggggatcccaagccctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgattgacaaaacccagcctgcttctacaagaacatcccaatgctctcacccgtagtcgacccctcgatttgcggctctatatgaccacaccctcaaacaaacatccccctctttcctccctccccctgctgtacaactccgcacgccctagataccacaggcacaccgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacagggcaagaccaaggtgctgagcgcagcatgggagaagcatgggagcatccagccaccggccagtcaagacaacctcgatcgacaggacagatctgacaaacaaccatccacacccgagcaaacgaccccgcacgacagcccgccggccacatccgctgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagccaggaaagactgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatgggggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccatctccctatgtgatacaaggaggcgaaatggcacttaataaactttcgcaaccagtgccacatccatctgaattgattaaacccgccactgcatgcgggcctgatataggagtggagagggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcagacccaaggtccaactctagaagcggcaatcctctctcgcttcctcagccccactgaatgatcgcgtaaccgtaattaatctagctacattaaggattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgactcgtggactgatagtaaggaagactcagtattcatcaccacctatggattcatctttcaagttgggaatgaggaagccactgtcggcatgatcgatgataaacccaagcgcgagttactttccgctgcgatgctctgcctaggaagcgtcccaaataccggagaccttgttgagctggcaagggcctgtctcactatgatggtcacatgcaagaagagtgcaactaatactgagagaatggttttctcagtagtgcaggcaccccaagtgctgcaaagctgtagggttgtggcaaataaatactcatcagtgaatgcagtcaagcacgtgaaagcgccagagaagatccccgggagtggaaccctagaatacaaggtgaactttgtctccttgactgtggtaccgaagaaggatgtctacaagatcccagctgcagtattgaagatttctggctcgagtctgtacaatcttgcgctcaatgtcactattaatgtggaggtagacccgaggagtcctttggttaaatctctgtctaagtctgacagcggatactatgctaacctcttcttgcatattggacttatgaccaccgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgctgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtccatcaagttagaaaaaacacgggtagaagattctggatcccggttggcgccctccaggtgcaggatgggctccagaccttctaccaagaacccagcacctatgatgctgactatccgggtcgcgctggtactgagttgcatctgcccggcaaactccattgatggcaggcctcttgcagctgcaggaattgtggttacaggagacaaagcagtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgccttaaacaagctgactattcaggcactttacaatctagctggtgggaatatggattacttattgactaagttaggtatagggaacaatcaactcagctcattaatcggtagcggcttaatcaccggtaaccctattctatacgactcacagactcaactcttgggtatacaggtaactctaccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttactcctgcttgagcggcaatacatcggcctgtatgtactcaaagaccgaaggcgcacttactacaccatatatgactatcaaaggctcagtcatcgctaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgaccagcacatctgctctcattacctatatcgttttgactatcatatctcttgtttttggtatacttagcctgattctagcatgctacctaatgtacaagcaaaaggcgcaacaaaagaccttattatggcttgggaataataccctagatcagatgagagccactacaaaaatgtgaacacagatgaggaacgaaggtttccctaatagtaatttgtgtgaaagttctggtagtctgtcagttcggagagttaagaaaaaactaccggttgtagatgaccaaaggacgatatacgggtagaacggtaagagaggccgcccctcaattgcgagccagacttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccattggcattgttaaatactgagaccacaattatgaacgcaataacatctctctcttatcagattaatggagctgcaaacaacagcgggtggggggcacctattcatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacactcacatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagccacggagacagaggaagaagattataactcagctgtccctacgcggatggtacatgggaggttagggttcgacggccaatatcacgaaaaggacctagatgtcacaacattattcggggactgggtggccaactacccaggagtagggggtggatcttttattgacagccgcgtatggttctcagtctacggagggttaaaacccaatacacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaagtgtcaacatccttaggcgaagacccggtactgactgtaccgcccaacacagtcacactcatgggggccgaaggcagaattctcacagtagggacatcccatttcttgtatcagcgagggtcatcatacttctctcccgcgttattatatcctatgacagtcagcgacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgaacaagcaagacttaaccctgcgtctgcagtattcgatagcacatcccgcagtcgcataactcgagtgagttcaagcagcatcaaagcagcatacacaacatcaacttgttttaaagtggtcaagaccaataagacctattgtctcagcattgctgaaatatctaatactctcttcggagaattcagaatcgtcccgttactagttgagatcctcaaagatgacggggttagagaagccaggtctggctagttgagtcaactatgaaagagttggaaagatggcattgtatcacctatcttctgcgacatcaagaatcaaaccgaatgccggcgcgtgctcgaattccatgtcgccagttgaccacaatcagccagtgctcatgcgatcagattaagccttgtcaatagtctcttgattaagaaaaaatgtaagtggcaatgagatacaaggcaaaacagctcacggtaaataatacgggtaggacatggcgagctccggtcctgaaagggcagagcatcagattatcctaccagagtcacacctgtcttcaccattggtcaagcacaaactactctattattggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtactccaccccaggtgtttagaagaactggctaatattgaggtccctgattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgattgtggcagctaggacaaggtctgcggccaacaaattggtgatgctaacccataaggtaggccaagtctttgtcactcctgaacttgttgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcgggagatttcttcgcattcaacctgcaggagcttaaagacattctaattggcctcctccccaatgatatagcagaatccgtgactcatgcaatcgctactgtattctctggtttagaacagaatcaagcagctgagatgttgtgcctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacggatacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatacgaccctgtcactaacctgagcatgttcctaaaagacaaggcaatcgcacaccccaacgataattggcttgcctcgtttaggcggaaccttctctccgaagaccagaagaaacatgtaaaggaagcgacttcgactaaccgcctcttgatagagtttttagagtcaaatgattttgatccatataaagagatggaatatctgacgacccttgagtaccttagagatgacaatgtggcagtatcatactcgctcaaagagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatgttcaaaccgcaatcatgatccgaaaagcaagaaccgtcggagagttgcaaccttcataacaactgacctgcaaaagtactgtcttaattggagatatcagacgatcaaattgttcgctcatgccatcaatcagttgatgggcctacctcatttcttcgagtggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagatgactctccggagatggtgttgacacagttgcatcaagccagtgataatttcttcaaggaattaatccatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggactactgagtcctaacattaggactaatatcttaactaggccgcctgggaatggagattgggccagtctgtgcaacgacccatactctttcaattttgagactgttgcaagcccaaacattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacactgtaattaagattgcgcttactaggaggccattaggcatcaagaggctgatgcggatagtcaattattctagcatgcatgcaatgctgtttagagacgatgttttttcctctagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcgaaaatagctcatatgtcgccacatgtgaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccagaacatatgatgagatcacactgcacctacatagtaaatttagttgctgtatcagggaagcacctgttgcggttcctttcgagctacttggggtggcaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgatgccataatagtgtatgacaatacccgaaattggatcagtgaagctcagaattcagatgtggtccgcctatttgaatatgcagcacttgaagtgctcctcgactgttcttaccaactctattacctgagagtaagagacctagacaatattgtcttatatatgggtgatttatacaagaatatgccaggaattctactttccaacattgcagctacaatatctcatcctgtcattcattcaaggttacatgcagtgggcctggtcaaccatgacggatcacaccaacttgcagatacggattttatcgaaatgtctgcaaaactgttagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatcccatctcctttctgtacccgaggtaagatgtgcaagacacgggaactccttatacttggctgaaggaagcggagccatcatgagtcttcttgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaggatggatttgtccaagagttccgtccattatggagagaaaatacagaggaaagtgacctgacctcagataaagcagtggggtatattacatctgcagtaccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggggatatggagtgttacctggtatttgtcatgggttacctgggcgggcctacatttgtacatgaggtggtgaggatggcaaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagtcatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcctaacgaaaatcacatattaataggctccttttttggccaattgtattcttgttgatttaattatattatgttagaaaaaagttgaactctgactccttaggactcgaattcgaactcaaataaatgtctttaaaaaaggttgcgcacaattattcttgagtgtagtctcgtcattcaccaaatctttgtttggt

SEQ ID NO: 3 - DNA, 15186 bp, synthetic construct
  source 1..15186: mol_type = other DNA; note = cDNA of genomic sequence of NDV strain LaSota (L289A mutation)
accaaacagagaatccgtgagttacgataaaaggcgaaggagcaattgaagtcgcacgggtagaaggtgtgaatctcgagtgcgagcccgaagcacaaactcgagaaagccttctgccaacatgtcttccgtatttgatgagtacgaacagctcctcgcggctcagactcgccccaatggagctcatggagggggagaaaaagggagtaccttaaaagtagacgtcccggtattcactcttaacagtgatgacccagaagatagatggagctttgtggtattctgcctccggattgctgttagcgaagatgccaacaaaccactcaggcaaggtgctctcatatctcttttatgctcccactcacaggtaatgaggaaccatgttgcccttgcagggaaacagaatgaagccacattggccgtgcttgagattgatggctttgccaacggcacgccccagttcaacaataggagtggagtgtctgaagagagagcacagagatttgcgatgatagcaggatctctccctcgggcatgcagcaacggaaccccgttcgtcacagccggggccgaagatgatgcaccagaagacatcaccgataccctggagaggatcctctctatccaggctcaagtatgggtcacagtagcaaaagccatgactgcgtatgagactgcagatgagtcggaaacaaggcgaatcaataagtatatgcagcaaggcagggtccaaaagaaatacatcctctaccccgtatgcaggagcacaatccaactcacgatcagacagtctcttgcagtccgcatctttttggttagcgagctcaagagaggccgcaacacggcaggtggtacctctacttattataacctggtaggggacgtagactcatacatcaggaataccgggcttactgcattcttcttgacactcaagtacggaatcaacaccaagacatcagcccttgcacttagtagcctctcaggcgacatccagaagatgaagcagctcatgcgtttgtatcggatgaaaggagataatgcgccgtacatgacattacttggtgatagtgaccagatgagctttgcgcctgccgagtatgcacaactttactcctttgccatgggtatggcatcagtcctagataaaggtactgggaaataccaatttgccagggactttatgagcacatcattctggagacttggagtagagtacgctcaggctcagggaagtagcattaacgaggatatggctgccgagctaaagctaaccccagcagcaaggaggggcctggcagctgctgcccaacgggtctccgaggagaccagcagcatagacatgcctactcaacaagtcggagtcctcactgggcttagcgagggggggtcccaagctctacaaggcggatcgaatagatcgcaagggcaaccagaagccggggatggggagacccaattcctggatctgatgagagcggtagcaaatagcatgagggaggcgccaaactctgcacagggcactccccaatcggggcctcccccaactcctgggccatcccaagataacgacaccgactgggggtattgatggacaaaacccagcctgcttccacaaaaacatcccaatgccctcacccgtagtcgacccctcgatttgcggctctatatgaccacaccctcaaacaaacatccccctctttcctccctccccctgctgtacaactacgtacgccctagataccacaggcacaatgcggctcactaacaatcaaaacagagccgagggaattagaaaaaagtacgggtagaagagggatattcagagatcagggcaagtctcccgagtctctgctctctcctctacctgatagaccaggacaaacatggccacctttacagatgcagagatcgacgagctatttgagacaagtggaactgtcattgacaacataattacagcccagggtaaaccagcagagactgttggaaggagtgcaatcccacaaggcaagaccaaggtgctgagcgcagcatgggagaagcatgggagcatccagccaccggccagtcaagacaaccccgatcgacaggacagatctgacaaacaaccatccacacccgagcaaacgaccccgcatgacagcccgccggccacatccgccgaccagccccccacccaggccacagacgaagccgtcgacacacagctcaggaccggagcaagcaactctctgctgttgatgcttgacaagctcagcaataaatcgtccaatgctaaaaagggcccatggtcgagcccccaagaggggaatcaccaacgtccgactcaacagcaggggagtcaacccagtcgcggaaacagtcaggaaagaccgcagaaccaagtcaaggccgcccctggaaaccagggcacagacgtgaacacagcatatcatggacaatgggaggagtcacaactatcagctggtgcaacccctcatgctctccgatcaaggcagagccaagacaatacccttgtatctgcggatcatgtccagccacctgtagactttgtgcaagcgatgatgtctatgatggaggcgatatcacagagagtaagtaaggttgactatcagctagatcttgtcttgaaacagacatcctccatccctatgatgcggtccgaaatccaacagctgaaaacatctgttgcagtcatggaagccaacttgggaatgatgaagattctggatcccggttgtgccaacatttcatctctgagtgatctacgggcagttgcccgatctcacccggttttagtttcaggccctggagacccctctccctatgtgacacaaggaggcgaaatggcacttaataaactttcgcaaccagtgccacatccatctgaattgattaaacccgccactgcatgcgggcctgatataggagtggaaaaggacactgtccgtgcattgatcatgtcacgcccaatgcacccgagttcttcagccaagctcctaagcaagttagatgcagccgggtcgatcgaggaaatcaggaaaatcaagcgccttgctctaaatggctaattactactgccacacgtagcgggtccctgtccactcggcatcacacggaatctgcaccgagttcccccccgcggacccaaggtccaactctccaagcggcaatcctctctcgcttcctcagccccactgaatgatcgcgtaaccgtaattaatctagctacatttaagattaagaaaaaatacgggtagaattggagtgccccaattgtgccaagatggactcatctaggacaattgggctgtactttgattctgcccattcttctagcaacctgttagcatttccgatcgtcctacaagacacaggagatgggaagaagcaaatcgccccgcaatataggatccagcgccttgacttgtggactgatagtaaggaggactcagtattcatcaccacctatggattcatctttcaagttgggaatgaagaagccaccgtcggcatgatcgatgataaacccaagcgcgagttactttccgctgcgatgctctgcctaggaagcgtcccaaataccggagaccttattgagctggcaagggcctgtctcactatgatagtcacatgcaagaagagtgcaactaatactgagagaatggttttctcagtagtgcaggcaccccaagtgctgcaaagctgtagggttgtggcaaacaaatactcatcagtgaatgcagtcaagcacgtgaaagcgccagagaagattcccgggagtggaaccctagaatacaaggtgaactttgtctccttgactgtggtaccgaagagggatgtctacaagatcccagctgcagtattgaaggtttctggctcgagtctgtacaatcttgcgctcaatgtcactattaatgtggaggtagacccgaggagtcctttggttaaatctctgtctaagtctgacagcggatactatgctaacctcttcttgcatattggacttatgaccactgtagataggaaggggaagaaagtgacatttgacaagctggaaaagaaaataaggagccttgatctatctgtcgggctcagtgatgtgctcgggccttccgtgttggtaaaagcaagaggtgcacggactaagcttttggcacctttcttctctagcagtgggacagcctgctatcccatagcaaatgcttctcctcaggtggccaagatactctggagtcaaaccgcgtgcctgcggagcgttaaaatcattatccaagcaggtacccaacgcgctgtcgcagtgaccgccgaccacgaggttacctctactaagctggagaaggggcacacccttgccaaatacaatccttttaagaaataagctgcgtctctgagattgcgctccgcccactcacccagatcatcatgacacaaaaaactaatctgtcttgattatttacagttagtttacctgtctatcaagttagaaaaaacacgggtagaagattctggatcccggttggcgccctccaggtgcaagatgggctccagaccttctaccaagaacccagcacctatgatgctgactatccgggttgcgctggtactgagttgcatctgtccggcaaactccattgatggcaggcctcttgcagctgcaggaattgtggttacaggagacaaagccgtcaacatatacacctcatcccagacaggatcaatcatagttaagctcctcccgaatctgcccaaggataaggaggcatgtgcgaaagcccccttggatgcatacaacaggacattgaccactttgctcaccccccttggtgactctatccgtaggatacaagagtctgtgactacatctggaggggggagacaggggcgccttataggcgccattattggcggtgtggctcttggggttgcaactgccgcacaaataacagcggccgcagctctgatacaagccaaacaaaatgctgccaacatcctccgacttaaagagagcattgccgcaaccaatgaggctgtgcatgaggtcactgacggattatcgcaactagcagtggcagttgggaagatgcagcagtttgttaatgaccaatttaataaaacagctcaggaattagactgcatcaaaattgcacagcaagttggtgtagagctcaacctgtacctaaccgaattgactacagtattcggaccacaaatcacttcacctgctttaaacaagctgactattcaggcactttacaatctagctggtggaaatatggattacttattgactaagttaggtgtagggaacaatcaactcagctcattaatcggtagcggcttaatcaccggtaaccctattctatacgactcacagactcaactcttgggtatacaggtaactgccccttcagtcgggaacctaaataatatgcgtgccacctacttggaaaccttatccgtaagcacaaccaggggatttgcctcggcacttgtcccaaaagtggtgacacaggtcggttctgtgatagaagaacttgacacctcatactgtatagaaactgacttagatttatattgtacaagaatagtaacgttccctatgtcccctggtatttattcctgcttgagcggcaatacgtcggcctgtatgtactcaaagaccgaaggcgcacttactacaccatacatgactatcaaaggttcagtcatcgccaactgcaagatgacaacatgtagatgtgtaaaccccccgggtatcatatcgcaaaactatggagaagccgtgtctctaatagataaacaatcatgcaatgttttatccttaggcgggataactttaaggctcagtggggaattcgatgtaacttatcagaagaatatctcaatacaagattctcaagtaataataacaggcaatcttgatatctcaactgagcttgggaatgtcaacaactcgatcagtaatgctttgaataagttagaggaaagcaacagaaaactagacaaagtcaatgtcaaactgactagcacatctgctctcattacctatatcgttttgactatcatatctcttgtttttggtatacttagcctgattctagcatgctacctaatgtacaagcaaaaggcgcaacaaaagaccttattatggcttgggaataatactctagatcagatgagagccactacaaaaatgtgaacacagatgaggaacgaaggtttccctaatagtaatttgtgtgaaagttctggtagtctgtcagttcagagagttaagaaaaaactaccggttgtagatgaccaaaggacgatatacgggtagaacggtaagagaggccgcccctcaattgcgagccaggcttcacaacctccgttctaccgcttcaccgacaacagtcctcaatcatggaccgcgccgttagccaagttgcgttagagaatgatgaaagagaggcaaaaaatacatggcgcttgatattccggattgcaatcttattcttaacagtagtgaccttggctatatctgtagcctcccttttatatagcatgggggctagcacacctagcgatcttgtaggcataccgactaggatttccagggcagaagaaaagattacatctacacttggttccaatcaagatgtagtagataggatatataagcaagtggcccttgagtctccgttggcattgttaaatactgagaccacaattatgaacgcaataacatctctctcttatcagattaatggagctgcaaacaacagtgggtggggggcacctatccatgacccagattatataggggggataggcaaagaactcattgtagatgatgctagtgatgtcacatcattctatccctctgcatttcaagaacatctgaattttatcccggcgcctactacaggatcaggttgcactcgaataccctcatttgacatgagtgctacccattactgctacacccataatgtaatattgtctggatgcagagatcactcacattcatatcagtatttagcacttggtgtgctccggacatctgcaacagggagggtattcttttctactctgcgttccatcaacctggacgacacccaaaatcggaagtcttgcagtgtgagtgcaactcccctgggttgtgatatgctgtgctcgaaagtcacggagacagaggaagaagattataactcagctgtccctacgcggatggtacatgggaggttagggttcgacggccagtaccacgaaaaggacctagatgtcacaacattattcggggactgggtggccaactacccaggagtagggggtggatcttttattgacagccgcgtatggttctcagtctacggagggttaaaacccaattcacccagtgacactgtacaggaagggaaatatgtgatatacaagcgatacaatgacacatgcccagatgagcaagactaccagattcgaatggccaagtcttcgtataagcctggacggtttggtgggaaacgcatacagcaggctatcttatctatcaaggtgtcaacatccttaggcgaagacccggtactgactgtaccgcccaacacagtcacactcatgggggccgaaggcagaattctcacagtagggacatctcatttcttgtatcaacgagggtcatcatacttctctcccgcgttattatatcctatgacagtcagcaacaaaacagccactcttcatagtccttatacattcaatgccttcactcggccaggtagtatcccttgccaggcttcagcaagatgccccaactcgtgtgttactggagtctatacagatccatatcccctaatcttctatagaaaccacaccttgcgaggggtattcgggacaatgcttgatggtgtacaagcaagacttaaccctgcgtctgcagtattcgatagcacatcccgcagtcgcattactcgagtgagttcaagcagtaccaaagcagcatacacaacatcaacttgttttaaagtggtcaagactaataagacctattgtctcagcattgctgaaatatctaatactctcttcggagaattcagaatcgtcccgttactagttgagatcctcaaagatgacggggttagagaagccaggtctggctagttgagtcaattataaaggagttggaaagatggcattgtatcacctatcttctgcgacatcaagaatcaaaccgaatgccggcgcgtgctcgaattccatgttgccagttgaccacaatcagccagtgctcatgcgatcagattaagccttgtcaatagtctcttgattaagaaaaaatgtaagtggcaatgagatacaaggcaaaacagctcatggttaacaatacgggtaggacatggcgagctccggtcctgaaagggcagagcatcagattatcctaccagagtcacacctgtcttcaccattggtcaagcacaaactactctattactggaaattaactgggctaccgcttcctgatgaatgtgacttcgaccacctcattctcagccgacaatggaaaaaaatacttgaatcggcctctcctgatactgagagaatgataaaactcggaagggcagtacaccaaactcttaaccacaattccagaataaccggagtgctccaccccaggtgtttagaagaactggctaatattgaggtcccagattcaaccaacaaatttcggaagattgagaagaagatccaaattcacaacacgagatatggagaactgttcacaaggctgtgtacgcatatagagaagaaactgctggggtcatcttggtctaacaatgtcccccggtcagaggagttcagcagcattcgtacggatccggcattctggtttcactcaaaatggtccacagccaagtttgcatggctccatataaaacagatccagaggcatctgatggtggcagctaggacaaggtctgcggccaacaaattggtgatgctaacccataaggtaggccaagtctttgtcactcctgaacttgtcgttgtgacgcatacgaatgagaacaagttcacatgtcttacccaggaacttgtattgatgtatgcagatatgatggagggcagagatatggtcaacataatatcaaccacggcggtgcatctcagaagcttatcagagaaaattgatgacattttgcggttaatagacgctctggcaaaagacttgggtaatcaagtctacgatgttgtatcactaatggagggatttgcatacggagctgtccagctactcgagccgtcaggtacatttgcaggagatttcttcgcattcaacctgcaggagcttaaagacattctaattggcctcctccccaatgatatagcagaatccgtgactcatgcaatcgctactgtattctctggtttagaacagaatcaagcagctgagatgttgtgtctgttgcgtctgtggggtcacccactgcttgagtcccgtattgcagcaaaggcagtcaggagccaaatgtgcgcaccgaaaatggtagactttgatatgatccttcaggtactgtctttcttcaagggaacaatcatcaacgggtacagaaagaagaatgcaggtgtgtggccgcgagtcaaagtggatacaatatatgggaaggtcattgggcaactacatgcagattcagcagagatttcacacgatatcatgttgagagagtataagagtttatctgcacttgaatttgagccatgtatagaatatgaccctgtcaccaacctgagcatgttcctaaaagacaaggcaatcgcacaccccaacgataattggcttgcctcgtttaggcggaaccttctctccgaagaccagaagaaacatgtaaaagaagcaacttcgactaatcgcctcttgatagagtttttagagtcaaatgattttgatccatataaagagatggaatatctgacgacccttgagtaccttagagatgacaatgtggcagtatcatactcgctcaaggagaaggaagtgaaagttaatggacggatcttcgctaagctgacaaagaagttaaggaactgtcaggtgatggcggaagggatcctagccgatcagattgcacctttctttcagggaaatggagtcattcaggatagcatatccttgaccaagagtatgctagcgatgagtcaactgtcttttaacagcaataagaaacgtatcactgactgtaaagaaagagtatcttcaaaccgcaatcatgatccgaaaagcaagaaccgtcggagagttgcaaccttcataacaactgacctgcaaaagtactgtcttaattggagatatcagacaatcaaattgttcgctcatgccatcaatcagttgatgggcctacctcacttcttcgaatggattcacctaagactgatggacactacgatgttcgtaggagaccctttcaatcctccaagtgaccctactgactgtgacctctcaagagtccctaatgatgacatatatattgtcagtgccagagggggtatcgaaggattatgccagaagctatggacaatgatctcaattgctgcaatccaacttgctgcagctagatcgcattgtcgtgttgcctgtatggtacagggtgataatcaagtaatagcagtaacgagagaggtaagatcagacgactctccggagatggtgttgacacagttgcatcaagccagtgataatttcttcaaggaattaattcatgtcaatcatttgattggccataatttgaaggatcgtgaaaccatcaggtcagacacattcttcatatacagcaaacgaatcttcaaagatggagcaatcctcagtcaagtcctcaaaaattcatctaaattagtgctagtgtcaggtgatctcagtgaaaacaccgtaatgtcctgtgccaacattgcctctactgtagcacggctatgcgagaacgggcttcccaaagacttctgttactatttaaactatataatgagttgtgtgcagacatactttgactctgagttctccatcaccaacaattcgcaccccgatcttaatcagtcgtggattgaggacatctcttttgtgcactcatatgttctgactcctgcccaattagggggactgagtaaccttcaatactcaaggctctacactagaaatatcggtgacccggggactactgcttttgcagagatcaagcgactagaagcagtgggattactgagtcctaacattatgactaatatcttaactaggccgcctgggaatggagattgggccagtctgtgcaacgacccatactctttcaattttgagactgttgcaagcccaaatattgttcttaagaaacatacgcaaagagtcctatttgaaacttgttcaaatcccttattgtctggagtgcacacagaggataatgaggcagaagagaaggcattggctgaattcttgcttaatcaagaggtgattcatccccgcgttgcgcatgccatcatggaggcaagctctgtaggtaggagaaagcaaattcaagggcttgttgacacaacaaacaccgtaattaagattgcgcttactaggaggccattaggcatcaagaggctgatgcggatagtcaattattctagcatgcatgcaatgctgtttagagacgatgttttttcctccagtagatccaaccaccccttagtctcttctaatatgtgttctctgacactggcagactatgcacggaatagaagctggtcacctttgacgggaggcaggaaaatactgggtgtatctaatcctgatacgatagaactcgtagagggtgagattcttagtgtaagcggagggtgtacaagatgtgacagcggagatgaacaatttacttggttccatcttccaagcaatatagaattgaccgatgacaccagcaagaatcctccgatgagggtaccatatctcgggtcaaagacacaggagaggagagctgcctcacttgcaaaaatagctcatatgtcgccacatgtaaaggctgccctaagggcatcatccgtgttgatctgggcttatggggataatgaagtaaattggactgctgctcttacgattgcaaaatctcggtgtaatgtaaacttagagtatcttcggttactgtcccctttacccacggctgggaatcttcaacatagactagatgatggtataactcagatgacattcacccctgcatctctctacagggtgtcaccttacattcacatatccaatgattctcaaaggctgttcactgaagaaggagtcaaagaggggaatgtggtttaccaacagatcatgctcttgggtttatctctaatcgaatcgatctttccaatgacaacaaccaggacatatgatgagatcacactgcacctacatagtaaatttagttgctgtatcagagaagcacctgttgcggttcctttcgagctacttggggtggtaccggaactgaggacagtgacctcaaataagtttatgtatgatcctagccctgtatcggagggagactttgcgagacttgacttagctatcttcaagagttatgagcttaatctggagtcatatcccacgatagagctaatgaacattctttcaatatccagcgggaagttgattggccagtctgtggtttcttatgatgaagatacctccataaagaatgacgccataatagtgtatgacaatacccgaaattggatcagtgaagctcagaattcagatgtggtccgcctatttgaatatgcagcacttgaagtgctcctcgactgttcttaccaactctattacctgagagtaagaggcctggacaatattgtcttatatatgggtgatttatacaagaatatgccaggaattctactttccaacattgcagctacaatatctcatcccgtcattcattcaaggttacatgcagtgggcctggtcaaccatgacggatcacaccaacttgcagatacggattttatcgaaatgtctgcaaaactattagtatcttgcacccgacgtgtgatctccggcttatattcaggaaataagtatgatctgctgttcccatctgtcttagatgataacctgaatgagaagatgcttcagctgatatcccggttatgctgtctgtacacggtactctttgctacaacaagagaaatcccgaaaataagaggcttaactgcagaagagaaatgttcaatactcactgagtatttactgtcggatgctgtgaaaccattacttagccccgatcaagtgagctctatcatgtctcctaacataattacattcccagctaatctgtactacatgtctcggaagagcctcaatttgatcagggaaagggaggacagggatactatcctggcgttgttgttcccccaagagccattattagagttcccttctgtgcaagatattggtgctcgagtgaaagatccattcacccgacaacctgcggcatttttgcaagagttagatttgagtgctccagcaaggtatgacgcattcacacttagtcagattcatcctgaactcacatctccaaatccggaggaagactacttagtacgatacttgttcagagggatagggactgcatcttcctcttggtataaggcatctcatctcctttctgtacccgaggtaagatgtgcaagacacgggaactccttatacttagctgaagggagcggagccatcatgagtcttctcgaactgcatgtaccacatgaaactatctattacaatacgctcttttcaaatgagatgaaccccccgcaacgacatttcgggccgaccccaactcagtttttgaattcggttgtttataggaatctacaggcggaggtaacatgcaaagatggatttgtccaagagttccgtccattatggagagaaaatacagaggaaagcgacctgacctcagataaagtagtggggtatattacatctgcagtgccctacagatctgtatcattgctgcattgtgacattgaaattcctccagggtccaatcaaagcttactagatcaactagctatcaatttatctctgattgccatgcattctgtaagggagggcggggtagtaatcatcaaagtgttgtatgcaatgggatactactttcatctactcatgaacttgtttgctccgtgttccacaaaaggatatattctctctaatggttatgcatgtcgaggagatatggagtgttacctggtatttgtcatgggttacctgggcgggcctacatttgtacatgaggtggtgaggatggcgaaaactctggtgcagcggcacggtacgcttttgtctaaatcagatgagatcacactgaccaggttattcacctcacagcggcagcgtgtgacagacatcctatccagtcctttaccaagattaataaagtacttgaggaagaatattgacactgcgctgattgaagccgggggacagcccgtccgtccattctgtgcggagagtctggtgagcacgctagcgaacataactcagataacccagatcatcgctagtcacattgacacagttatccggtctgtgatatatatggaagctgagggtgatctcgctgacacagtatttctatttaccccttacaatctctctactgacgggaaaaagaggacatcacttaaacagtgcacgagacagatcctagaggttacaatactaggtcttagagtcgaaaatctcaataaaataggcgatataatcagcctagtgcttaaaggcatgatctccatggaggaccttatcccactaaggacatacttgaagcatagtacctgccctaaatatttgaaggctgtcctaggtattaccaaactcaaagaaatgtttacagacacttctgtactgtacttgactcgtgctcaacaaaaattctacatgaaaactataggcaatgcagtcaaaggatattacagtaactgtgactcttaacgaaaatcacatattaataggctccttttttggccaattgtattcttgttgatttaatcatattatgttagaaaaaagttgaaccctgactccttaggactcgaattcgaactcaaataaatgtcttaaaaaaaggttgcgcacaattattcttgagtgtagtctcgtcattcaccaaatctttgtttggt

SEQ ID NO: 4 - protein, 553 aa, synthetic construct
  source 1..553: note = Amino acid sequence of F protein of NDV strain LaSota
MGSRPSTKNPAPMTLTIRVALVLSCICPANSIDGRPLAAAGIVVTGDKAVNIYTSSQTGSIIVKLLPNLPKDKEACAKAPLDAYNRTLTTLLTPLGDSIRRIQESVTTSGGGRQGRLIGAIIGGVALGVATAAQITAAAALIQAKQNAANILRLKESIAATNEAVHEVTDGLSQLAVAVGKMQQFVNDQFNKTAQELDCIKIAQQVGVELNLYLTELTTVFGPQITSPALNKLTIQALYNLAGGNMDYLLTKLGVGNNQLSSLIGSGLITGNPILYDSQTQLLGIQVTLPSVGNLNNMRATYLETLSVSTTRGFASALVPKVVTQVGSVIEELDTSYCIETDLDLYCTRIVTFPMSPGIYSCLSGNTSACMYSKTEGALTTPYMTIKGSVIANCKMTTCRCVNPPGIISQNYGEAVSLIDKQSCNVLSLGGITLRLSGEFDVTYQKNISIQDSQVIITGNLDISTELGNVNNSISNALNKLEESNRKLDKVNVKLTSTSALITYIVLTIISLVFGILSLILACYLMYKQKAQQKTLLWLGNNTLDQMRATTKM

SEQ ID NO: 5 - DNA, 3801 bp, synthetic construct
  source 1..3801: mol_type = other DNA; note = Nucleotide Sequence of the spike protein of NDV-HXP-S (Delta, P681)
atgttcgtgtttctggtgctgctgcctctggtgtccagccagtgtgtgaacctgagaacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtaccggctgttccggaagtccaatctgaagcccttcgagcgggacatctccaccgagatctatcaggccggcagcaagccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagcagagcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcagaacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 6 - protein, 1266 aa, synthetic construct
  source 1..1266: note = Amino Acid Sequence of the spike protein of NDV-HXP-S (Delta, P681)
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 7 - protein, 5 aa, synthetic construct
  source 1..5: note = Linker Sequence
GGGGS

SEQ ID NO: 8 - skipped

SEQ ID NO: 9 - DNA, 10 bp, synthetic construct
  source 1..10: mol_type = other DNA; note = NDV Gene End Sequence
ttagaaaaaa

SEQ ID NO: 10 - DNA, 10 bp, synthetic construct
  source 1..10: mol_type = other DNA; note = NDV Gene Start Sequence
acgggtagaa

SEQ ID NO: 11 - protein, 1268 aa, synthetic construct
  source 1..1268: note = S-F chimera HexaPro (Amino acid sequence)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 12 - protein, 1210 aa, synthetic construct
  source 1..1210: note = Ectodomain Portion of SEQ ID NO:11 (Amino Acid Sequence)
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 13 - protein, 1208 aa, synthetic construct
  source 1..1208: note = Ectodomain Portion of SEQ ID NO:6 (Amino Acid Sequence)
MFVFLVLLPLVSSQCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 14 - skipped

SEQ ID NO: 15 - protein, 13 aa, synthetic construct
  source 1..13: note = Signal Sequence
MFVFLVLLPLVSS

SEQ ID NO: 16 - protein, 1197 aa, synthetic construct
  source 1..1197: note = Ectodomain Portion of SEQ ID NO:11 Without the Signal Sequence (Amino Acid Sequence)
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 17 - protein, 1195 aa, synthetic construct
  source 1..1195: note = Ectodomain Portion of SEQ ID NO:6 Without the Signal Sequence (Amino Acid Sequence)
QCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 18 - protein, 1253 aa, synthetic construct
  source 1..1253: note = Amino Acid Sequence of the spike protein of NDV-HXP-S (Delta, P681) Without the Signal Sequence
QCVNLRTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLDVYYHKNNKSWMESGVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSKPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSRASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQNVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 19 - DNA, 3624 bp, synthetic construct
  source 1..3624: mol_type = other DNA; note = Nucleotide Sequence of Ectodomain Portion of NDV-HXP-S (Delta, P681) (SEQ ID NO:5) With Signal Sequence
atgttcgtgtttctggtgctgctgcctctggtgtccagccagtgtgtgaacctgagaacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtaccggctgttccggaagtccaatctgaagcccttcgagcgggacatctccaccgagatctatcaggccggcagcaagccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagcagagcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcagaacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 20 - DNA, 3585 bp, synthetic construct
  source 1..3585: mol_type = other DNA; note = Nucleotide Sequence of Ectodomain Portion of NDV-HXP-S (Delta, P681) (SEQ ID NO:5) Without Signal Sequence
cagtgtgtgaacctgagaacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtaccggctgttccggaagtccaatctgaagcccttcgagcgggacatctccaccgagatctatcaggccggcagcaagccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagcagagcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcagaacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggccc

SEQ ID NO: 21 - DNA, 3762 bp, synthetic construct
  source 1..3762: mol_type = other DNA; note = Nucleotide Sequence of NDV-HXP-S (Delta) (SEQ ID NO:5) Without Signal Sequence
cagtgtgtgaacctgagaacaagaacccagctgcctccagcctacaccaacagctttaccagaggcgtgtactaccccgacaaggtgttcagatccagcgtgctgcactctacccaggacctgttcctgcctttcttcagcaacgtgacctggttccacgccatccacgtgtccggcaccaatggcaccaagagattcgacaaccccgtgctgcccttcaacgacggggtgtactttgccagcaccgagaagtccaacatcatcagaggctggatcttcggcaccacactggacagcaagacccagagcctgctgatcgtgaacaacgccaccaacgtggtcatcaaagtgtgcgagttccagttctgcaacgaccccttcctggacgtctactatcacaagaacaacaagagctggatggaaagcggcgtgtacagcagcgccaacaactgcaccttcgagtacgtgtcccagcctttcctgatggacctggaaggcaagcagggcaacttcaagaacctgcgcgagttcgtgttcaagaacatcgacggctacttcaagatctacagcaagcacacccctatcaacctcgtgcgggatctgcctcagggcttctctgctctggaacccctggtggatctgcccatcggcatcaacatcacccggtttcagacactgctggccctgcacagaagctacctgacacctggcgatagcagcagcggatggacagctggtgccgccgcttactatgtgggctacctgcagcctagaacctttctgctgaagtacaacgagaacggcaccatcaccgacgccgtggattgtgctctggatcctctgagcgagacaaagtgcaccctgaagtccttcaccgtggagaagggcatctaccagaccagcaacttccgggtgcagcccaccgaatccatcgtgcggttccccaatatcaccaatctgtgccccttcggcgaggtgttcaatgccaccagattcgcctctgtgtacgcctggaaccggaagcggatcagcaattgcgtggccgactactccgtgctgtacaactccgccagcttcagcaccttcaagtgctacggcgtgtcccctaccaagctgaacgacctgtgcttcacaaacgtgtacgccgacagcttcgtgatccggggagatgaagtgcggcagattgcccctggacagacaggcaagatcgccgactacaactacaagctgcccgacgacttcaccggctgtgtgattgcctggaacagcaacaacctggactccaaagtcggcggcaactacaattaccggtaccggctgttccggaagtccaatctgaagcccttcgagcgggacatctccaccgagatctatcaggccggcagcaagccttgtaacggcgtggaaggcttcaactgctacttcccactgcagtcctacggctttcagcccacaaatggcgtgggctatcagccctacagagtggtggtgctgagcttcgaactgctgcatgcccctgccacagtgtgcggccctaagaagagcaccaatctcgtgaagaacaaatgcgtgaacttcaacttcaacggcctgaccggcaccggcgtgctgacagagagcaacaagaagttcctgccattccagcagtttggccgggatatcgccgataccacagacgccgttagagatccccagacactggaaatcctggacatcaccccttgcagcttcggcggagtgtctgtgatcacccctggcaccaacaccagcaatcaggtggcagtgctgtaccagggcgtgaactgtaccgaagtgcccgtggccattcacgccgatcagctgacacctacatggcgggtgtactccaccggcagcaatgtgtttcagaccagagccggctgtctgatcggagccgagcacgtgaacaatagctacgagtgcgacatccccatcggcgctggcatctgtgccagctaccagacacagacaaacagcagagcctctgtggccagccagagcatcattgcctacacaatgtctctgggcgccgagaacagcgtggcctactccaacaactctatcgctatccccaccaacttcaccatcagcgtgaccacagagatcctgcctgtgtccatgaccaagaccagcgtggactgcaccatgtacatctgcggcgattccaccgagtgctccaacctgctgctgcagtacggcagcttctgcacccagctgaatagagccctgacagggatcgccgtggaacaggacaagaacacccaagaggtgttcgcccaagtgaagcagatctacaagacccctcctatcaaggacttcggcggcttcaatttcagccagattctgcccgatcctagcaagcccagcaagcggagccctatcgaggacctgctgttcaacaaagtgacactggccgacgccggcttcatcaagcagtatggcgattgtctgggcgacattgccgccagggatctgatttgcgcccagaagtttaacggactgacagtgctgcctcctctgctgaccgatgagatgatcgcccagtacacatctgccctgctggccggcacaatcacaagcggctggacatttggagctggccctgctctgcagatcccctttccaatgcagatggcctaccggttcaacggcatcggagtgacccagaatgtgctgtacgagaaccagaagctgatcgccaaccagttcaacagcgccatcggcaagatccaggacagcctgagcagcacacccagcgccctgggaaagctgcagaacgtggtcaaccagaatgcccaggcactgaacaccctggtcaagcagctgtcctccaacttcggcgccatcagctctgtgctgaacgatatcctgagcagactggacccccctgaagccgaggtgcagatcgacagactgatcaccggaaggctgcagtccctgcagacctacgttacccagcagctgatcagagccgccgagattagagcctctgccaatctggccgccaccaagatgtctgagtgtgtgctgggccagagcaagagagtggacttttgcggcaagggctaccacctgatgagcttccctcagtctgcccctcacggcgtggtgtttctgcacgtgacatacgtgcccgctcaagagaagaatttcaccaccgctccagccatctgccacgacggcaaagcccactttcctagagaaggcgtgttcgtgtccaacggcacccattggttcgtgacccagcggaacttctacgagccccagatcatcaccaccgacaacaccttcgtgtctggcaactgcgacgtcgtgatcggcattgtgaacaataccgtgtacgaccctctgcagcccgagctggacagcttcaaagaggaactggataagtactttaagaaccacacaagccccgacgtggacctgggcgatatcagcggaatcaatgccagcgtcgtgaacatccagaaagagatcgaccggctgaacgaggtggccaagaatctgaacgagagcctgatcgacctgcaagaactggggaagtacgagcagtacatcaagtggcccggcggaggtgggtcgctcataacatacatcgtcctgactataatcagcttggtatttggtattttgtctttgattcttgcatgctatttgatgtataaacagaaagctcagcagaagactctcctgtggctcggtaacaacacactcgaccagatgagagcaactacaaagatgtga

SEQ ID NO: 22 - protein, 53 aa, synthetic construct
  source 1..53: note = Amino acid sequence of transmembrane and cytoplasmic domains of F protein of NDV strain LaSota
LITYIVLTIISLVFGILSLILACYLMYKQKAQQKTLLWLGNNTLDQMRATTKM

SEQ ID NO: 23 - protein, 1255 aa, synthetic construct
  source 1..1255: note = Amino Acid Sequence of S-F chimera HexaPro (SEQ ID NO:11) Without Signal Sequence
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGGGGSLITYIVLTIISLVFGILSLILACYLMYKQKAQQKTLLWLGNNTLDQMRATTKM

SEQ ID NO: 24 - protein, 5 aa, synthetic construct
  source 1..5: note = Linker
  REPEAT 1..5: note = GGGGS can be repeated n times, wherein n is 1, 2, 3, 4, 5 or more
GGGGS

SEQ ID NO: 25 - protein, 6 aa, synthetic construct
  source 1..6: note = His tag
HHHHHH